(19) United States
(12) Patent Application Publication
(10) Pub. No.: US 2021/0154317 A1
(43) Pub. Date: May 27, 2021

(54) COMPOSITIONS AND METHODS FOR THE TREATMENT OF VIRAL INFECTIONS

(71) Applicant: Silverback Therapeutics, Inc., Seattle, WA (US)

(72) Inventors: Peter Armstrong Thompson, Seattle, WA (US); Valerie Odegard, Seattle, WA (US); Robert Finley Dubose, Seattle, WA (US); Sean Wesley Smith, Seattle, WA (US); Craig Alan Coburn, Seattle, WA (US)

(21) Appl. No.: 16/911,358

(22) Filed: Jun. 24, 2020

Related U.S. Application Data
(63) Continuation of application No. PCT/US2018/065768, filed on Dec. 14, 2018.
(60) Provisional application No. 62/599,636, filed on Dec. 15, 2017.
(60) Provisional application No. 62/645,054, filed on Mar. 19, 2018.
(60) Provisional application No. 62/730,473, filed on Sep. 12, 2018.

(51) Int. Cl. A61K 47/68; C07K 16/28; A61P 1/16; A61P 31/20; A61P 31/14; C07D 401/12; C07D 223/16; C07D 403/12; C07D 401/14; C07F 5/02; C07D 471/04

(52) U.S. Cl. CPC A61K 47/6849; C07K 2317/92; A61K 47/6803; A61K 47/6889; A61P 1/16; A61P 31/20; A61P 31/14; C07D 401/12; C07D 223/16; C07D 403/12; C07D 401/14; C07F 5/027; C07D 471/04; C07K 2317/52; C07K 2317/622; C07K 16/2851

(57) ABSTRACT

Various compositions are disclosed for the treatment of viral infections. The compositions of conjugates comprise antibody constructs attached to myeloid cell agonists via a linker are also provided. Additionally provided are the methods of preparation and use of the conjugates. This includes methods for treating viral infections, such as viral liver diseases.

### CROSS-REFERENCE

This application is a Continuation Application of International Application No. PCT/US2018/065768, filed Dec. 14, 2018, which claims the benefit of U.S. Provisional Application No. 62/599,636, filed on Dec. 15, 2017, U.S. Provisional Application No. 62/645,054, filed on Mar. 19, 2018, and U.S. Provisional Application No. 62/730,473, filed on Sep. 12, 2018, each of which is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 860234_424C1_SEQUENCE_LISTING.txt. The text file is 2.6 KB, was created on Jan. 26, 2021, and is being submitted electronically via EFS-Web.

### BACKGROUND

Millions of people worldwide are affected by viral infections of the liver, such as Hepatitis B (HBV) or Hepatitis C (HCV). These infections can be chronic, leading to cirrhosis and significant liver damage. In 2015, 1.3 million individuals died from HBV and HCV infections. Although antiviral medications can be used to treat these infections, treatment is not always effective. This is due, in part, because viruses such as HBV can achieve immunological ignorance or tolerance and avoid detection by the innate immune system. Therefore, there is a need for alternative strategies to treat viral infections of the liver.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In various aspects, a conjugate comprises: a) an antibody construct comprising i) a target antigen binding domain that specifically binds to a first antigen on a liver cell, wherein the first antigen is a liver cell antigen or a viral antigen from a virus infecting the liver cell; and ii) an Fc binding domain covalently attached to the target antigen binding domain; b) a myeloid cell agonist selected from a TLR7 agonist or a TLR8 agonist, e.g., a compound selected from Category A or Category B; and c) a linker covalently attached to the myeloid cell agonist and to the antibody construct. In some aspects, the conjugate is represented by Formula (I):

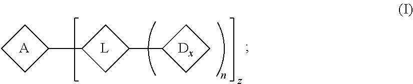

wherein: A is the antibody construct; L is the linker; Dₓ is the myeloid cell agonist; n is selected from 1 to 20; and z is selected from 1 to 20.

In some aspects, the first antigen is a liver cell antigen. In some aspects, the liver cell antigen is expressed on a canalicular cell, Kupffer cell, hepatocyte, or any combination thereof. In some aspects, the liver cell antigen is a hepatocyte antigen. In some aspects, the liver cell antigen is selected from the group consisting of ASGR1, ASGR2, TRF2, UGT1A1, SLC22A7, SLC13A5, SLC22A1, and C9. In some aspects, the liver cell antigen is selected from the group consisting of ASGR1, ASGR2, and TRF2. In some aspects, the liver cell antigen is expressed on a liver cell infected with a virus selected from the group consisting of HBV and HCV.

In some aspects, the first antigen is a viral antigen from a virus selected from the group consisting of HBV and HCV. In some aspects, the viral antigen is an HBV antigen. In some aspects, the viral antigen is HBsAg, HBcAg, or HBeAg. In some aspects, the viral antigen is HBsAg.

In some aspects, the antibody construct further comprises a second antigen binding domain that specifically binds to a second antigen on the liver cell, wherein the second antigen is a second liver cell antigen or a second viral antigen from a virus infecting the liver cell. In some aspects, the second antigen binding specifically binds to the second viral antigen from a virus infecting the liver cell. In some aspects, the second antigen binding domain is covalently attached to the antibody construct at a C-terminal end of the Fc binding domain. In some aspects, the second antigen binding domain is covalently attached to a C-terminal end of a light chain of the antibody construct. In some aspects, the second liver antigen is expressed on a canalicular cell, Kupffer cell, hepatocyte, or a combination thereof. In some aspects, the second liver antigen is a hepatocyte antigen. In some aspects, the second liver cell antigen is selected from the group consisting of ASGR1, ASGR2, TRF2, UGT1A1, SLC22A7, SLC13A5, SLC22A1, and C9. In some aspects, the second liver cell antigen is selected from the group consisting of ASGR1, ASGR2, and TRF2. In some aspects, the second viral antigen is from a virus selected from the group consisting of HBV and HCV. In some aspects, the viral antigen is an HBV antigen. In some aspects, the viral antigen is HBsAg, HBcAg, or HBeAg. In some aspects, the viral antigen is HBsAg. In some aspects, the second antigen binding domain specifically binds to the first liver cell antigen or the first viral antigen. In some aspects, the first antigen is different than the second antigen.

In some aspects, the myeloid cell agonist is a TLR7 agonist. In some aspects, the TLR7 agonist is selected from the group consisting of an imidazoquinoline amine, an imidazoquinoline amine, a thiazoquinoline, a guanosine analog, an adenosine analog, a thymidine homopolymer, ssRNA, CpG-A, PolyG10, and PolyG3. In some aspects, the TLR7 is selected from a Category B compound. In some aspects, the TLR7 agonist is selected from the group consisting of gardiquimod, imiquimod, resiquimod, GS-9620, and imidazoquinoline 852A.

In some cases the immune-stimulatory conjugate comprises a TLR7 agonist. In certain embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In some aspects, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide or a benzonaphthyridine, but is other than a guanosine analog, an adenosine analog, a thymidine homopolymer, ssRNA, CpG-A, PolyG10, and PolyG3. In some embodiments, a TLR7 agonist is a non-naturally occurring compound. In some aspects, a TLR7 agonist has an EC50 value of 100 nM or less by PBMC assay measuring TNFalpha or IFNalpha production. In some aspects, a TLR7 agonist has an EC50 value of 50 nM or less by PBMC assay measuring TNFalpha or IFNalpha production. In some embodiments, a TLR7 agonist has an EC50 value of 10 nM or less by PBMC assay measuring TNFalpha or IFNalpha production.

In some aspects, the myeloid cell agonist is a TLR8 agonist. In some aspects, the TLR8 agonist is selected from the group consisting of a benzazepine, a ssRNA, an imidazoquinoline, an aminoquinoline, and a thiazoloquinolone. In some aspects, the TLR8 is selected from a Category A compound. In some aspects, the TLR8 agonist is selected from the group consisting of VTX-2337, VTX-294, resiquimod, and compounds 1.1-1.67.

In some aspects, the TLR8 agonist is benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some aspects, a TLR8 agonist is selected from the group consisting of a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine and is other a ssRNA. In some aspects, a TLR8 agonist is a non-naturally occurring compound. In some aspects, a TLR8 agonist has an EC50 value of 500 nM or less by PBMC assay measuring TNFalpha production. In some aspects, a TLR8 agonist has an EC50 value of 100 nM or less by PBMC assay measuring TNFalpha production. In some aspects, a TLR8 agonist has an EC50 value of 50 nM or less by PBMC assay measuring TNFalpha production. In some aspects, a TLR8 agonist has an EC50 value of 10 nM or less by PBMC assay measuring TNFalpha production.

In some cases, the immune-stimulatory conjugate comprises two different agonists, a TLR7 myeloid cell agonist and a TLR8 myeloid cell agonist, also referred to herein as a mixed TLR7/TLR8 agonist conjugate or a dual payload TLR7/TLR8 conjugate. The TLR7 and TLR8 agonists can be any one of the agonists described herein.

In some aspects, the Fc binding domain is an IgG region. In some aspects, the Fc binding domain is an IgG1 Fc region. In some aspects, the Fc binding domain is an Fc binding domain variant comprising one or more amino acid substitutions in an IgG region as compared to an amino acid sequence of a wild-type IgG region. In some aspects, the Fc binding domain variant has increased affinity to one or more Fcγ receptors as compared to the wild-type IgG region. In some aspects, the Fc binding domain is a non-antibody scaffold.

In some aspects, the target antigen binding domain comprises an immunoglobulin heavy chain variable region or an antigen binding fragment thereof and an immunoglobulin light chain variable region or an antigen binding fragment thereof. In some aspects, the target antigen binding domain comprises a single chain variable region fragment (scFv). In some aspects, the second antigen binding domain comprises an immunoglobulin heavy chain variable region or an antigen binding fragment thereof and an immunoglobulin light chain variable region or an antigen binding fragment thereof. In some aspects, the second antigen binding domain comprises a single chain variable region fragment (scFv). In some aspects, the Fc binding domain is covalently attached to the targeting domain: a) as an Fc binding domain-targeting binding domain fusion protein; or b) by conjugation via a second linker. In some aspects, the antibody construct has a Kd for binding of the Fc binding domain to an Fc receptor in the presence of the myeloid cell agonist and wherein the K_{d} for binding of the Fc binding domain to the Fc receptor in the presence of the myeloid cell agonist is no greater than about 100 times a K_{d} for binding of the Fc binding domain to the Fc receptor in the absence of the myeloid cell agonist.

In various aspects, a pharmaceutical composition comprises the conjugate of any of the preceding embodiments and a pharmaceutically acceptable carrier.

In various aspects, a method of treating a subject having a liver viral infection comprises administering to the subject an effective dose of the conjugate of any of the preceding embodiments or the pharmaceutical composition of any of the preceding embodiments. In some aspects, the subject has a Hepatitis B infection. In some aspects, the subject does not have cancer. In some aspects, the conjugate is administered systemically. In some aspects, the conjugate is administered intravenously, cutaneously, subcutaneously, or injected at a site of the viral infection.

In various aspects, a kit comprises a pharmaceutically acceptable dosage unit of a pharmaceutically effective amount of the conjugate of any of the preceding embodiments or the pharmaceutical composition of any of the preceding embodiments.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings of which:

FIG. 1 illustrates the activity of an ASGR1-TLR8 agonist conjugate in a PBMC-hepatocyte co-culture assay in the presence of ASGR1-positive and ASGR1-negative control cells.

FIG. 2 shows that ASGR1-TLR8 agonist conjugates were active in the presence of PBMCs and HepG2 cells that express ASGR1, as measured by TNFα production.

FIG. 3 shows that ASGR1-TLR8 agonist cysteine engineered conjugates were active in the presence of PBMCs and HepG2 that express ASGR1, as measured by TNFα production.

FIG. 4 shows that ASGR1-TLR8 agonist conjugates with varying linkers were active in the presence of PBMCs and HepG2 that express ASGR1, as measured by TNFα production.

FIG. 5 shows that mixed TLR8-TLR7 agonist conjugates were active in the presence of PBMCs and HepG2 that express ASGR1, as measured by TNFα production.

FIG. 6A-FIG. 6C shows that both the TLR8 and TLR7 agonists were active in mixed TLR8-TLR7 agonist conjugates in the presence of PBMCs and HepG2 that express ASGR1, as measured by cytokine production. FIG. 6A shows IFNα production; FIG. 6B shows IL-12 production and FIG. 6C shows TNFα production.

FIG. 7 shows that TNFα production by PBMCs agonized by ASGR1 TLR8 conjugates is Fc dependent.

FIG. 8 shows that small molecule TLR7 and TLR8 compounds induce Marmota TNFα expression.

FIG. 9 shows that ASGR1-TLR8 agonist conjugates conditionally activate Marmota PBMCs.

### DEFINITIONS

Additional aspects and advantages of the present disclosure will become apparent to those skilled in this art from the following detailed description, wherein illustrative aspects of the present disclosure are shown and described. As will be appreciated, the present disclosure is capable of other and different aspects, and its several details are capable of modifications in various respects, all without departing from the disclosure. Accordingly, the descriptions are to be regarded as illustrative in nature, and not as restrictive.

As used herein, “identical” or “identity” refer to the similarity between a DNA, RNA, nucleotide, amino acid, or protein sequence to another DNA, RNA, nucleotide, amino acid, or protein sequence. Identity can be expressed in terms of a percentage of sequence identity of a first sequence to a second sequence. Percent (%) sequence identity with respect to a reference DNA sequence can be the percentage of DNA nucleotides in a candidate sequence that are identical with the DNA nucleotides in the reference DNA sequence after aligning the sequences. Percent (%) sequence identity with respect to a reference amino acid sequence can be the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity.

As used herein, the term “antibody” refers to an immunoglobulin molecule that specifically binds to, or is immunologically reactive toward, a specific antigen. The term antibody can include, for example, polyclonal, monoclonal, genetically engineered, and antigen binding fragments thereof. An antibody can be, for example, murine, chimeric, humanized, a heteroconjugate, bispecific, diabody, triabody, or tetrabody. An antigen binding fragment can include, for example, a Fab′, F(ab′)₂, Fab, Fv, rIgG, scFv, hcAbs (heavy chain antibodies), a single domain antibody, V_{HH}, V_{NAR}, sdAbs, or nanobody.

As used herein, “recognize” refers to the specific association or specific binding between an antigen binding domain and an antigen.

As used herein, “specifically binds” and the like refers to the specific association or specific binding between the antigen binding domain and the antigen, as compared with the interaction of the antigen binding domain with a different antigen (i.e., non-specific binding). In some embodiments, an antigen binding domain that recognizes or specifically binds to an antigen has a dissociation constant (KD) of <<100 nM, <10 nM, <1 nM, <0.1 nM, <0.01 nM, or <0.001 nM (e.g. 10⁻⁸M or less, e.g. from10⁻⁸ M to 10⁻¹³M, e.g., from 10⁻⁹ M to 10⁻¹³M).

As used herein, an “antigen” refers to an antigenic substance that can elicit an immune response in a host. An antigen can be a protein, polysaccharide, lipid, or glycolipid, which can be recognized by an immune cell, such as a T cell or a B cell. Exposure of immune cells to one or more of these antigens can elicit a rapid cell division and differentiation response resulting in the formation of clones of the exposed T cells and B cells. B cells can differentiate into plasma cells which in turn can produce antibodies which selectively bind to the antigens.

As used herein, a “liver cell antigen” refers to an antigenic substance expressed on a liver cell that can be recognized by an antibody or binding domain, and is preferentially expressed on a non-cancerous liver cell as compared to cells from other tissues.

As used herein, a “viral antigen” refers to an antigenic substance associated a virus, a viral infection, or combination thereof. A “viral antigen from a virus infecting a liver cell” refers to antigenic substance associated with a virus that is infecting or has infected a liver cell and that can trigger an immune response in a host.

As used herein, an “antibody construct” refers to a construct that contains an antigen binding domain and an Fc binding domain.

As used herein, an “antigen binding domain” refers to a binding domain from an antibody or from a non-antibody that can specifically bind to an antigen. Antigen binding domains can be numbered when there is more than one antigen binding domain in a given conjugate or antibody construct (e.g., first antigen binding domain, second antigen binding domain, third antigen binding domain, etc.). Different antigen binding domains in the same conjugate or construct can target the same antigen (e.g., a first antigen binding domain and a second antigen can specifically bind to the same liver cell antigen of the conjugate or construct) or a different antigen (e.g., a first antigen binding domain can specifically bind to a liver cell antigen and a second antigen binding domain can specifically bind to a viral antigen from a virus infecting the liver cell of the conjugate or construct).

As used herein, an “Fc binding domain” refers to a domain from an Fc portion of an antibody or a domain from a non-antibody molecule that can bind to an Fc receptor, such as a Fcgamma receptor or an FcRn.

As used herein, a “target antigen binding domain” refers to an antigen binding domain of a conjugate or construct that specifically binds an antigen.

As used herein, a “myeloid cell agonist” refers to a compound that agonizes a toll-like receptor 7 (TLR7), toll-like receptor 8 (TLR8), or a combination thereof.

As used herein, a “conjugate” refers to an antibody construct attached to at least one myeloid cell agonist via a linker(s).

As used herein, a “bispecific antibody construct” refers to an antibody construct further consisting of a second antigen binding domain.

As used herein, a “bispecific antibody conjugate” refers to an antibody construct further consisting of a second antigen binding domain and that is attached to at least one myeloid cell agonist via a linker(s).

As used herein, a “liver cell” refers to any cell type associated with normal liver tissue. For example, a liver cell can be a canalicular cell, a Kupffer cell, a hepatocyte, sinusoidal endothelial cell, or a stellate cell.

As used herein, an “immune cell” refers to a T cell, B cell, NK cell, NKT cell, or an antigen presenting cell. In some embodiments, an immune cell is a T cell, B cell, NK cell, or NKT cell. In some embodiments, an immune cell is an antigen presenting cell. In some embodiments, an immune cell is not an antigen presenting cell.

As used herein, the abbreviations for the natural L-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R).

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term —C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkenylene chain. For example, —C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C3 alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene chain is optionally substituted by one or more substituents such as those substituents described herein.

“Alkenylene” refers to a divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). Unless stated otherwise specifically in the specification, an alkenylene chain is optionally substituted by one or more substituents such as those substituents described herein.

“Alkynylene” refers to a divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene). Unless stated otherwise specifically in the specification, an alkynylene chain is optionally substituted by one or more substituents such as those substituents described herein.

“Heteroalkylene” refers to a divalent hydrocarbon chain including at least one heteroatom in the chain, containing no unsaturation, and preferably having from one to twelve carbon atoms and from one to 6 heteroatoms, e.g., —O—, —NH—, —S—. The heteroalkylene chain is attached to the rest of the molecule through a single bond and to the radical group through a single bond. The points of attachment of the heteroalkylene chain to the rest of the molecule and to the radical group are through the terminal atoms of the chain. In other embodiments, a heteroalkylene comprises one to five carbon atoms and from one to three heteroatoms. In other embodiments, a heteroalkylene comprises one to four carbon atoms and from one to three heteroatoms. In other embodiments, a heteroalkylene comprises one to three carbon atoms and from one to two heteroatoms. In other embodiments, a heteroalkylene comprises one to two carbon atoms and from one to two heteroatoms. In other embodiments, a heteroalkylene comprises one carbon atom and from one to two heteroatoms. In other embodiments, a heteroalkylene comprises five to eight carbon atoms and from one to four heteroatoms. In other embodiments, a heteroalkylene comprises two to five carbon atoms and from one to three heteroatoms. In other embodiments, a heteroalkylene comprises three to five carbon atoms and from one to three heteroatoms. Unless stated otherwise specifically in the specification, a heteroalkylene chain is optionally substituted by one or more substituents such as those substituents described herein.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl. The term “unsaturated carbocycle” refers to carbocycles with at least one degree of unsaturation and excluding aromatic carbocycles. Examples of unsaturated carbocycles include cyclohexadiene, cyclohexene, and cyclopentene.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more rings in which two or more carbons are common to two adjoining rings wherein at least one of the rings is heteroaromatic, e.g., the other rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or substitutable heteroatoms, e.g., —NH—, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each Ra is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

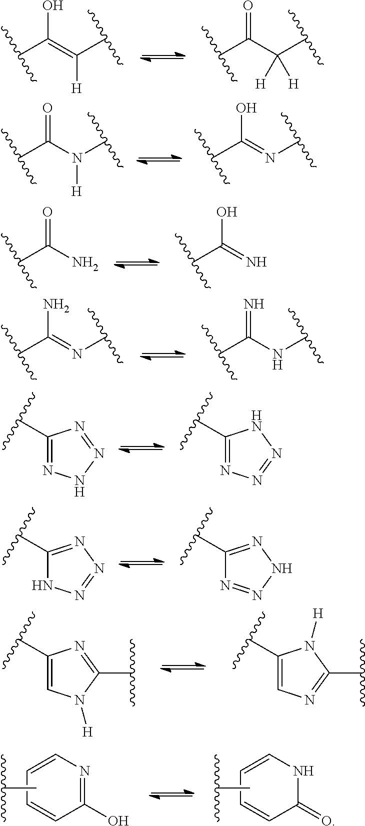

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

### DETAILED DESCRIPTON

Viral liver diseases, such as Hepatitis B and Hepatitis C, are infectious diseases caused by viruses (Hepatitis B virus (HBV) and Hepatitis C (HCV), respectively). Millions of people worldwide are affected by these types of viral infections of the liver, in which can lead to liver cirrhosis, liver damage, and liver failure. HBV belongs to the Hepadnaviridae virus family, and is an enveloped double stranded DNA virus. HCV belongs to the Flaviviridae virus family and is a small, enveloped, positive sense single-stranded RNA virus. Although antiviral medications can be used to treat infections, they are not always effective. Therefore, there is a need for alternative strategies to treat HBV and HCV and other viral liver infections.

Furthermore, immune-stimulatory molecular motifs, such as Pathogen-Associated Molecular Pattern molecules (PAMPs), can be recognized by receptors of the innate immune system, such as Toll-like receptors (TLRs), Nod-like receptors, C-type lectins, and RIG-I-like receptors. These receptors can be transmembrane and intra-endosomal proteins which can prime activation of the immune system in response to infectious agents such as viruses. Like other protein families, there are many different TLRs, including TLR7 and TLR8. However, therapeutic use of PAMPs or other mechanisms of intervention can be limited because systemic activation of PAMP signaling pathways can have life-threatening consequences due to cytokine syndrome-induced or cytokine storm-induced toxic shock syndrome. Accordingly, there is a need for therapeutic, clinically relevant targeted delivery of PAMP and DAMP agonists as effective strategies to enhance immune responses against viruses.

The presently described conjugates can be utilized to enhance liver-localized immune responses against viruses that cause liver disease, such as Hepatitis B and Hepatitis C. A conjugate as described herein comprises an antibody construct linked to at least one myeloid cell agonist via linker(s). In some embodiments, the antibody construct of the conjugate can comprise a target antigen binding domain that specifically binds to a liver cell antigen. In other embodiments, the antibody construct of the conjugate can comprise a target antigen binding domain that specifically binds to a viral antigen from a virus infecting a liver cell. In further embodiments, the antibody construct of the conjugate further comprises a second antigen binding domain, in addition to the target antigen binding domain. The second antigen binding domain can specifically bind to a liver cell antigen or a viral antigen from a virus infecting a liver cell. The myeloid cell agonist can be a toll-like receptor 7 (TLR7) agonist, a toll-like receptor 8 (TLR8) agonist, or a combination thereof.

### Antibody Construct

A conjugate as described herein comprises an antibody construct. An antibody construct comprises one or more antigen binding domains and an Fc binding domain. In some embodiments, an antibody construct comprises an antigen binding domain that specifically binds to an antigen and an Fc binding domain. An antibody construct can comprise a first antigen binding domain that specifically binds to a first antigen, second antigen binding domain that specifically binds to a second antigen, and an Fc domain. An antibody construct can comprise a target antigen binding domain that specifically binds a first antigen and an Fc domain. An antibody construct can comprise a target antigen binding domain that specifically binds a first antigen, and a second antigen binding domain that specifically binds a second antigen, and an Fc domain. An antibody construct can comprise an antibody, wherein the antibody comprises an antigen binding domain that specifically binds to an antigen and an Fc binding domain. An antibody construct can comprise a bispecific antibody, wherein the bispecific antibody comprises a first antigen binding domain that specifically binds to a first antigen, a second antigen binding domain that specifically binds to a second antigen, and an Fc domain. An antibody construct can comprise a bispecific antibody, wherein the bispecific antibody comprises a target antigen binding domain that specifically binds to a first antigen, a second antigen binding domain that specifically binds to a second antigen, and an Fc domain.

### Antigen Binding Domain

An antigen binding domain can be an antigen-binding portion of an antibody or an antibody fragment. An antigen binding domain can be one or more fragments of an antibody that can retain the ability to specifically bind to an antigen. An antigen binding domain can be any antigen binding fragment. An antigen binding domain typically recognizes a single antigen. An antibody construct typically comprises, for example, one or two antigen binding domains although more can be included in an antibody construct. An antibody construct can comprise two antigen binding domains in which each antigen binding domain recognize the same antigen. An antibody construct can comprise two antigen binding domains in which each antigen binding domain recognize the same epitope on the antigen. An antibody construct can comprise two antigen binding domains in which each antigen binding domain recognize different epitopes on the same antigen. An antibody construct can comprise two antigen binding domains in which each antigen binding domain can recognize different antigens. An antibody construct can comprise three antigen binding domains in which each antigen binding domain can recognize different antigens. An antibody construct can comprise three antigen binding domains in which two of the antigen binding domains can recognize the same antigen. An antigen binding domain can be in a scaffold, in which a scaffold is a supporting framework for the antigen binding domain. An antigen binding domain can be in a non-antibody scaffold. An antigen binding domain can be in an antibody scaffold or antibody-like scaffold. An antibody construct can comprise an antigen binding domain in a scaffold.

An antigen binding domain of an antibody construct can be selected from any domain that specifically binds to an antigen including, but not limited to, an antibody or from a non-antibody molecule. In some embodiments, an antigen binding domain can be selected from any domain of an antibody that specifically binds to an antigen including, but not limited to, from a monoclonal antibody, a polyclonal antibody, a recombinant antibody, or a functional fragment thereof, for example, a heavy chain variable domain (VH) and a light chain variable domain (VL), a Fab′, F(ab′)₂, Fab, Fv, rIgG, scFv, hcAbs (heavy chain antibodies), a single domain antibody, V_{HH}, V_{NAR}, sdAbs, or nanobody. In some embodiments, an antigen binding domain can be selected from any domain of a non-antibody molecule that specifically binds to an antigen including, but not limited to, from a non-antibody scaffold, such as a DARPin, an affimer, an avimer, a knottin, a monobody, lipocalin, an anticalin, ‘T-body’, an affibody, a peptibody, an affinity clamp, an ectodomain, a receptor ectodomain, a receptor, a ligand, or a centryin

An antigen binding domain of an antibody construct, for example an antigen binding domain from a monoclonal antibody, can comprise a light chain and a heavy chain. In one aspect, the monoclonal antibody specifically binds to an antigen present on the surface of a liver cell and comprises the light chain of an anti-liver cell antigen antibody and the heavy chain of an anti-liver cell antigen antibody, that form the antigen binding domain that specifically binds to the liver cell antigen.

An antigen binding domain of an antibody construct can be a target antigen binding domain that specifically binds to a first antigen, such as a liver cell antigen or a viral antigen expressed on a liver cell. In some embodiments, the first antigen can be expressed by a liver cell. For example, a first antigen can be a liver cell antigen, a molecular marker is preferentially expressed on a liver cell as compared to cells from other normal tissues. For example, a liver cell antigen can be expressed on a canalicular cell, Kupffer cell, hepatocyte, a stellate cell, or a combination thereof. The liver cell antigen can be a liver cell surface receptor. The liver cell antigen can be a hepatocyte antigen. In some embodiments, the liver cell antigen can include, but is not limited to, asialoglycoprotein receptor 1 (ASGR1), asialoglycoprotein receptor 2 (ASGR2), transferrin receptor 2 (TRF2), UDP glucuronosyltransferase 1 family, polypeptide A1 (UGT1A1), solute carrier family 22 member 7 (SLC22A7), solute carrier family 13 member 5 (SLC13A5), solute carrier family 22 member 1 (SLC22A1), and complement component 9 (C9). In some embodiments, the liver cell antigen is selected from the group consisting of ASGR1, ASGR2, TRF2, UGT1A1, SLC22A7, SLC13A5, SLC22A1, and C9. In some embodiments, the liver cell antigen is selected from the group consisting of ASGR1, ASGR2, TRF2, UGT1A1, SLC22A7, SLC13A5, and SLC22A1. In some embodiments, the liver cell antigen is selected from the group consisting of ASGR1, ASGR2 and TRF2.

Asialoglycoprotein receptor 1 (ASGR1) can have the amino acid sequence set forth in accession NP_001184145.1 or NP_001662.1. Asialoglycoprotein receptor 2 (ASGR2) can have the amino acid sequence set forth in accession NP_001172.1, NP_001188281.1, NP_550434.1, NP_550435.1, or NP_550436.1. Transferrin receptor 2 (TRF2) can have the amino acid sequence set forth in accession NP_001193784.1 or NP_003218.1. UDP glucuronosyltransferase 1 family, polypeptide A1 (UGT1A1) can have the amino acid sequence set forth in accession NP_000454.1. Solute carrier family 22 member 7 (SLC22A7) can have the amino acid sequence set forth in accession NP_006663.2 or NP_696961.2. Solute carrier family 13 member 5 (SLC13A5) can have the amino acid sequence set forth in accession NP_001137310.1, NP_001271438.1, NP_001271439.1 or NP_808218.1. Solute carrier family 22 member 1(SLC22A1) can have the amino acid sequence set forth in accession NP_003048.1 or NP_694857.1. Complement component 9 (C9) can have the amino acid sequence set forth in accession NP_001728.1.

The target antigen binding domain can specifically bind to a liver cell antigen, wherein the liver cell antigen has an amino acid sequence that comprises at least 80% identity to an amino acid sequence of an antigen selected from the group consisting of ASGR1, ASGR2, TRF2, UGT1A1, SLC22A7, SLC13A5, SLC22A1, and C9. The target antigen binding domain can specifically bind to a liver cell antigen, wherein the liver cell antigen has an amino acid sequence that comprises at least 80% identity to an amino acid sequence of an antigen selected from the group consisting of ASGR1, ASGR2, TRF2, UGT1A1, SLC22A7, SLC13A5, and SLC22A1. The target antigen binding domain can specifically bind to a liver cell antigen, wherein the liver cell antigen has an amino acid sequence that comprises at least 80% identity to an amino acid sequence of an antigen selected from the group consisting of ASGR1, ASGR2 and TRF2. The target antigen binding domain can specifically bind to a liver cell antigen, wherein the liver cell antigen has an amino acid sequence that comprises at least 85%, 90%, 95%, 98%, or 100% identity to an amino acid sequence of an antigen selected from the group consisting of ASGR1, ASGR2, TRF2, UGT1A1, SLC22A7, SLC13A5, SLC22A1, and C9. The target antigen binding domain can specifically bind to a liver cell antigen, wherein the liver cell antigen has an amino acid sequence that comprises at least 85%, 90%, 95%, 98%, or 100% identity to an amino acid sequence of an antigen selected from the group consisting of ASGR1, ASGR2, TRF2, UGT1A1, SLC22A7, SLC13A5, and SLC22A1. The target antigen binding domain can specifically bind to a liver cell antigen, wherein the liver cell antigen has an amino acid sequence that comprises at least 85%, 90%, 95%, 98%, or 100% identity to an amino acid sequence of an antigen selected from the group consisting of ASGR1, ASGR2 and TRF2.

Asialoglycoprotein receptor 1 (ASGR1) can have an amino acid sequence that is 90% identical to the amino acid sequence set forth in accession NP_001184145.1 or NP_001662.1. Asialoglycoprotein receptor 2 (ASGR2) can have an amino acid sequence that is 90% identical to the amino acid sequence set forth in accession NP_001172.1, NP_001188281.1, NP_550434.1, NP_550435.1, or NP_550436.1. Transferrin receptor 2 (TRF2) can have an amino acid sequence that is 90% identical to the amino acid sequence set forth in accession NP_001193784.1 or NP_003218.1. UDP glucuronosyltransferase 1 family, polypeptide A1 (UGT1A1) can have an amino acid sequence that is 90% identical to the amino acid sequence set forth in accession NP_000454.1. Solute carrier family 22 member 7 (SLC22A7) can have an amino acid sequence that is 90% identical to the amino acid sequence set forth in accession NP_006663.2 or NP_696961.2. Solute carrier family 13 member 5 (SLC13A5) can have an amino acid sequence that is 90% identical to the amino acid sequence set forth in accession NP_001137310.1, NP_001271438.1, NP_001271439.1 or NP_808218.1. Solute carrier family 22 member 1(SLC22A1) can have an amino acid sequence that is 90% identical to the amino acid sequence set forth in accession NP_003048.1 or NP_694857.1. Complement component 9 (C9) can have an amino acid sequence that is 90% identical to the amino acid sequence set forth in accession NP_001728.1.

In some embodiments, the liver cell antigen can be expressed on a cell that is infected with a virus. The virus can be a hepatitis virus, such as HBV or HCV. In some embodiments, the virus is HBV and not HCV. In some embodiments, the virus is HCV.

In other embodiments, a first antigen can be a viral antigen from a virus infecting a liver cell. A viral antigen can be a molecular marker of a virus, which is expressed on a liver cell when the liver cell is infected with the virus. For example, a viral antigen can be expressed on a canalicular cell, Kupffer cell, hepatocyte, a stellate cell, or a combination thereof, when infected by a virus. The virus can be a hepatitis virus. The virus can be HBV. The virus can be HCV. The viral antigen can be expressed on a non-cancerous liver cell infected with a virus. A viral antigen can include, but is not limited to, the components of HBV such as HBsAg, HBcAg, HBeAg, Hepatitis B virus DNA polymerase, or HBx, and components of HCV such as Core protein, E1 and E2, NS2, NS3, NS4A, NS4B, NS5A, or NS5B. In some embodiments, the viral antigen is HBsAg, HBcAg or HBeAg. In some embodiments, the viral antigen is HBsAg.

HBaAg can have the amino acid sequence set forth in accession Q773S4_HBV. HBcAg can have the amino acid sequence set forth in accession Q2I360_HBV. HBeAg can have the amino acid sequence set forth in accessions P0C573, P0C625, Q05495, P0C767, P0C6H2, P0C699, P0C6G9, or P0C692.

The target antigen binding domain can specifically bind to a viral antigen from a virus infecting a liver cell, wherein the viral antigen has an amino acid sequence that comprises at least 80% identity to an amino acid sequence of an antigen selected from the group consisting of HBsAg, HBcAg, HBeAg, Hepatitis B virus DNA polymerase, HBx, Core protein, E1 and E2, NS2, NS3, NS4A, NS4B, NS5A, and NS5B. In some embodiments, the viral antigen is HBsAg, HBcAg or HBeAg. In some embodiments, the viral antigen is HBsAg. The target antigen binding domain can specifically bind to a viral antigen for a virus infecting a liver cell, wherein the viral antigen has an amino acid sequence that comprises at least 85%, 90%, 95%, 98%, or 100% identity to an amino acid sequence of an antigen selected from the group consisting of HBsAg, HBcAg, HBeAg, Hepatitis B virus DNA polymerase, HBx, Core protein, E1 and E2, NS2, NS3, NS4A, NS4B, NS5A, and NS5B.

An antibody construct can have a second antigen binding domain. The second antigen binding domain can specifically bind to the first antigen. The second antigen binding domain can specifically bind to a second antigen. The second antigen can be expressed by a liver cell. For example, a second antigen can be a liver cell antigen. A liver cell antigen can be a molecular marker is preferentially expressed on a liver cell as compared to cells from other normal tissues. For example, a liver cell antigen can be expressed on a canalicular cell, Kupffer cell, hepatocyte, a stellate cell, or a combination thereof. The liver cell antigen can be a liver cell surface receptor. The liver cell antigen can be a hepatocyte antigen. The liver cell antigen can be expressed on a non-cancerous liver cell. The liver cell antigen can be expressed on a cell infected with a virus. The virus can be a liver virus. The virus can be a hepatitis virus. The virus can be HBV. The virus can be HCV.

A liver cell antigen can include, but is not limited to, ASGR1, ASGR2, TRF2, UGT1A1, SLC22A7, SLC13A5, SLC22A1, and C9. In some embodiments, the liver cell antigen is selected from the group consisting of ASGR1, ASGR2, TRF2, UGT1A1, SLC22A7, SLC13A5, SLC22A1, and C9. In some embodiments, the liver cell antigen is selected from the group consisting of ASGR1, ASGR2, TRF2, UGT1A1, SLC22A7, SLC13A5 and SLC22A1. In some embodiments, the liver cell antigen is selected from the group consisting of ASGR1, ASGR2 and TRF2.

Asialoglycoprotein receptor 1 (ASGR1) can have the amino acid sequence set forth in accession NP_001184145.1 or NP_001662.1. Asialoglycoprotein receptor 2 (ASGR2) can have the amino acid sequence set forth in accession NP_001172.1, NP_001188281.1, NP_550434.1, NP_550435.1, or NP_550436.1. Transferrin receptor 2 (TRF2) can have the amino acid sequence set forth in accession NP_001193784.1 or NP_003218.1. UDP glucuronosyltransferase 1 family, polypeptide A1 (UGT1A1) can have the amino acid sequence set forth in accession NP_000454.1. Solute carrier family 22 member 7 (SLC22A7) can have the amino acid sequence set forth in accession NP_006663.2 or NP_696961.2. Solute carrier family 13 member 5 (SLC13A5) can have the amino acid sequence set forth in accession NP_001137310.1, NP_001271438.1, NP_001271439.1 or NP_808218.1. Solute carrier family 22 member 1 (SLC22A1) can have the amino acid sequence set forth in accession NP_003048.1 or NP_694857.1. Complement component 9 (C9) can have the amino acid sequence set forth in accession NP_001728.1.

The second antigen binding domain can specifically bind to a liver cell antigen, wherein the liver cell antigen has an amino acid sequence that comprises at least 80% identity to an amino acid sequence of an antigen selected from the group consisting of ASGR1, ASGR2, TRF2, UGT1A1, SLC22A7, SLC13A5, SLC22A1, and C9. The second antigen binding domain can specifically bind to a liver cell antigen, wherein the liver cell antigen has an amino acid sequence that comprises at least 80% identity to an amino acid sequence of an antigen selected from the group consisting of ASGR1, ASGR2, TRF2, UGT1A1, SLC22A7, SLC13A5 and SLC22A1. The second antigen binding domain can specifically bind to a liver cell antigen, wherein the liver cell antigen has an amino acid sequence that comprises at least 80% identity to an amino acid sequence of an antigen selected from the group consisting of ASGR1, ASGR2 and TRF2. The second antigen binding domain can specifically bind to a liver cell antigen, wherein the liver cell antigen has an amino acid sequence that comprises at least 85%, 90%, 95%, 98%, or 100% identity to an amino acid sequence of an antigen selected from the group consisting of ASGR1, ASGR2, TRF2, UGT1A1, SLC22A7, SLC13A5, SLC22A1, and C9. The second antigen binding domain can specifically bind to a liver cell antigen, wherein the liver cell antigen has an amino acid sequence that comprises at least 85%, 90%, 95%, 98%, or 100% identity to an amino acid sequence of an antigen selected from the group consisting of ASGR1, ASGR2, TRF2, UGT1A1, SLC22A7, SLC13A5, and SLC22A1. The second antigen binding domain can specifically bind to a liver cell antigen, wherein the liver cell antigen has an amino acid sequence that comprises at least 85%, 90%, 95%, 98%, or 100% identity to an amino acid sequence of an antigen selected from the group consisting of ASGR1, ASGR2 and TRF2.

In other embodiments, a second antigen can be a viral antigen from a virus infecting a liver cell. A viral antigen can be a molecular marker of a virus, which is expressed on a liver cell when the liver cell is infected with the virus. For example, a viral antigen can be expressed on a canalicular cell, Kupffer cell, hepatocyte, a stellate cell, or a combination thereof, when infected by a virus. The virus can be a liver virus. The virus can be a hepatitis virus. The virus can be HBV. The virus can be HCV. The viral antigen can be expressed on a non-cancerous liver cell infected with a virus. A viral antigen can include, but is not limited to, the components of HBV such as HBsAg, HBcAg, HBeAg, Hepatitis B virus DNA polymerase, or HBx, and components of HCV such as Core protein, E1 and E2, NS2, NS3, NS4A, NS4B, NS5A, or NS5B. In some embodiments, the viral antigen is HBsAg, HBcAg or HBeAg. In some embodiments, the viral antigen is HBsAg. The second antigen binding domain can specifically bind to a viral antigen from a virus infecting a liver cell, wherein the viral antigen has an amino acid sequence that comprises at least 80% identity to an amino acid sequence of an antigen selected from the group consisting of HBsAg, HBcAg, HBeAg, Hepatitis B virus DNA polymerase, HBx, Core protein, E1 and E2, NS2, NS3, NS4A, NS4B, NS5A, and NS5B. The second antigen binding domain can specifically bind to a viral antigen for a virus infecting a liver cell, wherein the viral antigen has an amino acid sequence that comprises at least 85%, 90%, 95%, 98%, or 100% identity to an amino acid sequence of an antigen selected from the group consisting of HBsAg, HBcAg, HBeAg, Hepatitis B virus DNA polymerase, HBx, Core protein, E1 and E2, NS2, NS3, NS4A, NS4B, NS5A, and NS5B.

### Fc Binding Domain

An antibody construct includes an Fc binding domain. An Fc binding domain is a structure that can bind to one or more Fc receptors (FcRs). FcRs can bind to an Fc binding domain of an antibody. FcRs can bind to an Fc binding domain of an antibody bound to an antigen. FcRs are organized into classes (e.g., gamma (γ), alpha (α) and epsilon (ε)) based on the class of antibody that the FcR recognizes. The FcαR class binds to IgA and includes several isoforms, FcαRI (CD89) and FcαμR. The FcγR class binds to IgG and includes several isoforms, FcγRT (CD64), FcγRIIA (CD32a), FcγRIIB (CD32b), FcγRIIIA (CD16a), and FcγRIIIB (CD16b). An FcγRIIIA (CD16a) can be an FcγRIIIA (CD16a) F158 variant or a V158 variant. Each FcγR isoform can differ in binding affinity to the Fc binding domain of the IgG antibody. For example, FcγRT can bind to IgG with greater affinity than FcγRII or FcγRIII. The affinity of a particular FcγR isoform to IgG can be controlled, in part, by a glycan (e.g., oligosaccharide) at position CH2 84.4 of the IgG antibody. For example, fucose containing CH2 84.4 glycans can reduce IgG affinity for FcγRIIIA In addition, G0 glucans can have increased affinity for FcγRIIIA due to the lack of galactose and terminal GlcNAc moiety.

Binding of an Fc binding domain to an FcR can enhance an immune response. FcR-mediated signaling that can result from an Fc binding domain binding to an FcR can lead to the maturation of immune cells. FcR-mediated signaling that can result from an Fc binding domain binding to an FcR can lead to the maturation of dendritic cells (DCs). FcR-mediated signaling that can result from an Fc binding domain binding to an FcR can lead to antibody dependent cellular cytotoxicity. FcR-mediated signaling that can result from an Fc binding domain binding to an FcR can lead to more efficient immune cell antigen uptake and processing. FcR-mediated signaling that can result from an Fc binding domain binding to an FcR can promote the expansion and activation of T cells. FcR-mediated signaling that can result from an Fc binding domain binding to an FcR can promote the expansion and activation of CD8+ T cells. FcR-mediated signaling that can result from an Fc binding domain binding to an FcR can influence immune cell regulation of T cell responses. FcR-mediated signaling that can result from an Fc binding domain binding to an FcR can influence immune cell regulation of T cell responses. FcR-mediated signaling that can result from an Fc binding domain binding to an FcR can influence dendritic cell regulation of T cell responses. FcR-mediated signaling that can result from an Fc binding domain binding to an FcR can influence functional polarization of T cells (e.g., polarization can be toward a TH1 cell response).

The profile of FcRs on a DC can impact the ability of the DC to respond upon stimulation. For example, most DC can express both CD32A and CD32B, which can have opposing effects on IgG-mediated maturation and function of DCs: binding of IgG to CD32A can mature and activate DCs in contrast with CD32B, which can mediate inhibition due to phosphorylation of immunoreceptor tyrosine-based inhibition motif (ITIM), after CD32B binding of IgG. Therefore, the activity of these two receptors can establish a threshold of DC activation. Furthermore, difference in functional avidity of these receptors for IgG can shift their functional balance. Hence, altering the Fc binding domain binding to FcRs can also shift their functional balance, allowing for manipulation (either enhanced activity or enhanced inhibition) of the DC immune response.

A modification in the amino acid sequence of an Fc binding domain can alter the recognition of an FcR for the Fc binding domain. However, such modifications can still allow for FcR-mediated signaling. A modification can be a substitution of an amino acid at a residue of an Fc binding domain (e.g., wildtype) for a different amino acid at that residue. A modification can permit binding of an FcR to a site on the Fc binding domain that the FcR may not otherwise bind to. A modification can increase binding affinity of an FcR to the Fc binding domain. A modification can decrease binding affinity of an FcR to a site on the Fc binding domain that the FcR may have increased binding affinity for. A modification can increase the subsequent FcR-mediated signaling after Fc binding domain binding to an FcR.

An Fc binding domain can be a naturally occurring or a variant of a naturally occurring Fc binding domain and can comprise at least one amino acid change as compared to the sequence of a wild-type Fc binding domain. An amino acid change in an Fc binding domain can allow the antibody construct or conjugate to bind to at least one Fc receptor with greater affinity compared to a wild-type Fc binding domain. An Fc binding domain variant can comprise an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. An Fc binding domain variant can comprise a sequence of the IgG1 isoform that has been modified from an wildtype IgG1 sequence to increase Fc receptor binding. A modification can comprise a substitution at one or more one amino acid residues of an Fc binding domain such as at 5 different amino acid residues including L235V/F243L/R292P/Y300L/P396L (IgG1VLPLL). The numbering of amino acids residues described herein is according to the EU index. This modification can be located in a portion of an antibody construct which can includes an Fc binding domain and in particular, can be located in a portion of the Fc binding domain that can bind to Fc receptors. A modification can comprise a substitution at one or more amino acid residues such as at 2 different amino acid residues of an Fc binding domain, including S239D/I332E (IgG1DE). This modification can be located in a portion of an antibody sequence which includes an Fc binding domain of the antibody and in particular, are located in portions of the Fc binding domain that can bind to Fc receptors. A modification can comprise a substitution at one or more amino acid residues such as at 3 different amino acid residues of an Fc binding domain including S298A/E333A/K334A (IgG1AAA). The modification can be located in a portion of an antibody sequence which includes an Fc binding domain of the antibody and in particular, can be located in portions of the Fc binding domain that can bind Fc receptors.

Binding of Fc receptors to an Fc binding domain can be affected by amino acid substitutions. For example, binding of some Fc receptors to an Fc binding domain variant comprising the IgG1VLPLL modifications can be enhanced compared to wild-type by as result of the L235V/F243L/R292P/Y300L/P396L amino acid modifications. However, binding of other Fc receptors to the Fc binding domain variant comprising the IgG1VLPLL modifications can be reduced compared to wild-type by the L235V/F243L/R292P/Y300L/P396L amino acid modifications. For example, the binding affinities of the Fc binding domain variant comprising the IgG1VLPLL modifications to FcγRIIIA and to FcγRIIA can be enhanced compared to wild-type whereas the binding affinity of the Fc binding domain variant comprising the IgG1VLPLL modifications to FcγRIM can be reduced compared to wild-type. Binding of Fc receptors to an Fc binding domain variant comprising the IgG1DE modifications can be enhanced compared to wild-type as a result of the S239D/I332E amino acid modification. However, binding of some Fc receptors to the Fc binding domain variant comprising the IgG1DE modifications can be reduced compared to wild-type by S239D/I332E amino acid modification. For example, the binding affinities of the Fc binding domain variant comprising the IgG1DE modifications to FcγRIIIA and to FcγRIM can be enhanced compared to wild-type. Binding of Fc receptors to an Fc binding domain variant comprising the IgG1AAA modifications can be enhanced compared to wild-type as a result of the S298A/E333A/K334A amino acid modification. However, binding of some Fc receptors to Fc binding domain variant comprising the IgG1AAA modifications can be reduced compared to wild-type by S298A/E333A/K334A amino acid modification. Binding affinities of the Fc binding domain variant comprising the IgG1AAA modifications to FcγRIIIA can be enhanced compared to wild-type whereas the binding affinity of the Fc binding domain variant comprising the IgG1AAA modifications to FcγRIIB can be reduced compared to wildtype.

In some embodiments, the heavy chain of a human IgG2 antibody can be mutated at cysteines as positions 127, 232, or 233. In some embodiments, the light chain of a human IgG2 antibody can be mutated at a cysteine at position 214. The mutations in the heavy and light chains of the human IgG2 antibody can be from a cysteine residue to a serine residue.

While an antibody construct can comprise a first binding domain and a second binding domain with wild-type or modified amino acid sequences encoding the Fc binding domain, the modifications of the Fc binding domain from the wild-type sequence may not significantly alter binding and/or affinity of the Fc binding domain or the antigen binding domain(s). For example, binding and/or affinity of an antibody construct comprising a first binding domain and a second binding domain (or, in some cases, a third binding domain) and having the Fc binding domain modifications of IgG1VLPLL, IgG1DE, or IgG1AAA may not be significantly altered by modification of an Fc binding domain amino acid sequence compared to a wild-type sequence. Modifications of an Fc binding domain from a wild-type sequence may not alter binding and/or affinity of a first binding domain or target binding domain that binds, for example, to a liver cell antigen or a viral antigen from a virus infecting liver cell. Additionally, the binding and/or affinity of the binding domains described herein, for example a first binding domain, a second binding domain (or, in some cases, a third binding domain), and an Fc binding domain variant selected from IgG1VLPLL, IgG1DE, and IgG1AAA, may be comparable to the binding and/or affinity of wild-type antibodies.

An Fc binding domain can be from an antibody. An Fc binding domain can be from an IgG antibody. An Fc binding domain can be from an IgG1, IgG2, or IgG4 antibody. An Fc binding domain can be at least 80% identical to an Fc binding domain from an antibody. An Fc binding domain can be a portion of the Fc binding domain of an antibody.

An antibody construct can comprise an Fc binding domain in an antibody. An antibody construct can comprise an Fc binding domain in a scaffold. An antibody construct can comprise an Fc binding domain in an antibody scaffold. An antibody construct can comprise an Fc binding domain in a non-antibody scaffold. An antibody construct can comprise an Fc binding domain covalently attached to an antigen binding domain.

An antibody construct can comprise an antigen binding domain and an Fc binding domain, wherein the Fc binding domain can be covalently attached to the antigen binding domain. An antibody construct can comprise a target antigen binding domain and Fc binding domain, wherein the Fc binding domain can be covalently attached to the target antigen binding domain. An antibody construct can comprise an antigen binding domain and Fc binding domain, wherein the Fc binding domain is covalently attached to the antigen binding domain as an Fc binding domain-antigen binding domain fusion protein. An antibody construct can comprise an antigen binding domain and Fc binding domain, wherein the Fc binding domain is covalently attached to the antigen binding domain by a linker. An antibody construct can comprise a target antigen binding domain and Fc binding domain, wherein the Fc binding domain is covalently attached to the target antigen binding domain as an Fc binding domain-target antigen binding domain fusion protein. An antibody construct can comprise a target antigen binding domain and Fc binding domain, wherein the Fc binding domain is covalently attached to the target antigen binding domain via a linker.

### Antibody

An antibody construct can comprise an antibody, which can comprise an antigen binding domain and an Fc binding domain. An antibody molecule can consist of two identical light protein chains (light chains) and two identical heavy protein chains (heavy chains), all held together covalently by precisely located disulfide linkages. The N-terminal regions of the light and heavy chains together can form the antigen recognition site of each antibody. Structurally, various functions of an antibody can be confined to discrete protein domains (i.e., regions). The sites that can recognize and can bind to antigen consist of three complementarity determining regions (CDRs) that can lie within the variable heavy chain regions and variable light chain regions at the N-terminal ends of the two heavy and two light chains. The constant domains can provide the general framework of the antibody and may not be involved directly in binding the antibody to an antigen, but can be involved in various effector functions, such as participation of the antibody in antibody-dependent cellular cytotoxicity (ADCC).

The domains of natural light chain variable regions and heavy chain variable regions can have the same general structures, and each domain can comprise four framework regions, whose sequences can be somewhat conserved, connected by three hyper-variable regions or CDRs. The four framework regions can largely adopt a β-sheet conformation and the CDRs can form loops connecting, and in some aspects forming part of, the β-sheet structure. The CDRs in each chain can be held in close proximity by the framework regions and, with the CDRs from the other chain, can contribute to the formation of the antigen binding site.

An antibody of an antibody construct can comprise an antibody of any type, which can be assigned to different classes of immunoglobins, e.g., IgA, IgD, IgE, IgG, and IgM. Several different classes can be further divided into isotypes, e.g., IgG1, IgG2, IgG3, IgG4, IgA1, and IgA2. An antibody can further comprise a light chain and a heavy chain, often more than one chain. The heavy-chain constant regions (Fc) that corresponds to the different classes of immunoglobulins can be α, δ, ε, γ, and μ, respectively. The light chains can be one of either kappa or κ and lambda or λ, based on the amino acid sequences of the constant domains. The Fc region can comprise an Fc binding domain. An Fc receptor can bind to an Fc binding domain. A conjugate can also comprise any fragment or recombinant form thereof, including but not limited to a scFv, Fab, variable Fc fragment, domain antibody, and any other fragment thereof that can specifically bind to an antigen.

An antibody can comprise an antigen binding domain which refers to a portion of an antibody comprising the antigen recognition portion, i.e., an antigenic determining variable region of an antibody sufficient to confer recognition and specific binding of the antigen recognition portion to a target, such as an antigen, i.e., at an epitope. Examples of antibody binding domains can include, but are not limited to, Fab, variable Fv fragment and other fragments, combinations of fragments or types of fragments known or knowable to one of ordinary skill in the art.

An antigen binding domain of an antibody can comprise one or more light chain (LC) CDRs (LCDRs) and one or more heavy chain (HC) CDRs (HCDRs), one or more LCDRs or one or more HCDRs. For example, an antibody binding domain of an antibody can comprise one or more of the following: a light chain complementary determining region 1 (LCDR1), a light chain complementary determining region 2 (LCDR2), or a light chain complementary determining region 3 (LCDR3). For another example, an antibody binding domain can comprise one or more of the following: a heavy chain complementary determining region 1 (HCDR1), a heavy chain complementary determining region 2 (HCDR2), or a heavy chain complementary determining region 3 (HCDR3). In some embodiments an antibody binding domain comprises all of the following: a light chain complementary determining region 1 (LCDR1), a light chain complementary determining region 2 (LCDR2), a light chain complementary determining region 3 (LCDR3), a heavy chain complementary determining region 1 (HCDR1), a heavy chain complementary determining region 2 (HCDR2), and a heavy chain complementary determining region 3 (HCDR3). Unless stated otherwise, the CDRs described herein can be defined according to the IMGT (the international ImMunoGeneTics information system). An antigen binding domain can comprise only the heavy chain of an antibody (e.g., does not include any other portion of the antibody). An antigen binding domain can comprise only the variable domain of the heavy chain of an antibody. Alternatively, an antigen binding domain can comprise only the light chain of an antibody. An antigen binding domain can comprise only the variable light chain of an antibody.

An antibody construct can comprise an antibody fragment, such as a Fab, a Fab′, a F(ab′)₂ or an Fv fragment. An antibody used herein can be “humanized.” Humanized forms of non-human (e.g., murine) antibodies can be intact (full length) chimeric immunoglobulins, immunoglobulin chains or antigen binding fragments thereof (such as Fv, Fab, Fab′, F(ab′)₂ or other target-binding subdomains of antibodies), which can contain sequences derived from non-human immunoglobulin. In general, the humanized antibody can comprise substantially all of at least one, and typically two, variable domains, in which all or substantially all of the CDR regions correspond to those of a non-human immunoglobulin and all or substantially all of the framework (FR) regions are those of a human immunoglobulin sequence. The humanized antibody can also comprise at least a portion of an immunoglobulin constant region (Fc), typically that of a human immunoglobulin consensus sequence.

An antibody described herein can be a human antibody. As used herein, “human antibodies” can include antibodies having, for example, the amino acid sequence of a human immunoglobulin and include antibodies isolated from human immunoglobulin libraries or from animals transgenic for one or more human immunoglobulins and that typically do not express endogenous immunoglobulins. Human antibodies can be produced using transgenic mice which are incapable of expressing functional endogenous immunoglobulins, but which can express human immunoglobulin genes. Completely human antibodies that recognize a selected epitope can be generated using guided selection. In this approach, a selected non-human monoclonal antibody, e.g., a mouse antibody, is used to guide the selection of a completely human antibody recognizing the same epitope

An antibody described herein can be a bispecific antibody or a dual variable domain antibody (DVD). Bispecific and DVD antibodies are monoclonal, often human or humanized, antibodies that have binding specificities for at least two different antigens.

An antibody described herein can be derivatized or otherwise modified. For example, derivatized antibodies can be modified by glycosylation, acetylation, pegylation, phosphorylation, amidation, derivatization by known protecting/blocking groups, proteolytic cleavage, or the like.

An antibody described herein can specifically bind to an antigen that is expressed on a liver cell. For example, an antibody can specifically bind to a liver cell antigen. A liver cell antigen can be expressed on a canalicular cell, Kupffer cell, hepatocyte, a stellate cell, or other liver cell type, or a combination thereof. The liver cell antigen can be a liver cell surface receptor. The liver cell antigen can be a hepatocyte antigen. The liver cell antigen can be expressed on a non-cancerous liver cell. The liver cell antigen can be expressed on a cell infected with a virus. The virus can be a hepatitis virus, such as HBV or HCV. The virus can be HBV. The virus can be HCV. A liver cell antigen can include, but is not limited to, asialoglycoprotein receptor 1 (ASGR1), asialoglycoprotein receptor 2 (ASGR2), transferrin receptor 2 (TRF2), UDP glucuronosyltransferase 1 family, polypeptide A1 (UGT1A1), solute carrier family 22 member 7 (SLC22A7), solute carrier family 13 member 5 (SLC13A5), solute carrier family 22 member 1 (SLC22A1), and complement component 9 (C9).

In other embodiments, an antibody can bind to a viral antigen from a virus infecting a liver cell. A viral antigen can be expressed on a canalicular cell, Kupffer cell, hepatocyte, a stellate cell, or other liver cell type, or a combination thereof, when infected by a virus. The virus can be a hepatitis virus, such as HBV or HCV. The virus can be HBV. The virus can be HCV. The viral antigen can be expressed on a non-cancerous liver cell infected with a virus. A viral antigen can include, but is not limited to, the components of HBV such as HBsAg, HBcAg, HBeAg, Hepatitis B virus DNA polymerase, or HBx, and components of HCV such as Core protein, E1 and E2, NS2, NS3, NS4A, NS4B, NS5A, or NS5B. In some embodiments, the viral antigen is HBsAg, HBcAg or HBeAg. In some embodiments, the viral antigen is HBsAg.

An antibody construct can comprise an antibody with modifications occurring at least at one amino acid residue. Modifications can be substitutions, additions, mutations, deletions, or the like. An antibody modification can be an insertion of an unnatural amino acid.

An antibody construct can comprise a light chain of an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. A conjugate or antibody construct can comprise a heavy chain of an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence.

An antibody construct can comprise an Fc domain of an IgG1 isotype. An antibody construct can comprise an Fc binding domain of an IgG2 isotype. An antibody construct can comprise an Fc binding domain of an IgG3 isotype. An antibody construct can comprise an Fc domain of an IgG4 isotype. An antibody construct can have a hybrid isotype comprising constant regions from two or more isotypes.

An antibody described herein can have a sequence that has been modified to alter at least one constant region-mediated biological effector function relative to the corresponding wild type sequence. For example, in some embodiments, the antibody can be modified to increase or decrease at least one constant region-mediated biological effector function relative to an unmodified antibody, e.g., increased binding to an Fc receptor (FcR). FcR binding can be reduced or increased by, for example, mutating the immunoglobulin constant region segment of the antibody at particular regions necessary for FcR interactions.

An antibody described herein can be modified to acquire or improve at least one constant region-mediated biological effector function relative to an unmodified antibody, e.g., to enhance FcγR interactions. For example, an antibody with a constant region that binds FcγRIIA, FcγRIIB and/or FcγRIIIA with greater affinity than the corresponding wild type constant region can be produced according to the methods described herein.

An antibody construct can comprise a first binding domain, a second binding domain, and an Fc domain, wherein the first binding domain is attached to the Fc domain. A conjugate or antibody construct can comprise a first binding domain, a second binding domain, and an Fc domain, wherein the second binding domain is attached to the Fc domain. A first binding domain can be attached to an Fc domain as a fusion protein. A second binding domain can be attached to an Fc domain as a fusion protein. A first binding domain can be attached to an Fc domain via a linker. A second binding domain can be attached to an Fc domain via a linker.

### Fusion Proteins

The first antigen binding domain and the second antigen binding domain (if present) can be attached to the Fc domain as a fusion protein. The first antigen binding domain can be attached to the Fc binding domain at an N-terminal end of the Fc binding domain, wherein the second antigen binding domain can be attached to the Fc binding domain at a C-terminal end. The first antigen binding domain can be attached to the Fc binding domain at an N-terminal end of the Fc binding domain, wherein the second antigen binding domain can be attached to the Fc binding domain at a C-terminal end via a polypeptide linker. In some embodiments, the polypeptide linker ranges from 10 to 25 amino acids and can, for example, comprise the sequence [G4S]n where n=2 to 5. Alternatively, the first antigen binding domain can be attached to the Fc binding domain at a C-terminal end of the Fc binding domain, wherein the second antigen binding domain can be attached to the Fc binding domain at an N-terminal end. A second antigen binding domain and an Fc binding domain can comprise an antibody and a first binding domain can comprise a single chain variable fragment (scFv). A single chain variable fragment can comprise a heavy chain variable domain and a light chain variable domain of an antibody. The first antigen binding domain of the fusion protein can be attached to the second antigen binding domain at a heavy chain variable domain of the single chain variable fragment of the first antigen binding domain (HL orientation). Alternatively, the first antigen binding domain of the fusion protein can be attached to the second antigen binding domain at a light chain variable domain of the single chain variable fragment of the first binding domain (LH orientation). In either orientation, the first antigen binding domain and the second antigen binding domain can be attached via a polypeptide linker. In some embodiments, the polypeptide linker can vary in length from 15 to 25 amino acids, and can, for example, comprise the sequence [G4S]n where n=3 to 5.

Alternatively, a first antigen binding domain and an Fc binding domain can comprise an antibody and the second antigen binding domain can comprise a single chain variable fragment (scFv). The second antigen binding domain of the fusion protein can be attached to the first antigen binding domain at a heavy chain variable domain of the single chain variable fragment of the first antigen binding domain (HL orientation). Alternatively, the second antigen binding domain of the fusion protein can be attached to the first antigen binding domain at a light chain variable domain of the single chain variable fragment of the first antigen binding domain (LH orientation).

An antibody construct can comprise a first antigen binding domain and a second antigen binding domain, wherein the second antigen binding domain can be attached to the first antigen binding domain. The antibody construct can comprise an antibody comprising a light chain and a heavy chain. The first antigen binding domain can comprise a Fab fragment of the light and heavy chains. The second antigen binding domain can be attached to the light chain at a C-terminus or C-terminal end of the light chain as a fusion protein. The second antigen binding domain can comprise a single chain variable fragment (scFv).

An antibody construct can comprise a first antigen binding domain, a second antigen binding domain, and an Fc binding domain, wherein the first antigen binding domain and the second antigen binding domain are attached to the Fc binding domain as a fusion protein. The second antigen binding domain of the fusion protein can specifically bind to a second antigen on a liver cell, wherein the second antigen is a second liver cell antigen or a second viral antigen from a virus infecting a liver cell. In some embodiments, the second antigen binding domain of the fusion protein can specifically bind to an antigen selected from the group consisting of ASGR1, ASGR2, TRF2, UGT1A1, SCL22A7, SCL12A5, SLC22A1, C9. The second antigen binding domain of the fusion protein can specifically bind to an antigen selected from the group consisting of ASGR1, ASGR2, TRF2, UGT1A1, SCL22A7, SCL12A5 and SLC22A1. The second antigen binding domain of the fusion protein can specifically bind to an antigen selected from the group consisting of ASGR1, ASGR2 and TRF2. The second liver cell antigen can be ASGR1. The second liver cell antigen can be ASGR2. The second liver cell antigen can be TRF2. The second antigen binding domain of the fusion protein can specifically bind to an antigen with an amino acid sequence comprising at least 80% identity to an antigen selected from the group consisting of ASGR1, ASGR2, TRF2, UGT1A1, SCL22A7, SCL12A5, SLC22A1, and C9.

In some embodiments, the second antigen binding domain of the fusion protein can specifically bind to a viral antigen selected from the group consisting of HBsAg, HBcAg, HBeAg, Hepatitis B virus DNA polymerase, HBx, Core protein, E1 and E2, NS2, NS3, NS4A, NS4B, NS5A, and NS5B. In some embodiments, the second antigen binding domain of the fusion protein can specifically bind to HBsAg, HBcAg or HBeAg. In some embodiments, the second antigen binding domain of the fusion protein can specifically bind to HBsAg.

The first binding domain of the fusion protein can specifically bind to a first antigen, wherein the first antigen is a liver cell antigen or a viral antigen from a virus infecting the liver cell. In some embodiments, the first antigen binding domain of the fusion protein can specifically bind to a liver cell antigen. The first antigen binding domain of the fusion protein can specifically bind to a liver cell antigen selected from the group consisting of ASGR1, ASGR2, TRF2, UGT1A1, SCL22A7, SCL12A5, SLC22A1 and C9. The first antigen binding domain of the fusion protein can specifically bind to a liver cell antigen selected from the group consisting of ASGR1, ASGR2, TRF2, UGT1A1, SCL22A7, SCL12A5 and SLC22A1. The first antigen binding domain of the fusion protein can specifically bind to a liver cell antigen selected from the group consisting of ASGR1, ASGR2, and TRF2. The first antigen binding domain of the fusion protein can specifically bind to ASGR1. The first antigen binding domain of the fusion protein can specifically bind to ASGR2. The first antigen binding domain of the fusion protein can specifically bind to TRF2. The first antigen binding domain of the fusion protein can specifically bind to a liver cell antigen with an amino acid sequence comprising at least 80% identity to an antigen selected from the group consisting of ASGR1, ASGR2, TRF2, UGT1A1, SCL22A7, SCL12A5, SLC22A1 and C9. The first antigen binding domain of the fusion protein can specifically bind to a viral antigen selected from the group consisting of HBsAg, HBcAg, HBeAg, Hepatitis B virus DNA polymerase, HBx, Core protein, E1 and E2, NS2, NS3, NS4A, NS4B, NS5A, and NS5B. The first antigen binding domain of the fusion protein can specifically bind to HBsAg, HBcAg or HBeAg. The first antigen binding domain of the fusion protein can specifically bind to HBsAg. The first antigen binding domain of the fusion protein can specifically bind to HBcAg. The first antigen binding domain of the fusion protein can specifically bind to a viral antigen with an amino acid sequence comprising at least 80% identity to an antigen selected from the group consisting of HBsAg, HBcAg, HBeAg, Hepatitis B virus DNA polymerase, HBx, Core protein, E1 and E2, NS2, NS3, NS4A, NS4B, NS5A, and NS5B.

The first antigen binding domain and the second antigen binding domain each can specifically bind to a different antigen. The first antigen binding domain and the second antigen binding domain each can specifically bind to a different epitope of the same antigen.

An antibody construct can comprise a first antigen binding domain, a second antigen binding domain, and an Fc binding domain, wherein the second antigen binding domain can be attached to the first antigen binding domain. The second antigen binding domain can be attached at a C-terminal end of the first antigen binding domain as a fusion protein. The first antigen binding domain can comprise a Fab fragment comprising a light chain, wherein the second antigen binding domain can be attached at a C-terminal end of the light chain as a fusion protein. The second antigen binding domain of the fusion protein can comprise a single chain variable fragment (scFv). The second antigen binding domain of the fusion protein can be attached to the first antigen binding domain at a heavy chain variable domain of the single chain variable fragment of the first binding domain (HL orientation). Alternatively, the second antigen binding domain of the fusion protein can be attached to the first antigen binding domain at a light chain variable domain of the single chain variable fragment of the first binding domain (LH orientation). All fusion sequences comprising a scFv sequence are in the HL orientation unless indicated otherwise (e.g., sequence name recites “(LH)” indicating light heavy orientation).

An antibody construct can comprise a first antigen binding domain targeting a liver cell antigen and a second antigen binding domain targeting a viral antigen from a virus infecting a liver cell. Alternatively, an antibody construct can comprise a first antigen binding domain targeting a viral antigen from a virus infecting a liver cell and a second antigen binding domain targeting a liver cell antigen. The first antigen binding domain and the second antigen binding domain can be attached to the Fc binding domain. The first antigen binding domain can be attached to the Fc binding domain at an N-terminal end of the Fc binding domain, wherein the second antigen binding domain is attached to the Fc binding domain at a C-terminal end of the Fc binding domain. Alternatively, second antigen binding domain can be attached to the Fc binding domain at an N-terminal end of the Fc binding domain, wherein the first antigen binding domain is attached to the Fc binding domain at a C-terminal end of the Fc binding domain.

Additionally, antibody constructs as described herein can have a dissociation constant (Kd) that is less than 10 nM for the antigen of the first antigen binding domain. The antibody constructs can have a dissociation constant (Kd) that is less than 10 nM for the antigen of the second antigen binding domain. The antibody constructs can have a dissociation constant (Kd) for the antigen of the first antigen binding domain that is less than 1 nM, less than 100 pM, less than 10 pM, less than 1 pM, or less than 0.1 pM. The antibody constructs can have a dissociation constant (Kd) for the antigen of the second antigen binding domain that is less than 1 nM, less than 100 pM, less than 10 pM, less than 1 pM, or less than 0.1 pM.

An antibody construct disclosed herein can be non-natural, designed, and/or engineered. Antibody constructs disclosed herein can be non-natural, designed, and/or engineered scaffolds comprising an antigen binding domain. Antibody constructs disclosed herein can be non-natural, designed, and/or engineered antibodies. Antibody constructs can include monoclonal antibodies.

Antibody constructs can comprise human antibodies. Antibody constructs can comprise humanized antibodies. Antibody constructs can comprise monoclonal humanized antibodies. Conjugates and antibody constructs can comprise recombinant antibodies.

### Myeloid Cell Agonists

The antibody constructs described herein are attached to a myeloid cell agonist to form a conjugate. The myeloid cell agonist can provide a direct or indirect effect. In certain embodiments, the myeloid cell agonist can be coupled to the antibody construct, such as to the Fc binding domain of the antibody construct. A myeloid cell agonist can be any compound that directly or indirectly stimulates an anti-viral response. For example, a myeloid cell agonist can directly stimulate an anti-viral response by causing the release of cytokines by myeloid cells, which results in the activation of immune cells. As another example, a myeloid cell agonist can indirectly stimulate an immune response by suppressing IL-10 production and secretion by the myeloid cell and/or by suppressing the activity of regulatory T cells, resulting in an increased anti-viral response by immune cells. The stimulation of an immune response by a myeloid cell agonist can be measured by the upregulation of proinflammatory cytokines and/or increased activation of immune cells. This effect can be measured in vitro by co-culturing immune cells with liver cells targeted by the conjugate and measuring cytokine release, chemokine release, proliferation of immune cells, upregulation of immune cell activation markers, and/or ADCC. ADCC can be measured by determining the percentage of remaining virus or infected cells in the co-culture after administration of the conjugate with the liver cells, myeloid cells, and other immune cells.

In certain embodiments, the myeloid cell agonist is a TLR7 agonist and/or a TLR8 agonist. In certain embodiments, the myeloid cell agonist can be a TLR7 agonist. In some embodiments, the myeloid agonist selectively agonizes TLR7 and not TLR8. In other embodiments, the myeloid agonist selectively agonizes TLR8 and not TLR7.

In certain embodiments, the TLR7 agonist is selected from an imidazoquinoline, an imidazoquinoline amine, a thiazoquinoline, a guanosine analog, an adenosine analog, a thymidine homopolymer, ssRNA, CpG-A, PolyG10, and PolyG3. In some embodiments, the TLR7 agonist is selected from the group consisting of gardiquimod, imiquimod, resiquimod, GS-9620, or imidazoquinoline 852A.

In certain embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In certain embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide or a benzonaphthyridine, but is other than a guanosine analog, an adenosine analog, a thymidine homopolymer, ssRNA, CpG-A, PolyG10, and PolyG3. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20160168164 (Janssen), US 20150299194 (Roche), US20110098248 (Gilead Sciences), US20100143301 (Gilead Sciences), and US20090047249 (Gilead Sciences). In some embodiments, a TLR7 agonist has an EC50 value of 500 nM or less by PBMC assay measuring TNFalpha or IFNalpha production. In some embodiments, a TLR7 agonist has an EC50 value of 100 nM or less by PBMC assay measuring TNFalpha or IFNalpha production. In some embodiments, a TLR7 agonist has an EC50 value of 50 nM or less by PBMC assay measuring TNFalpha or IFNalpha production. In some embodiments, a TLR7 agonist has an EC50 value of 10 nM or less by PBMC assay measuring TNFalpha or IFNalpha production.

In certain embodiments the myeloid cell agonist can be a TLR8 agonist. In certain embodiments, a TLR8 agonist is selected from the group consisting of a benzazepine, a ssRNA, an imidazoquinoline, a thiazoloquinolone and an aminoquinoline. In certain embodiments, the TLR8 agonist is selected from VTX-2337, VTX-294, and resiquimod.

In certain embodiments, the TLR8 agonist is benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In certain embodiments, a TLR8 agonist is selected from the group consisting of a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine and is other than a ssRNA. In some embodiments, a TLR8 agonist is a non-naturally occurring compound. Examples of TLR8 agonists include motolimod, resiquimod, 3M-051, 3M-052, MCT-465, IMO-4200, VTX-763, VTX-1463. In some embodiments, a TLR8 agonist has an EC50 value of 500 nM or less by PBMC assay measuring TNFalpha production. In some embodiments, a TLR8 agonist has an EC50 value of 100 nM or less by PBMC assay measuring TNFalpha production. In some embodiments, a TLR8 agonist has an EC50 value of 50 nM or less by PBMC assay measuring TNFalpha production. In some embodiments, a TLR8 agonist has an EC50 value of 10 nM or less by PBMC assay measuring TNFalpha production.

In some embodiments, a TLR8 agonist is any of compounds 1.1-1.2, 1.4-1.20, 1.23-1.27, 1.29-1.46, 1.48, and 1.50-1.67, as shown in the Examples.

Other TLR7 and TLR8 agonists are disclosed in, for example, WO 2016142250, WO2017046112, WO2007024612, WO2011022508, WO2011022509, WO2012045090, WO2012097173, WO2012097177, WO2017079283, US20160008374, US20160194350, US20160289229, U.S. Pat. No. 6043238, US20180086755 (Gilead), WO2017216054 (Roche), WO2017190669 (Shanghai De Novo Pharmatech), WO2017202704 (Roche), WO2017202703 (Roche), WO20170071944 (Gilead), US20140045849 (Janssen), US20140073642 (Janssen), WO2014056953 (Janssen), WO2014076221 (Janssen), WO2014128189 (Janssen), US20140350031 (Janssen), WO2014023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics) incorporated herein by reference for all purposes.

In certain aspects, TLR8 agonists and TLR7 agonists are selected from Category A or Category B, respectively. Variables and Formula of the Compounds of Category A (TLR8 agonists) are described in the section entitled Compounds of Category A, and variables and Formula of the Compounds of Category B (TLR7 agonists) are described in the subsequent section, entitled Compounds of Category B. Formulas and variables of the Compounds of Category A and the Compounds of Category B may overlap in nomenclature, e.g., Formula IA for both Compounds of Category A and Category B; however variables and Formula descriptions are not intended to be interchangeable between the catagories.

Compounds of Category A, TLR8 Agonists

In some aspects, the present disclosure provides a TLR8 agonist represented by the structure of Formula (IIA):

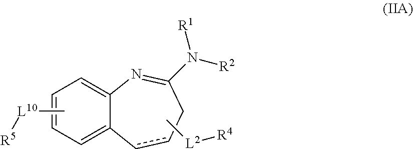

or a pharmaceutically acceptable salt thereof, wherein:

[symbol] represents an optional double bond;

L¹⁰ is —X¹⁰—;

L² is selected from —X²—, —X²—C₁₋₆ alkylene-X²—, —X²—C₂₋₆ alkenylene-X²—, and —X²—C₂₋₆ alkynylene-X²—, each of which is optionally substituted on alkylene, alkenylene or alkynylene with one or more R¹²;

X¹⁰ is selected from —C(O)—, and —C(O)N(R¹⁰)—*, wherein * represents where X¹⁰ is bound to R⁵;

X² at each occurrence is independently selected from a bond, —O—, —S—, —N(R¹⁰)—, —C(O)—, —C(O)O—, —OC(O)—, —OC(O)O—, —C(O)N(R¹⁰)—, —C(O)N(R¹⁰)C(O)—, —C(O)N(R¹⁰)C(O)N(R¹⁰), —N(R¹⁰)C(O)—, —N(R¹⁰)C(O)N(R¹⁰)—, —N(R¹⁰)C(O)O—, —OC(O)N(R¹⁰)—, —C(NR¹⁰)—, —N(R¹⁰)C(NR¹⁰)—, —C(NR¹⁰)N(R¹⁰)—, —N(R¹⁰)C(NR¹⁰)N(R¹⁰)—, —S(O)₂—, —OS(O)—, —S(O)O—, —S(O), —OS(O)₂—, —S(O)₂O, —N(R¹⁰)S(O)₂—, —S(O)₂N(R¹⁰)—, —N(R¹⁰)S(O)—, —S(O)N(R¹⁰)—, —N(R¹⁰)S(O)₂N(R¹⁰)—, and —N(R¹⁰)S(O)N(R¹⁰)—;

R¹ and R² are independently selected from hydrogen; and C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), and —CN;

R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)R¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, wherein each C₃₋₁₂ carbocycle, and 3- to 12-membered heterocycle in R⁴ is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;

R⁵ is selected from unsaturated C₄₋₈ carbocycle; bicyclic carbocycle; and fused 5-5, fused 5-6, and fused 6-6 bicyclic heterocycle, wherein R⁵ is optionally substituted and wherein substituents are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, wherein each C₃₋₁₂ carbocycle, and 3- to 12-membered heterocycle in R⁵ is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;

R¹⁰ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and haloalkyl;

R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹² is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, C₂₋₆ alkynyl; and

wherein any substitutable carbon on the benzazepine core is optionally substituted by a substituent independently selected from R¹² or two substituents on a single carbon atom combine to form a 3- to 7-membered carbocycle.

In some embodiments, the compound of Formula (IIA) is represented by Formula (IIB):

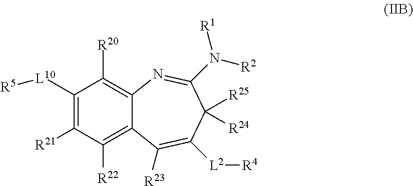

or a pharmaceutically acceptable salt thereof, wherein:

R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and

R²⁴, and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments, R²⁰, R²¹, R²², and R²³ are independently selected from hydrogen, halogen, —OH, —OR¹⁰, —NO₂, —CN, and C₁₋₁₀ alkyl. R²⁰, R²¹, R²², and R²³ may be each hydrogen. In certain embodiments, R²¹ is halogen. In certain embodiments, R²¹ is hydrogen. In certain embodiments, R²¹ is —OR¹⁰. For example, R²¹ may be —OCH₃.

In some embodiments, R²⁴ and R²⁵ are independently selected from hydrogen, halogen, —OH, —NO₂, —CN, and C₁₋₁₀ alkyl, or R²⁴ and R²⁵ taken together form an optionally substituted saturated C₃₋₇ carbocycle. In certain embodiments, R²⁴ and R²⁵ are each hydrogen. In other embodiments, R²⁴ and R²⁵ taken together form an optionally substituted saturated C₃₋₅ carbocycle, wherein substituents are selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; and C₁₋₁₀ alkyl, C₂₋₁₀ alkenyl, C₂₋₁₀ alkynyl, each of which is independently optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle.

In some embodiments, R¹ is hydrogen. In some embodiments, R² is hydrogen. In some embodiments, R² is —C(O)—.

In some embodiments, L¹⁰ is selected from —C(O)N(R¹⁰)—*. In certain embodiments, R¹⁰ of —C(O)N(R¹⁰)—* is selected from hydrogen and C₁₋₆ alkyl. For example, L¹⁰ may be —C(O)NH—*.

In some embodiments, R⁵ is an optionally substituted bicyclic carbocycle. In certain embodiments, R⁵ is an optionally substituted 8- to 12-membered bicyclic carbocycle. R⁵ may be an optionally substituted 8- to 12-membered bicyclic carbocycle substituted with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. In certain embodiments, R⁵ is an optionally substituted 8- to 12-membered bicyclic carbocycle substituted with one or more substituents independently selected from —OR¹⁰, —N(R¹⁰)₂, and ═O. In some embodiments, R⁵ is an optionally substituted indane, and optionally substituted tetrahydronaphthalene. R⁵ may be selected from:

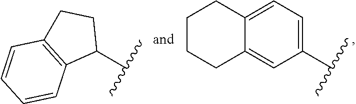 [SMILES: *C1CCc2ccccc21 ; "and" ; *c1ccc2c(c1)CCCC2 ; ","]

any one of which is optionally substituted. For example, the R⁵ is selected from:

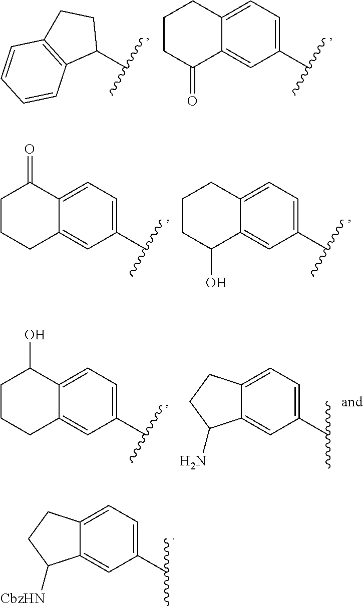

In some embodiments, R⁵ is an optionally substituted unsaturated C₄₋₈ carbocycle. In certain embodiments, R⁵ is an optionally substituted unsaturated C₄₋₆ carbocycle. In certain embodiments, R⁵ is an optionally substituted unsaturated C₄₋₆ carbocycle with one or more substituents independently selected from optionally substituted C₃₋₁₂ carbocycle, and optionally substituted 3- to 12-membered heterocycle. R⁵ may be an optionally substituted unsaturated C₄₋₆ carbocycle with one or more substituents independently selected from optionally substituted phenyl, optionally substituted 3- to 12-heterocycle, optionally substituted C₁₋₁₀ alkyl, optionally substituted C₂₋₁₀ alkenyl, and halogen.

In some embodiments, R⁵ is selected from an optionally substituted fused 5-5, fused 5-6, and fused 6-6 bicyclic heterocycle. In certain embodiments, R⁵ is an optionally substituted fused 5-5, fused 5-6, and fused 6-6 bicyclic heterocycle with one or more substituents independently selected from)—C(O)OR¹⁰, —N(R¹⁰)₂, —OR¹⁰, and optionally substituted C₁₋₁₀ alkyl. In certain embodiments, R⁵ is an optionally substituted fused 5-5, fused 5-6, and fused 6-6 bicyclic heterocycle substituted with —C(O)OR¹⁰. In certain embodiments, R⁵ is an optionally substituted fused 6-6 bicyclic heterocycle. For example, the fused 6-6 bicyclic heterocycle may be an optionally substituted pyridine-piperidine. In some embodiments, L¹⁰ is bound to a carbon atom of the pyridine of the fused pyridine-piperidine. In certain embodiments, R⁵ is selected from tetrahydroquinoline, tetrahydroisoquinoline, tetrahydronaphthyridine, cyclopentapyridine, and dihydrobenzoxaborole, any one of which is optionally substituted. R⁵ may be an optionally substituted tetrahydronaphthyridine. In some embodiments, R⁵ is selected from:

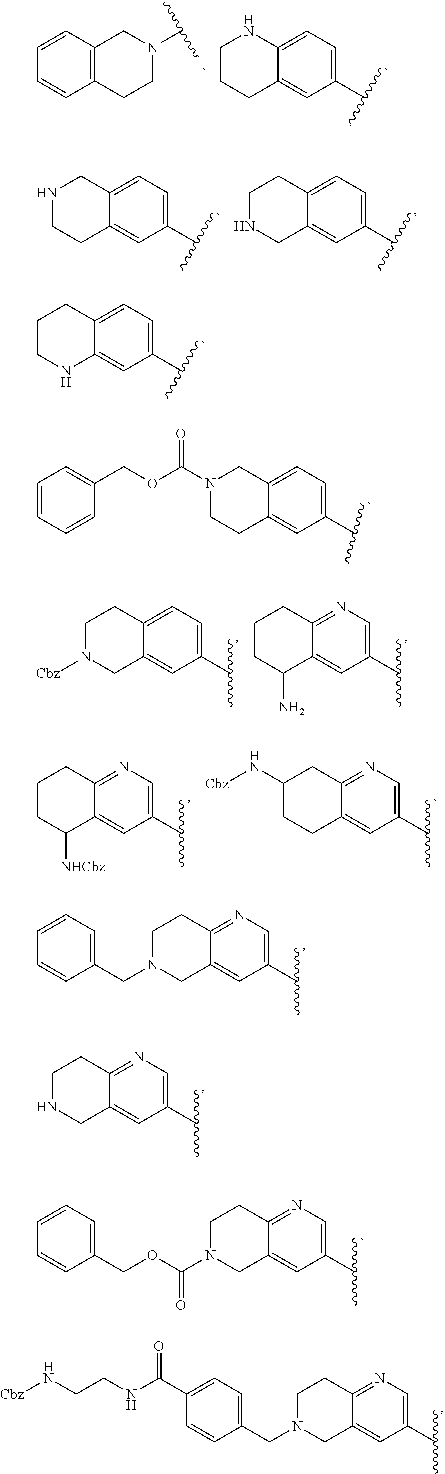

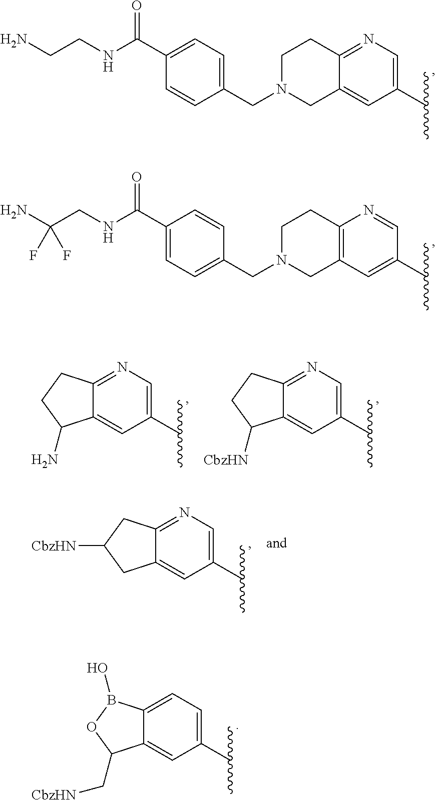 [SMILES: *c1cnc2c(c1)CN(Cc1ccc(C(=O)NCCN)cc1)CC2 ; *c1cnc2c(c1)CN(Cc1ccc(C(=O)NCC(N)(F)F)cc1)CC2 ; *c1cnc2c(c1)C(N)CC2 ; *c1cnc2c(c1)C(NC(=O)OCc1ccccc1)CC2 ; *c1cnc2c(c1)CC(NC(=O)OCc1ccccc1)C2 ; *c1ccc2c(c1)C(CNC(=O)OCc1ccccc1)OB2O]

In some embodiments, when R⁵ is substituted, substituents on R⁵ are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl. In certain embodiments, the substituents on R⁵ are independently selected at each occurrence from: halogen, —OR¹⁰, —SR¹⁰, —C(O)N(R¹⁰)₂, ——N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle. In certain embodiments, the substituents on R⁵ are independently selected at each occurrence from: halogen, —OR¹⁰, —SR¹⁰, —C(O)N(R¹⁰)₂, —N(R¹⁰)C(O)R¹⁰, —N(R¹⁰)₂, —C(O)R¹⁰, —C(O)OR¹⁰, —OC(O)R¹⁰, —NO₂, ═O, and —CN; and C₁₋₁₀ alkyl optionally substituted with one or more substituents independently selected from halogen, —OR¹⁰, —SR¹⁰, —N(R¹⁰)₂, —C(O)R¹⁰, —C(O)OR¹⁰, —NO₂, ═O, and —CN. In some embodiments, R⁵ is not substituted.

In some embodiments, L² is selected from —C(O)—, and —C(O)NR¹⁰—. In some embodiments, L² is —C(O)—. In some embodiments, L² is selected from —C(O)NR¹⁰—. R¹⁰ of —C(O)NR¹⁰— may be selected from hydrogen and C₁₋₆ alkyl. For example, L² may be —C(O)NH—.

In some embodiments, R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle and 3- to 12-membered, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl. In some embodiments, R⁴ is selected from: —OR¹⁰, and —N(R¹⁰)₂; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl. In certain embodiments, R⁴ is —N(R¹⁰)₂. R¹⁰ of —N(R¹⁰)₂ may be independently selected at each occurrence from optionally substituted C₁₋₆ alkyl. In certain embodiments, R¹⁰ of —N(R¹⁰)₂ is independently selected at each occurrence from methyl, ethyl, propyl, and butyl, any one of which is optionally substituted. For example, R⁴ may be

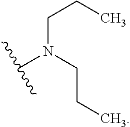 [SMILES: *N(CCC)CCC]

In certain embodiments, -L²-R⁴ is

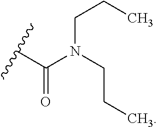 [SMILES: *C(=O)N(CCC)CCC]

In some embodiments, R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰. —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, and C₂₋₆ alkynyl. In certain embodiments, R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; and C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle.

In some embodiments, the compound is selected from:

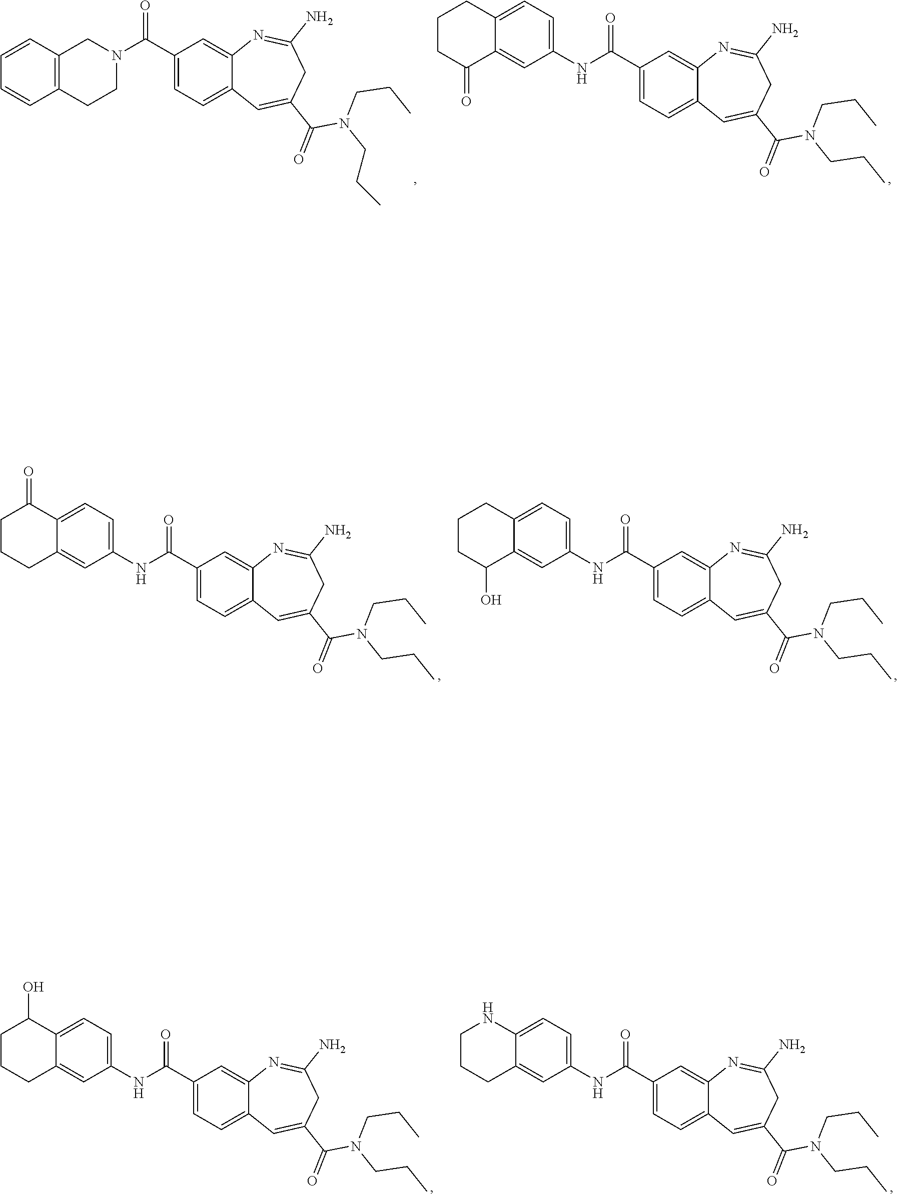 [SMILES: CCCN(CCC)C(=O)C1=Cc2ccc(C(=O)N3CCc4ccccc4C3)cc2N=C(N)C1 ; CCCN(CCC)C(=O)C1=Cc2ccc(C(=O)Nc3ccc4c(c3)C(=O)CCC4)cc2N=C(N)C1 ; CCCN(CCC)C(=O)C1=Cc2ccc(C(=O)Nc3ccc4c(c3)CCCC4=O)cc2N=C(N)C1 ; CCCN(CCC)C(=O)C1=Cc2ccc(C(=O)Nc3ccc4c(c3)C(O)CCC4)cc2N=C(N)C1 ; CCCN(CCC)C(=O)C1=Cc2ccc(C(=O)Nc3ccc4c(c3)CCCC4O)cc2N=C(N)C1 ; CCCN(CCC)C(=O)C1=Cc2ccc(C(=O)Nc3ccc4c(c3)CCCN4)cc2N=C(N)C1]

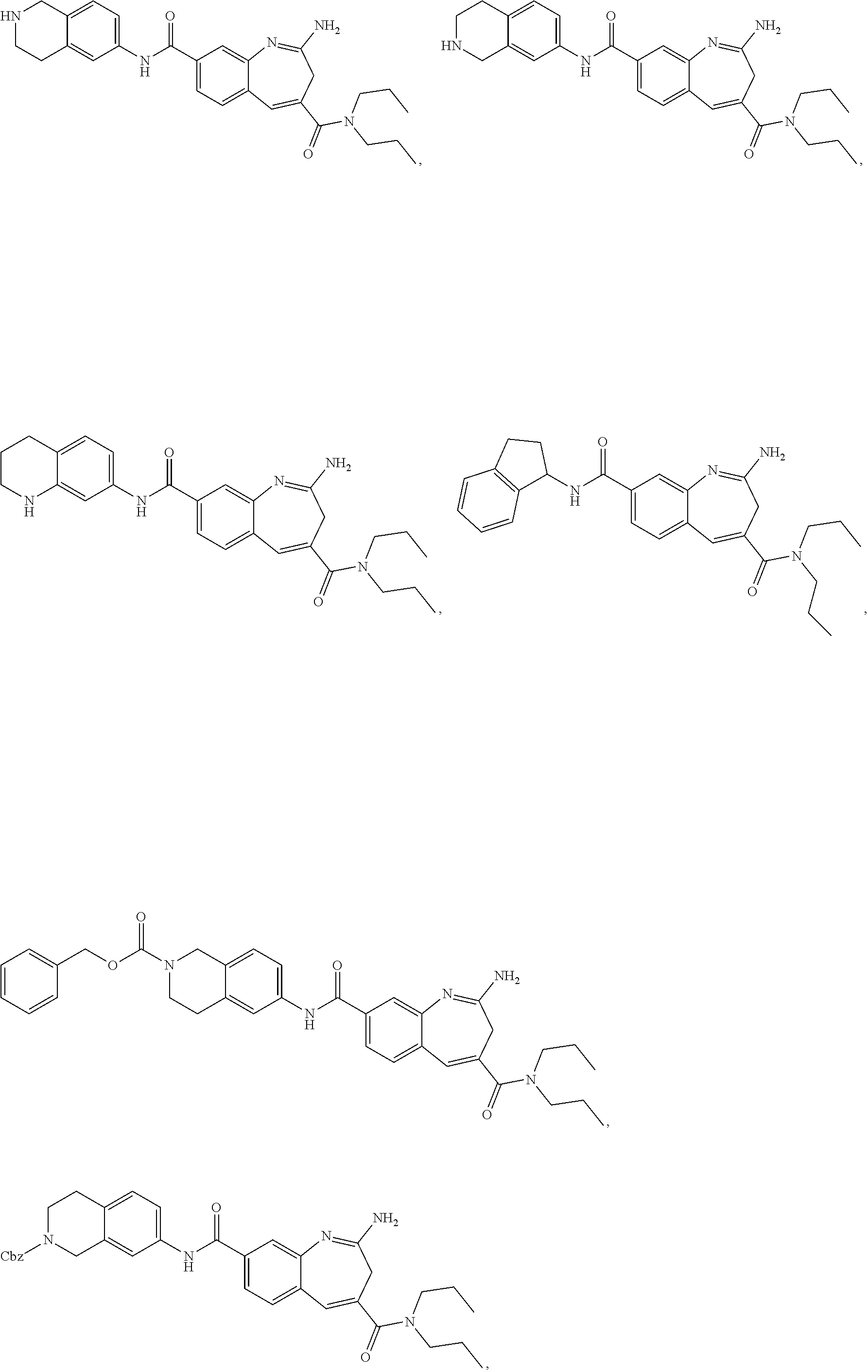 [SMILES: CCCN(CCC)C(=O)C1=Cc2ccc(C(=O)Nc3ccc4c(c3)CCNC4)cc2N=C(N)C1 ; CCCN(CCC)C(=O)C1=Cc2ccc(C(=O)Nc3ccc4c(c3)CNCC4)cc2N=C(N)C1 ; CCCN(CCC)C(=O)C1=Cc2ccc(C(=O)Nc3ccc4c(c3)NCCC4)cc2N=C(N)C1 ; CCCN(CCC)C(=O)C1=Cc2ccc(C(=O)NC3CCc4ccccc43)cc2N=C(N)C1 ; CCCN(CCC)C(=O)C1=Cc2ccc(C(=O)Nc3ccc4c(c3)CCN(C(=O)OCc3ccccc3)C4)cc2N=C(N)C1 ; CCCN(CCC)C(=O)C1=Cc2ccc(C(=O)Nc3ccc4c(c3)CN(C(=O)OCc3ccccc3)CC4)cc2N=C(N)C1]

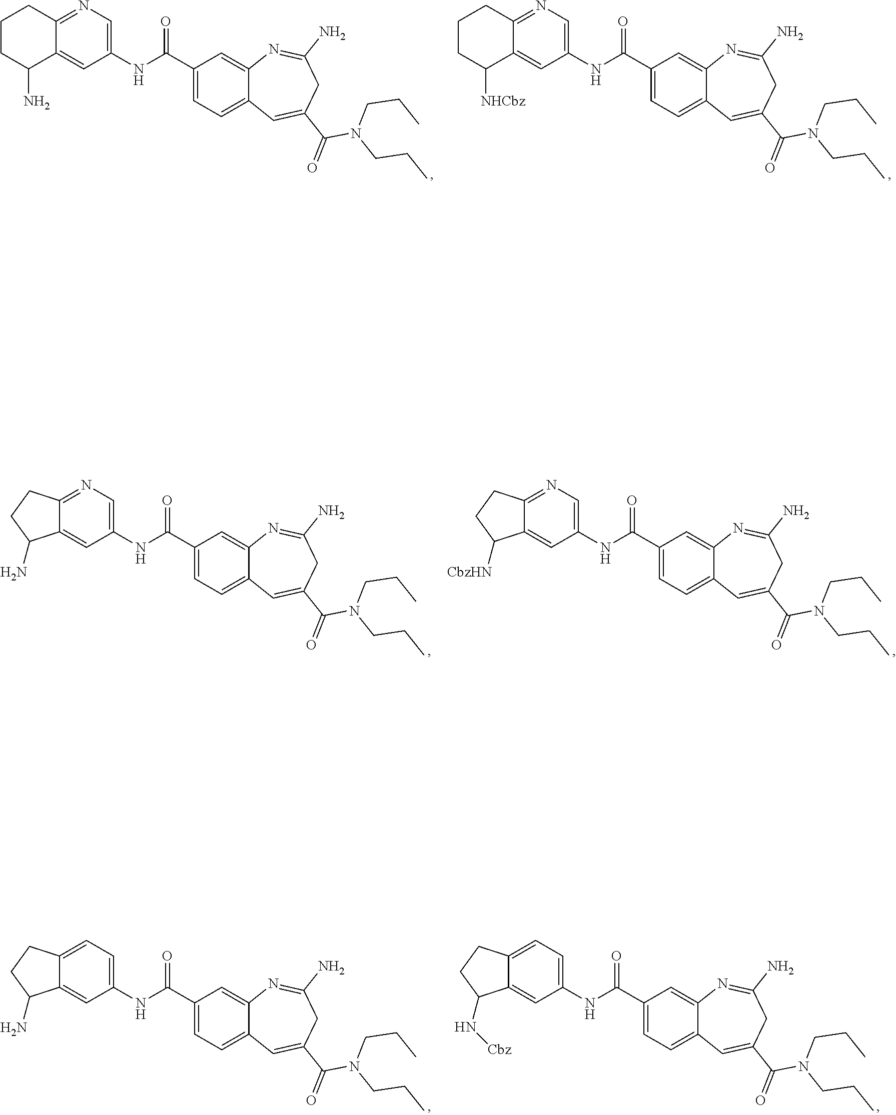 [SMILES: CCCN(CCC)C(=O)C1=Cc2ccc(C(=O)Nc3cnc4c(c3)C(N)CCC4)cc2N=C(N)C1 ; CCCN(CCC)C(=O)C1=Cc2ccc(C(=O)Nc3cnc4c(c3)C(NC(=O)OCc3ccccc3)CCC4)cc2N=C(N)C1 ; CCCN(CCC)C(=O)C1=Cc2ccc(C(=O)Nc3cnc4c(c3)C(N)CC4)cc2N=C(N)C1 ; CCCN(CCC)C(=O)C1=Cc2ccc(C(=O)Nc3cnc4c(c3)C(NC(=O)OCc3ccccc3)CC4)cc2N=C(N)C1 ; CCCN(CCC)C(=O)C1=Cc2ccc(C(=O)Nc3ccc4c(c3)C(N)CC4)cc2N=C(N)C1 ; CCCN(CCC)C(=O)C1=Cc2ccc(C(=O)Nc3ccc4c(c3)C(NC(=O)OCc3ccccc3)CC4)cc2N=C(N)C1]

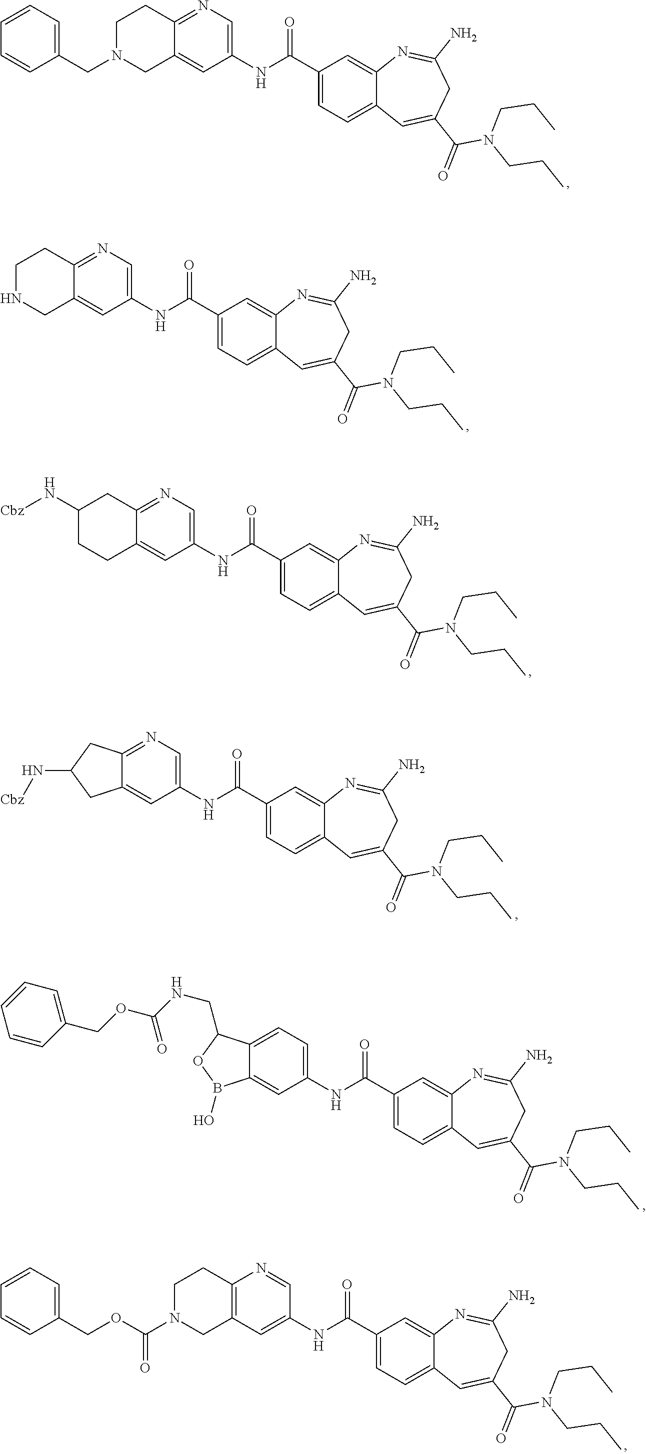 [SMILES: CCCN(CCC)C(=O)C1=Cc2ccc(C(=O)Nc3cnc4c(c3)CN(Cc3ccccc3)CC4)cc2N=C(N)C1 ; CCCN(CCC)C(=O)C1=Cc2ccc(C(=O)Nc3cnc4c(c3)CNCC4)cc2N=C(N)C1 ; CCCN(CCC)C(=O)C1=Cc2ccc(C(=O)Nc3cnc4c(c3)CCC(NC(=O)OCc3ccccc3)C4)cc2N=C(N)C1 ; CCCN(CCC)C(=O)C1=Cc2ccc(C(=O)Nc3cnc4c(c3)CC(NC(=O)OCc3ccccc3)C4)cc2N=C(N)C1 ; CCCN(CCC)C(=O)C1=Cc2ccc(C(=O)Nc3ccc4c(c3)B(O)OC4CNC(=O)OCc3ccccc3)cc2N=C(N)C1 ; CCCN(CCC)C(=O)C1=Cc2ccc(C(=O)Nc3cnc4c(c3)CN(C(=O)OCc3ccccc3)CC4)cc2N=C(N)C1]

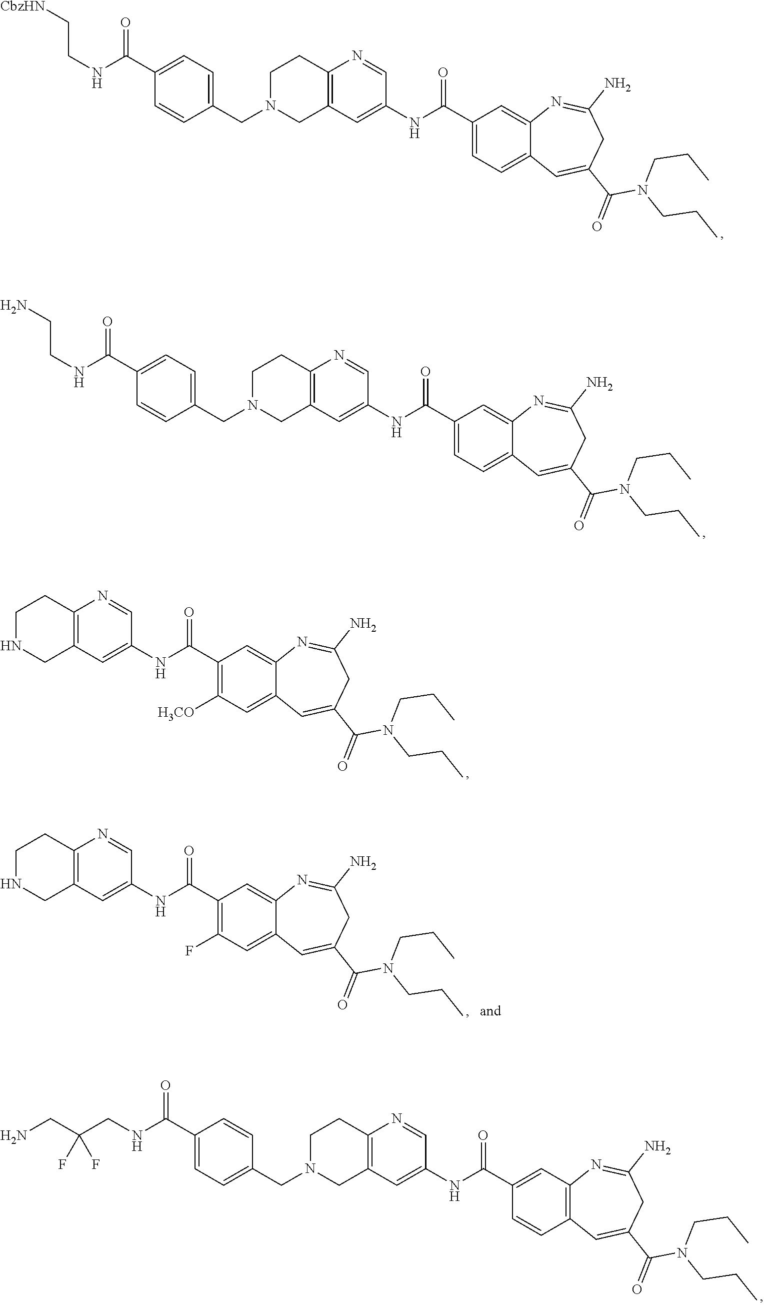 [SMILES: CCCN(CCC)C(=O)C1=Cc2ccc(C(=O)Nc3cnc4c(c3)CN(Cc3ccc(C(=O)NCCNC(=O)OCc5ccccc5)cc3)CC4)cc2N=C(N)C1 ; CCCN(CCC)C(=O)C1=Cc2ccc(C(=O)Nc3cnc4c(c3)CN(Cc3ccc(C(=O)NCCN)cc3)CC4)cc2N=C(N)C1 ; CCCN(CCC)C(=O)C1=Cc2cc(OC)c(C(=O)Nc3cnc4c(c3)CNCC4)cc2N=C(N)C1 ; CCCN(CCC)C(=O)C1=Cc2cc(F)c(C(=O)Nc3cnc4c(c3)CNCC4)cc2N=C(N)C1 ; CCCN(CCC)C(=O)C1=Cc2ccc(C(=O)Nc3cnc4c(c3)CN(Cc3ccc(C(=O)NCC(F)(F)CN)cc3)CC4)cc2N=C(N)C1]

and a salt of any one thereof.

In some aspects, the present disclosure provides a compound represented by the structure of Formula (IIIA):

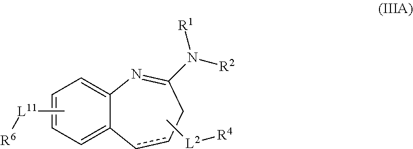

or a pharmaceutically acceptable salt thereof, wherein:

[symbol] represents an optional double bond;

L¹¹ is —X¹¹—;

L² is selected from —X²—, —X²—C₁₋₆ alkylene-X²—, —X²—C₂₋₆ alkenylene-X²—, and —X²—C₂₋₆ alkynylene-X²—, each of which is optionally substituted on alkylene, alkenylene or alkynylene with one or more R¹²;

X¹¹ is selected from —C(O)— and —C(O)N(R¹⁰)—*, wherein * represents where X¹¹ is bound to R⁶;

X² at each occurrence is independently selected from a bond, —O—, —S—, —N(R¹⁰)—, —C(O)—, —C(O)O—, —OC(O)—, —OC(O)O—, —C(O)N(R¹⁰)—, —C(O)N(R¹⁰)C(O)—, —C(O)N(R¹⁰)C(O)N(R¹⁰)—, —N(R¹⁰)C(O)—, —N(R¹⁰)C(O)N(R¹⁰)—, —N(R¹⁰)C(O)O—, —OC(O)N(R¹⁰)—, —C(NR¹⁰)—, —N(R¹⁰)C(NR¹⁰)—, —C(NR¹⁰)N(R¹⁰)—, —N(R¹⁰)C(NR¹⁰)N(R¹⁰)—, —S(O)₂—, —OS(O)—, —S(O)O—, —S(O)—, —OS(O)₂—, —S(O)₂O—, —N(R¹⁰)S(O)₂—, —S(O)₂N(R¹⁰)—, —N(R¹⁰)S(O)—, —S(O)N(R¹⁰)—, —N(R¹⁰)S(O)₂N(R¹⁰)—, and —N(R¹⁰)S(O)N(R¹⁰)—;

R¹ and R² are independently selected from hydrogen; C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), and —CN;

R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, wherein each C₃₋₁₂ carbocycle, and 3- to 12-membered heterocycle in R⁴ is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;

R⁶ is selected from phenyl and 5- or 6-membered heteroaryl, any one of which is substituted with one or more substituents selected from R⁷ and R⁶ is further optionally substituted by one or more additional substituents independently selected from R¹²;

R⁷ is selected from —C(O)NHNH₂, —C(O)NH—C₁₋₃alkylene-NH(R¹⁰), —C(O)CH₃, —C₁₋₃ alkylene—NHC(O)OR¹¹, —C₁₋₃alkylene—NHC(O)R¹⁰, —C₁₋₃alkylene—NHC(O)NHR¹⁰, —C₁₋₃alkylene—NHC(O)—C₁₋₃alkylene-R¹⁰, and a 3- to 12-membered heterocycle optionally substituted with one or more substituents independently selected from R¹²;

R¹⁰ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, —C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;

R¹¹ is selected from C₃₋₁₂ carbocycle and 3- to 12-membered heterocycle, each of which is optionally substituted with one or more substituents independently selected from R¹²,

R¹² is independently selected at each occurrence from halogen, —OR¹⁰, —SR¹⁰, —N(R₁₀)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹² is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, and C₂₋₆ alkynyl; and

wherein any substitutable carbon on the benzazepine core is optionally substituted by a substituent independently selected from R¹² or two substituents on a single carbon atom combine to form a 3- to 7-membered carbocycle. In some embodiments, the compound of Formula (IIIA) is represented by Formula (TIM):

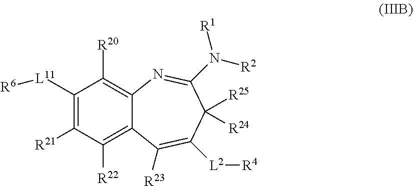

or a pharmaceutically acceptable salt thereof, wherein:

R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and

R²⁴, and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments, R²⁰, R²¹, R^{22,} and R²³ are independently selected from hydrogen, halogen, —OH, —NO₂, —CN, and C₁₋₁₀ alkyl. In certain embodiments, R²⁰, R²¹, R^{22,} and R²³ are each hydrogen. In some embodiments, R²⁴ and R²⁵ are independently selected from hydrogen, halogen, —OH, —NO₂, —CN, and C₁₋₁₀ alkyl, or R²⁴ and R²⁵ taken together form an optionally substituted saturated C₃₋₇ carbocycle. In certain embodiments, R²⁴ and R²⁵ are each hydrogen. In certain embodiments, R²⁴ and R²⁵ taken together form an optionally substituted saturated C₃₋₅ carbocycle.

In some embodiments, R¹ is hydrogen. In some embodiments, R² is hydrogen.

In some embodiments, L¹¹ is selected from) —C(O)N(R¹⁰)—*. In some embodiments, R¹⁰ of —C(O)N(R¹⁰)—* is selected from hydrogen and C₁₋₆ alkyl. For example, L¹¹ may be —C(O)NH—*.

In some embodiments, R⁶ is phenyl substituted with R⁷ and R⁶ is further optionally substituted with one or more additional substituents independently selected from R¹². In some embodiments, R⁶ is selected from phenyl substituted with one or more substituents independently selected from —C(O)NHNH₂, —C(O)NH—C₁₋₃alkylene-NH(R¹⁰), —C₁₋₃alkylene—NHC(O)R¹⁰, and —C(O)CH₃; and 3- to 12-membered heterocycle, which is optionally substituted with one or more substituents selected from —OH, —N(R¹⁰)₂, —NHC(O)(R¹⁰), —NHC(O)O(R¹⁰), —NHC(O)N(R¹⁰)₂, —C(O)R¹⁰, —C(O)N(R¹⁰)₂, —C(O)₂R¹⁰, and —C₁₋₃alkylene-(R¹⁰) and R⁶ is further optionally substituted with one or more additional substituents independently selected from R¹². For example, R⁶ may be selected from:

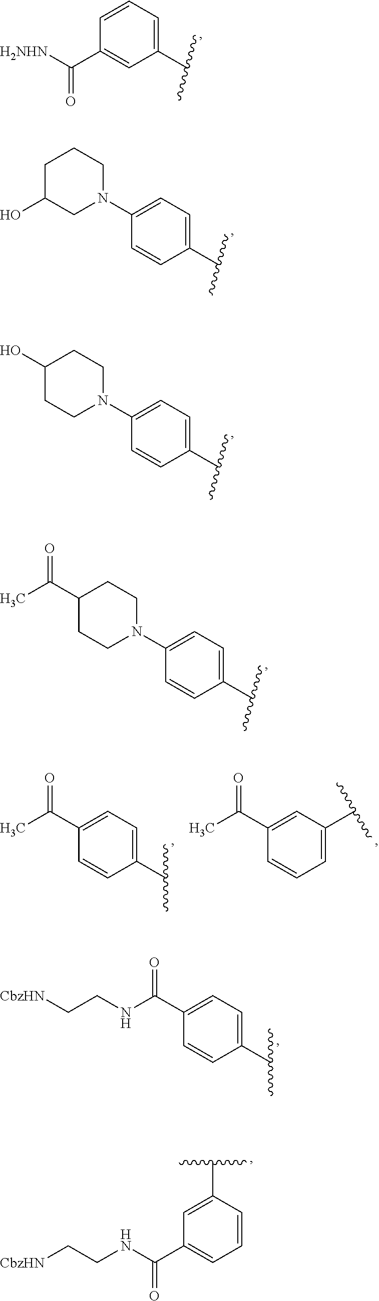

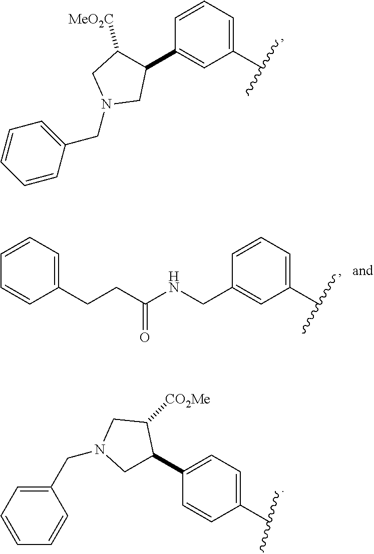

In some embodiments, R⁶ is selected from a 5- and 6-membered heteroaryl substituted with one or more substituents independently selected from R⁷, and R⁶ is further optionally substituted with one or more additional substituents selected from R¹². In certain embodiments, R⁶ is selected from 5- and 6-membered heteroaryl substituted with one or more substituents independently selected from —C(O)CH₃, —C₁₋₃alkylene—NHC(O)OR¹⁰, —C₁₋₃alkylene—NHC(O)R¹⁰, —C₁₋₃alkylene—NHC(O)NHR¹⁰, and —C₁₋₃alkylene—NHC(O)—C₁₋₃alkylene-(R¹⁰); and 3- to 12-membered heterocycle, which is optionally substituted with one or more substituents selected from —OH, —N(R¹⁰)₂, —NHC(O)(R¹⁰), —NHC(O)O(R¹⁰), —NHC(O)N(R¹⁰)₂, —C(O)R¹⁰, —C(O)N(R¹⁰)₂, —C(O)₂R¹⁰, and —C₁₋₃alkylene-(R¹⁰), and R⁶ is optionally further substituted with one or more additional substituents independently selected from R¹². R⁶ may be selected from substituted pyridine, pyrazine, pyrimidine, pyridazine, furan, pyran, oxazole, thiazole, imidazole, pyrazole, oxadiazole, oxathiazole, and triazole, and R⁶ is optionally further substituted with one or more additional substituents independently selected from R¹². In some embodiments, R⁶ is substituted pyridine and R⁶ is optionally further substituted with one or more additional substituents independently selected from R¹². R⁶ may be represented as follows:

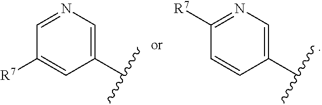

In some embodiments, R⁶ is substituted pyridine, and wherein R⁷ is —C₁₋₃alkylene—NHC(O)—C₁₋₃alkylene-R¹⁰. In certain embodiments, R⁷ is —C₁alkylene—NHC(O)—C₁alkylene-R¹⁰. In certain embodiments, R⁷ is —C₁alkylene—NHC(O)—C₁alkylene-NH₂. In some embodiments, R⁶ is selected from:

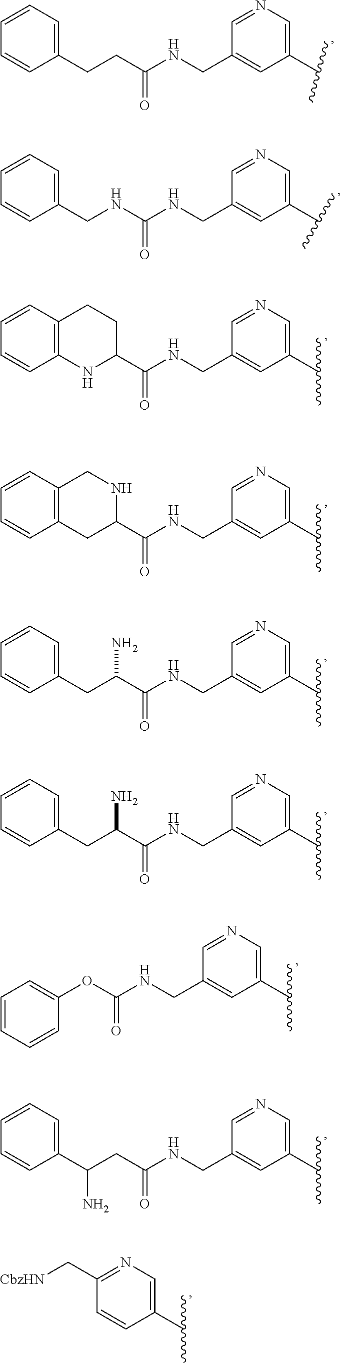

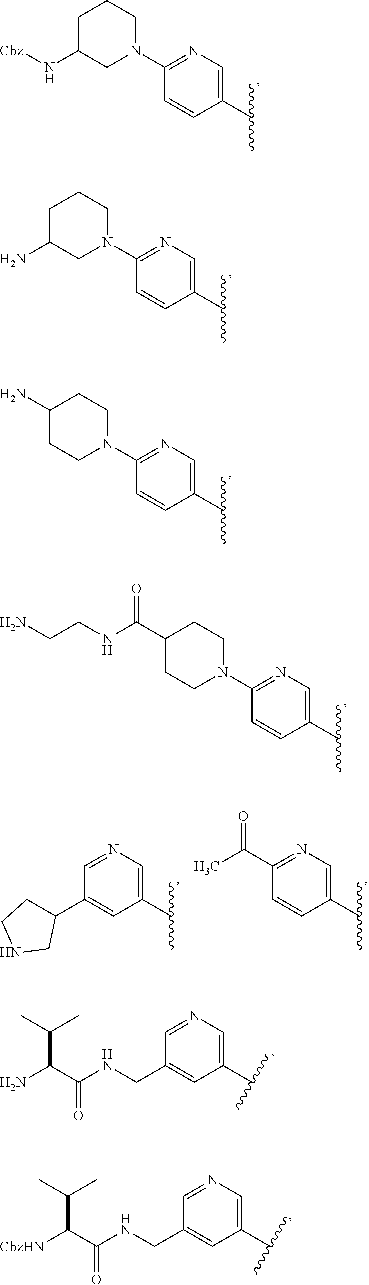

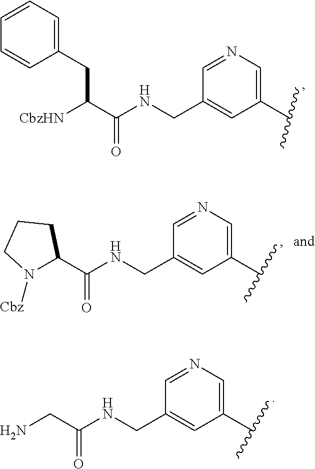 [SMILES: *c1cncc(CNC(=O)[C@H](Cc2ccccc2)NC(=O)OCc2ccccc2)c1 ; *c1cncc(CNC(=O)[C@@H]2CCCN2C(=O)OCc2ccccc2)c1 ; *c1cncc(CNC(=O)CN)c1]

In certain embodiments, R⁶ is

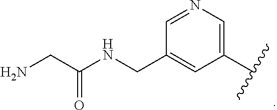 [SMILES: *c1cncc(CNC(=O)CN)c1]

In some embodiments, L² is selected from —C(O)—, and —C(O)NR¹⁰—. In some embodiments, L² is selected from —C(O)NR¹⁰—. R¹⁰ of —C(O)NR¹⁰— may be selected from hydrogen and C₁₋₆ alkyl. For example, L² may be —C(O)NH—. In some embodiments, L² is —C(O)—.

In some embodiments, R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle and 3- to 12-membered, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl. In some embodiments, R⁴ is selected from: —OR¹⁰and —N(R¹⁰)₂; and C₁₋₁₀ alkyl, C₂₋₁₀ alkenyl, C₂₋₁₀ alkynyl, C₃₋₁₂ carbocycle and 3- to 12-membered heterocycle, each of which is independently optionally substituted at each occurrence with one or more substituents selected from halogen, —OR¹⁰, —SR¹⁰, —N(R¹⁰)₂, —S(O)R¹⁰, —S(O)₂R¹⁰—C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl. In certain embodiments, R⁴ is —N(R¹⁰)₂. R¹⁰ of —N(R¹⁰)₂ may be independently selected at each occurrence from optionally substituted C₁₋₆ alkyl. In some embodiments, R¹⁰ of —N(R¹⁰)₂ is independently selected at each occurrence from methyl, ethyl, propyl, and butyl, any of which are optionally substituted. For example, R⁴ may be

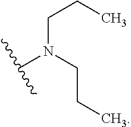 [SMILES: *N(CCC)CCC]

In some embodiments, -L²-R⁴ is

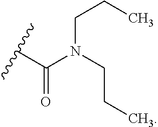 [SMILES: *C(=O)N(CCC)CCC]

In some embodiments, R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is independently optionally substituted at each occurrence with one or more substituents selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, each of which is independently optionally substituted at each occurrence with one or more substituents selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, and C₂₋₆ alkynyl. In certain embodiments, R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is independently optionally substituted at each occurrence with one or more substituents selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle. In some embodiments, the compound is selected from:

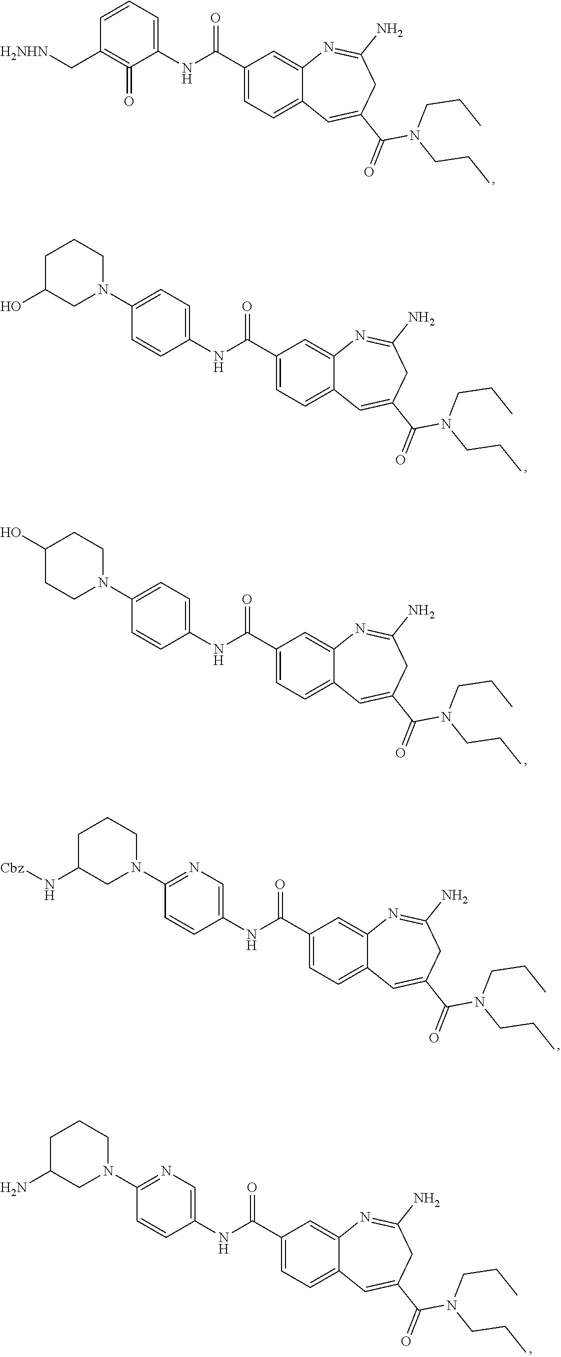

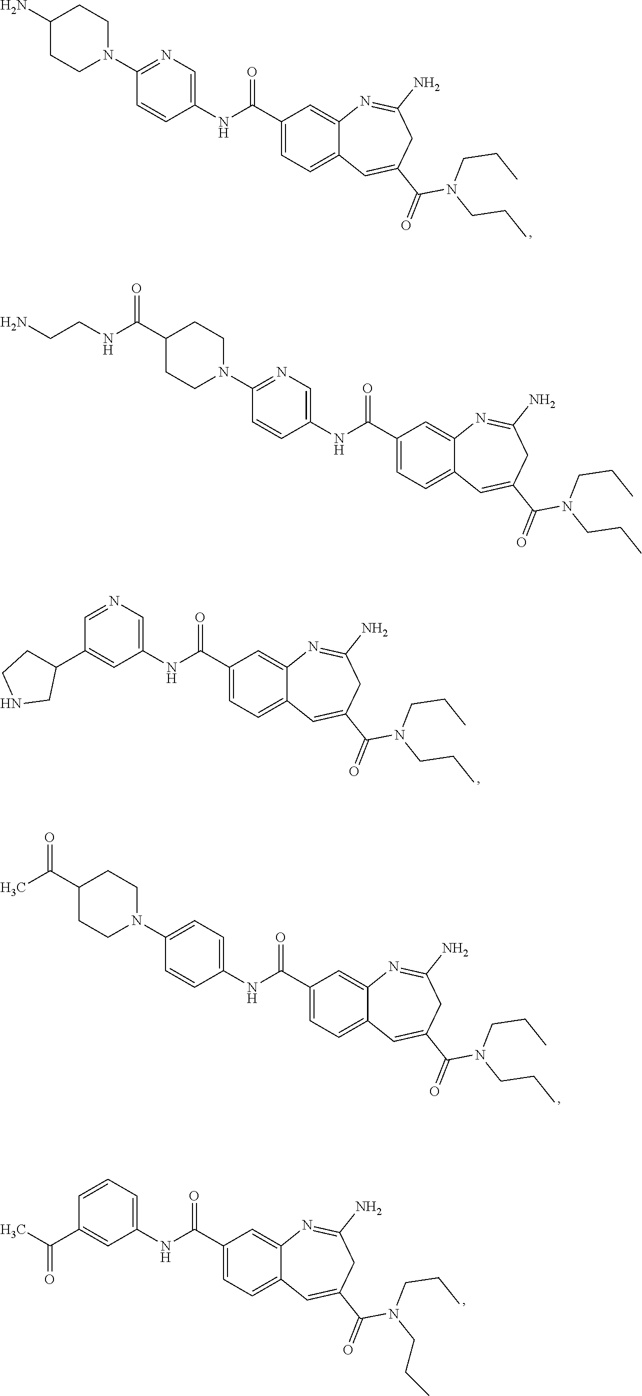

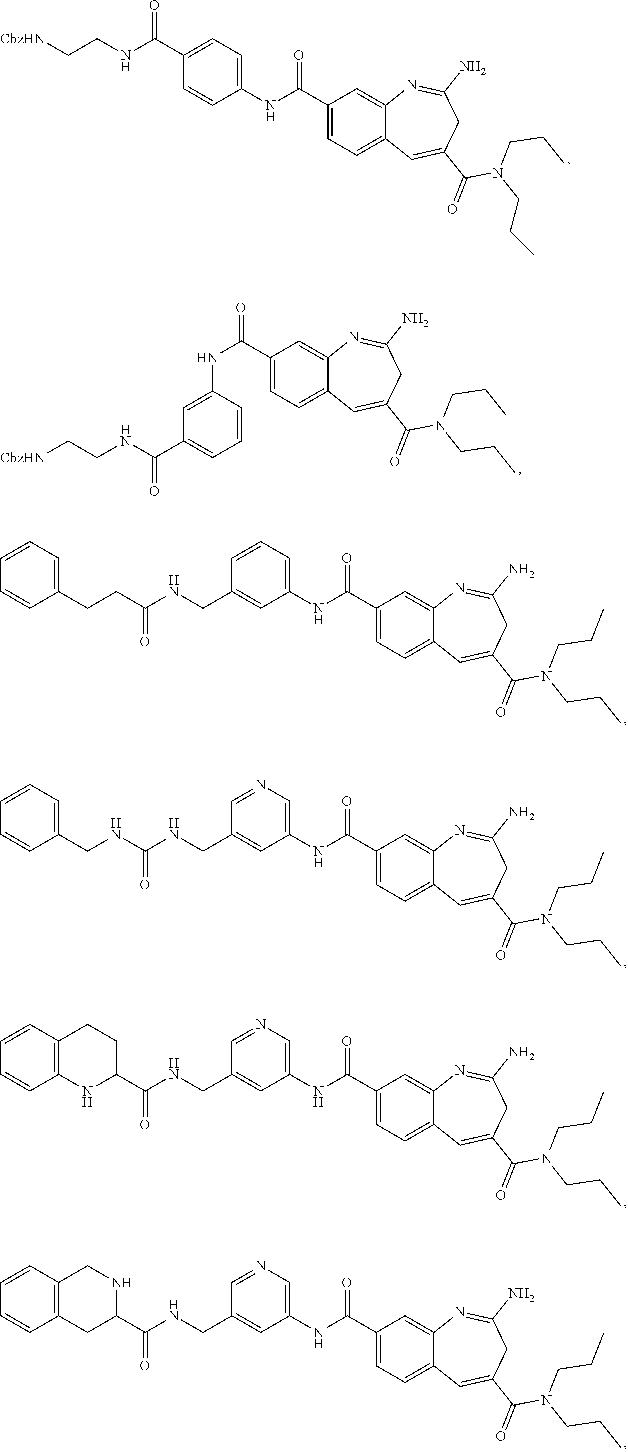 [SMILES: CCCN(CCC)C(=O)C1=Cc2ccc(C(=O)Nc3ccc(C(=O)NCCNC(=O)OCc4ccccc4)cc3)cc2N=C(N)C1 ; CCCN(CCC)C(=O)C1=Cc2ccc(C(=O)Nc3cccc(C(=O)NCCNC(=O)OCc4ccccc4)c3)cc2N=C(N)C1 ; CCCN(CCC)C(=O)C1=Cc2ccc(C(=O)Nc3cccc(CNC(=O)CCc4ccccc4)c3)cc2N=C(N)C1 ; CCCN(CCC)C(=O)C1=Cc2ccc(C(=O)Nc3cncc(CNC(=O)NCc4ccccc4)c3)cc2N=C(N)C1 ; CCCN(CCC)C(=O)C1=Cc2ccc(C(=O)Nc3cncc(CNC(=O)C4CCc5ccccc5N4)c3)cc2N=C(N)C1 ; CCCN(CCC)C(=O)C1=Cc2ccc(C(=O)Nc3cncc(CNC(=O)C4Cc5ccccc5CN4)c3)cc2N=C(N)C1]

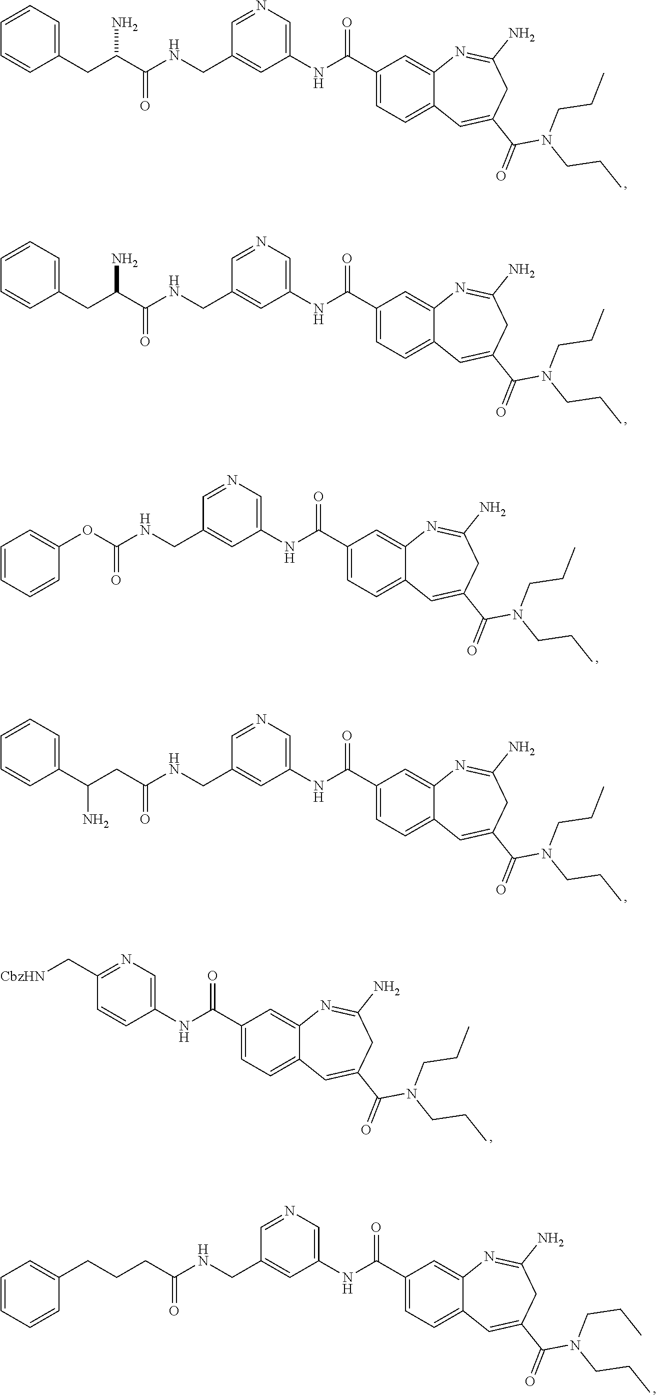 [SMILES: CCCN(CCC)C(=O)C1=Cc2ccc(C(=O)Nc3cncc(CNC(=O)[C@@H](N)Cc4ccccc4)c3)cc2N=C(N)C1 ; CCCN(CCC)C(=O)C1=Cc2ccc(C(=O)Nc3cncc(CNC(=O)[C@H](N)Cc4ccccc4)c3)cc2N=C(N)C1 ; CCCN(CCC)C(=O)C1=Cc2ccc(C(=O)Nc3cncc(CNC(=O)Oc4ccccc4)c3)cc2N=C(N)C1 ; CCCN(CCC)C(=O)C1=Cc2ccc(C(=O)Nc3cncc(CNC(=O)CC(N)c4ccccc4)c3)cc2N=C(N)C1 ; CCCN(CCC)C(=O)C1=Cc2ccc(C(=O)Nc3ccc(CNC(=O)OCc4ccccc4)nc3)cc2N=C(N)C1 ; CCCN(CCC)C(=O)C1=Cc2ccc(C(=O)Nc3cncc(CNC(=O)CCCc4ccccc4)c3)cc2N=C(N)C1]

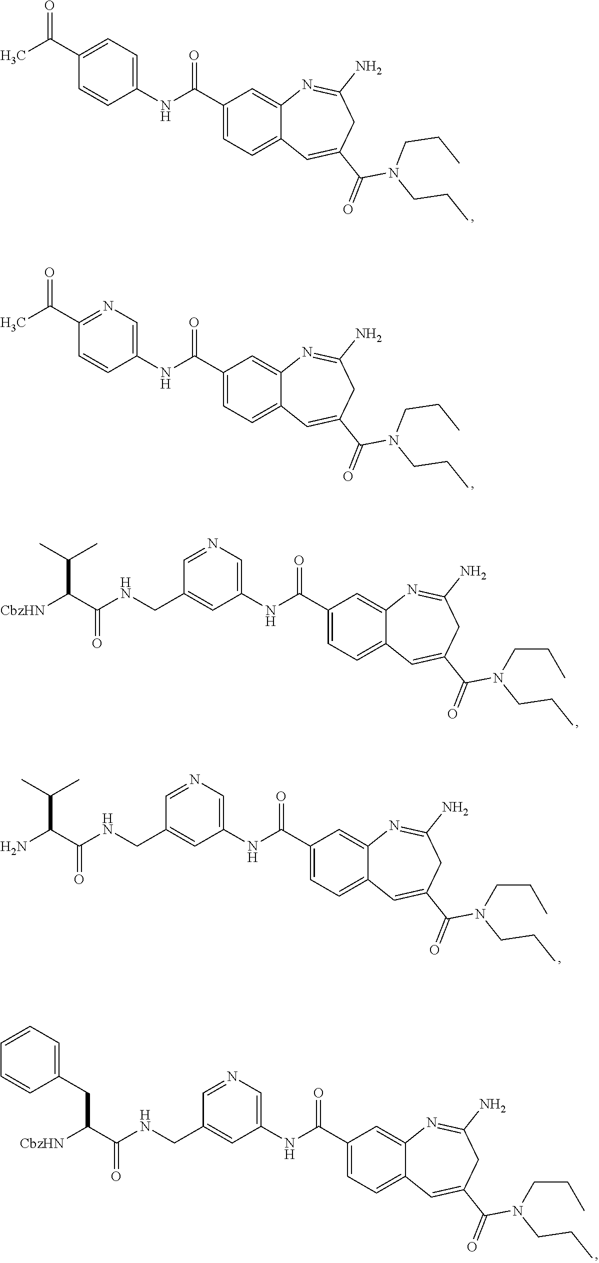

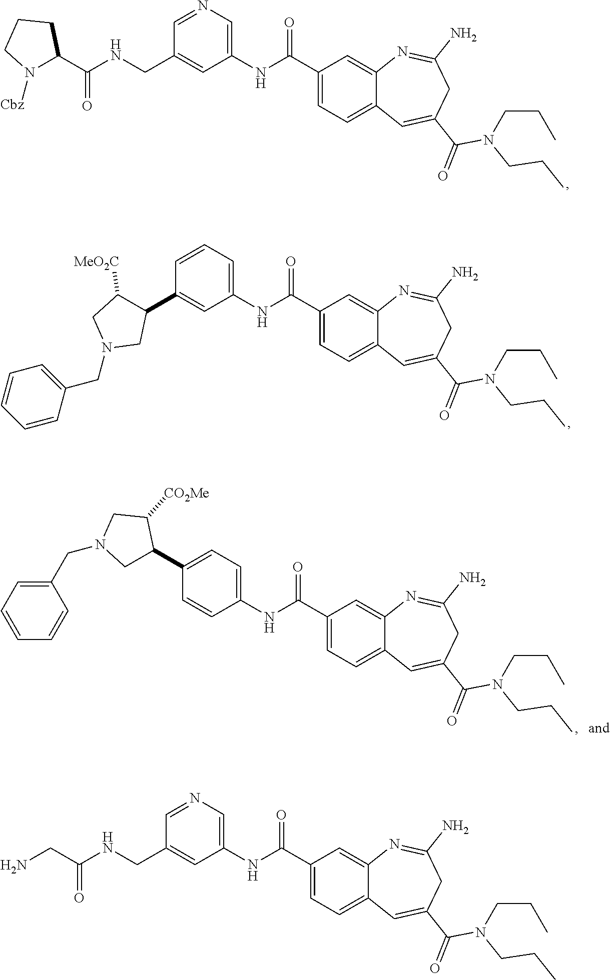

and a salt of any one thereof.

In some aspects, the present disclosure provides a compound represented by the structure of Formula (IA):

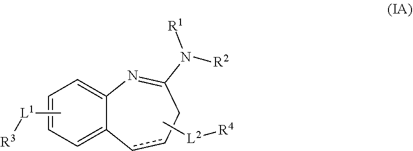

or a pharmaceutically acceptable salt thereof, wherein:

[symbol] represents an optional double bond;

L¹ is selected from —X¹—, —X²—C₁₋₆ alkylene-X²—C₁₋₆ alkylene-, —X²—C₂₋₆ alkenylene-X²—, and —X²—C₂₋₆ alkynylene-X²—, each of which is optionally substituted on alkylene, alkenylene or alkynylene with one or more R¹²;

L² is selected from —X²—, —X²—C₁₋₆ alkylene-X²—, —X²—C₂₋₆ alkenylene-X²—, and —X²—C₂₋₆ alkynylene-X²—, each of which is optionally substituted on alkylene, alkenylene or alkynylene with one or more R¹²;

X¹ is selected from —S—*, —N(R¹⁰)—*, —C(O)O—*, —OC(O)—*, —OC(O)O—*, —C(O)N(R¹⁰)C(O)—*, —C(O)N(R¹⁰)C(O)N(R¹⁰)*, —N(R¹⁰)C(O)—*, —CR¹⁰₂N(R¹⁰)C(O)—*, —N(R¹⁰)C(O)N(R¹⁰)—*, —N(R¹⁰)C(O)O—*, —OC(O)N(R¹⁰)—*, —C(NR¹⁰)—*, —N(R¹⁰)C(NR¹⁰)—*, —C(NR¹⁰)N(R¹⁰)—*, —N(R¹⁰)C(NR¹⁰)N(R¹⁰)—*, —S(O)₂—*, —OS(O)—*, —S(O)O—*, —S(O), —OS(O)₂—*, —S(O)₂O*, —N(R¹⁰)S(O)₂—*, —S(O)₂N(R¹⁰)—*, —N(R¹⁰)S(O)—*, —S(O)N(R¹⁰)—*, —N(R¹⁰)S(O)₂N(R¹⁰)—*, and —N(R¹⁰)S(O)N(R¹⁰)—*, wherein * represents where X¹ is bound to R³;

X² is independently selected at each occurrence from —O—, —S—, —N(R¹⁰)—, —C(O)—, —C(O)O—, —OC(O)—, —OC(O)O—, —C(O)N(R¹⁰)—, —C(O)N(R¹⁰)C(O)—, —C(O)N(R¹⁰)C(O)N(R¹⁰), —N(R¹⁰)C(O)—, —N(R¹⁰)C(O)N(R¹⁰)—, —N(R¹⁰)C(O)O—, —OC(O)N(R¹⁰)—, —C(NR¹⁰)—, —N(R¹⁰)C(NR¹⁰)—, —C(NR¹⁰)N(R¹⁰)—, —N(R¹⁰)C(NR¹⁰)N(R¹⁰)—, —S(O)₂—, —OS(O)—, —S(O)O—, —S(O), —OS(O)₂—, —S(O)₂O, —N(R¹⁰)S(O)₂—, —S(O)₂N(R¹⁰)—, —N(R¹⁰)S(O)—, —S(O)N(R¹⁰)—, —N(R¹⁰)S(O)₂N(R¹⁰)—, and —N(R¹⁰)S(O)N(R¹⁰)—;

R¹ and R² are independently selected from hydrogen; C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), and —CN;

R³ is selected from optionally substituted C₃₋₁₂ carbocycle, and optionally substituted 3- to 12-membered heterocycle, wherein substituents on R³ are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, wherein each C₃₋₁₂ carbocycle, and 3- to 12-membered heterocycle in R³ is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;

R⁴ is selected from: —OR¹⁰, —N(R¹⁰)₂, —C(O)N(R¹⁰)², —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, wherein each C₃₋₁₂ carbocycle, and 3- to 12-membered heterocycle in R⁴ is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;

R¹⁰ is independently selected at each occurrence from: hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;

R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹² is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, and C₂₋₆ alkynyl; and

wherein any substitutable carbon on the benzazepine core is optionally substituted by a substituent independently selected from R¹² or two substituents on a single carbon atom combine to form a 3- to 7-membered carbocycle. In some embodiments, the compound of Formula (IA) is represented by Formula (IB):

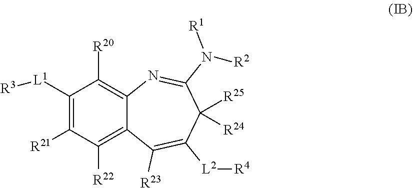

or a pharmaceutically acceptable salt thereof, wherein:

R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and

R²⁴ and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments, R²⁰, R²¹, R²², and R²³ are independently selected from hydrogen, halogen, —OH, —NO₂, —CN, and C₁₋₁₀ alkyl. In certain embodiments, R²⁰, R²¹, R²², and R²³ are each hydrogen.

In some embodiments, R²⁴ and R²⁵ are independently selected from hydrogen, halogen, —OH, —NO₂, —CN, and C₁₋₁₀ alkyl, or R²⁴ and R²⁵ taken together form an optionally substituted saturated C₃₋₇ carbocycle. In some embodiments, R²⁴ and R²⁵ are each hydrogen. In some embodiments, R²⁴ and R²⁵ taken together form an optionally substituted saturated C₃₋₅ carbocycle.

In some embodiments, R¹ is hydrogen. In some embodiments, R² is hydrogen.

In some embodiments, L¹ is selected from —N(R¹⁰)C(O)—*, —S(O)₂N(R¹⁰)—*, —CR¹⁰₂N(R¹⁰)C(O)—* and —X²—C₁₋₆ alkylene-X²—C₁₋₆ alkylene-. In some embodiments, L¹ is selected from —N(R¹⁰)C(O)—*. In certain embodiments, R¹⁰ of —N(R¹⁰)C(O)—* is selected from hydrogen and C₁₋₆ alkyl. For example, L¹ may be —NHC(O)—*. In some embodiments, L¹ is selected from —S(O)₂N(R¹⁰)—*. In certain embodiments, R¹⁰ of —S(O)₂N(R¹⁰)—* is selected from hydrogen and C₁₋₆ alkyl. For example, L¹ is —S(O)₂NH—*. In some embodiments, L¹ is —CR¹⁰₂N(R¹⁰)C(O)—*. In certain embodiments, L¹ is selected from —CH₂N(H)C(O)—* and —CH(CH₃)N(H)C(O)—*.

In some embodiments, R³ is selected from optionally substituted C₃₋₁₂ carbocycle, and optionally substituted 3- to 12-membered heterocycle, wherein substituents on R³ are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl. In certain embodiments, R³ is selected from optionally substituted C₃₋₁₂ carbocycle, and optionally substituted 3- to 12-membered heterocycle, wherein substituents on R³ are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle.

In some embodiments, R³ is selected from an optionally substituted aryl and an optionally substituted heteroaryl. In some embodiments, R³ is an optionally substituted heteroaryl. R³ may be an optionally substituted heteroaryl substituted with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. In certain embodiments, R³ is selected from an optionally substituted 6-membered heteroaryl. For example, R³ may be an optionally substituted pyridine. In some embodiments, R³ is an optionally substituted aryl. In certain embodiments, R³ is an optionally substituted aryl substituted with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. R³ may be an optionally substituted phenyl. In certain embodiments, R³ is selected from pyridine, phenyl, tetrahydronaphthalene, tetrahydroquinoline, tetrahydroisoquinoline, indane, cyclopropylbenzene, cyclopentapyridine, and dihydrobenzoxaborole, any one of which is optionally substituted. R³ may be selected from:

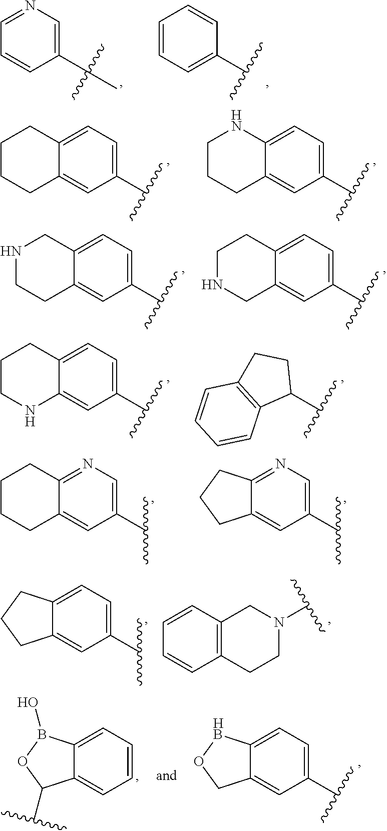

any one of which is optionally substituted. For example, R³ may be selected from:

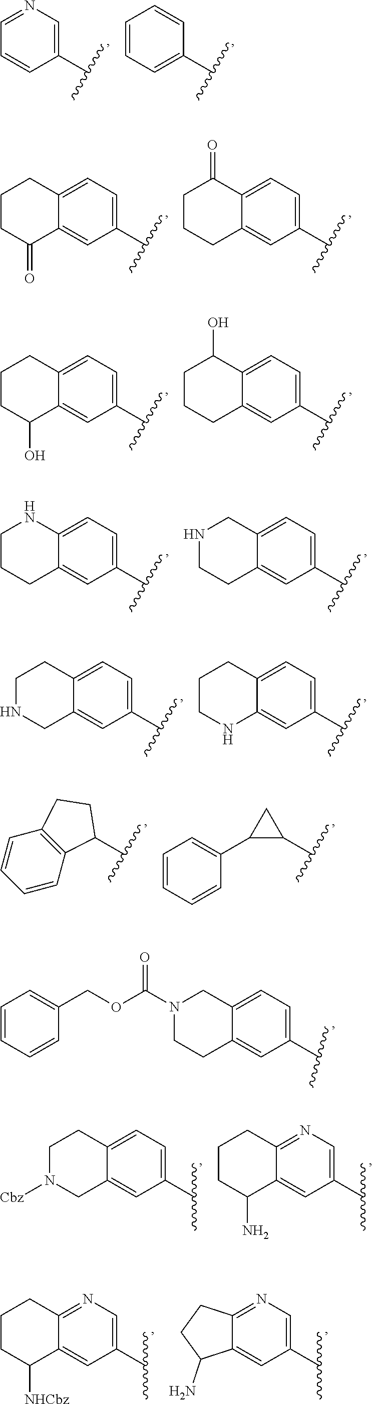

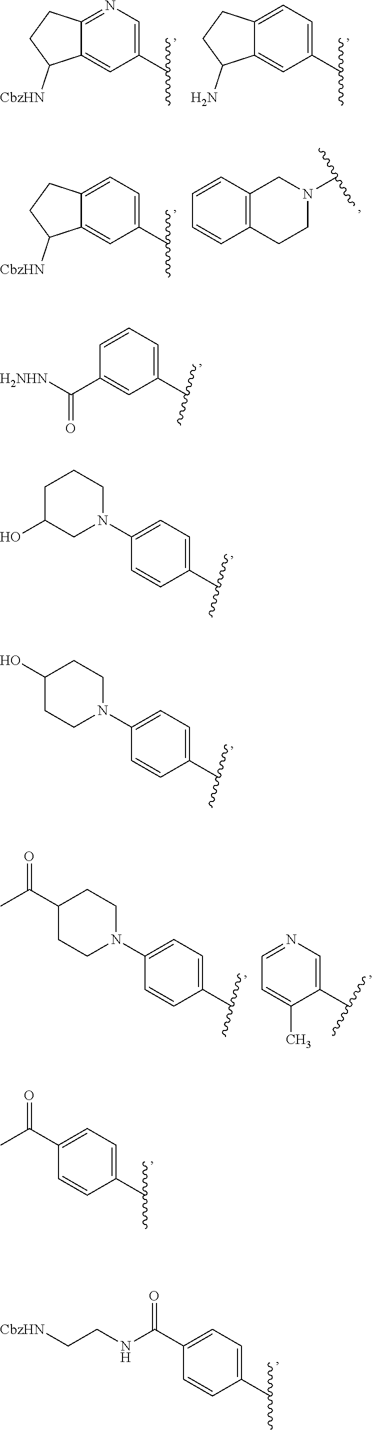

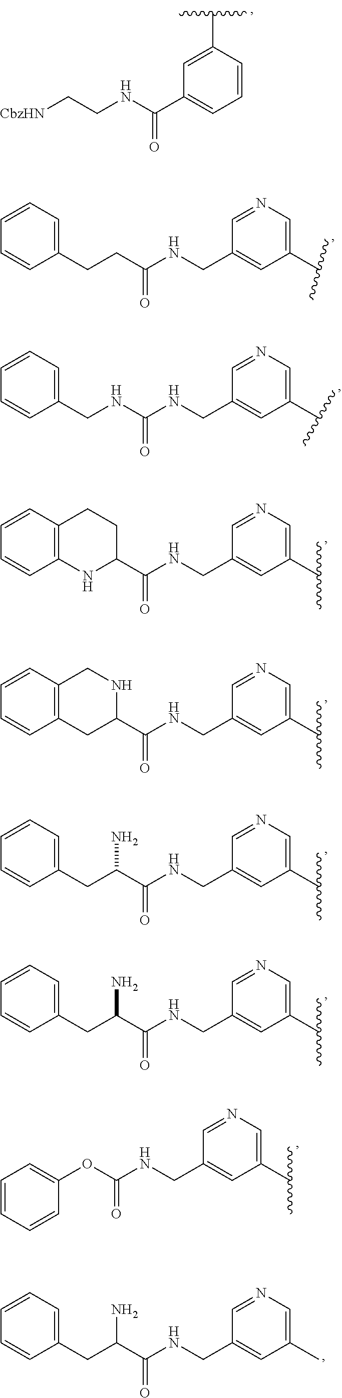

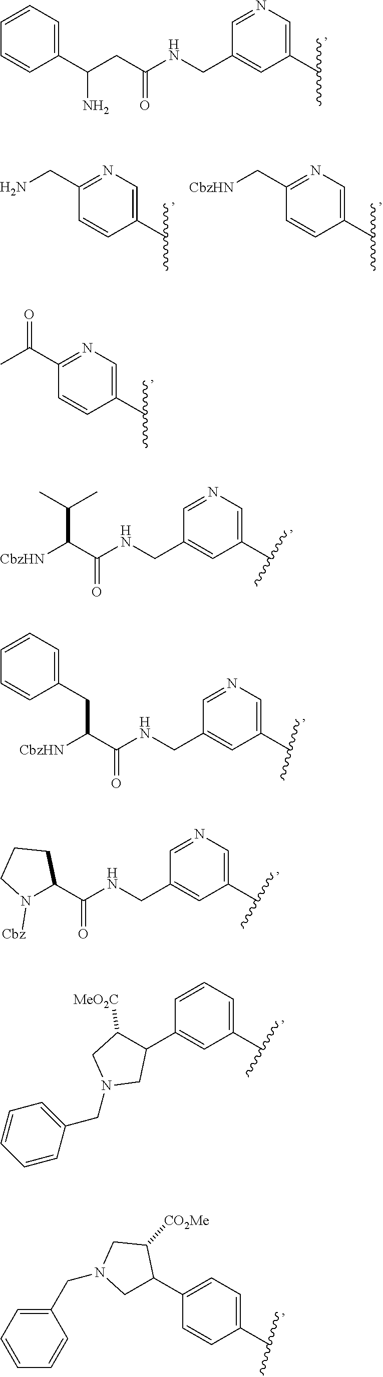

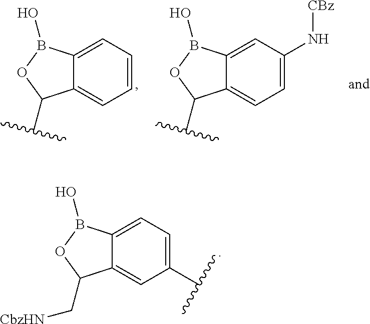

In some embodiments, L² is selected from —C(O)—, and —C(O)NR¹⁰—. In certain embodiments, L² is —C(O)—. In certain embodiments, L² is selected from —C(O)NR¹⁰—. R¹⁰ of —C(O)NR¹⁰— may be selected from hydrogen and C₁₋₆ alkyl. For example, L² may be —C(O)NH—.

In some embodiments, R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl.

In some embodiments, R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle. In some embodiments, R⁴ is selected from: —OR¹⁰, and —N(R¹⁰)₂; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl. In certain embodiments, R⁴ is —N(R¹⁰)₂. R¹⁰ of —N(R¹⁰)₂ may be independently selected at each occurrence from optionally substituted C₁₋₆ alkyl. In certain embodiments, R¹⁰ of —N(R¹⁰)₂ is independently selected at each occurrence from methyl, ethyl, propyl, and butyl, any one of which is optionally substituted. For example, R⁴ may be

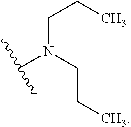 [SMILES: *N(CCC)CCC]

In certain embodiments, L²-R⁴ is

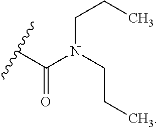 [SMILES: *C(=O)N(CCC)CCC]

In some embodiments, R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, C₂₋₆ alkynyl. In some embodiments, R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle.

In some embodiments, the compound is selected from:

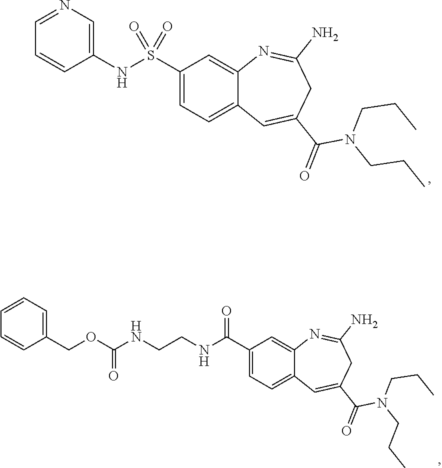 [SMILES: CCCN(CCC)C(=O)C1=Cc2ccc(S(=O)(=O)Nc3cccnc3)cc2N=C(N)C1 ; CCCN(CCC)C(=O)C1=Cc2ccc(C(=O)NCCNC(=O)OCc3ccccc3)cc2N=C(N)C1]

and a salt of any one thereof.

In some aspects, the present disclosure provides a compound represented by the structure of Formula (IVA):

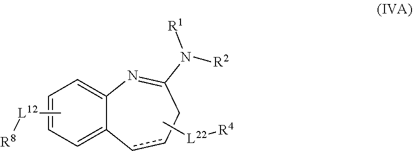

or a pharmaceutically acceptable salt thereof, wherein:

[symbol] represents an optional double bond;

L¹² is selected from —X³—, —X³—C₁₋₆ alkylene-X³—, —X³—C₂₋₆alkenylene-X³—, and —X³—C₂₋₆alkynylene-X³—, each of which is optionally substituted on alkylene, alkenylene, or alkynylene with one or more substituents independently selected from R¹²;

L²² is independently selected from —X⁴—, —X⁴—C₁₋₆ alkylene-X⁴—, —X⁴—C₂₋₆ alkenylene-X⁴—, and —X⁴—C₂₋₆ alkynylenc-X⁴—, each of which is optionally substituted on alkylene. alkenylene, or alkynylene with one or more substituents independently selected from R¹⁰;

X³ and X⁴ are independently selected at each occurrence from a bond, —O—, —S—, —N(R¹⁰)—, —C(O)—, —C(O)O—, —OC(O)—, —OC(O)O—, —C(O)N(R¹⁰)—, —C(O)N(R¹⁰)C(O)—, —C(O)N(R¹⁰)C(O)N(R¹⁰)—, —N(R¹⁰)C(O)—, —N(R¹⁰)C(O)N(R¹⁰)—, —N(R¹⁰)C(O)O—, —OC(O)N(R¹⁰)—, —C(NR¹⁰)—, —N(R¹⁰)C(NR¹⁰)—, —C(NR¹⁰)N(R¹⁰)—, —N(R¹⁰)C(NR¹⁰)N(R¹⁰)—, —S(O)₂—, —OS(O)—, —S(O)O—, —S(O)—, —OS(O)₂—, —S(O)₂O—, —N(R¹⁰)S(O)₂—, —S(O)₂N(R¹⁰)—, —N(R¹⁰)S(O)—, —S(O)N(R¹⁰)—, —N(R¹⁰)S(O)₂N(R¹⁰)—, and —N(R¹⁰)S(O)N(R¹⁰)—;

R¹ and R² are independently selected from L³, and hydrogen; and C₁₋₁₀ alkyl, C₂₋₁₀ alkenyl, and C₂₋₁₀ alkynyl, each of which is optionally substituted with one or more substituents independently selected from L³, halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), and —CN;

R⁴ and R⁸ are independently selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from L³, halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, wherein each C₃₋₁₂ carbocycle, and 3- to 12-membered heterocycle in R⁴ and R⁸ is optionally substituted with one or more substituents independently selected from L³, halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;

R¹⁰ is independently selected at each occurrence from L³, hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;

L³ is a linker moiety, wherein at least one of R¹, R², and R¹⁰ is L³ or at least one substituent on a group selected from R¹, R², R⁴, R⁸, X³ and X⁴ is L³; and

R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹² is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, C₂₋₆ alkynyl; and

wherein any substitutable carbon on the benzazepine core is optionally substituted by a substituent independently selected from R¹² or two substituents on a single carbon atom combine to form a 3- to 7-membered carbocycle. In some embodiments, the compound of Formula (IVA) is represented by Formula (IVB):

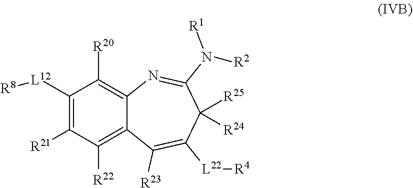

or a pharmaceutically acceptable salt thereof, wherein:

R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and

R²⁴, and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments, R¹ is L³. In some embodiments, R² is L³.

In some embodiments, L¹² is —C(O)N(R¹⁰)—. In some embodiments, R¹⁰ of —C(O)N(R¹⁰)— is selected from hydrogen, C₁₋₆ alkyl, and L³. For example, L¹² may be —C(O)NH—.

In some embodiments, R⁸ is an optionally substituted 5- or 6-membered heteroaryl. may be an optionally substituted 5- or 6-membered heteroaryl, substituted with L³. In some embodiments, R⁸ is an optionally substituted pyridine, substituted with L³.

In some embodiments, L²² is selected from —C(O)—, and —C(O)NR¹⁰—. In certain embodiments, L²² is —C(O)—. In certain embodiments, L₂₂ is —C(O)NR¹⁰—. R¹⁰ of —C(O)NR¹⁰— may be selected from hydrogen, C₁₋₆ alkyl, and -L³. For example, L²² may be —C(O)NH—.

In some embodiments, R⁴ is selected from: —OR¹⁰, and —N(R¹⁰)₂; and C₁₋₁₀ alkyl, C₂₋₁₀ alkenyl, C₂₋₁₀ alkynyl, C₃₋₁₂ carbocycle, 3- to 12-membered heterocycle, aryl, and heteroaryl, each of which is optionally substituted with one or more substituents independently selected from L³, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl. In some embodiments, R⁴ is —N(R¹⁰)₂ and R¹⁰ of —N(R¹⁰)₂ is selected from L³ and hydrogen, and wherein at least one R¹⁰ of —N(R¹⁰)₂ is L³.

In some embodiments, the compound is further covalently bound to a linker, L³. In some embodiments, L³ is a noncleavable linker. In some embodiments, L³ is a cleavable linker. L³ may be cleavable by a lysosomal enzyme. In some embodiments, the compound is covalently attached to an antibody construct. In some embodiments, the compound is covalently attached to a targeting moiety, optionally through the linker. In some embodiments, the targeting moiety or antibody construct specifically binds to a tumor antigen. In some embodiments, the antibody construct or targeting moiety further comprises a target binding domain.

In some embodiments, L³ is represented by the formula:

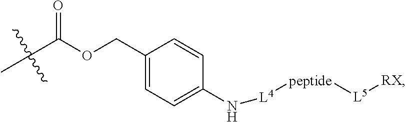

wherein:

L⁴ represents the C-terminus of the peptide and L⁵ is selected from a bond, alkylene and heteroalkylene, wherein L⁵ is optionally substituted with one or more groups independently selected from R³², and RX is a reactive moiety; and

R³² is independently selected at each occurrence from halogen, —OH, —CN, —O-alkyl, —SH, ═O, ═S, —NH₂, —NO₂; and C₁₋₁₀ alkyl, C₂₋₁₀ alkenyl, C₂₋₁₀ alkynyl, each of which is optionally substituted with one or more substituents independently selected from halogen, —OH, —CN, —O-alkyl, —SH, ═O, ═S, —NH₂, —NO₂. In some embodiments, RX comprises a leaving group. In some embodiments, RX comprises a maleimide. In some embodiments, L³ is further covalently bound to an antibody construct. In some embodiments, the antibody construct is directed against a tumor antigen. In some embodiments, the antibody construct further comprises target binding domain.

In some embodiments, L³ is represented by the formula:

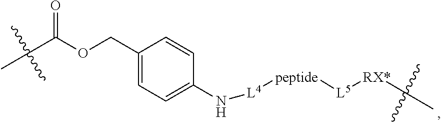

wherein L⁴ represents the C-terminal of the peptide and L⁵ is selected from a bond, alkylene and heteroalkylene, wherein L⁵ is optionally substituted with one or more groups independently selected from R³²; RX* comprises a bond, a succinimide moiety, or a hydrolyzed succinimide moiety bound to a residue of an antibody construct, wherein

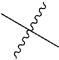

on RX* represents the point of attachment to the residue of the antibody construct; and,

R³² is independently selected at each occurrence from halogen, —OH, —CN, —O-alkyl, —SH, ═O, ═S, —NH₂, —NO₂; and C₁₋₁₀ alkyl, C₂₋₁₀ alkenyl, C₂₋₁₀ alkynyl, each of which is optionally substituted with one or more substituents independently selected from halogen, —OH, —CN, —O-alkyl, —SH, ═O, ═S, —NH₂, —NO₂. In some embodiments, the peptide of L³ comprises Val-Cit or Val-Ala.

In some aspects, the present disclosure provides a compound or salt selected from:

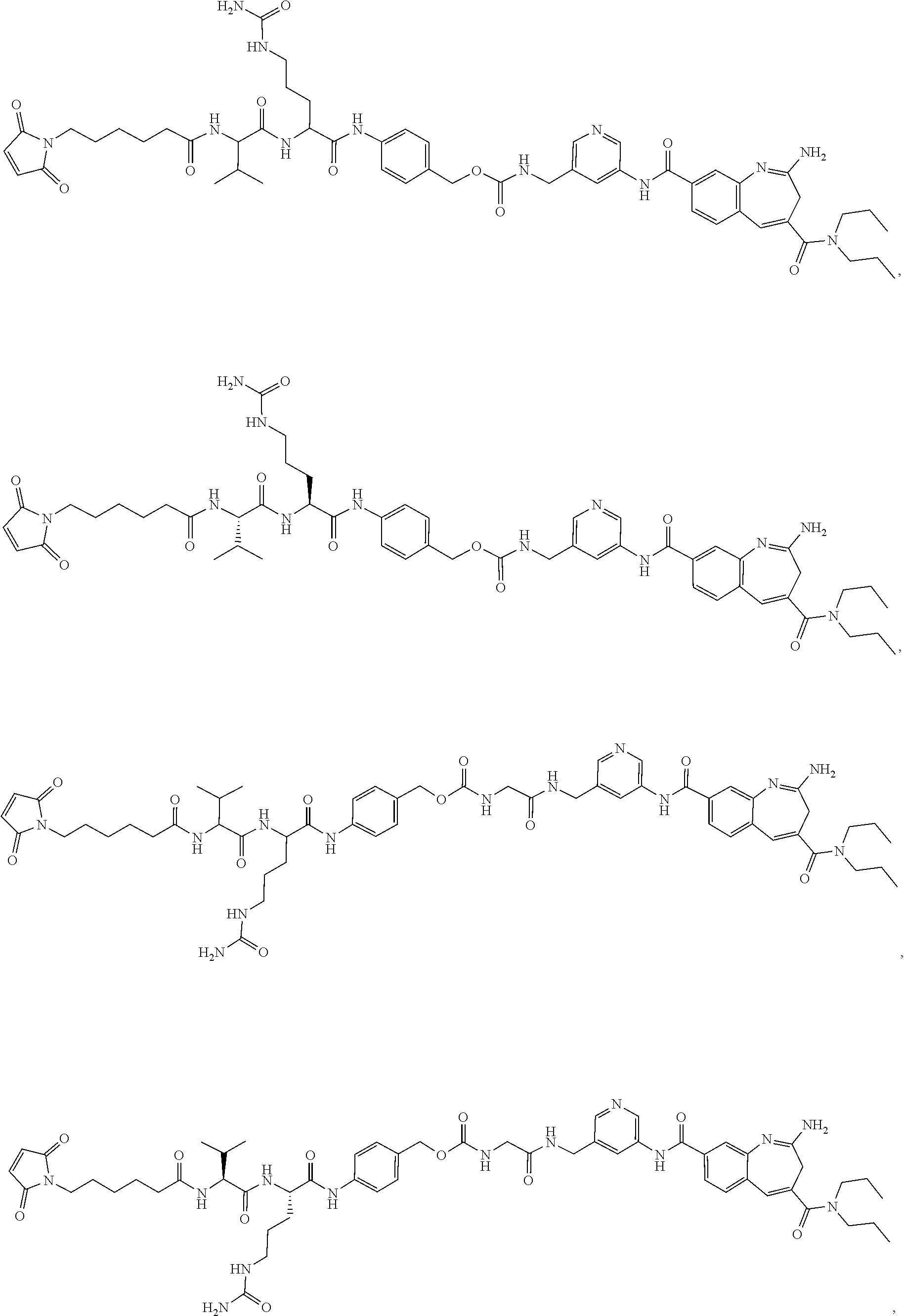

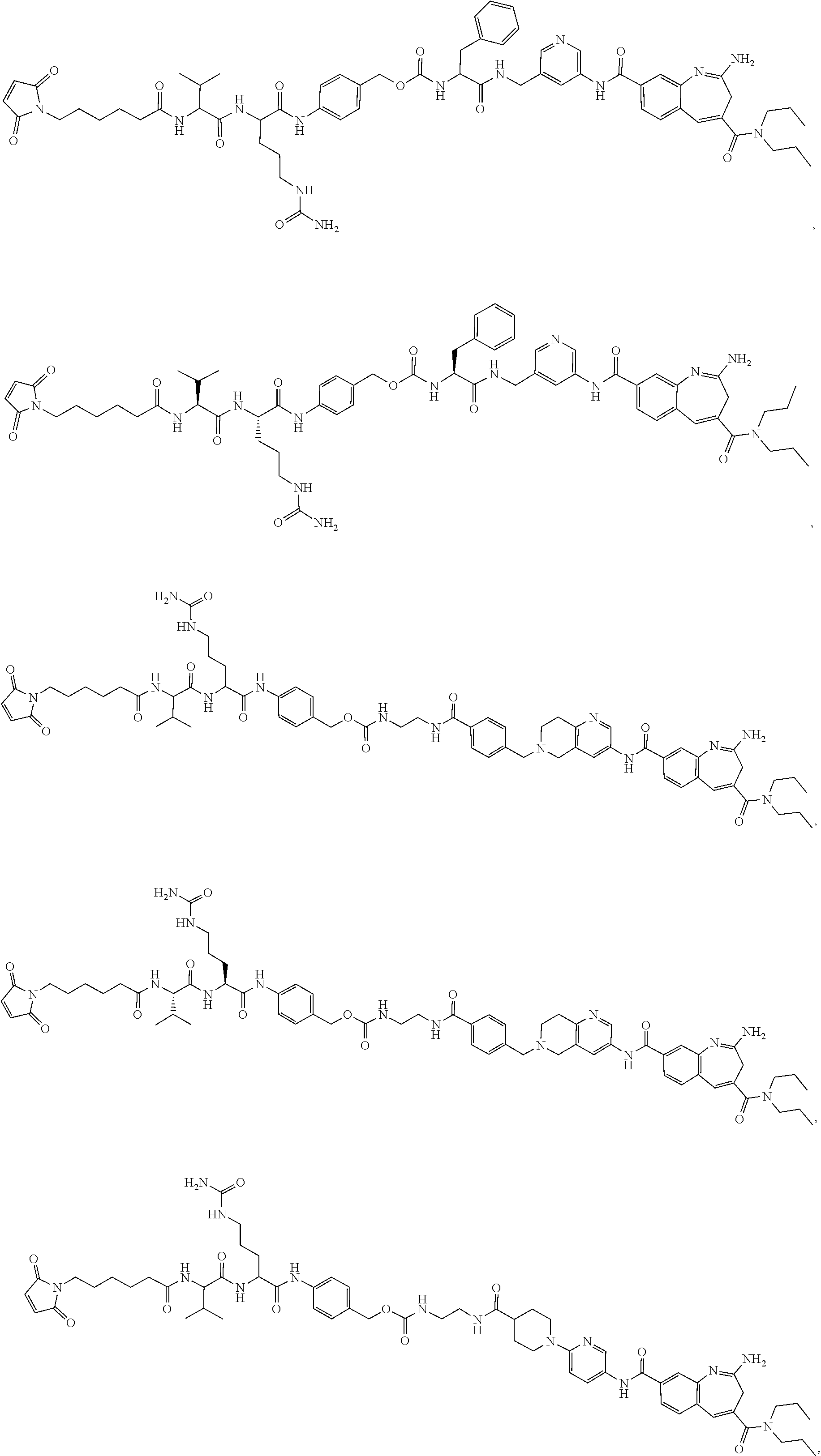

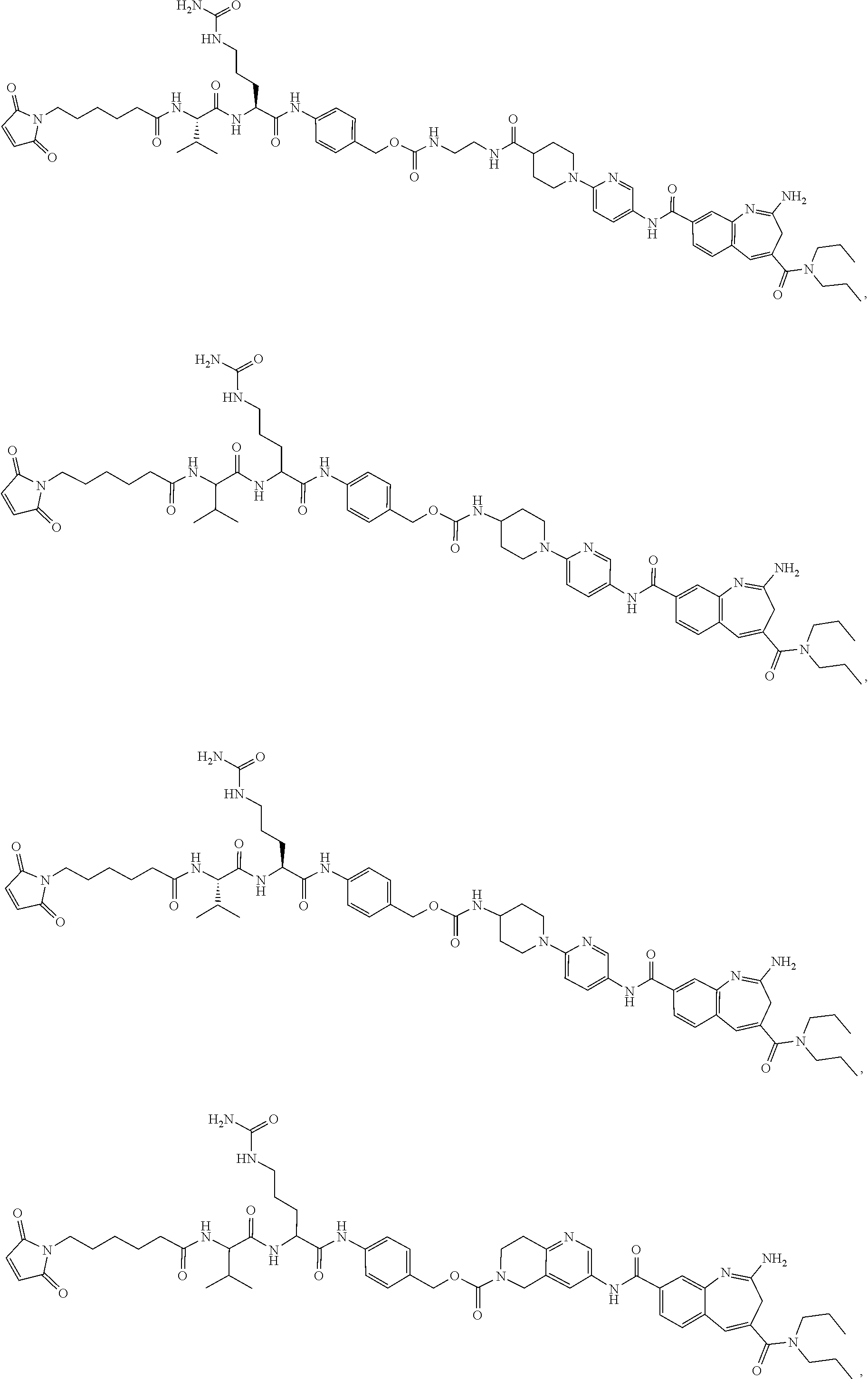

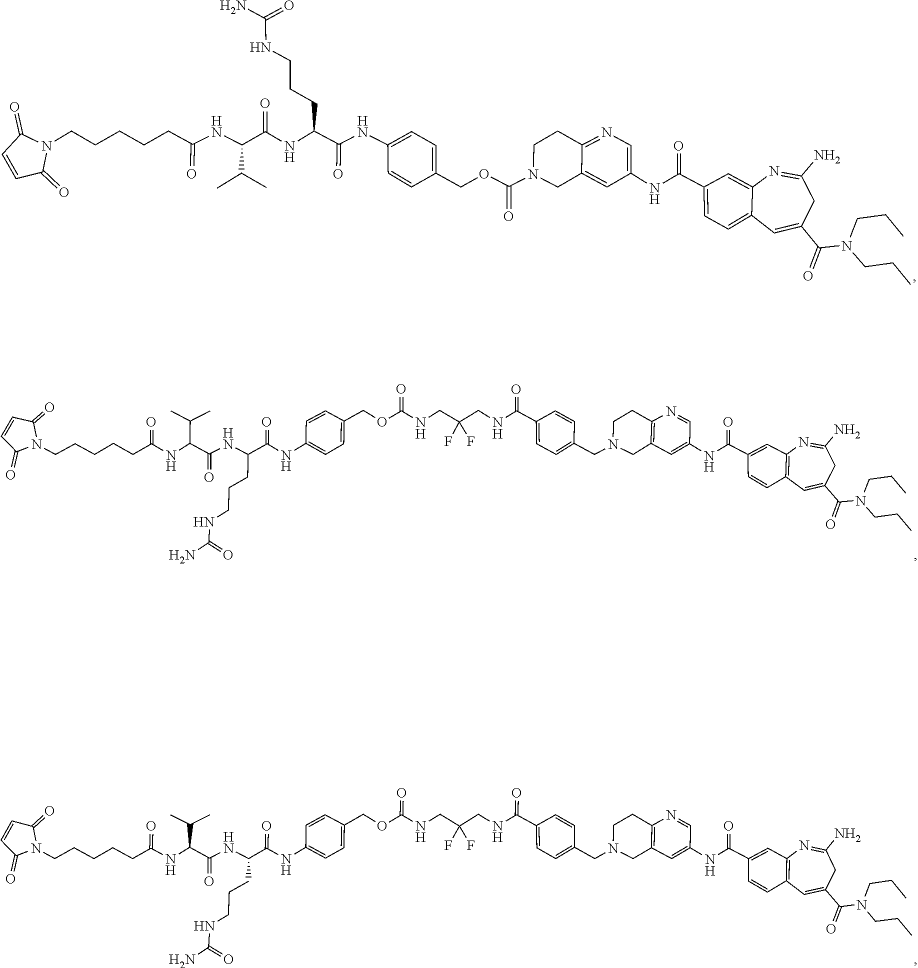 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1 ; CCCN(CCC)C(=O)C1=Cc2ccc(C(=O)Nc3cnc4c(c3)CN(Cc3ccc(C(=O)NCC(F)(F)CNC(=O)OCc5ccc(NC(=O)C(CCCNC(N)=O)NC(=O)C(NC(=O)CCCCCN6C(=O)C=CC6=O)C(C)C)cc5)cc3)CC4)cc2N=C(N)C1 ; CCCN(CCC)C(=O)C1=Cc2ccc(C(=O)Nc3cnc4c(c3)CN(Cc3ccc(C(=O)NCC(F)(F)CNC(=O)OCc5ccc(NC(=O)[C@H](CCCNC(N)=O)NC(=O)[C@@H](NC(=O)CCCCCN6C(=O)C=CC6=O)C(C)C)cc5)cc3)CC4)cc2N=C(N)C1]

and a salt of any one thereof.

In some aspects, the present disclosure provides a compound or salt selected from:

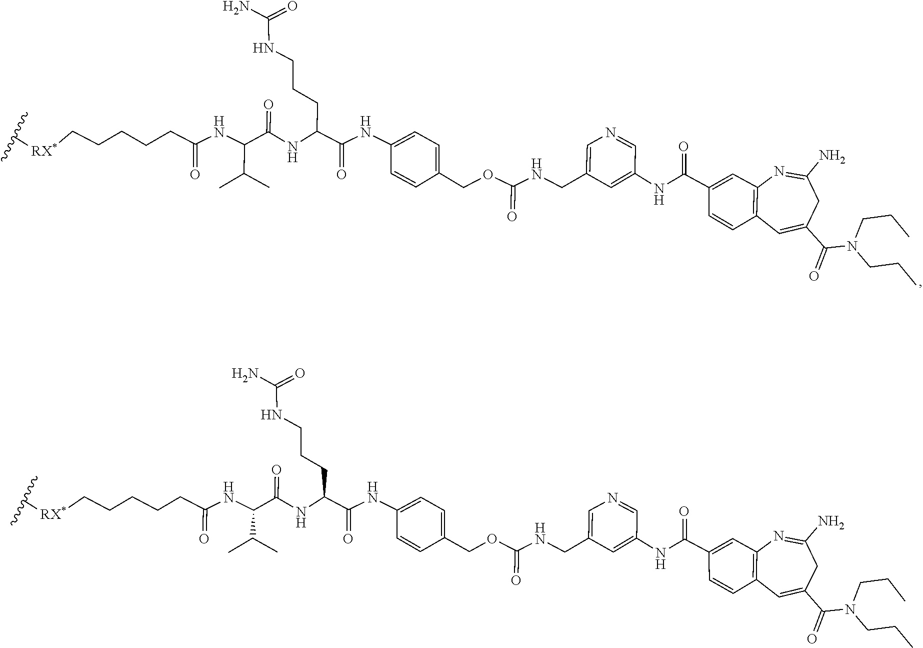

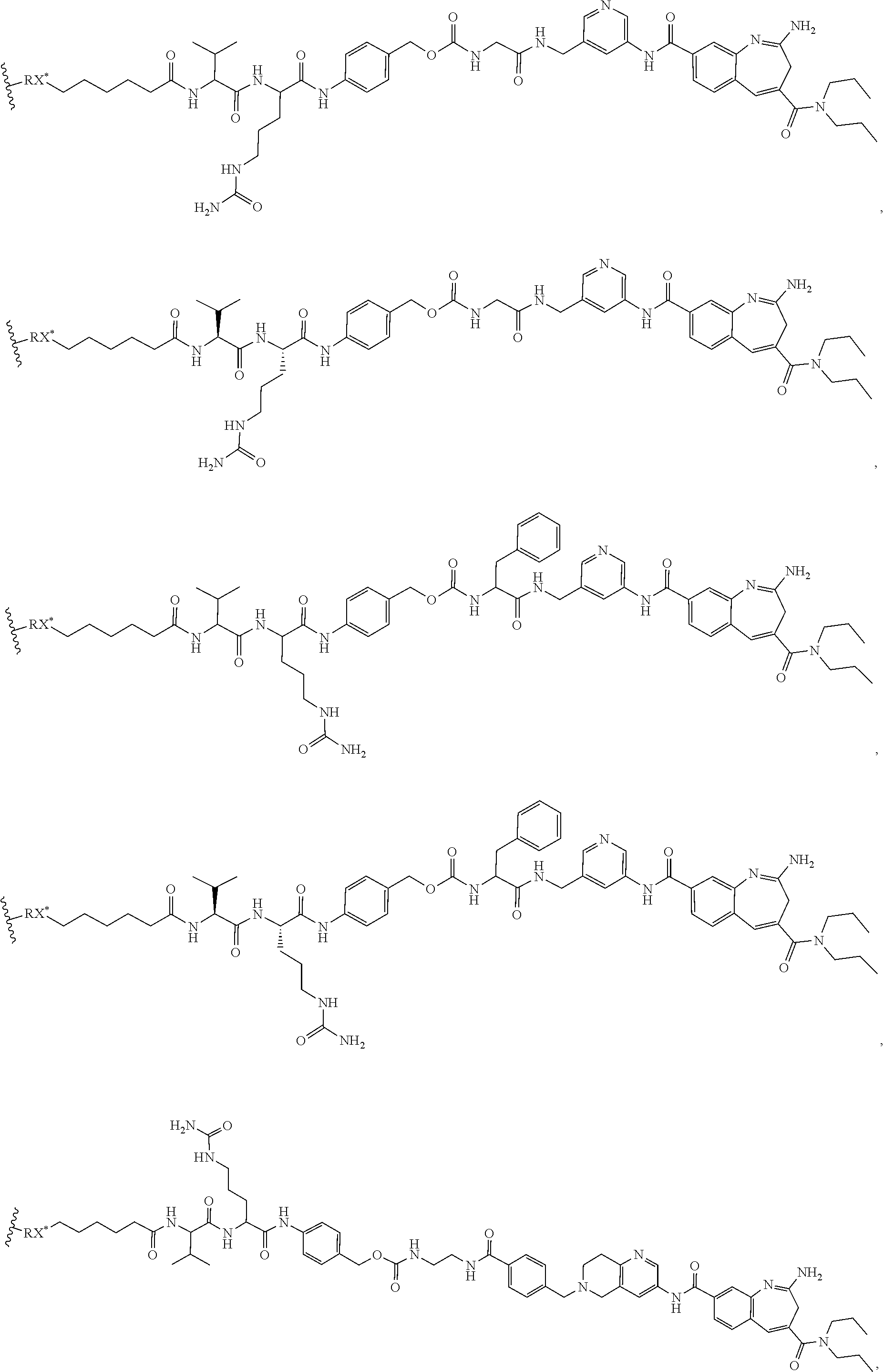

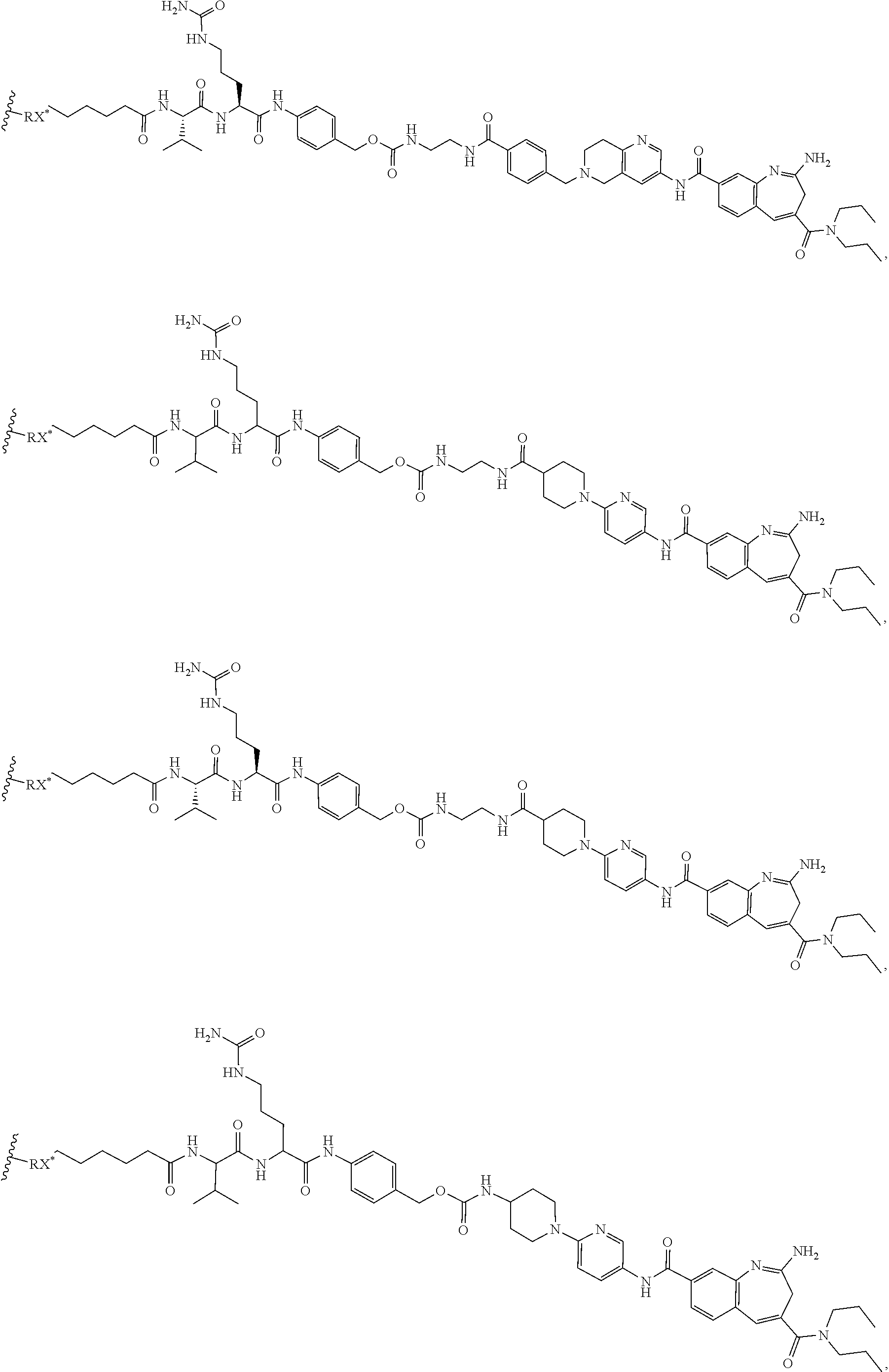

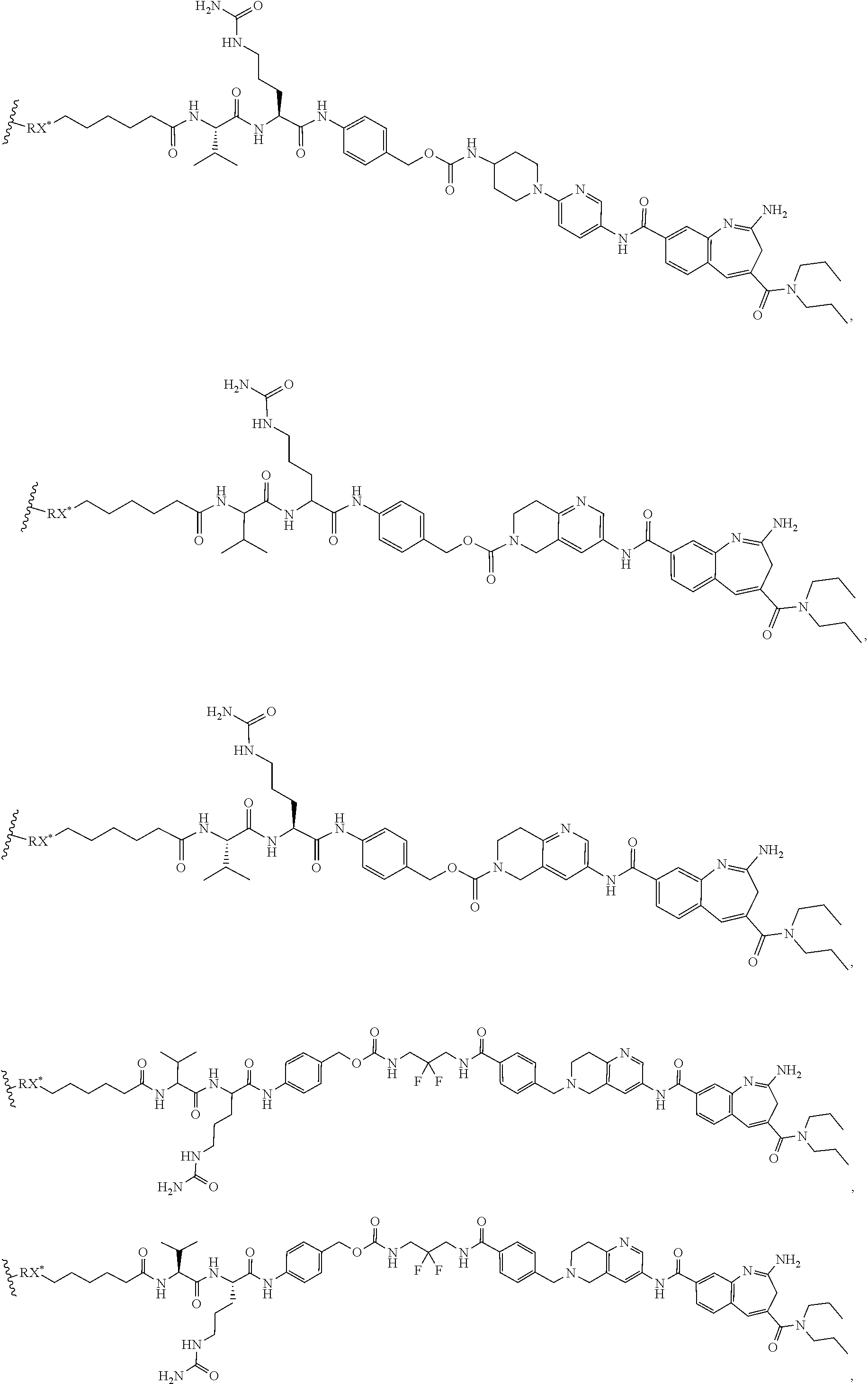

and a salt of any one thereof, wherein the RX* is a bond, a succinimide moiety, or a hydrolyzed succinimide moiety bound to a residue of an antibody construct, wherein

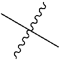

on RX* represents the point of attachment to the residue of the antibody construct.

In some embodiments, L³ is represented by the formula:

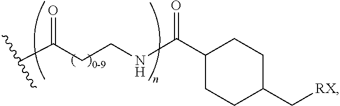

wherein RX comprises a reactive moiety, and n=0-9. In some embodiments, RX comprises a leaving group. In some embodiments, RX comprises a maleimide. In some embodiments, L³ is represented as follows:

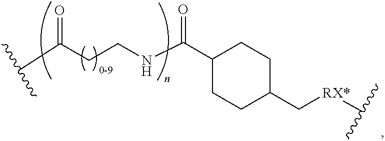

wherein RX* comprises a bond, a succinimide moiety, or a hydrolyzed succinimide moiety bound to a residue of an antibody construct, wherein

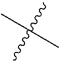

on RX* represents the point of attachment to the residue of the antibody construct, and n=0-9.

In some aspects, the present disclosure provides a compound or salt selected from:

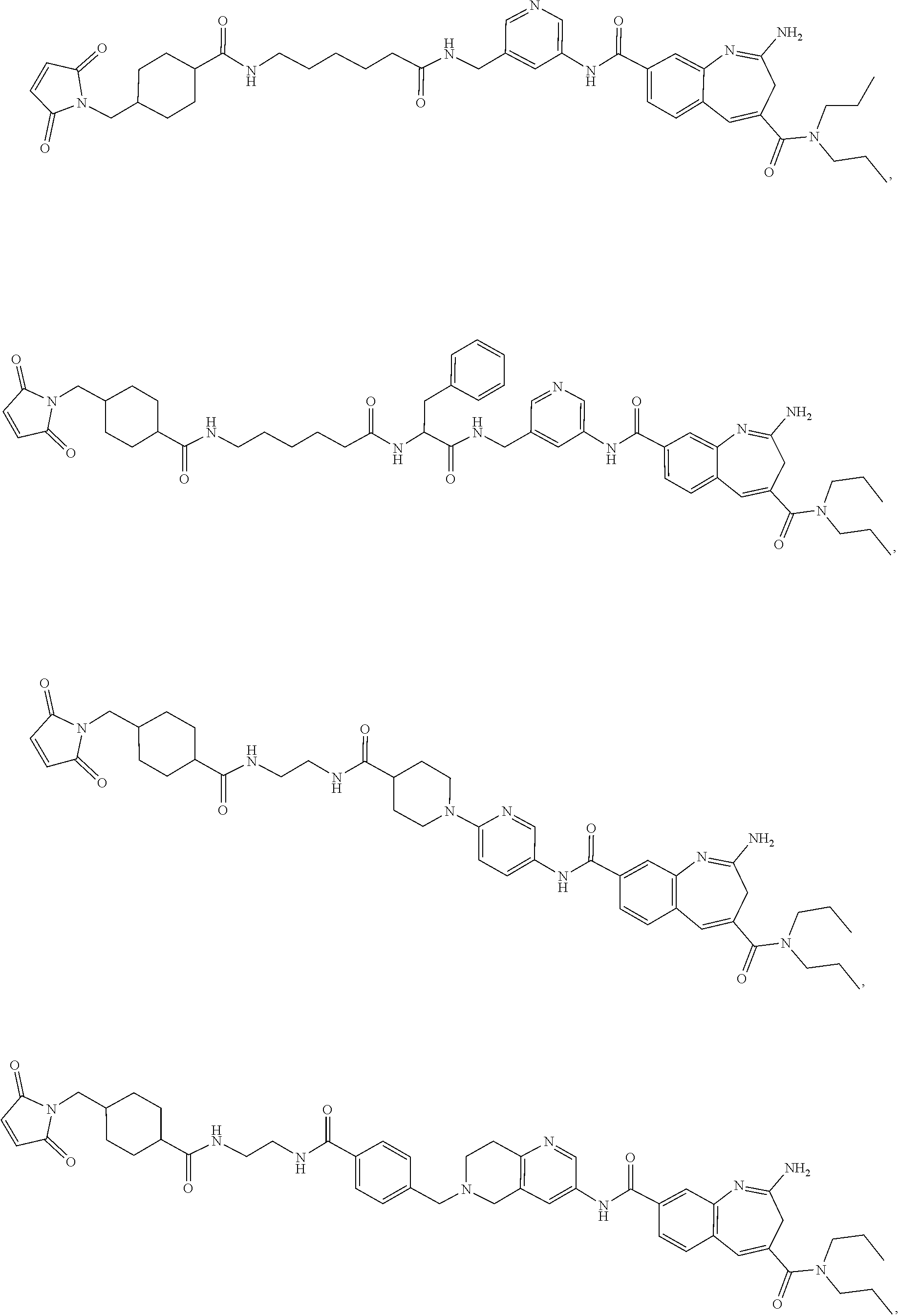 [SMILES: CCCN(CCC)C(=O)C1=Cc2ccc(C(=O)Nc3cncc(CNC(=O)CCCCCNC(=O)C4CCC(CN5C(=O)C=CC5=O)CC4)c3)cc2N=C(N)C1 ; CCCN(CCC)C(=O)C1=Cc2ccc(C(=O)Nc3cncc(CNC(=O)C(Cc4ccccc4)NC(=O)CCCCCNC(=O)C4CCC(CN5C(=O)C=CC5=O)CC4)c3)cc2N=C(N)C1 ; CCCN(CCC)C(=O)C1=Cc2ccc(C(=O)Nc3ccc(N4CCC(C(=O)NCCNC(=O)C5CCC(CN6C(=O)C=CC6=O)CC5)CC4)nc3)cc2N=C(N)C1 ; CCCN(CCC)C(=O)C1=Cc2ccc(C(=O)Nc3cnc4c(c3)CN(Cc3ccc(C(=O)NCCNC(=O)C5CCC(CN6C(=O)C=CC6=O)CC5)cc3)CC4)cc2N=C(N)C1]

and a salt of any one thereof.

In some aspects, the present disclosure provides a compound or salt selected from:

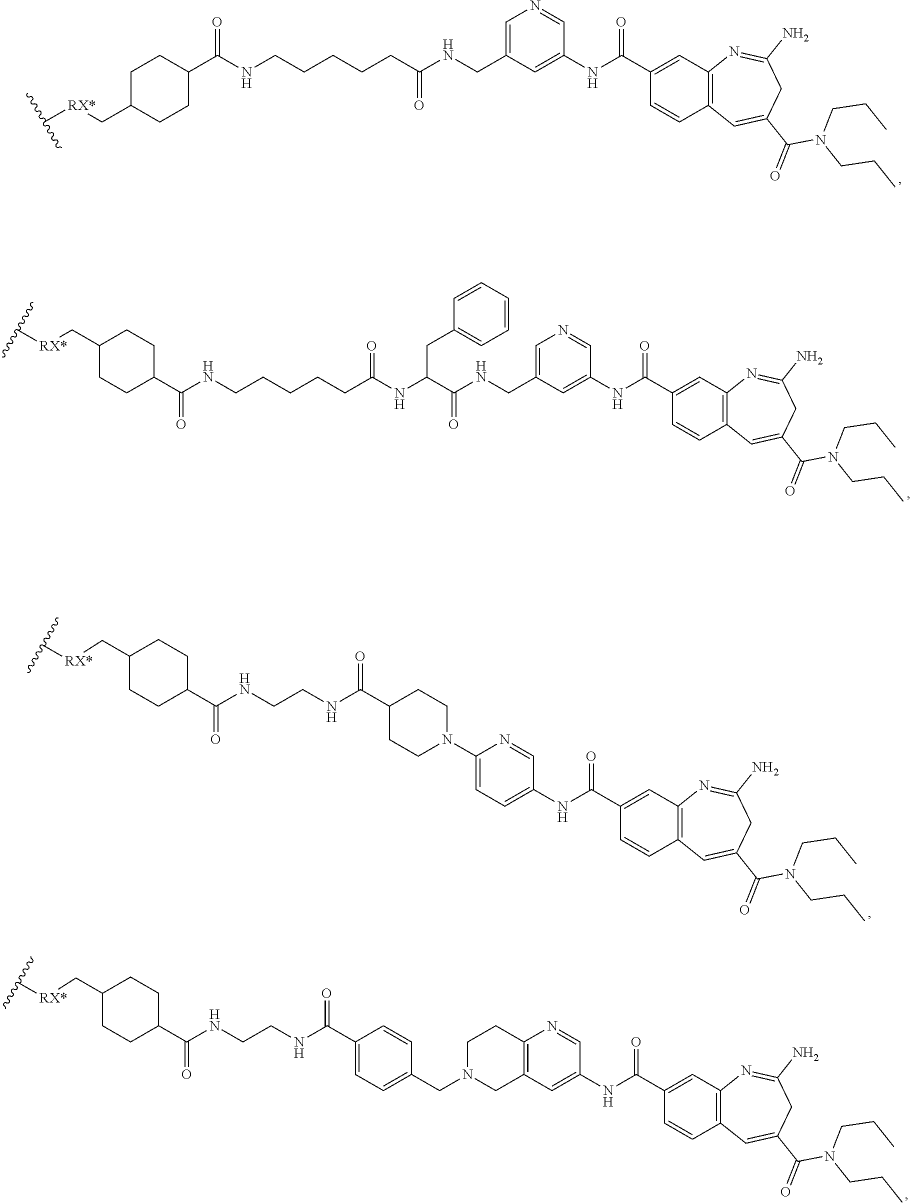

and a salt of any one thereof, wherein the RX* comprises a bond, a succinimide moiety, or a hydrolyzed succinimide moiety bound to a residue of an antibody construct, wherein

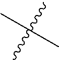

on RX* represents the point of attachment to the residue of the antibody construct.

In some embodiments, RX* comprises a succinamide moiety and is bound to a cysteine residue of an antibody construct. In some embodiments, RX* comprises a hydrolyzed succinamide moiety and is bound to a cysteine residue of an antibody construct.

In some aspects, the present disclosure provides a conjugate represented by the formula:

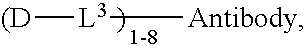

wherein Antibody is an antibody construct, D is a Category A compound or salt disclosed herein, and L³ is a linker moiety.

In some aspects, the present disclosure provides a conjugate represented by the formula:

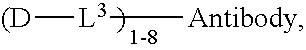

wherein Antibody is an antibody construct and D-L³ is a Category A compound or salt disclosed herein.

In some aspects, the present disclosure provides a pharmaceutical composition, comprising the conjugate disclosed herein and at least one pharmaceutically acceptable excipient.

In some embodiments, the average DAR of the conjugate is from about 2 to about 8, or about 1 to about 3, or about 3 to about 5.

### Compounds of Category B, TLR7 Agonists

In some aspects, the present disclosure provides a compound represented by the structure of Formula (IA):

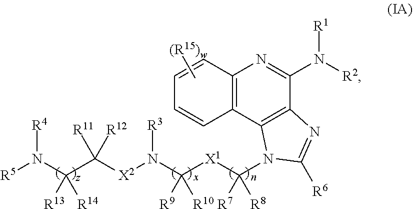

or a salt thereof, wherein:

R¹, R², R³, R⁴, and R⁵ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; or R³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁷, R⁸, R⁹, and R¹⁰ are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, —N(R²⁰), —CN, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂;

n is 1, 2, or 3;

x is 1, 2, or 3;

w is 0, 1, 2, 3, or 4; and

z is 0, 1, or 2.

In certain embodiments, for a compound of Formula (IA), wherein X¹ is O. In certain embodiments, for a compound of Formula (IA), n is 2. In certain embodiments, for a compound of Formula (IA), x is 2. In certain embodiments, for a compound of Formula (IA), z is 0. In certain embodiments, for a compound of Formula (IA), z is 1.

In certain embodiments, a compound of Formula (IA) is represented by Formula (IB):

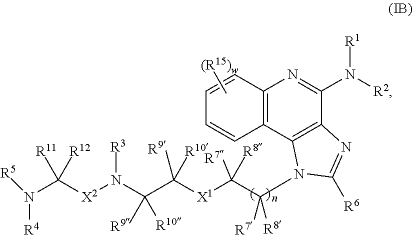

or a salt thereof, wherein:

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

In certain embodiments, a compound of Formula (IA) is represented by Formula (IC):

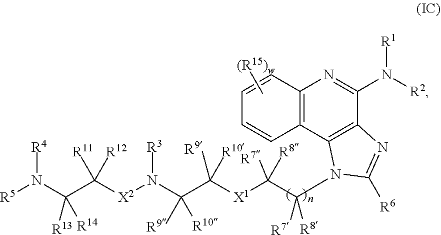

or a salt thereof, wherein:

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

In certain embodiments, for a compound or salt of any one of Formulas (IA), (IB), or (IC), R¹, R², R³, R⁴, and R⁵ are independently selected from hydrogen and C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN.

In certain embodiments, for a compound or salt of any one of Formulas (IA), (IB), or (IC), R¹ and R² are independently selected from hydrogen and C₁₋₆ alkyl. In certain embodiments, for a compound or salt of any one of Formulas (IA), (IB), or (IC), R¹ and R² are each hydrogen.

In certain embodiments, for a compound or salt of any one of Formulas (IA), (IB), or (IC), R³ is selected from hydrogen and C₁₋₆ alkyl optionally substituted with one or more halogens.

In certain embodiments, for a compound or salt of any one of Formulas (IA), (IB), or (IC), R³ is hydrogen.

In certain embodiments, for a compound or salt of any one of Formulas (IA), (IB), or (IC), R⁴ is selected from hydrogen and C₁₋₆ alkyl optionally substituted with one or more halogens.

In certain embodiments, for a compound or salt of any one of Formulas (IA), (IB), or (IC), R⁴ is hydrogen.

In certain embodiments, for a compound or salt of any one of Formulas (IA), (IB), or (IC), R⁵ is selected from hydrogen and C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN. In certain embodiments, for a compound or salt of any one of Formulas (IA), (IB), or (IC), R⁵ is hydrogen.

In certain embodiments, for a compound or salt of any one of Formulas (IA), (IB), or (IC), R⁶ is selected from halogen, —OR²⁰, and —N(R²⁰)₂; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle.

In certain embodiments, for a compound or salt of any one of Formulas (IA), (IB), or (IC), R⁶ is C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰; and

R²⁰ is independently selected at each occurrence from hydrogen; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle.

In certain embodiments, for a compound or salt of any one of Formulas (IA), (IB), or (IC), R⁶ is C₁₋₆ alkyl substituted with —OR²⁰, and

R²⁰ is selected from hydrogen and C₁₋₆ alkyl optionally substituted with one or more substituents independently selected from halogen, —OH, and —NH₂.

In certain embodiments, for a compound or salt of any one of Formulas (IA), (IB), or (IC), R^{7′}, R^{7″}, R^{8′}, R^{8″}, R^{9′}, and R^{10″} are independently selected at each occurrence from hydrogen and halogen; and C₁₋₆ alkyl, optionally substituted with one or more substituents independently selected from halogen.

In certain embodiments, for a compound or salt of any one of Formulas (IB) or (IC), wherein R^{7′} and R^{8′} are each hydrogen. In certain embodiments, for a compound or salt of any one of Formulas (IB) or (IC), wherein R^{7″} and R^{8″} are each C₁₋₆ alkyl. In certain embodiments, for a compound or salt of any one of Formulas (IB) or (IC), R^{7″} and R^{8″} are each methyl.

In certain embodiments, for a compound or salt of any one of Formulas (IB) or (IC), R^{9′}, R^{9″}, R^{10′}, and R^{10″} are independently selected at each occurrence from hydrogen and C₁₋₆ alkyl.

In certain embodiments, for a compound or salt of any one of Formulas (IB) or (IC), R^{9′}, R^{9″}, R^{10′}, and R^{10″} are each hydrogen.

In certain embodiments, for a compound or salt of any one of Formulas (IA), (IB), or (IC), R¹¹ and R¹² are independently selected from hydrogen, halogen, —OR²⁰, —SR²⁰, —C(O)N(R²⁰)₂, —N(R²⁰)₂, —C(O)R²⁰, —C(O)OR²⁰, —OC(O)R²⁰; and C₁₋₆ alkyl, optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle.

In certain embodiments, for a compound or salt of any one of Formulas (IA) or (IC), R¹³ and R¹⁴ are independently selected from hydrogen, halogen, —OR²⁰, —SR²⁰, —C(O)N(R²⁰)₂, —N(R²⁰)₂, —C(O)R²⁰, —C(O)OR²⁰, —OC(O)R²⁰; and C₁₋₆ alkyl optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle.

In certain embodiments, for a compound or salt of any one of Formulas (IA), (IB), or (IC), R³ and R¹¹ taken together form an optionally substituted 5- to 6-membered heterocycle.

In certain embodiments, for a compound or salt of any one of Formulas (IA), (IB), or (IC), R¹¹ and R¹² taken together form an optionally substituted C₃₋₆ carbocycle.

In certain embodiments, for a compound or salt of any one of Formulas (IA), (IB), or (IC), X² is C(O).

In certain embodiments, the compound is represented by:

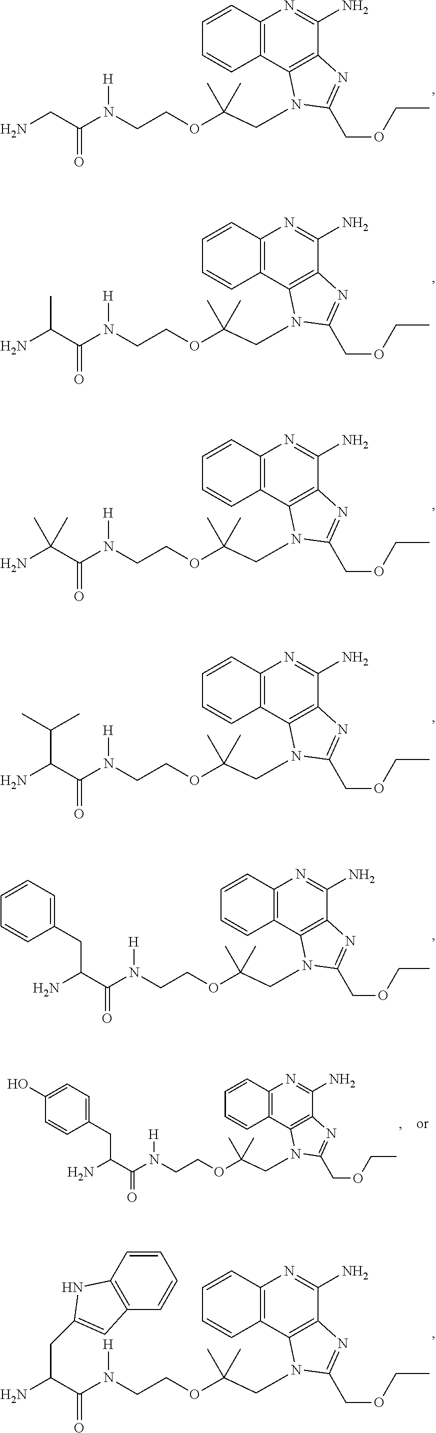

or a salt of any one thereof.

In certain aspects, the disclosure provides a pharmaceutical composition of a compound or salt of any one of Formulas (IA), (IB), or (IC), and a pharmaceutically acceptable excipient.

In certain embodiments, for a compound or salt of any one of Formulas (IA), (IB), or (IC), the compound or salt is further covalently bound to a linker, L³.

In certain aspects the disclosure provides a compound represented by Formula (IIA):

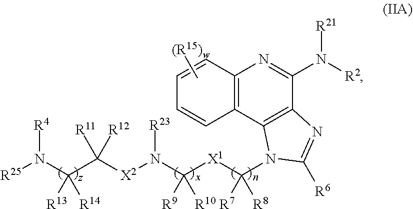

or a salt thereof, wherein:

R² and R⁴ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R²¹, R²³, and R²⁵ are independently selected from hydrogen; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and L³; or R²³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and wherein one of R²¹, R²³, and R²⁵ is L³;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁷, R⁸, R⁹, and R¹⁰ are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and 10² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, —CN, C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

L³ is a linker;

X¹ is O, S, or NR¹⁶;

X² is C(O) or S(O)₂;

n is 1, 2, or 3;

x is 1, 2, or 3;

w is 0, 1, 2, 3, or 4; and

z is 0, 1, or 2.

In certain embodiments, for a compound or salt of Formula (IIA), X¹ is O. In certain embodiments, for a compound or salt of Formula (IIA), n is 2. In certain embodiments, for a compound or salt of Formula (IIA), x is 2. In certain embodiments, for a compound or salt of Formula (IIA), z is 0. In certain embodiments, for a compound or salt of Formula (IIA), z is 1.

In certain embodiments, the compound of Formula (IIA) is represented by (IIB) or (IIC):

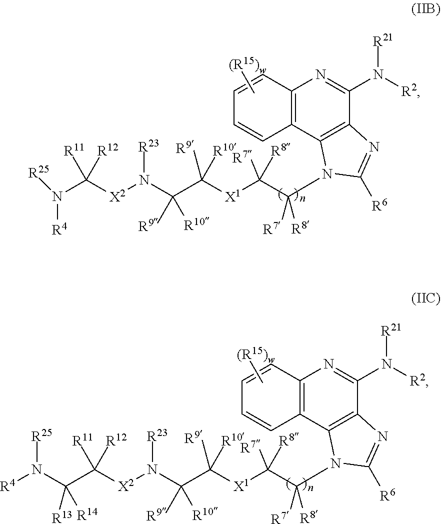

or a salt thereof, wherein:

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

In certain embodiments, for a compound or salt of any one of Formulas (IIA), (IIB), or (IIC), R² and R⁴ are independently selected from hydrogen and C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN.

In certain embodiments, for a compound or salt of any one of Formulas (IIA), (IIB), or (IIC), R² and R⁴ are independently selected from hydrogen and C₁₋₆ alkyl. In certain embodiments, for a compound or salt of any one of Formulas (IIA), (IIB), or (IIC), R² and R⁴ are each hydrogen.

In certain embodiments, for a compound or salt of any one of Formulas (IIA), (IIB), or (IIC), R²³ is selected from hydrogen and C₁₋₆ alkyl optionally substituted with one or more halogens. In certain embodiments, for a compound or salt of any one of Formulas (IIA), (IIB), or (IIC), R²³ is hydrogen.

In certain embodiments, for a compound or salt of any one of Formulas (IIA), (IIB), or (IIC), R²¹ is selected from hydrogen and C₁₋₆ alkyl optionally substituted with one or more halogens. In certain embodiments, for a compound or salt of any one of Formulas (IIA), (IIB), or (IIC), R²¹ is hydrogen.

In certain embodiments, for a compound or salt of any one of Formulas (IIA), (IIB), or (IIC), R²¹ is L³.

In certain embodiments, for a compound or salt of any one of Formulas (IIA), (IIB), or (IIC), R²⁵ is selected from hydrogen and C₁₋₆alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN. In certain embodiments, for a compound or salt of any one of Formulas (IIA), (IIB), or (IIC), R²⁵ is hydrogen.

In certain embodiments, for a compound or salt of any one of Formulas (IIA), (IIB), or (IIC), R²⁵ is L³.

In certain embodiments, for a compound or salt of any one of Formulas (IIA), (IIB), or (IIC), R⁶ is selected from halogen, —OR²⁰, and —N(R²⁰)₂; and C₁₋₆alkyl, C₂₋₆alkenyl, C₂₋₆alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and

R²⁰ is independently selected at each occurrence from hydrogen; and C₁₋₆ alkyl, C₂₋₆ alkenyl, C₂₋₆alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆alkynyl, C₃₋₁₂ carbocycle, and 3- to 12-membered heterocycle.

In certain embodiments, for a compound or salt of any one of Formulas (IIA), (IIB), or (IIC), R⁶ is C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰; and

R²⁰ is independently selected at each occurrence from hydrogen, —NH₂, —C(O)OCH₂C₆H₅; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆alkynyl, C₃₋₁₂ carbocycle, and 3- to 12-membered heterocycle.

In certain embodiments, for a compound or salt of any one of Formulas (IIA), (IIB), or (IIC), R⁶ is C₁₋₆ alkyl substituted with —OR²⁰, and

R²⁰ is selected from hydrogen and C₁₋₆ alkyl, which is optionally substituted with one or more substituents independently selected from halogen, —OH, and —NH₂.

In certain embodiments, for a compound or salt of any one of Formulas (IIB) or (IIC), R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl optionally substituted with one or more substituents independently selected from halogen.

In certain embodiments, for a compound or salt of any one of Formulas (IIB) or (IIC), R^{7′} and R^{8′} are hydrogen.

In certain embodiments, for a compound or salt of any one of Formulas (IIB) or (IIC), R^{7″} and R^{8″} are C₁₋₆ alkyl.

In certain embodiments, for a compound or salt of any one of Formulas (IIB) or (IIC), R^{7″} and R^{8″} are methyl.

In certain embodiments, for a compound or salt of any one of Formulas (IIB) or (IIC), R^{9′}, R^{9″}, R^{10′}, and R^{10″} are independently selected at each occurrence from hydrogen and C₁₋₆ alkyl.

In certain embodiments, for a compound or salt of any one of Formulas (IIB) or (IIC), R^{9′}, R^{9″}, R^{10′}, and R^{10″} are each hydrogen.

In certain embodiments, for a compound or salt of any one of Formulas (IIA), (IIB), or (IIC), R¹¹ and R¹² are independently selected from hydrogen, halogen, —OR²⁰, —SR²⁰, —C(O)N(R²⁰)₂, —N(R²⁰)₂, —C(O)R²⁰, —C(O)OR²⁰, and —OC(O)R²⁰; and C₁₋₆ alkyl optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle.

In certain embodiments, for a compound or salt of any one of Formulas (IIA) or (IIC), R¹³ and R¹⁴ are independently selected from hydrogen, halogen, —OR²⁰, —SR²⁰, —C(O)N(R²⁰)₂, —N(R²⁰)₂, —C(O)R²⁰, —C(O)OR²⁰, and —OC(O)R²⁰, and C₁₋₆ alkyl optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle.

In certain embodiments, for a compound or salt of any one of Formulas (IIA), (IIB), or (IIC), R²³ and R¹¹ taken together form an optionally substituted 5- to 6-membered heterocycle.

In certain embodiments, for a compound or salt of any one of Formulas (IIA), (IIB), or (IIC), R¹¹ and R² taken together form an optionally substituted C₃₋₆ carbocycle.

In certain embodiments, for a compound or salt of any one of Formulas (IIA), (IIB), or (IIC), X² is C(O).

In certain embodiments, for a compound or salt of any one of Formulas (IIA), (IIB), or (IIC), L³ is a cleavable linker. In certain embodiments, for a compound or salt of any one of Formulas (IIA), (IIB), or (IIC), L³ is cleavable by a lysosomal enzyme.

In certain embodiments, for a compound or salt of any one of Formulas (IIA), (IIB), or (IIC), L³ is represented by the formula:

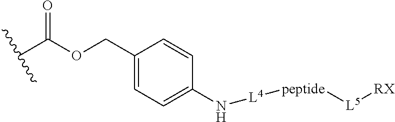

wherein:

L⁴ represents the C-terminus of the peptide and L⁵ is selected from a bond, alkylene and heteroalkylene, wherein L⁵ is optionally substituted with one or more groups independently selected from R³⁰, and RX is a reactive moiety; and

R³⁰ is independently selected at each occurrence from halogen, —OH, —CN, —O-alkyl, —SH, ═O, ═S, —NH₂, —NO₂; and C₁-C₁₀ alkyl, C₂-C₁₀ alkenyl, and C₂-C₁₀ alkynyl, each of which is independently optionally substituted at each occurrence with one or more substituents selected from halogen, —OH, —CN, —O-alkyl, —SH, ═O, ═S, —NH₂, and —NO₂.

In certain embodiments, for a compound or salt of any one of Formulas (IIA), (IIB), or (IIC), RX comprises a leaving group. In certain embodiments, for a compound or salt of any one of Formulas (IIA), (IIB), or (IIC), RX is a maleimide or an alpha-halo carbonyl. In certain embodiments, for a compound or salt of any one of Formulas (IIA), (IIB), or (IIC), the peptide of L³ comprises Val-Cit or Val-Ala.

In certain embodiments, for a compound or salt of any one of Formulas (IIA), (IIB), or (IIC), L³ is represented by the formula:

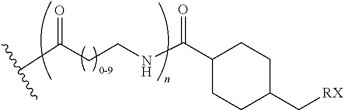

wherein:

RX comprises a reactive moiety; and

n is 0-9.

In certain embodiments, for a compound or salt of any one of Formulas (IIA), (IIB), or (IIC), RX comprises a leaving group. In certain embodiments, for a compound or salt of any one of Formulas (IIA), (IIB), or (IIC), RX is a maleimide or an alpha-halo carbonyl. In certain embodiments, for a compound or salt of any one of Formulas (IIA), (IIB), or (IIC), L³ is further covalently bound to an antibody construct to form a conjugate. In certain embodiments, the disclosure provides a conjugate represented by the formula:

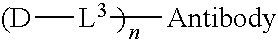

wherein:

Antibody is an antibody construct;

n is 1 to 20;

D is a compound or salt of any one of a Category B compound of Formulas (IA), (IB), or (IC); and L³ is a linker moiety; or

D-L³ is a compound or salt of any one of a Category B compound of Formulas (IIA), (IIB), or (IIC).

In certain embodiments, for a conjugate of a compound or salt of any one of Formulas (IA), (IB), (IC), (IIA), (IIB), and (IIC), n is selected from 1 to 8. In certain embodiments, for a conjugate of a compound or salt of any one of Formulas (IA), (IB), (IC), (IIA), (IIB), and (IIC), n is selected from 2 to 5. In certain embodiments, for a conjugate of a compound or salt of any one of Formulas (IA), (IB), (IC), (IIA), (IIB), and (IIC), n is 2.

In certain embodiments, for a compound or salt of any one of Formulas (IIA), (IIB), and (IIC), -L³ is represented by the formula:

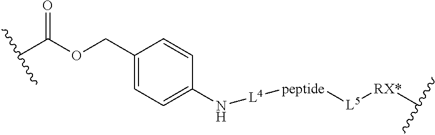

wherein:

L⁴ represents the C-terminus of the peptide and L⁵ is selected from a bond, alkylene and heteroalkylene, wherein L⁵ is optionally substituted with one or more groups independently selected from R³⁰; RX* is a bond, a succinimide moiety, or a hydrolyzed succinimide moiety bound to a residue of an antibody construct, wherein

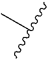

on RX* represents the point of attachment to the residue of the antibody construct; and

R³⁰ is independently selected at each occurrence from halogen, —OH, —CN, —O-alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂.

In certain embodiments, for a compound or salt of any one of Formulas (IIA), (IIB), or (IIC), RX* is a succinamide moiety, hydrolyzed succinamide moiety or a mixture thereof and is bound to a cysteine residue of an antibody construct.

In certain embodiments for a compound of Formulas (IIA), (IIB) and (IIC), -L³ is represented by the formula:

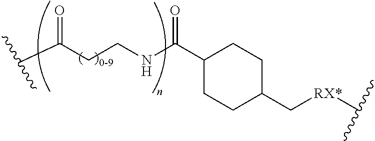

wherein:

RX* is a bond, a succinimide moiety, or a hydrolyzed succinimide moiety bound to a residue of an antibody construct, wherein

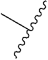

on RX* represents the point of attachment to the residue of the antibody construct; and

n is 0-9.

### Category A and Category B Conjugates

In certain embodiments, the disclosure provides a pharmaceutical composition, comprising a conjugate of a compound of any one of Category A Formulas (IA), (IB), (IIA), (IIB), (IIIA), and (IIIB), and a pharmaceutically acceptable excipient. In certain embodiments, the disclosure provides a pharmaceutical composition, comprising a conjugate of a compound of any one of Category B Formulas (IA), (IB), or (IC), and a pharmaceutically acceptable excipient. In certain embodiments, the average Drug-to-Antibody Ratio (DAR) of the pharmaceutical composition is selected from 1 to 8.

In certain embodiments, the disclosure provides a method for the treatment of HBV or HCV viral infection, comprising administering an effective amount of the conjugate of a compound of any one of Category A Formulas (IA), (IB), (IIA), (IIB), (IIIA), and (IIIB), or a pharmaceutical composition thereof to a subject in need thereof. In certain embodiments, the disclosure provides a method for the treatment of HBV or HCV viral infection, comprising administering an effective amount of the conjugate of a compound of any one of Category B Formulas (IA), (IB), or (IC), or a pharmaceutical composition thereof to a subject in need thereof.

In certain embodiments, the disclosure provides a method for killing HBV or HCV infected liver cells in vivo, comprising contacting HBV or HCV infected liver cells with the conjugate of a compound of any one of Category A Formulas (IA), (IB), (IIA), (IIB), (IIIA), and (IIIB), or a pharmaceutical composition thereof. In certain embodiments, the disclosure provides a method for killing HBV or HCV infected liver cells in vivo, comprising contacting HBV or HCV infected liver cells with the conjugate of a compound of any one of B Formulas (IA), (IB), or (IC), or a pharmaceutical composition thereof.

In certain embodiments, the disclosure provides a method for treatment, comprising administering to a subject the conjugate of a compound of any one of Category A Formulas (IA), (IB), (IIA), (IIB), (IIIA), and (IIIB), or a pharmaceutical composition thereof. In certain embodiments, the disclosure provides a method for treatment, comprising administering to a subject the conjugate of a compound of any one of Category B Formulas (IA), (IB), or (IC) or a pharmaceutical composition thereof.

In certain embodiments, the disclosure provides a method for the treatment of HBV or HCV infections, comprising administering to a subject in need thereof the conjugate of a compound of any one of Category A Formulas (IA), (IB), (IIA), (IIB), (IIIA), and (IIIB), or a pharmaceutical composition thereof. In certain embodiments, the disclosure provides a method for the treatment of HBV or HCV infections, comprising administering to a subject in need thereof the conjugate of a compound of any one of Category B Formulas (IA), (IB), or (IC), or a pharmaceutical composition thereof.

The disclosure provides a conjugate of a compound of any one of Category A Formulas (IA), (IB), (IIA), (IIB), (IIIA), and (IIIB), or a pharmaceutical composition thereof for use in a method of treatment of a viral infection, such as HBV or HCV described herein. The disclosure provides a conjugate of a compound of any one of Category B Formulas (IA), (IB), or (IC) or a pharmaceutical composition thereof for use in a method of treatment of a viral infection, such as HBV or HCV described herein.

The disclosure provides a method of preparing an antibody conjugate of the formula:

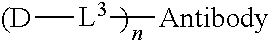

wherein:

Antibody is an antibody construct;

n is selected from 1 to 20;

L³ is a linker; and

D is selected from a compound or salt of a compound of any one of Category A Formulas (IA), (IB), (IIA), (IIB), (IIIA), and (IIIB) and Category B Formulas (IA), (IB), or (IC),

comprising contacting D-L³ with an antibody construct.

The disclosure provides a method of preparing an antibody conjugate of the formula:

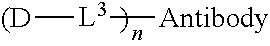

wherein:

Antibody is an antibody construct;

n is selected from 1 to 20;

L³ is a linker; and

D is selected from a compound of any one of Category A Formulas (IA), (IB), (IIA), (IIB), (IIIA), and (IIIB) and Category B Formulas (IA), (IB), or (IC),

comprising contacting L³ with the antibody construct to form L³-antibody and contacting L³ antibody with D to form the conjugate.

The compounds disclosed herein, in some embodiments, are used in different enriched isotopic forms, e.g., enriched in the content of ²H, ³H, ¹¹C ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds described herein may be prodrugs attached to antibody constructs to form conjugates. The term “prodrug” is intended to encompass compounds which, under physiologic conditions, are converted into active compounds, e.g., TLR8 or TLR7 agonists. One method for making a prodrug is to include one or more selected moieties which are hydrolyzed or otherwise cleaved under physiologic conditions to reveal the desired molecule. In other embodiments, the prodrug is converted by an enzymatic activity of the host animal such as specific target cells in the host animal.

Prodrug forms of the herein described compounds, wherein the prodrug is metabolized in vivo to produce a compound described herein are included within the scope of the claims. In some cases, some of the herein-described compounds may be a prodrug for another derivative or active compound.

In certain embodiments, a compound such as a TLR8 agonist or TLR7 agonist is modified as a prodrug with a masking group, such that the TLR8 agonist or TLR7 agonist has limited activity or is inactive until it reaches an environment where the masking group is removed to reveal the active compound. For example, the TLR8 agonist or TLR7 agonist is covalently modified at an amine involved in binding to the active site of a TLR8 receptor such that the compound is unable to bind the active site of the receptor in its modified (prodrug) form. In such an example, the masking group may be removed under physiological conditions, e.g., enzymatic or acidic conditions, specific to the site of delivery, e.g., intracellular or extracellular adjacent to target cells. Masking groups may be removed from the amine of the compound or salt described herein due to the action of lysosomal proteases, e.g., cathepsin and plasmin. These proteases can be present at elevated levels in certain tumor tissues. The masking group may be removed by a lysosomal enzyme. The lysosomal enzyme can be, for example, cathepsin B, cathepsin S, β-glucuronidase, or β-galactosidase.

In certain embodiments, the amine masking group inhibits binding of the amine group of the compound with residues of a TLR8 receptor. The amine masking group may be removable under physiological conditions within a cell but remains covalently bound to the amine outside of a cell. Masking groups that may be used to inhibit or attenuate binding of an amine group of a compound with residues of a TLR8 receptor include, for example, peptides and carbamates.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Linkers

The conjugates include a linker that attaches an antibody construct to at least one myeloid agonist. The linker can be, for example, a cleavable or a non-cleavable linker. Linkers of the conjugates and methods described herein may not affect the binding of active portions of a conjugate (e.g., antigen binding domains and Fc binding domains) to a target or an Fc receptor. A conjugate can comprise multiple linkers. These linkers can be the same linkers or different linkers.

As will be appreciated by skilled artisans, a linker connects a myeloid cell agonist to the antibody construct of the conjugate by forming a covalent linkage to the myeloid cell agonist at one location and a covalent linkage to the antibody construct of the conjugate at another location. The covalent linkages can be formed by reaction between functional groups on the linker and functional groups on the myeloid cell agonist and antibody construct. As used herein, the expression “linker” can include (i) unconjugated forms of the linker that can include a functional group capable of covalently linking the linker to a myeloid cell agonist and a functional group capable of covalently linking the linker to an antibody construct; (ii) partially conjugated forms of the linker that can include a functional group capable of covalently linking the linker to an antibody construct of the conjugate and that can be covalently linked to a myeloid cell agonist, or vice versa; and (iii) fully conjugated forms of the linker that can be covalently linked to both a myeloid cell agonist and an antibody construct. In some specific embodiments, conjugates described herein, the functional groups on the linker and covalent linkages formed between the linker and antibody construct of the conjugate can be specifically illustrated as Rx and LK, respectively. One embodiment pertains to a conjugate formed by contacting an antibody construct that specifically binds to a liver antigen(s), a viral antigen(s) expressed on a liver cell or both, with a linker described herein under conditions in which the linker covalently links to the antibody construct. One embodiment pertains to a method of making a conjugate formed by contacting a linker described herein under conditions in which the linker covalently links to an antibody construct.

Attachment via a linker can involve incorporation of a linker between parts of a conjugate. A linker can be short, flexible, rigid, cleavable, non-cleavable, hydrophilic, or hydrophobic. A linker can contain segments that have different characteristics, such as segments of flexibility or segments of rigidity. The linker can be chemically stable to extracellular environments, for example, chemically stable in the blood stream, or may include linkages that are not stable. The linker can include linkages that are designed to cleave and/or immolate or otherwise breakdown specifically or non-specifically inside cells. A cleavable linker can be sensitive to (i.e., cleavable by) enzymes at a specific site. A cleavable linker can be cleaved by enzymes such as proteases. A cleavable linker can be a valine-citrulline peptide or a valine-alanine peptide. A valine-citrulline- or valine-alanine-containing linker can contain a pentafluorophenyl group. A valine-citrulline or valine-alanine-containing linker can contain a succimide or a maleimide group. A valine-citrulline- or valine-alanine-containing linker can contain a para aminobenzoic acid (PABA) group. A valine-citrulline- or valine-alanine-containing linker can contain a PABA group and a pentafluorophenyl group. A valine-citrulline- or valine-alanine-containing linker can contain a PABA group and a succinimide group. A valine-citrulline- or valine-alanine-containing linker can contain a PABA group and a maleimide group. A non-cleavable linker can be protease insensitive (i.e., non-cleavable). A non-cleavable linker can contain a maleimide group. A non-cleavable linker can contain a succinimide group. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group.

A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonyl) linker. A linker can also be an alkylene, alkenylene, alkynylene, polyether, polyester, polyamide, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LXPTG recognition motif (SEQ ID NO: 1) to an N-terminal GGG motif to regenerate a native amide bond. The linker created can therefore link a moiety attached to the LXPTG recognition motif (SEQ ID NO: 1) with a moiety attached to the N-terminal GGG motif. A linker can be a link created between an unnatural amino acid on one moiety reacting with oxime bond that was formed by modifying a ketone group with an alkoxyamine on another moiety. A moiety can be part of a conjugate. A moiety can be part of an antibody construct, such as an antibody. A moiety can be part of a myeloid cell agonist. A moiety can be part of a binding domain. A linker can be unsubstituted or substituted, for example, with a substituent. A substituent can include, for example, hydroxyl groups, amino groups, nitro groups, cyano groups, azido groups, carboxyl groups, carboxaldehyde groups, imine groups, alkyl groups, alkenyl groups, alkynyl groups, alkoxy groups, acyl groups, acyloxy groups, amide groups, and ester groups.

In a conjugate as described herein, the myeloid cell agonist is linked to the antibody construct of the conjugate by way of linkers. The linker attaching a myeloid cell agonist to the antibody construct of the conjugate can be short, long, hydrophobic, hydrophilic, flexible or rigid, or may be composed of segments that each independently have one or more of the above-mentioned properties such that the linker may include segments having different properties. A linker can be polyvalent such that it covalently links more than one myeloid cell agonist to a single site on the antibody construct, or monovalent such that covalently it links a single myeloid cell agonist to a single site on the antibody construct of the conjugate.

Exemplary polyvalent linkers that may be used to link many myeloid cell agonists to an antibody construct of the conjugate are described. For example, Fleximer® linker technology has the potential to enable high-DAR conjugate with good physicochemical properties. As shown below, the Fleximer® linker technology is based on incorporating drug molecules into a solubilizing poly-acetal backbone via a sequence of ester bonds. The methodology renders highly-loaded conjugates (DAR up to 20) whilst maintaining good physicochemical properties. This methodology could be utilized with myeloid cell agonist as shown in the Scheme below.

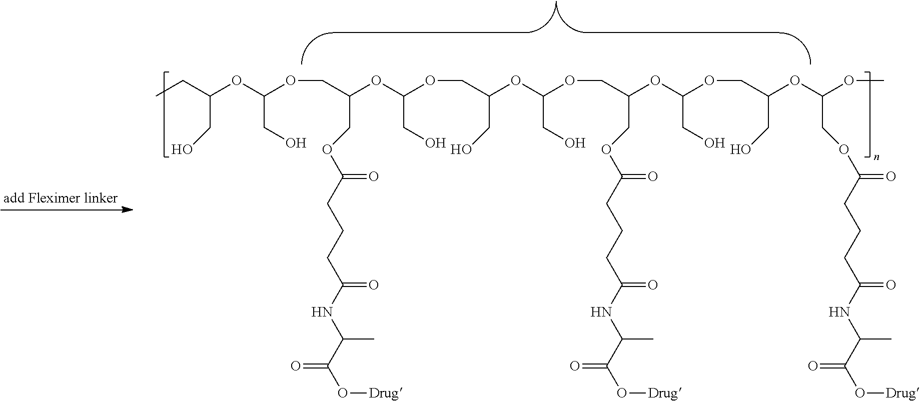

To utilize the Fleximer® linker technology depicted in the scheme above, an aliphatic alcohol can be present or introduced into the myeloid cell agonist. The alcohol moiety is then conjugated to an alanine moiety, which is then synthetically incorporated into the Fleximer® linker. Liposomal processing of the conjugate in vitro releases the parent alcohol-containing drug.

By way of example and not limitation, some cleavable and noncleavable linkers that may be included in the conjugates described herein are described below.

Cleavable linkers can be cleavable in vitro and in vivo. Cleavable linkers can include chemically or enzymatically unstable or degradable linkages. Cleavable linkers can rely on processes inside the cell to liberate a myeloid agonist, such as reduction in the cytoplasm, exposure to acidic conditions in the lysosome, or cleavage by specific proteases or other enzymes within the cell. Cleavable linkers can incorporate one or more chemical bonds that are either chemically or enzymatically cleavable while the remainder of the linker can be non-cleavable.

A linker can contain a chemically labile group such as hydrazone and/or disulfide groups. Linkers comprising chemically labile groups can exploit differential properties between the plasma and some cytoplasmic compartments. The intracellular conditions that can facilitate myeloid cell agonist release for hydrazone containing linkers can be the acidic environment of endosomes and lysosomes, while the disulfide containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release the myeloid cell agonist once the conjugate is internalized into mildly acidic endosomal (pH 5.0-6.5) and lysosomal (pH 4.5-5.0) compartments of the cell. This pH dependent release mechanism can be associated with nonspecific release of the drug. To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites. Conjugates including exemplary hydrazone-containing linkers can include, for example, the following structures:

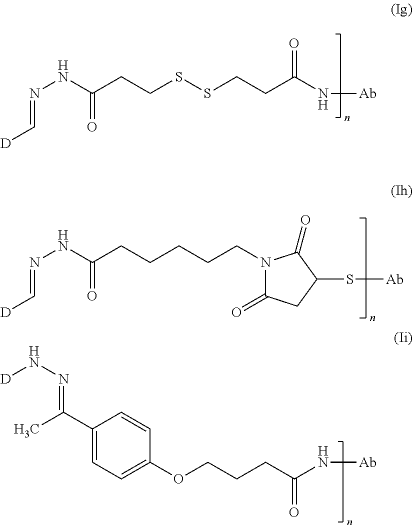

wherein D and Ab represent the myeloid cell agonist and antibody construct, respectively, and n represents the number of myeloid cell agonists-linkers linked to the antibody construct. In certain linkers such as linker (Ig), the linker can comprise two cleavable groups a disulfide and a hydrazone moiety. For such linkers, effective release of the unmodified free myeloid cell agonist can require acidic pH or disulfide reduction and acidic pH. Linkers such as (Ih) and (Ii) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release the myeloid cell agonist upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing the myeloid cell agonist in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Conjugates including exemplary disulfide-containing linkers can include the following structures:

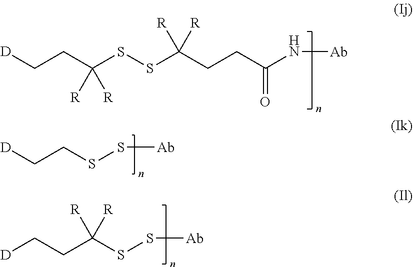

wherein D and Ab represent the myeloid cell agonist and antibody construct, respectively, n represents the number of myeloid cell agonist-linkers linked to the antibody construct and R is independently selected at each occurrence from hydrogen or alkyl, for example. Increasing steric hindrance adjacent to the disulfide bond can increase the stability of the linker. Structures such as (Ij) and (Il) can show increased in vivo stability when one or more R groups is selected from a lower alkyl such as methyl.

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of a myeloid cell agonist from a conjugate can occur due to the action of lysosomal proteases, e.g., cathepsin and/or plasmin. These proteases can be present at elevated levels in certain tumor tissues. The linker can be cleavable by a lysosomal enzyme. The lysosomal enzyme can be, for example, cathepsin B, β-glucuronidase, or β-galactosidase.

In a linker, a cleavable peptide can be selected from tetrapeptides such as Gly-Phe-Leu-Gly (SEQ ID NO: 5), Ala-Leu-Ala-Leu (SEQ ID NO: 6) or dipeptides such as Val-Cit, Val-Ala, and Phe-Lys. Dipeptides can have lower hydrophobicity compared to longer peptides, depending on the composition of the peptide. A variety of dipeptide-based cleavable linkers can be used in the conjugates described herein.

Enzymatically cleavable linkers can include a self-immolative spacer to spatially separate the myeloid cell agonist from the site of enzymatic cleavage. The direct attachment of a myeloid cell agonist to a peptide linker can result in proteolytic release of an amino acid adduct of the myeloid cell agonist, thereby impairing its activity. The use of a self-immolative spacer can allow for the elimination of the fully active, chemically unmodified myeloid cell agonist upon amide bond hydrolysis.

One self-immolative spacer can be a bifunctional para-aminobenzyl alcohol group, which can link to the peptide through the amino group, forming an amide bond, while amine containing myeloid cell agonists can be attached through carbamate functionalities to the benzylic hydroxyl group of the linker (to give a p-amidobenzylcarbamate, PABC). The resulting pro-myeloid cell agonist can be activated upon protease-mediated cleavage, leading to a 1,6-elimination reaction releasing the unmodified myeloid cell agonist, carbon dioxide, and remnants of the linker group. The following scheme depicts the fragmentation of p-amidobenzyl carbamate and release of the myeloid cell agonist:

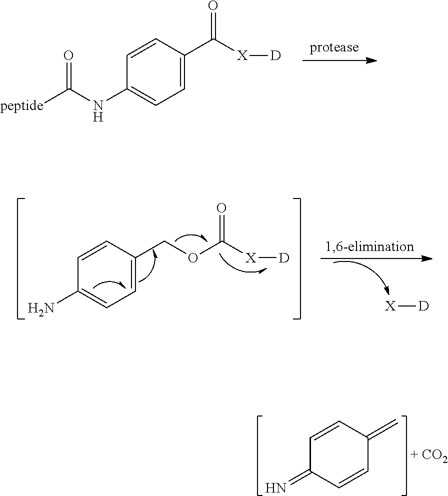

wherein X-D represents the unmodified myeloid cell agonist.

The enzymatically cleavable linker can be a β-glucuronic acid-based linker. Facile release of the myeloid cell agonist can be realized through cleavage of the β-glucuronide glycosidic bond by the lysosomal enzyme β-glucuronidase. This enzyme can be abundantly present within lysosomes and can be overexpressed in some tumor types, while the enzyme activity outside cells can be low. β-Glucuronic acid-based linkers can be used to circumvent the tendency of a conjugate to undergo aggregation due to the hydrophilic nature of β-glucuronides. In certain embodiments, β-glucuronic acid-based linkers can link an antibody construct to a hydrophobic myeloid cell agonist. The following scheme depicts the release of a myeloid cell agonist (D) from an antibody construct of the conjugate (Ab) containing a β-glucuronic acid-based linker:

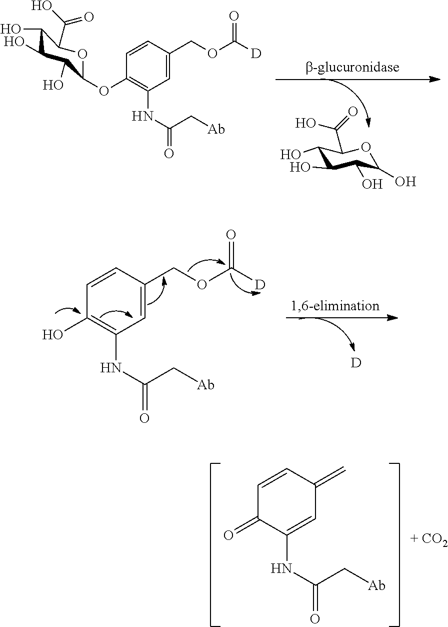

A variety of cleavable β-glucuronic acid-based linkers useful for linking drugs such as auristatins, camptothecin and doxorubicin analogues, CBI minor-groove binders, and psymberin to antibodies have been described. All of these β-glucuronic acid-based linkers may be used in the conjugates comprising a myeloid cell agonist described herein. In certain embodiments, the enzymatically cleavable linker is a β-galactoside-based linker. β-Galactoside is present abundantly within lysosomes, while the enzyme activity outside cells is low.

Additionally, myeloid cell agonists containing a phenol group can be covalently bonded to a linker through the phenolic oxygen. One such linker relies on a methodology in which a diamino-ethane “Space Link” is used in conjunction with traditional “PABO”-based self-immolative groups to deliver phenols.

Myeloid cell agonists containing an aromatic or aliphatic hydroxyl group can be covalently bonded to a linker through the hydroxyl group using a methodology that relies on a methylene carbamate linkage, as described in WO 2015/095755.

Cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on a myeloid cell agonist, wherein such ester groups can hydrolyze under physiological conditions to release the myeloid cell agonist. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

A linker can comprise an enzymatically cleavable peptide moiety, for example, a linker comprising structural formula (IVa), (IVb), (IVc), or (IVd):

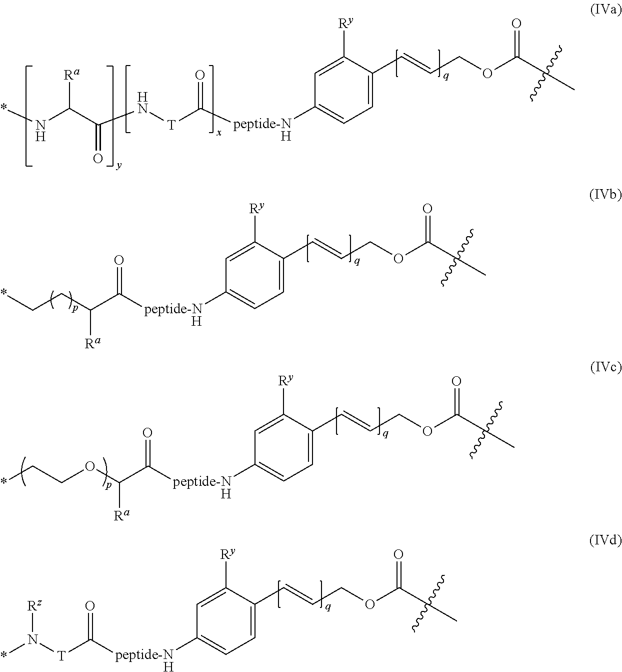

or a salt thereof, wherein: peptide represents a peptide (illustrated N→C, wherein peptide includes the amino and carboxy “termini”) a cleavable by a lysosomal enzyme; T represents a polymer comprising one or more ethylene glycol units or an alkylene chain, or combinations thereof; R^{a} is selected from hydrogen, alkyl, sulfonate and methyl sulfonate; R^{y} is hydrogen or C₁₋₄ alkyl-(O)ᵣ-(C₁₋₄ alkylene)ₛ-G¹ or C₁₋₄ alkyl-(N)—[(C₁₋₄ alkylene)-G¹]₂; R^{z} is C₁₋₄ alkyl-(O)ᵣ—(C₁₋₄ alkylene)ₛ-G²; G¹ is SO₃H, CO₂H, PEG 4-32, or sugar moiety; G² is SO₃H, CO₂H, or PEG 4-32 moiety; r is 0 or 1; s is 0 or 1; p is an integer ranging from 0 to 5; q is 0 or 1; x is 0 or 1; y is 0 or 1; [symbol] represents the point of attachment of the linker to the myeloid cell agonist; and * represents the point of attachment to the remainder of the linker.

In certain embodiments, the peptide can be selected from a tripeptide or a dipeptide. In particular embodiments, the dipeptide can be selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Exemplary embodiments of linkers according to structural formula (IVa) that can be included in the conjugates described herein can include the linkers illustrated below (as illustrated, the linkers include a group suitable for covalently linking the linker in a conjugate and the wavy line or unlinked bond indicates an attachment site for a myeloid cell agonist):

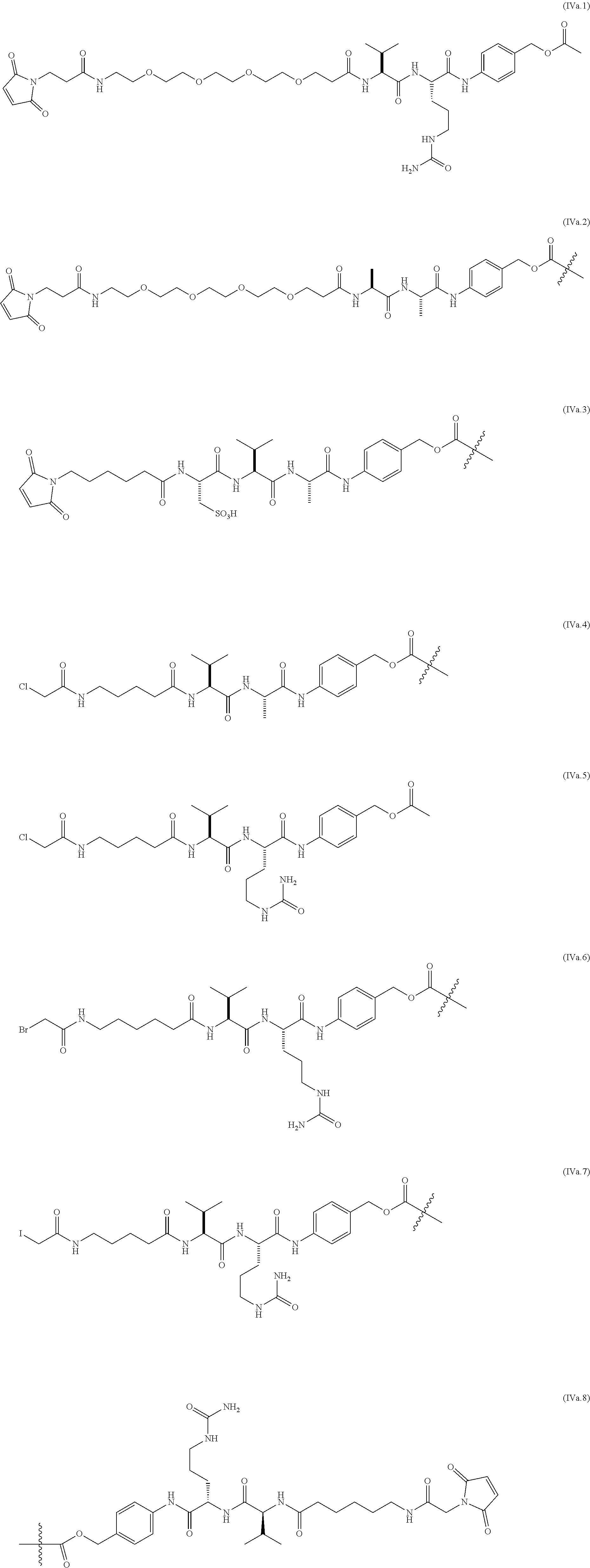

Exemplary embodiments of linkers according to structural formula (IVb), (IVc), or (IVd) that can be included in the conjugates described herein can include the linkers illustrated below (as illustrated, the linkers can include a group suitable for covalently linking the linker to a conjugate and the wavy line indicates an attachment site for a myeloid cell agonist):

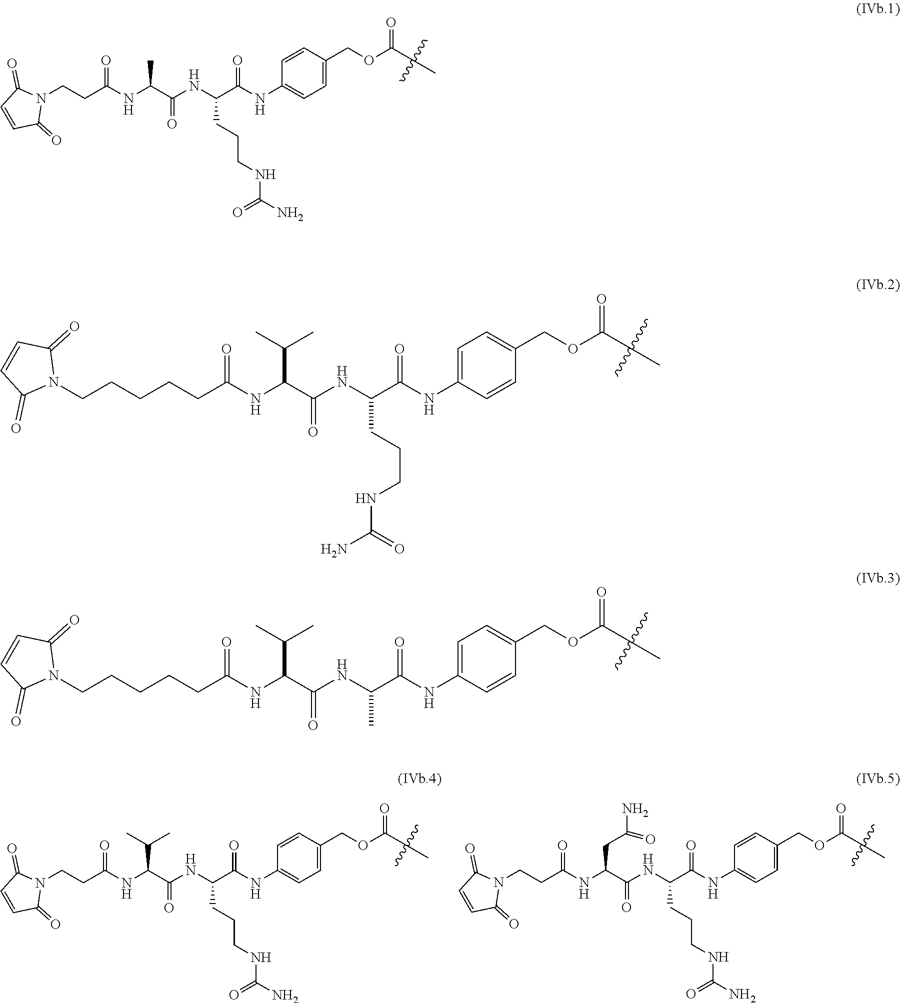 [SMILES: *C(=O)OCc1ccc(NC(=O)[C@H](CCCNC(N)=O)NC(=O)[C@H](C)NC(=O)CCN2C(=O)C=CC2=O)cc1 ; *C(=O)OCc1ccc(NC(=O)[C@H](CCCNC(N)=O)NC(=O)[C@@H](NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)CCN2C(=O)C=CC2=O)C(C)C)cc1 ; *C(=O)OCc1ccc(NC(=O)[C@H](CCCNC(N)=O)NC(=O)[C@H](CC(N)=O)NC(=O)CCN2C(=O)C=CC2=O)cc1]

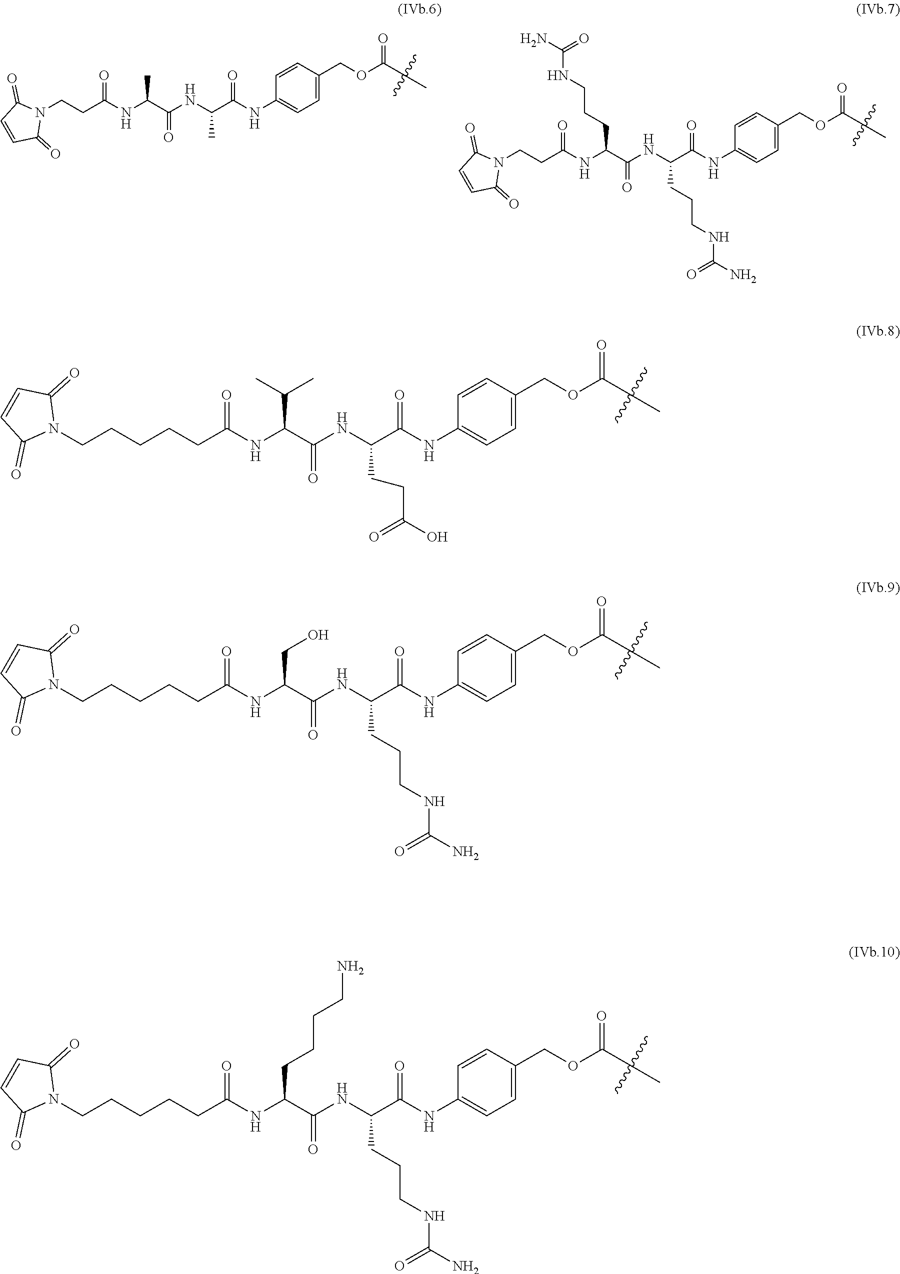 [SMILES: *C(=O)OCc1ccc(NC(=O)[C@H](C)NC(=O)[C@H](C)NC(=O)CCN2C(=O)C=CC2=O)cc1 ; *C(=O)OCc1ccc(NC(=O)[C@H](CCCNC(N)=O)NC(=O)[C@H](CCCNC(N)=O)NC(=O)CCN2C(=O)C=CC2=O)cc1 ; *C(=O)OCc1ccc(NC(=O)[C@H](CCC(=O)O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1]

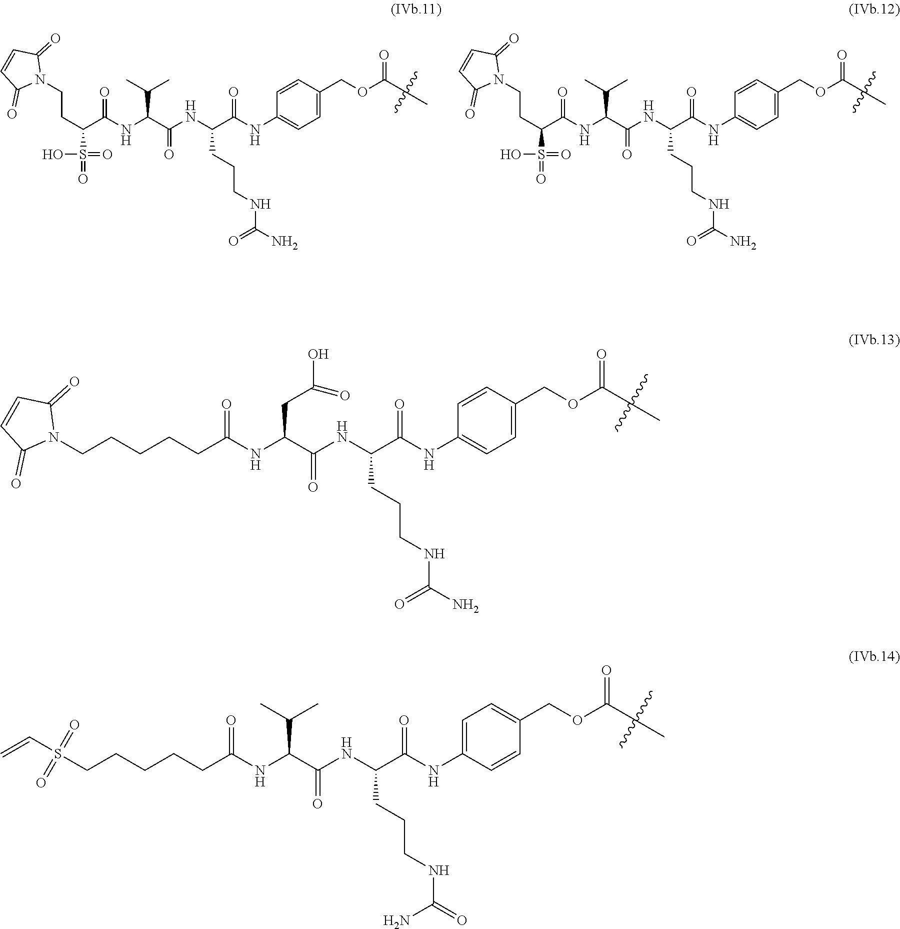 [SMILES: *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1 ; *C(=O)OCc1ccc(NC(=O)[C@H](CCCNC(N)=O)NC(=O)[C@H](CC(=O)O)NC(=O)CCCCCN2C(=O)C=CC2=O)cc1 ; *C(=O)OCc1ccc(NC(=O)[C@H](CCCNC(N)=O)NC(=O)[C@@H](NC(=O)CCCCCS(=O)(=O)C=C)C(C)C)cc1]

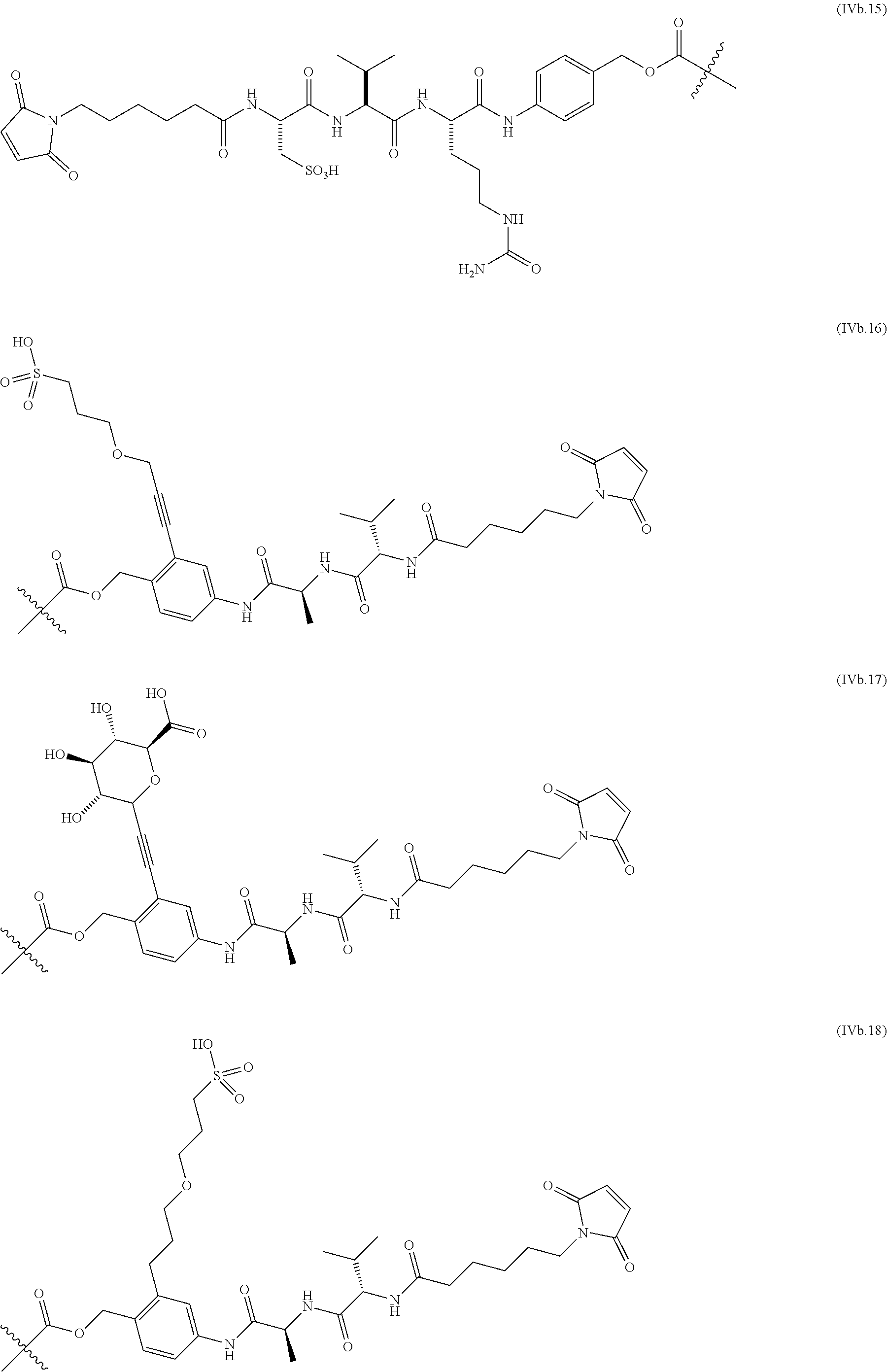 [SMILES: *C(=O)OCc1ccc(NC(=O)[C@H](CCCNC(N)=O)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1C#CCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1C#CC1O[C@H](C(=O)O)[C@@H](O)[C@H](O)[C@H]1O ; *C(=O)OCc1ccc(NC(=O)[C@H](C)NC(=O)[C@@H](NC(=O)CCCCCN2C(=O)C=CC2=O)C(C)C)cc1CCCOCCCS(=O)(=O)O]

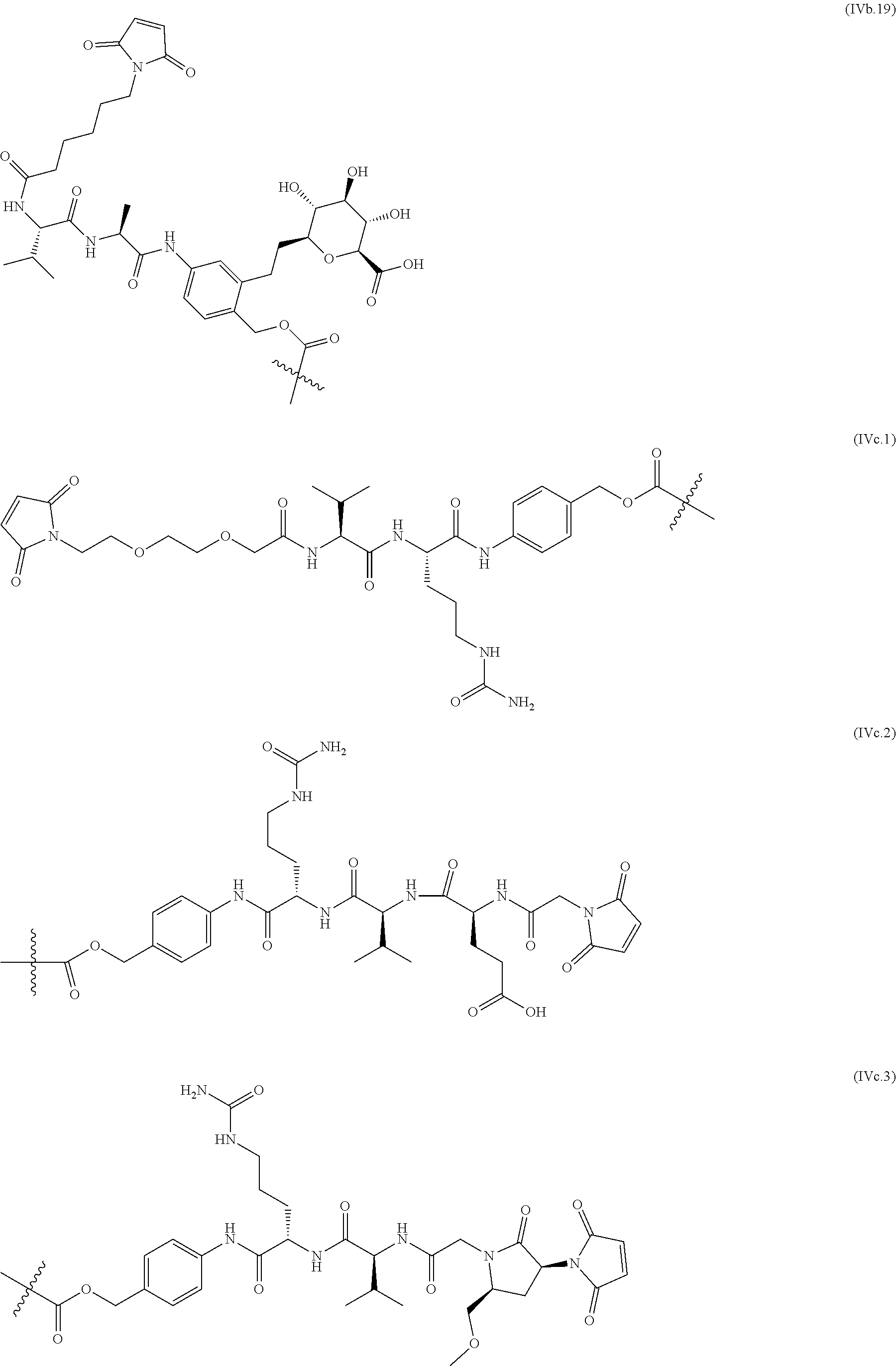 [SMILES: *C(=O)OCc1ccc(NC(=O)[C@H](C)NC(=O)[C@@H](NC(=O)CCCCCN2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)COCCOCCN2C(=O)C=CC2=O)C(C)C)cc1 ; *C(=O)OCc1ccc(NC(=O)[C@H](CCCNC(N)=O)NC(=O)[C@@H](NC(=O)[C@H](CCC(=O)O)NC(=O)CN2C(=O)C=CC2=O)C(C)C)cc1 ; *C(=O)OCc1ccc(NC(=O)[C@H](CCCNC(N)=O)NC(=O)[C@@H](NC(=O)CN2C(=O)[C@@H](N3C(=O)C=CC3=O)C[C@H]2COC)C(C)C)cc1]

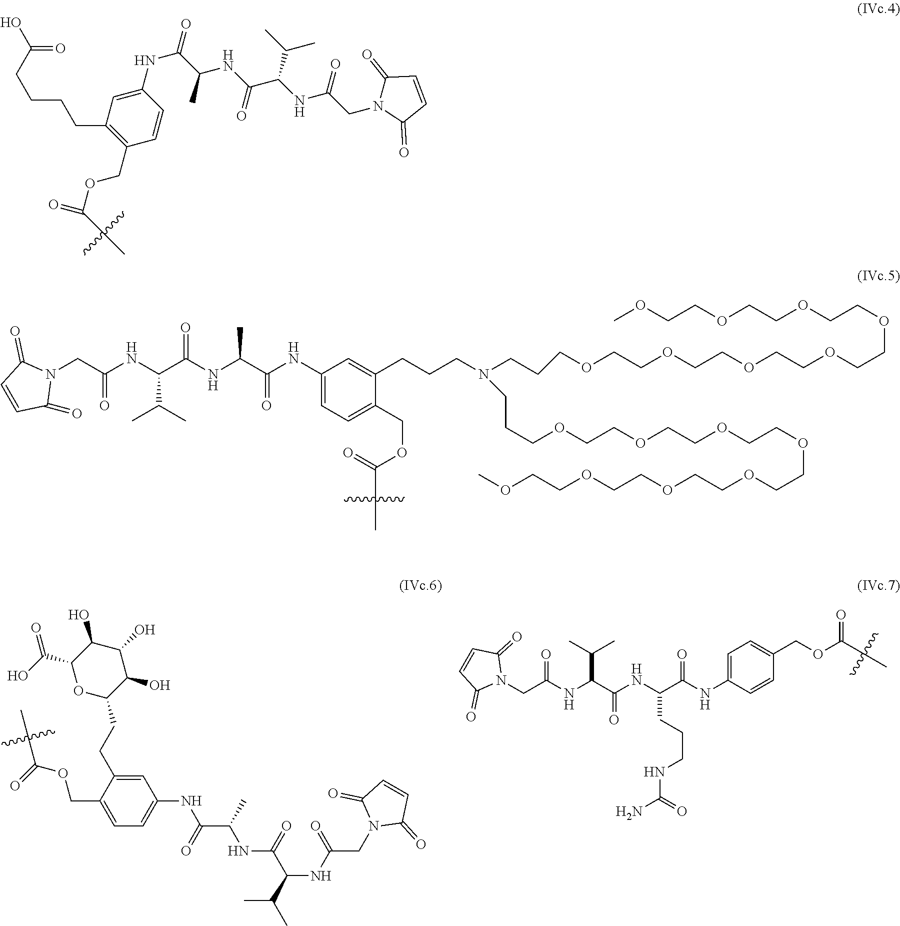 [SMILES: *C(=O)OCc1ccc(NC(=O)[C@H](C)NC(=O)[C@@H](NC(=O)CN2C(=O)C=CC2=O)C(C)C)cc1CCCCC(=O)O ; *C(=O)OCc1ccc(NC(=O)[C@H](C)NC(=O)[C@@H](NC(=O)CN2C(=O)C=CC2=O)C(C)C)cc1CCCN(CCCOCCOCCOCCOCCOCCOCCOCCOC)CCCOCCOCCOCCOCCOCCOCCOCCOC ; *C(=O)OCc1ccc(NC(=O)[C@H](C)NC(=O)[C@@H](NC(=O)CN2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)CN2C(=O)C=CC2=O)C(C)C)cc1]

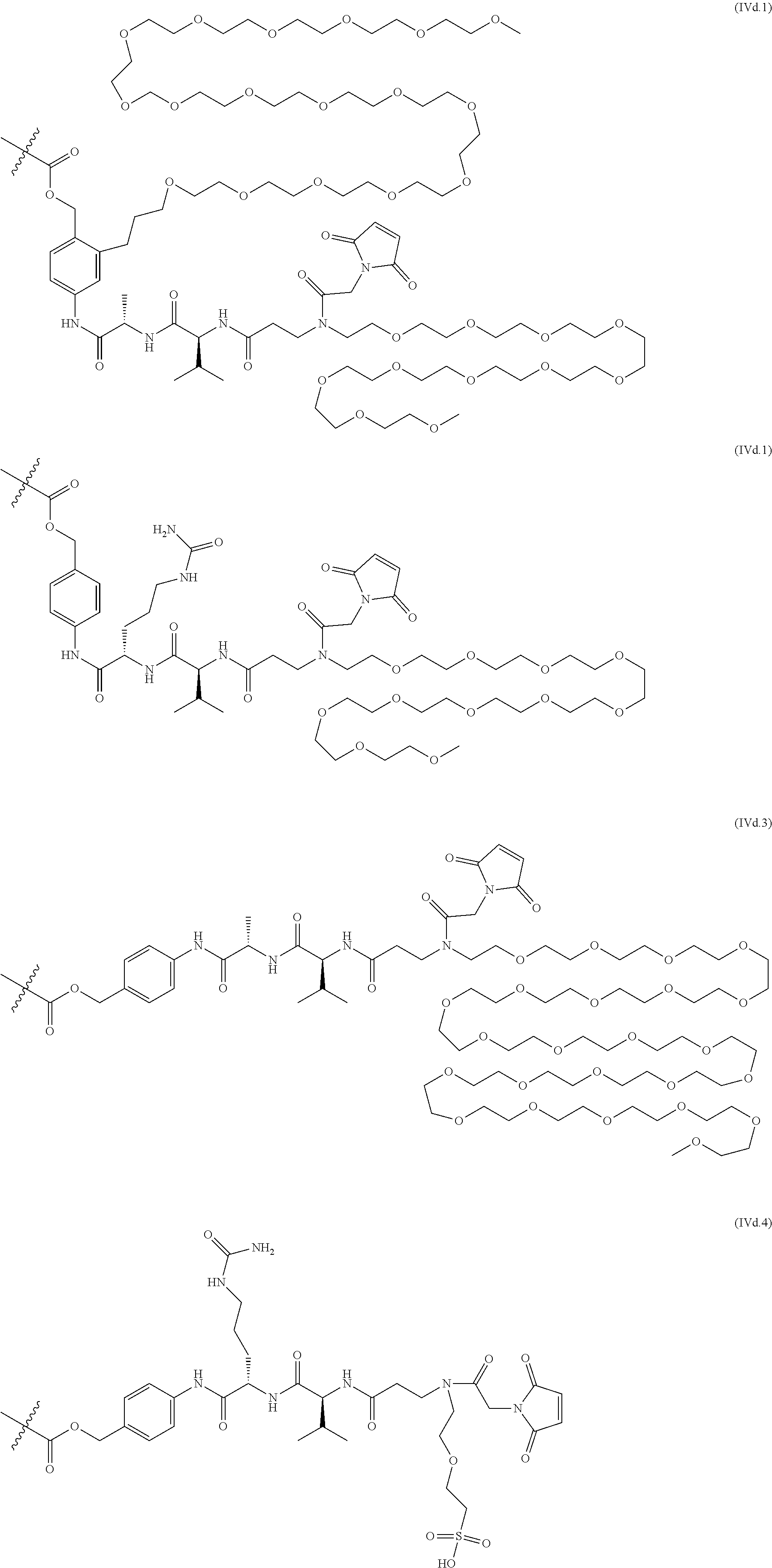

The linker can contain an enzymatically cleavable sugar moiety, for example, a linker comprising structural formula (Va), (Vb), (Vc), (Vd), or (Ve):

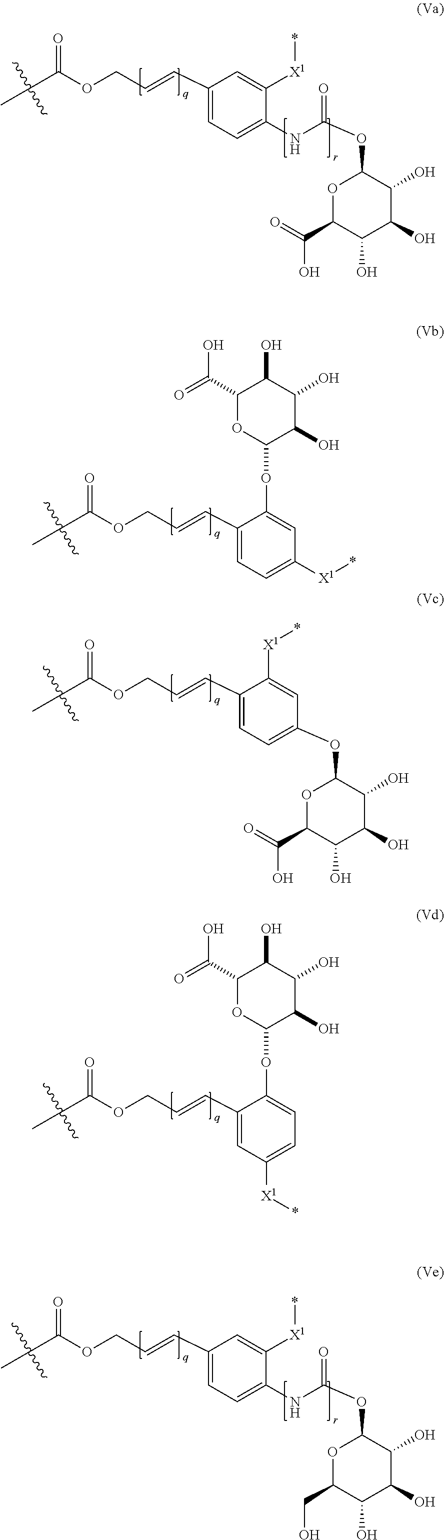

or a salt thereof, wherein: q is 0 or 1; r is 0 or 1; X¹ is CH₂, O or NH; [symbol] represents the point of attachment of the linker to the myeloid cell agonist; and * represents the point of attachment to the remainder of the linker.

Exemplary embodiments of linkers according to structural formula (Va) that may be included in the conjugates described herein can include the linkers illustrated below (as illustrated, the linkers include a group suitable for covalently linking the linker in a conjugate and the wavy line indicates an attachment site for a myeloid cell agonist):

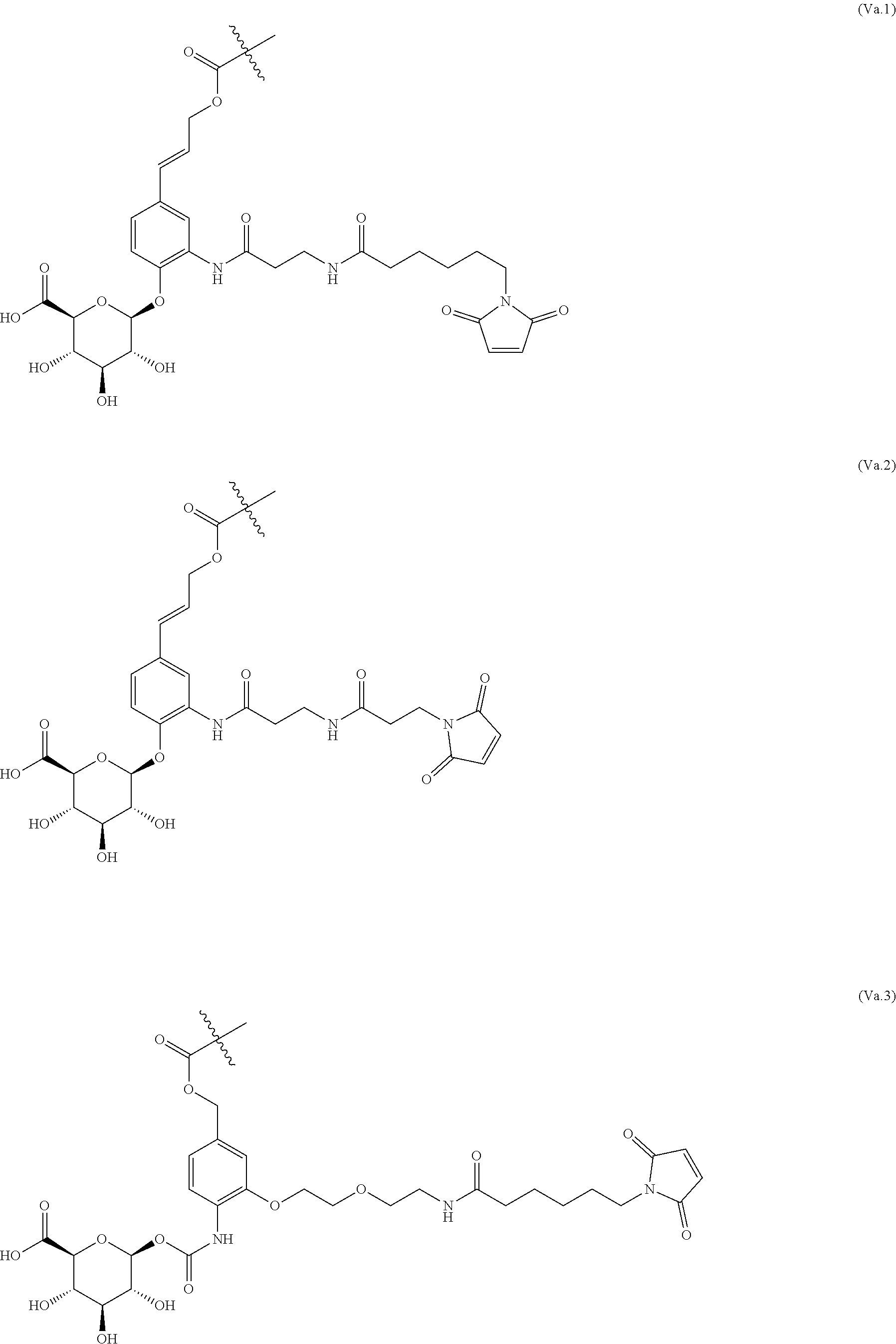 [SMILES: *C(=O)OC/C=C/c1ccc(O[C@@H]2O[C@H](C(=O)O)[C@@H](O)[C@H](O)[C@H]2O)c(NC(=O)CCNC(=O)CCCCCN2C(=O)C=CC2=O)c1 ; *C(=O)OC/C=C/c1ccc(O[C@@H]2O[C@H](C(=O)O)[C@@H](O)[C@H](O)[C@H]2O)c(NC(=O)CCNC(=O)CCN2C(=O)C=CC2=O)c1 ; *C(=O)OCc1ccc(NC(=O)O[C@@H]2O[C@H](C(=O)O)[C@@H](O)[C@H](O)[C@H]2O)c(OCCOCCNC(=O)CCCCCN2C(=O)C=CC2=O)c1]

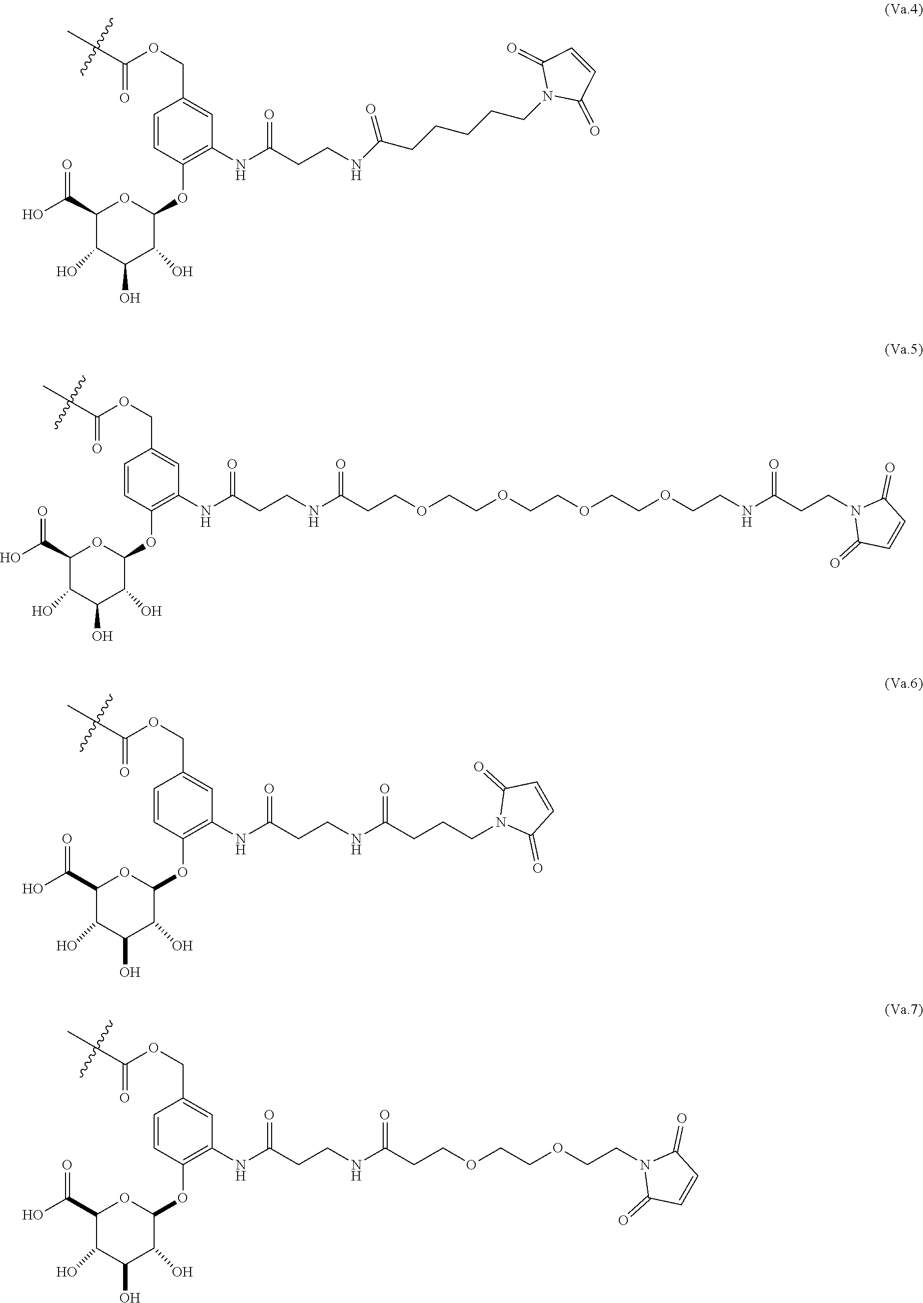 [SMILES: *C(=O)OCc1ccc(O[C@@H]2O[C@H](C(=O)O)[C@@H](O)[C@H](O)[C@H]2O)c(NC(=O)CCNC(=O)CCCCCN2C(=O)C=CC2=O)c1 ; *C(=O)OCc1ccc(O[C@@H]2O[C@H](C(=O)O)[C@@H](O)[C@H](O)[C@H]2O)c(NC(=O)CCNC(=O)CCOCCOCCOCCOCCNC(=O)CCN2C(=O)C=CC2=O)c1 ; *C(=O)OCc1ccc(O[C@@H]2O[C@H](C(=O)O)[C@@H](O)[C@H](O)[C@H]2O)c(NC(=O)CCNC(=O)CCCN2C(=O)C=CC2=O)c1 ; *C(=O)OCc1ccc(O[C@@H]2O[C@H](C(=O)O)[C@@H](O)[C@H](O)[C@H]2O)c(NC(=O)CCNC(=O)CCOCCOCCN2C(=O)C=CC2=O)c1]

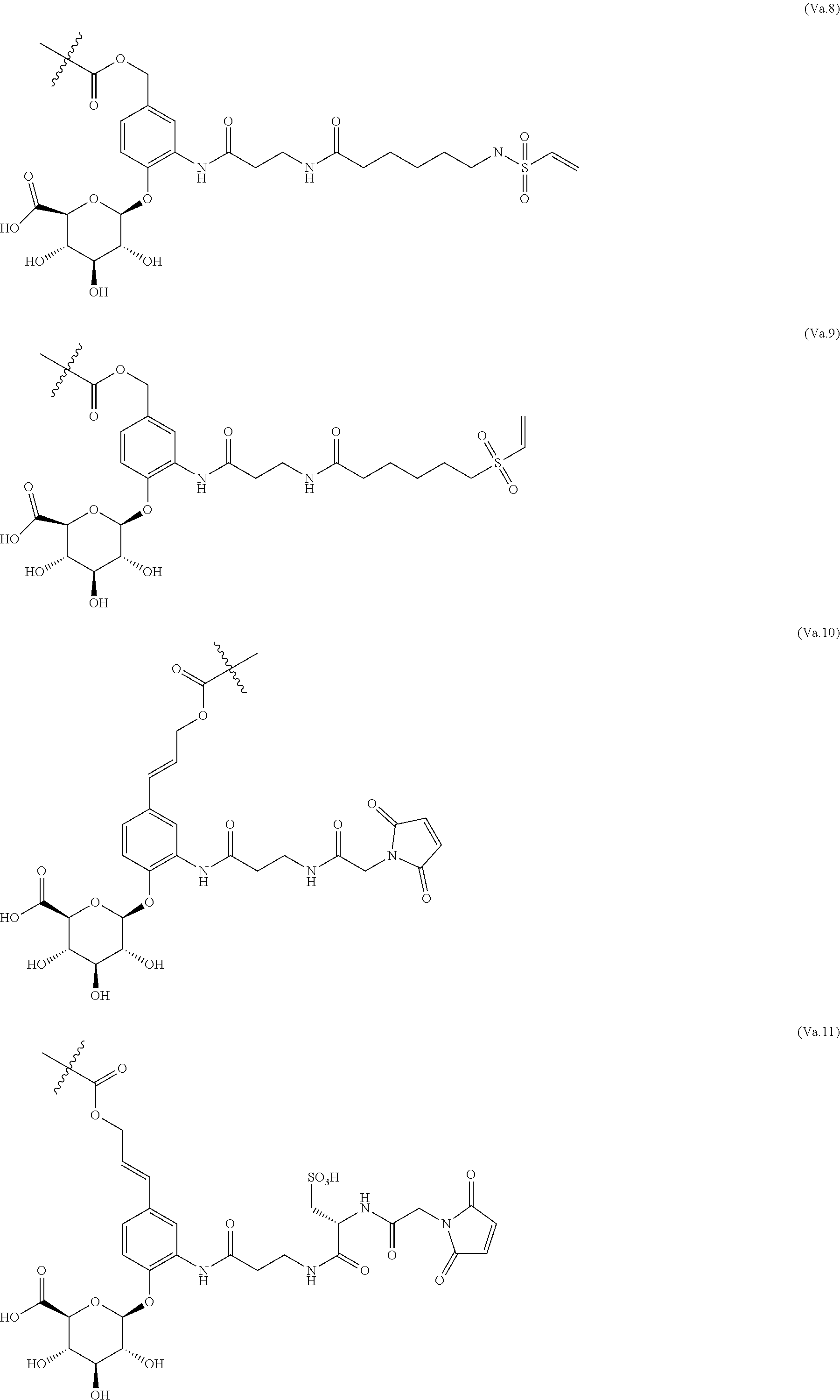 [SMILES: *C(=O)OCc1ccc(O[C@@H]2O[C@H](C(=O)O)[C@@H](O)[C@H](O)[C@H]2O)c(NC(=O)CCNC(=O)CCCCCNS(=O)(=O)C=C)c1 ; *C(=O)OCc1ccc(O[C@@H]2O[C@H](C(=O)O)[C@@H](O)[C@H](O)[C@H]2O)c(NC(=O)CCNC(=O)CCCCCS(=O)(=O)C=C)c1 ; *C(=O)OC/C=C/c1ccc(O[C@@H]2O[C@H](C(=O)O)[C@@H](O)[C@H](O)[C@H]2O)c(NC(=O)CCNC(=O)CN2C(=O)C=CC2=O)c1 ; *C(=O)OC/C=C/c1ccc(O[C@@H]2O[C@H](C(=O)O)[C@@H](O)[C@H](O)[C@H]2O)c(NC(=O)CCNC(=O)[C@H](CS(=O)(=O)O)NC(=O)CN2C(=O)C=CC2=O)c1]

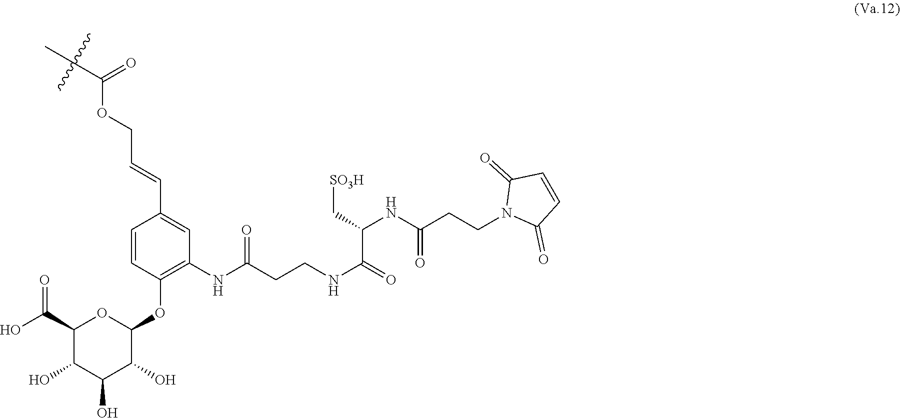 [SMILES: *C(=O)OC/C=C/c1ccc(O[C@@H]2O[C@H](C(=O)O)[C@@H](O)[C@H](O)[C@H]2O)c(NC(=O)CCNC(=O)[C@H](CS(=O)(=O)O)NC(=O)CCN2C(=O)C=CC2=O)c1]

Exemplary embodiments of linkers according to structural formula (Vb) that may be included in the conjugates described herein include the linkers illustrated below (as illustrated, the linkers include a group suitable for covalently linking the linker in a conjugate and the wavy line indicates an attachment site for a myeloid cell agonist):

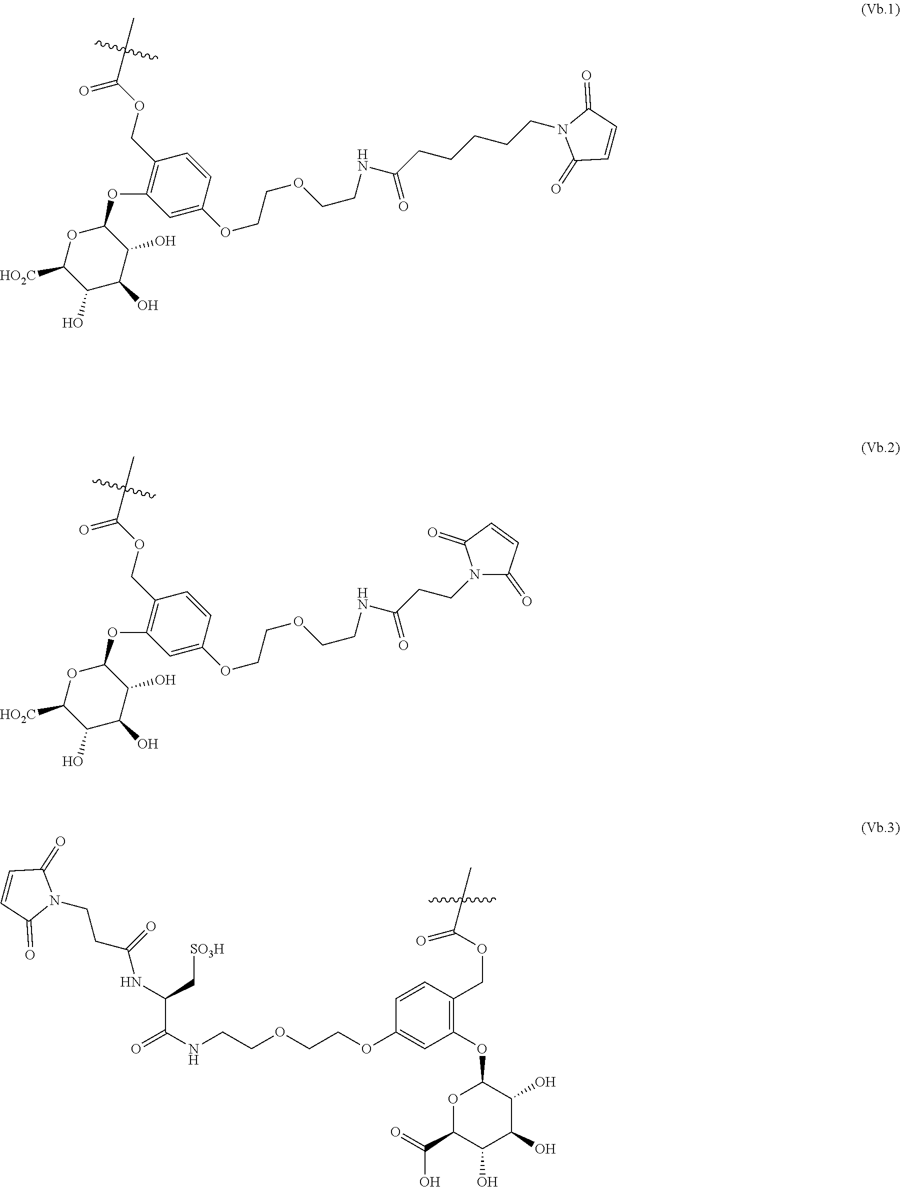 [SMILES: *C(=O)OCc1ccc(OCCOCCNC(=O)CCCCCN2C(=O)C=CC2=O)cc1O[C@@H]1O[C@H](C(=O)O)[C@@H](O)[C@H](O)[C@H]1O ; *C(=O)OCc1ccc(OCCOCCNC(=O)CCN2C(=O)C=CC2=O)cc1O[C@@H]1O[C@H](C(=O)O)[C@@H](O)[C@H](O)[C@H]1O ; *C(=O)OCc1ccc(OCCOCCNC(=O)[C@H](CS(=O)(=O)O)NC(=O)CCN2C(=O)C=CC2=O)cc1O[C@@H]1O[C@H](C(=O)O)[C@@H](O)[C@H](O)[C@H]1O]

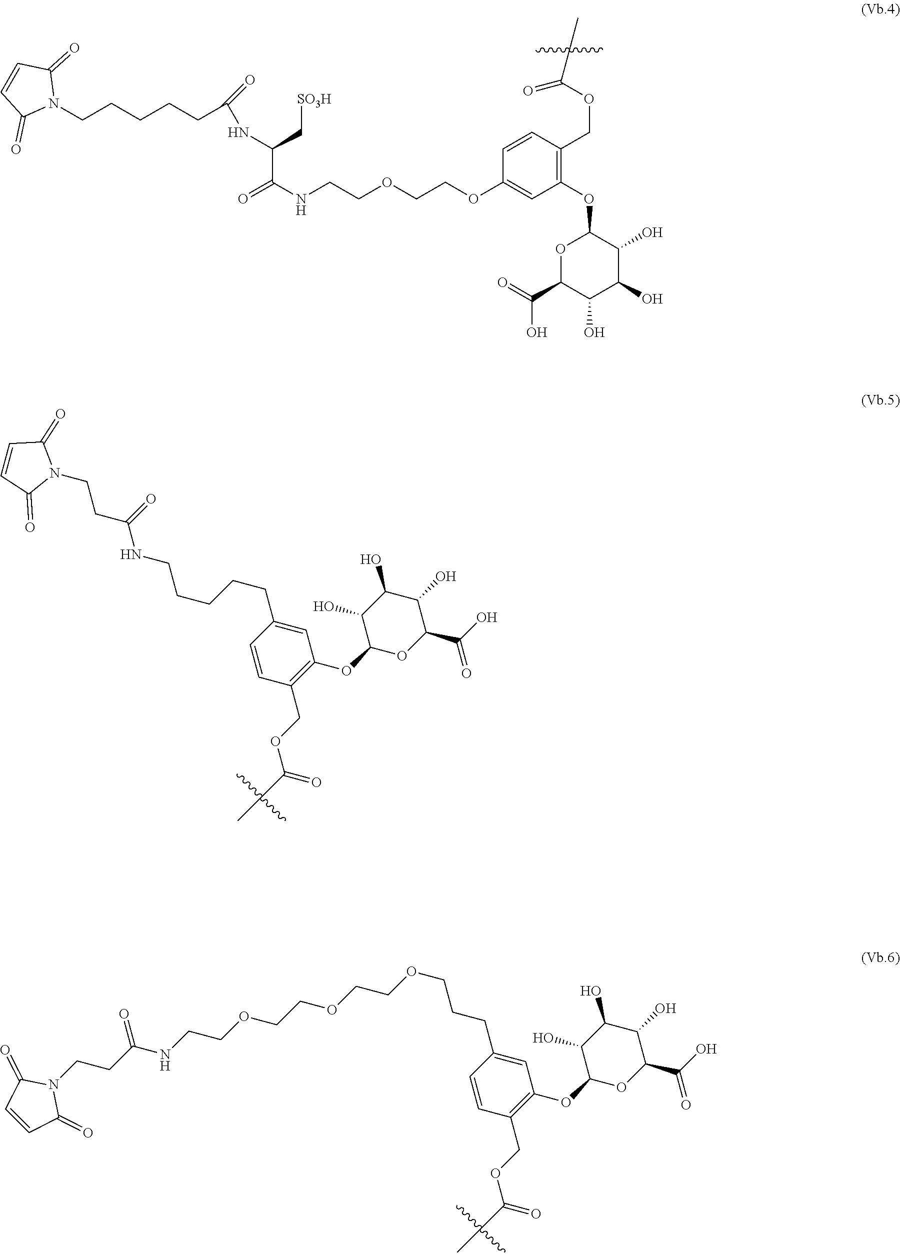 [SMILES: *C(=O)OCc1ccc(OCCOCCNC(=O)[C@H](CS(=O)(=O)O)NC(=O)CCCCCN2C(=O)C=CC2=O)cc1O[C@@H]1O[C@H](C(=O)O)[C@@H](O)[C@H](O)[C@H]1O ; *C(=O)OCc1ccc(CCCCCNC(=O)CCN2C(=O)C=CC2=O)cc1O[C@@H]1O[C@H](C(=O)O)[C@@H](O)[C@H](O)[C@H]1O ; *C(=O)OCc1ccc(CCCOCCOCCOCCNC(=O)CCN2C(=O)C=CC2=O)cc1O[C@@H]1O[C@H](C(=O)O)[C@@H](O)[C@H](O)[C@H]1O]

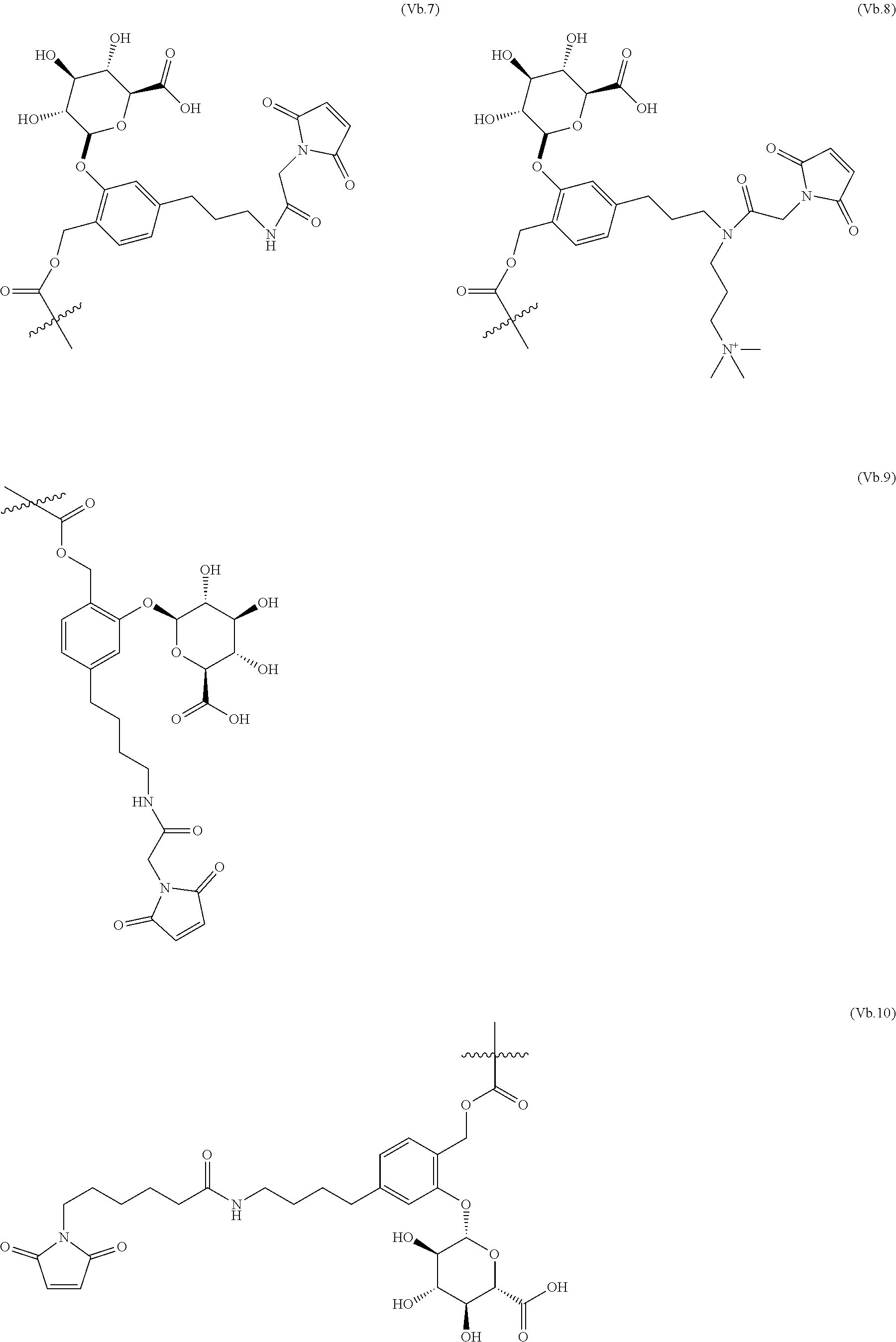 [SMILES: *C(=O)OCc1ccc(CCCNC(=O)CN2C(=O)C=CC2=O)cc1O[C@@H]1O[C@H](C(=O)O)[C@@H](O)[C@H](O)[C@H]1O ; *C(=O)OCc1ccc(CCCN(CCC[N+](C)(C)C)C(=O)CN2C(=O)C=CC2=O)cc1O[C@@H]1O[C@H](C(=O)O)[C@@H](O)[C@H](O)[C@H]1O ; *C(=O)OCc1ccc(CCCCNC(=O)CN2C(=O)C=CC2=O)cc1O[C@@H]1O[C@H](C(=O)O)[C@@H](O)[C@H](O)[C@H]1O ; *C(=O)OCc1ccc(CCCCNC(=O)CCCCCN2C(=O)C=CC2=O)cc1O[C@@H]1O[C@H](C(=O)O)[C@@H](O)[C@H](O)[C@H]1O]

Exemplary embodiments of linkers according to structural formula (Vc) that may be included in the conjugates described herein include the linkers illustrated below (as illustrated, the linkers include a group suitable for covalently linking the linker in a conjugate and the wavy line indicates an attachment site for a myeloid cell agonist):

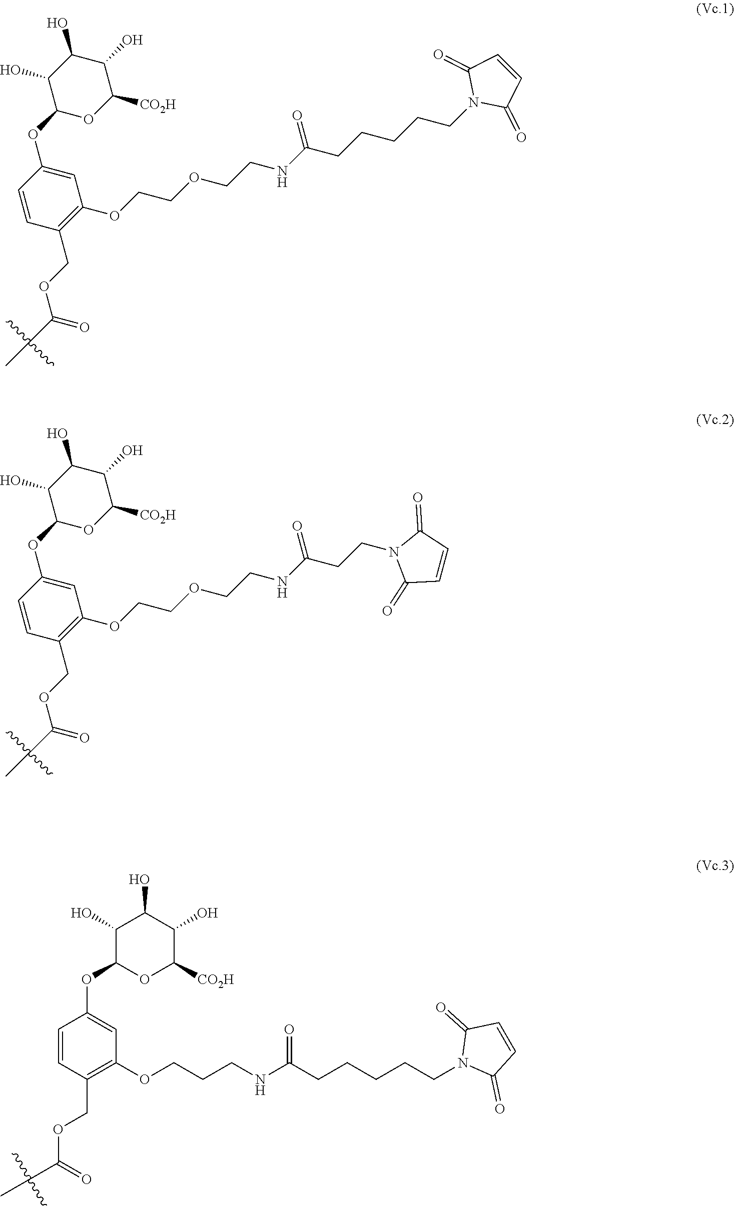 [SMILES: *C(=O)OCc1ccc(O[C@@H]2O[C@H](C(=O)O)[C@@H](O)[C@H](O)[C@H]2O)cc1OCCOCCNC(=O)CCCCCN1C(=O)C=CC1=O ; *C(=O)OCc1ccc(O[C@@H]2O[C@H](C(=O)O)[C@@H](O)[C@H](O)[C@H]2O)cc1OCCOCCNC(=O)CCN1C(=O)C=CC1=O ; *C(=O)OCc1ccc(O[C@@H]2O[C@H](C(=O)O)[C@@H](O)[C@H](O)[C@H]2O)cc1OCCCNC(=O)CCCCCN1C(=O)C=CC1=O]

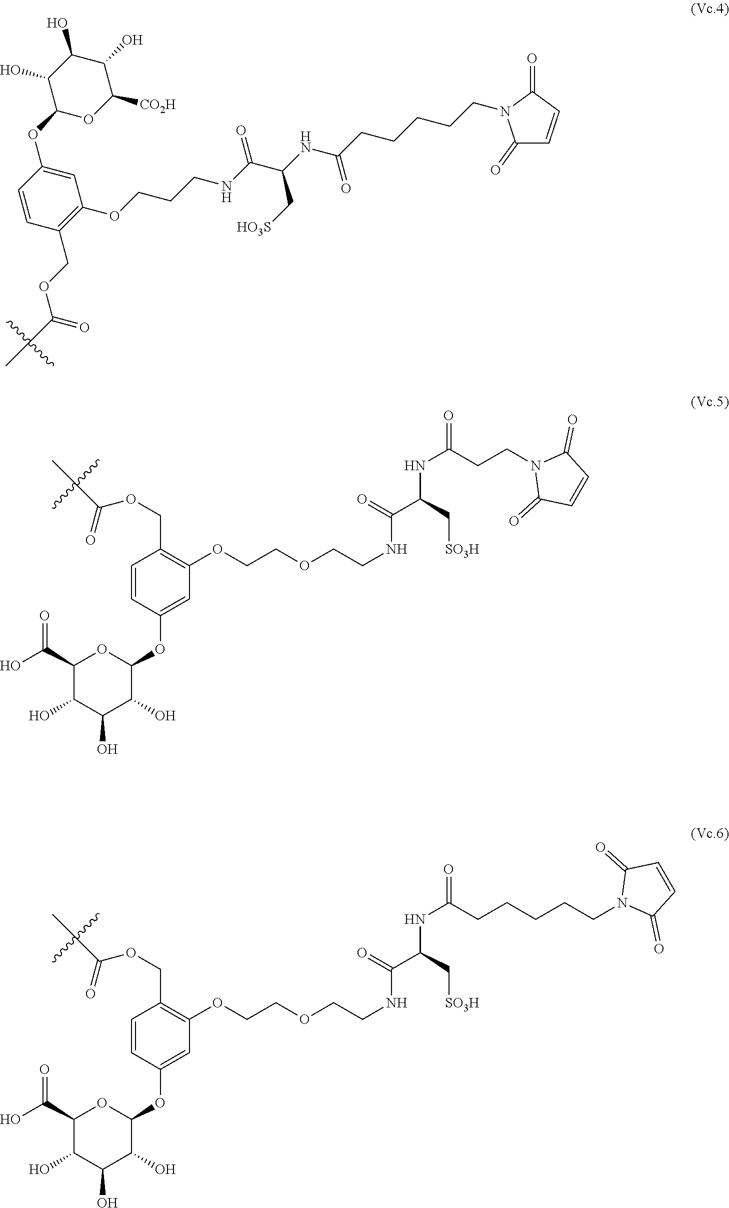 [SMILES: *C(=O)OCc1ccc(O[C@@H]2O[C@H](C(=O)O)[C@@H](O)[C@H](O)[C@H]2O)cc1OCCCNC(=O)[C@H](CS(=O)(=O)O)NC(=O)CCCCCN1C(=O)C=CC1=O ; *C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CCN1C(=O)C=CC1=O ; *C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CCCCCN1C(=O)C=CC1=O]

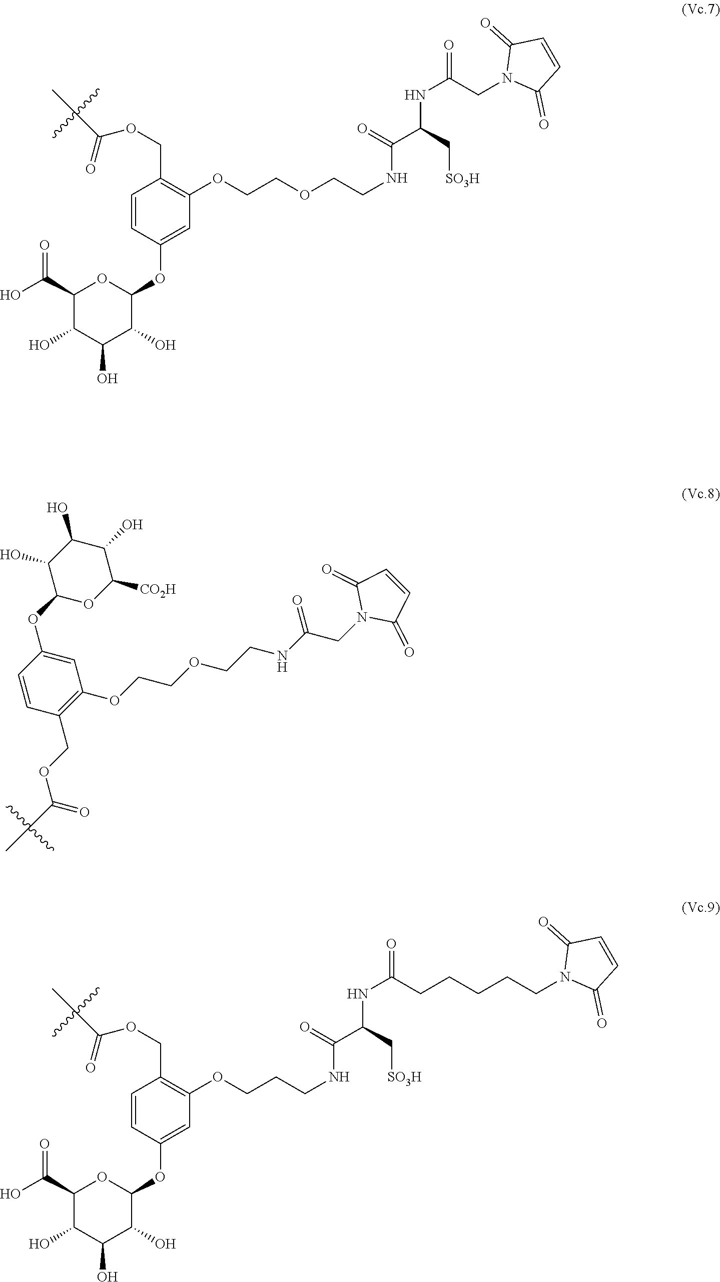 [SMILES: *C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CN1C(=O)C=CC1=O ; *C(=O)OCc1ccc(O[C@@H]2O[C@H](C(=O)O)[C@@H](O)[C@H](O)[C@H]2O)cc1OCCOCCNC(=O)CN1C(=O)C=CC1=O ; *C(=O)OCc1ccc(O[C@@H]2O[C@H](C(=O)O)[C@@H](O)[C@H](O)[C@H]2O)cc1OCCCNC(=O)[C@H](CS(=O)(=O)O)NC(=O)CCCCCN1C(=O)C=CC1=O]

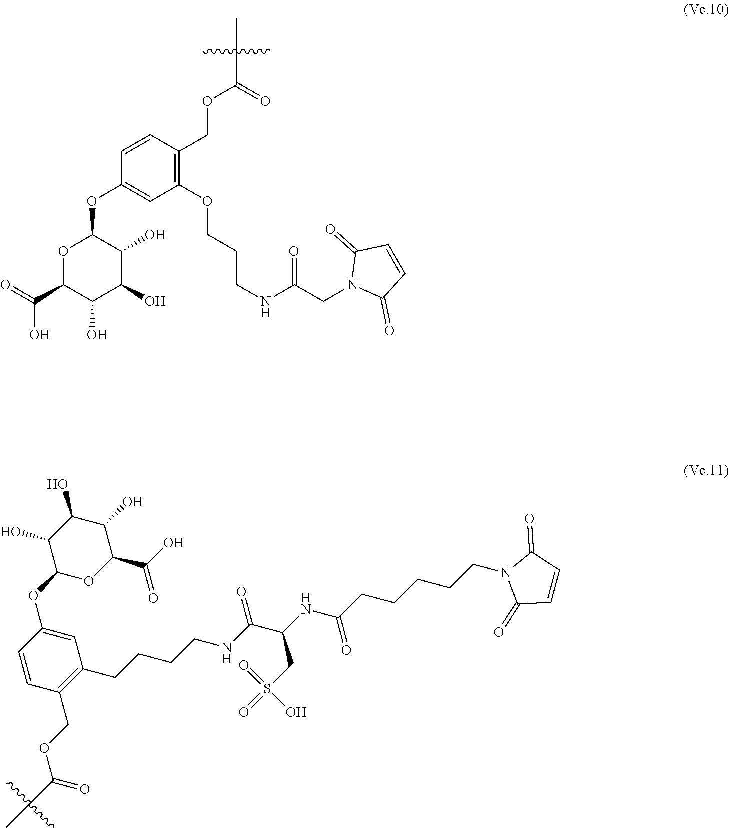 [SMILES: *C(=O)OCc1ccc(O[C@@H]2O[C@H](C(=O)O)[C@@H](O)[C@H](O)[C@H]2O)cc1OCCCNC(=O)CN1C(=O)C=CC1=O ; *C(=O)OCc1ccc(O[C@@H]2O[C@H](C(=O)O)[C@@H](O)[C@H](O)[C@H]2O)cc1CCCCNC(=O)[C@H](CS(=O)(=O)O)NC(=O)CCCCCN1C(=O)C=CC1=O]

Exemplary embodiments of linkers according to structural formula (Vd) that may be included in the conjugates described herein include the linkers illustrated below (as illustrated, the linkers include a group suitable for covalently linking the linker in a conjugate and the wavy line indicates an attachment site for a myeloid cell agonist):

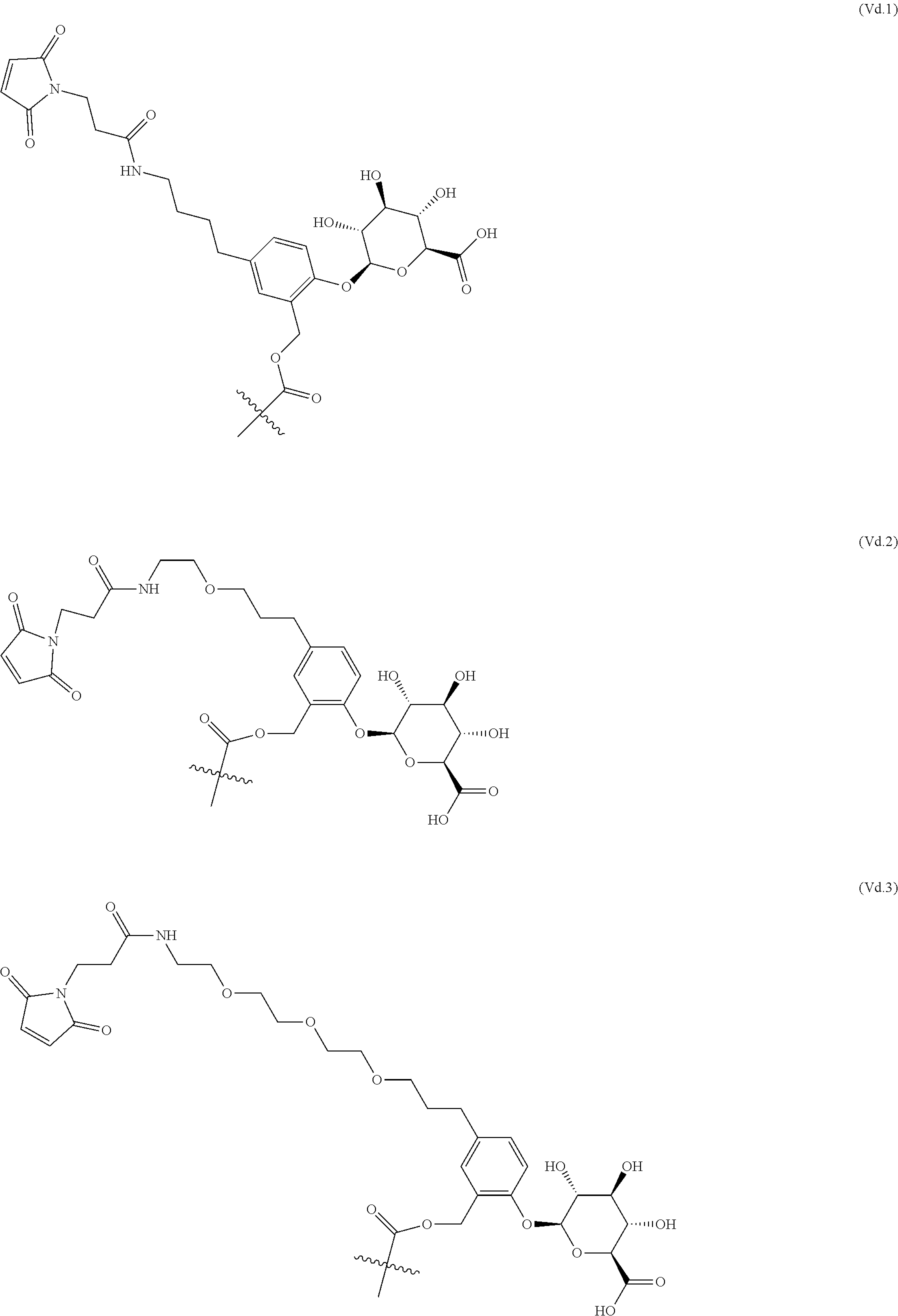 [SMILES: *C(=O)OCc1cc(CCCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCN2C(=O)C=CC2=O)ccc1O[C@@H]1O[C@H](C(=O)O)[C@@H](O)[C@H](O)[C@H]1O]

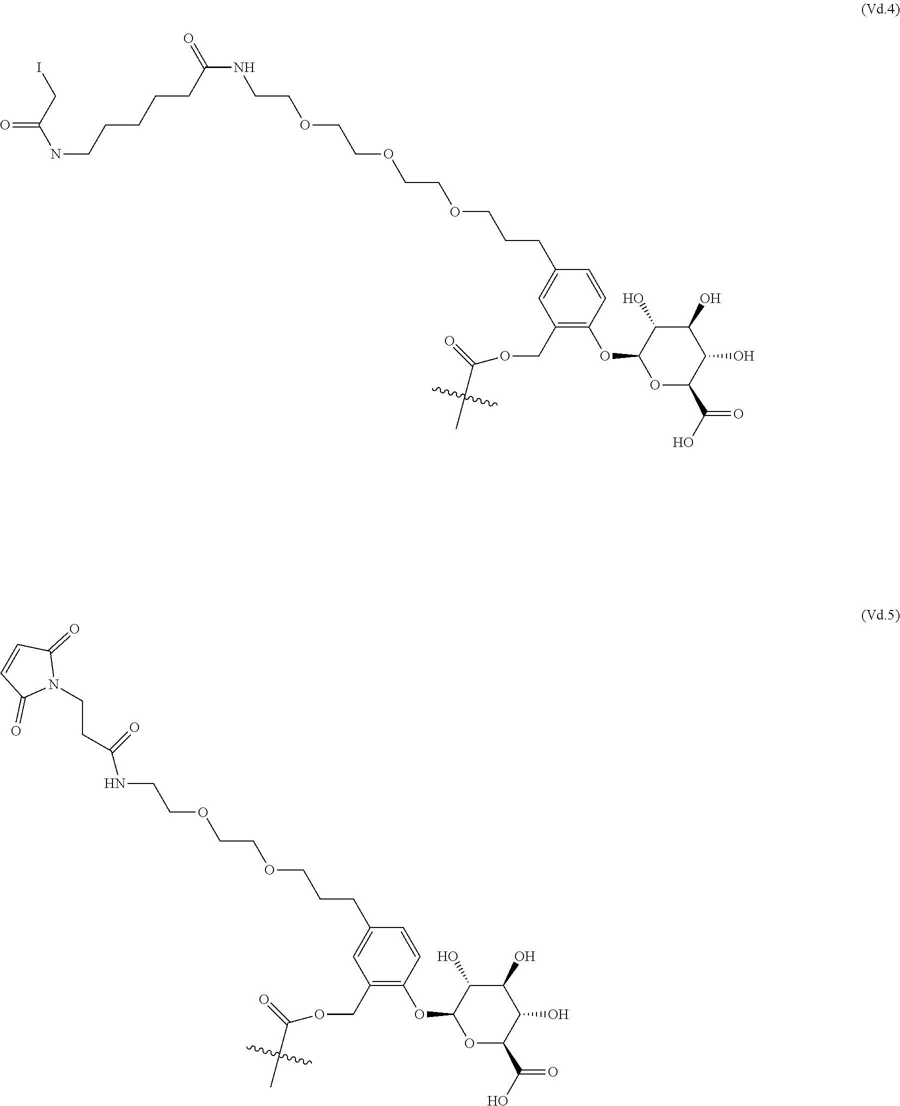 [SMILES: *C(=O)OCc1cc(CCCOCCOCCOCCNC(=O)CCCCCNC(=O)CI)ccc1O[C@@H]1O[C@H](C(=O)O)[C@@H](O)[C@H](O)[C@H]1O ; *C(=O)OCc1cc(CCCOCCOCCNC(=O)CCN2C(=O)C=CC2=O)ccc1O[C@@H]1O[C@H](C(=O)O)[C@@H](O)[C@H](O)[C@H]1O]

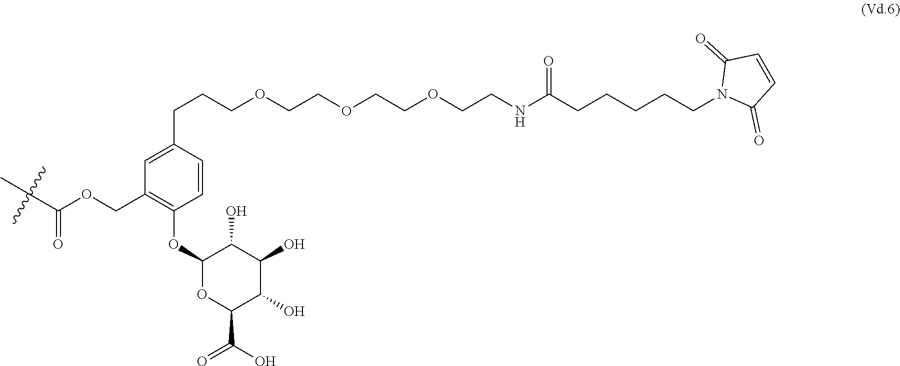 [SMILES: *C(=O)OCc1cc(CCCOCCOCCOCCNC(=O)CCCCCN2C(=O)C=CC2=O)ccc1O[C@@H]1O[C@H](C(=O)O)[C@@H](O)[C@H](O)[C@H]1O]

Exemplary embodiments of linkers according to structural formula (Ve) that may be included in the conjugates described herein include the linkers illustrated below (as illustrated, the linkers include a group suitable for covalently linking the linker in a conjugate and the wavy line indicates an attachment site for a myeloid cell agonist):

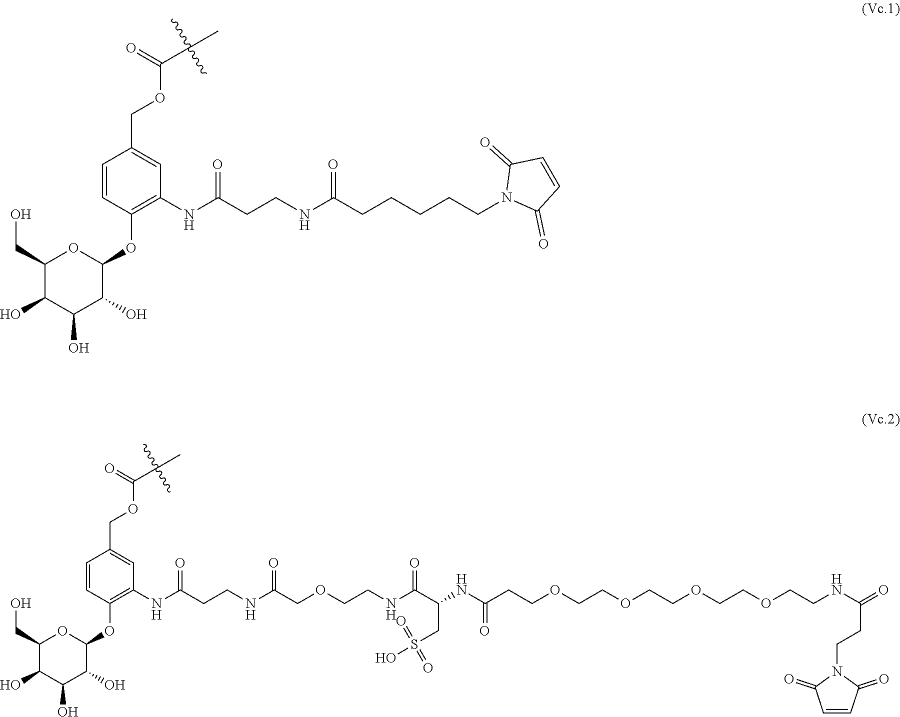 [SMILES: *C(=O)OCc1ccc(O[C@@H]2O[C@H](CO)[C@H](O)[C@H](O)[C@H]2O)c(NC(=O)CCNC(=O)CCCCCN2C(=O)C=CC2=O)c1 ; *C(=O)OCc1ccc(O[C@@H]2O[C@H](CO)[C@H](O)[C@H](O)[C@H]2O)c(NC(=O)CCNC(=O)COCCNC(=O)[C@@H](CS(=O)(=O)O)NC(=O)CCOCCOCCOCCOCCNC(=O)CCN2C(=O)C=CC2=O)c1]

Although cleavable linkers can provide certain advantages, the linkers comprising the conjugate described herein need not be cleavable. For non-cleavable linkers, the myeloid cell agonist release may not depend on the differential properties between the plasma and some cytoplasmic compartments. The release of the myeloid cell agonist can occur after internalization of the conjugate via antigen-mediated endocytosis and delivery to lysosomal compartment, where the conjugate can be degraded to the level of amino acids through intracellular proteolytic degradation. This process can release an active form of the myeloid cell agonist (a derivative), which is formed by the myeloid cell agonist, the linker, or a portion thereof, and in some instances the amino acid residue to which the linker was covalently attached. The myeloid cell agonist derivative from conjugates with non-cleavable linkers can be more hydrophilic and less membrane permeable, which can lead to less bystander effects and less nonspecific toxicities compared to conjugates with a cleavable linker. Conjugates with non-cleavable linkers can have greater stability in circulation than conjugates with cleavable linkers. Non-cleavable linkers can be alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units.

The linker can be non-cleavable in vivo, for example, a linker according to the formulations below:

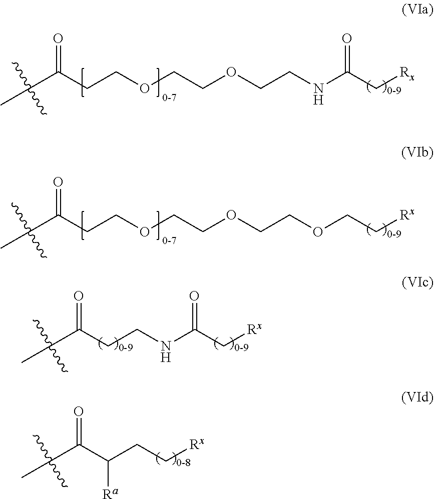

or salts thereof, wherein: R^{a} is selected from hydrogen, alkyl, sulfonate and methyl sulfonate; R^{x} is a moiety including a functional group capable of covalently linking the linker to an antibody construct of the conjugate; and [symbol] represents the point of attachment of the linker to the myeloid cell agonist.

Exemplary embodiments of linkers according to structural formula (VIa)-(VId) that may be included in the conjugates described herein include the linkers illustrated below (as illustrated, the linkers include a group suitable for covalently linking the linker in a conjugate, and [symbol] represents the point of attachment in a conjugate):

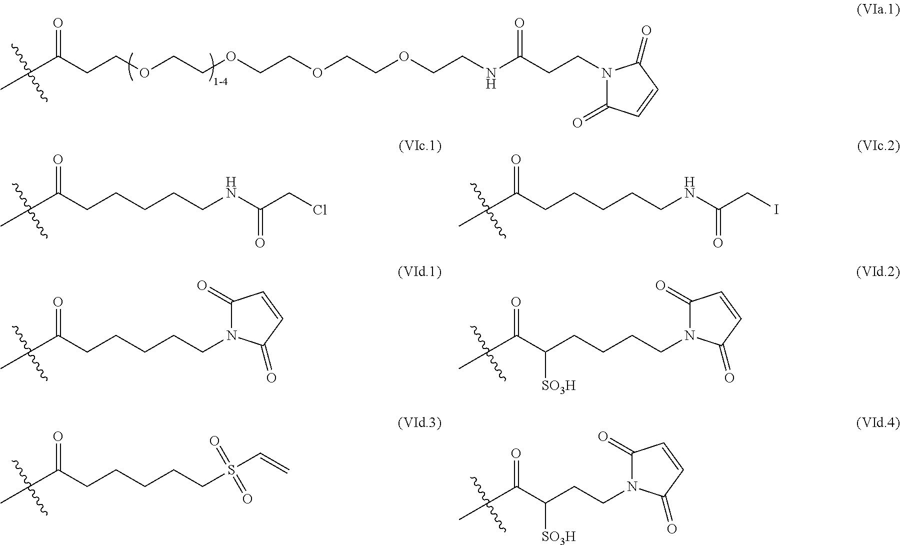

Attachment groups that are used to attach the linkers in a conjugate can be electrophilic in nature and include, for example, maleimide groups, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

One example of a “self-stabilizing” maleimide group that hydrolyzes spontaneously under conjugation conditions to give a conjugate species with improved stability is depicted in the schematic below. Thus, the maleimide attachment group is reacted with a sulfhydryl of an antibody construct to give an intermediate succinimide ring. The hydrolyzed (open ring) form of the attachment group is resistant to deconjugation in the presence of plasma proteins.

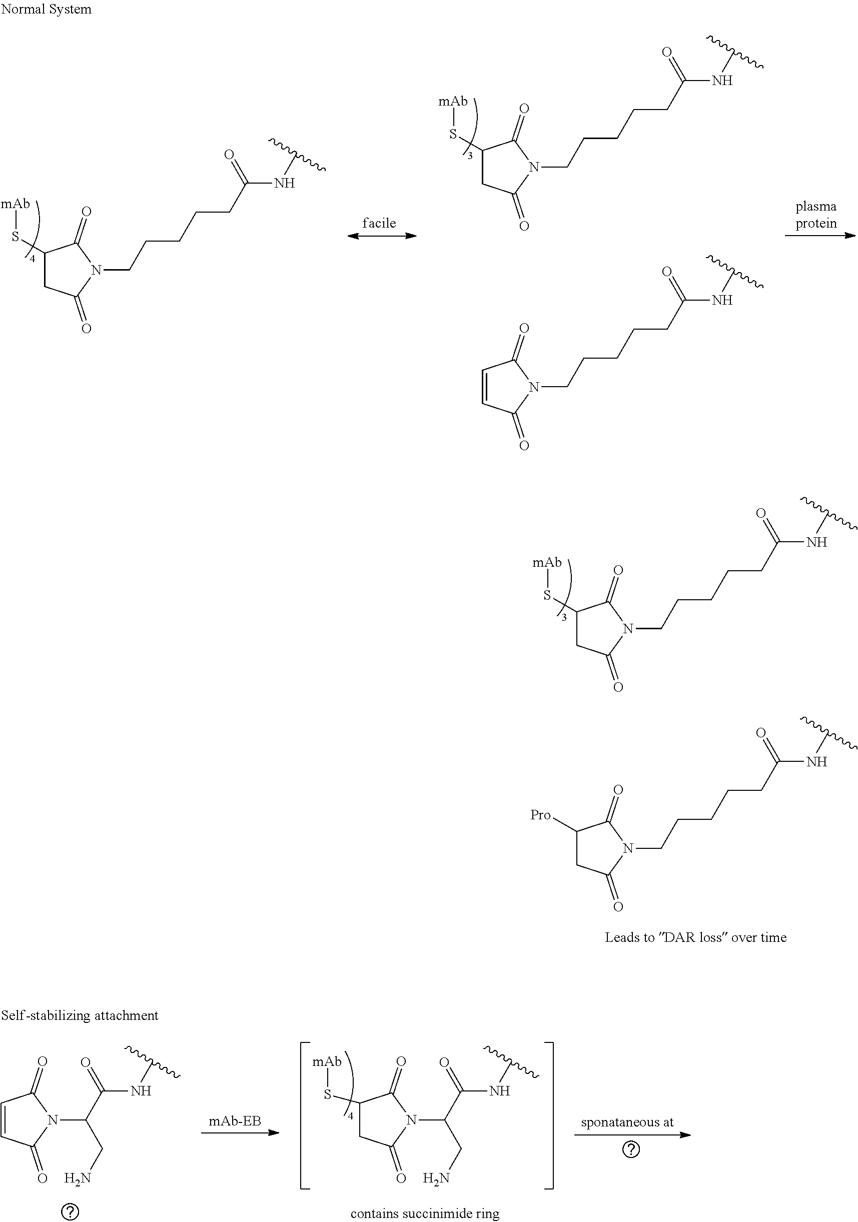

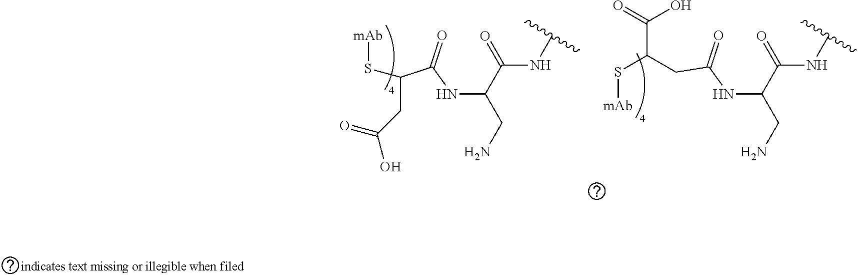

A method for bridging a pair of sulfhydryl groups derived from reduction of a native hinge disulfide bond has been disclosed and is depicted in the schematic below. An advantage of this methodology can be the ability to synthesize homogenous DAR4 conjugates by full reduction of IgGs (to give 4 pairs of sulfhydryls) followed by reaction with 4 equivalents of the alkylating agent. Conjugates containing “bridged disulfides” can also have increased stability.

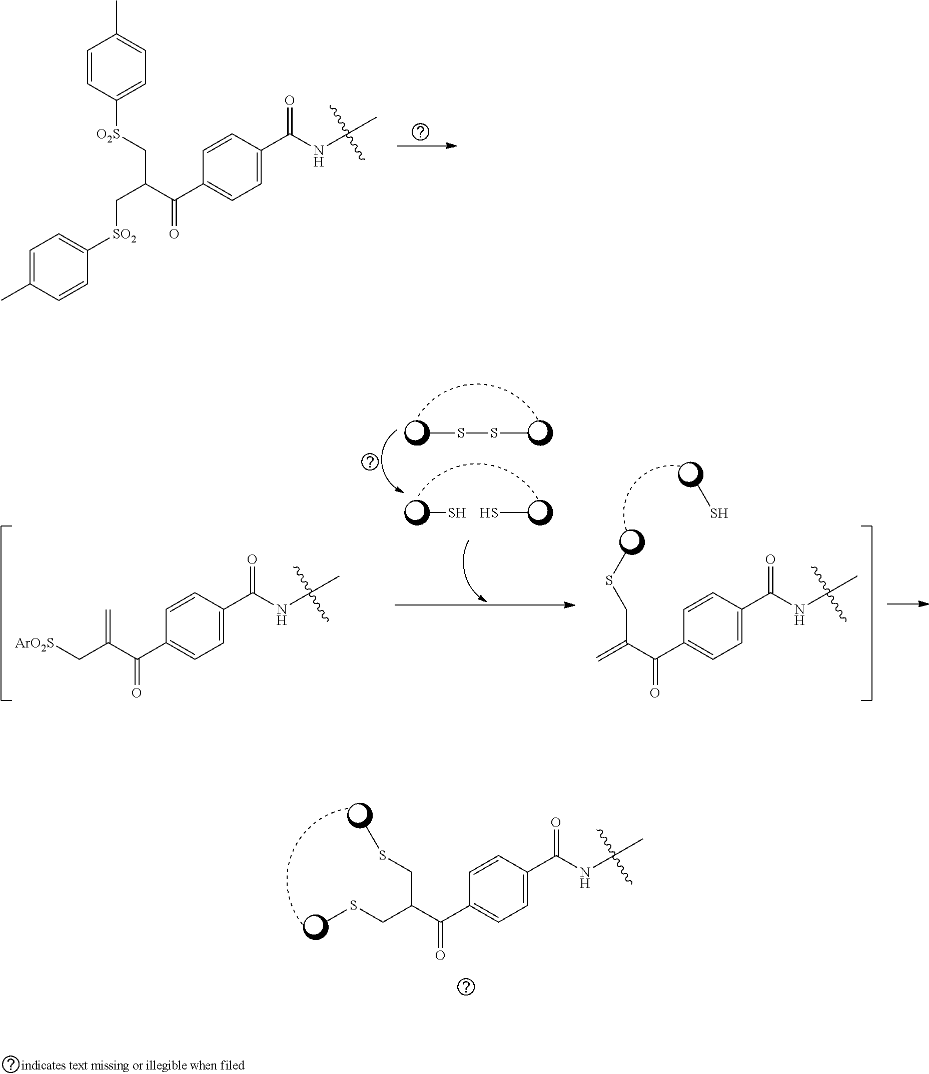

Similarly, as depicted below, a maleimide derivative that can bridge a pair of sulfhydryl groups has been developed.

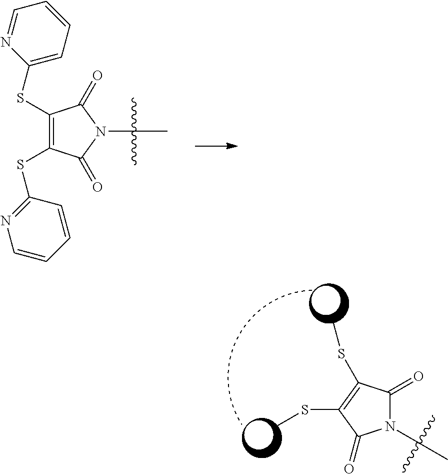 [SMILES: *N1C(=O)C(Sc2ccccn2)=C(Sc2ccccn2)C1=O ; *N1C(=O)C(SC)=C(SC)C1=O]

The attachment moiety can contain the following structural formulas (VIIa), (VIIb), or (VIIc):

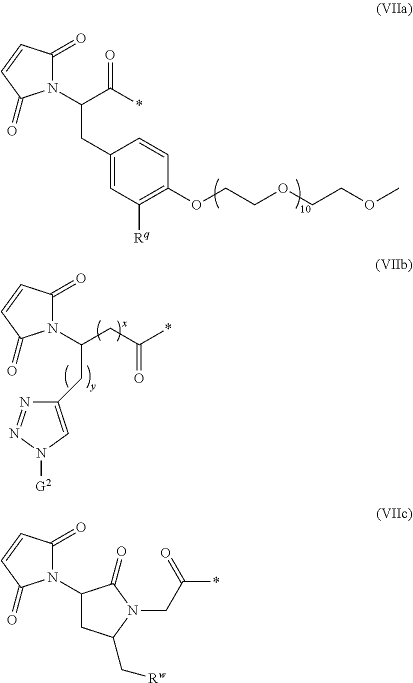

or salts thereof, wherein: R^{q} is H or O—(CH₂CH₂O)₁₁—CH₃; x is 0 or 1; y is 0 or 1; G² is —CH₂CH₂CH₂SO₃H or —CH₂CH₂—(CH₂CH₂O)₁₁—CH₃; R^{w} is —O—CH₂CH₂SO₃H or —NH(CO)—CH₂CH₂O—(CH₂CH₂O)₁₂—CH₃; and * represents the point of attachment to the remainder of the linker.

Exemplary embodiments of linkers according to structural formula (VIIa) and (VIIb) that can be included in the conjugates described herein can include the linkers illustrated below (as illustrated, the linkers can include a group suitable for covalently linking the linker in a conjugate and the wavy line or unlinked bond indicates an attachment site for a myeloid cell agonist):

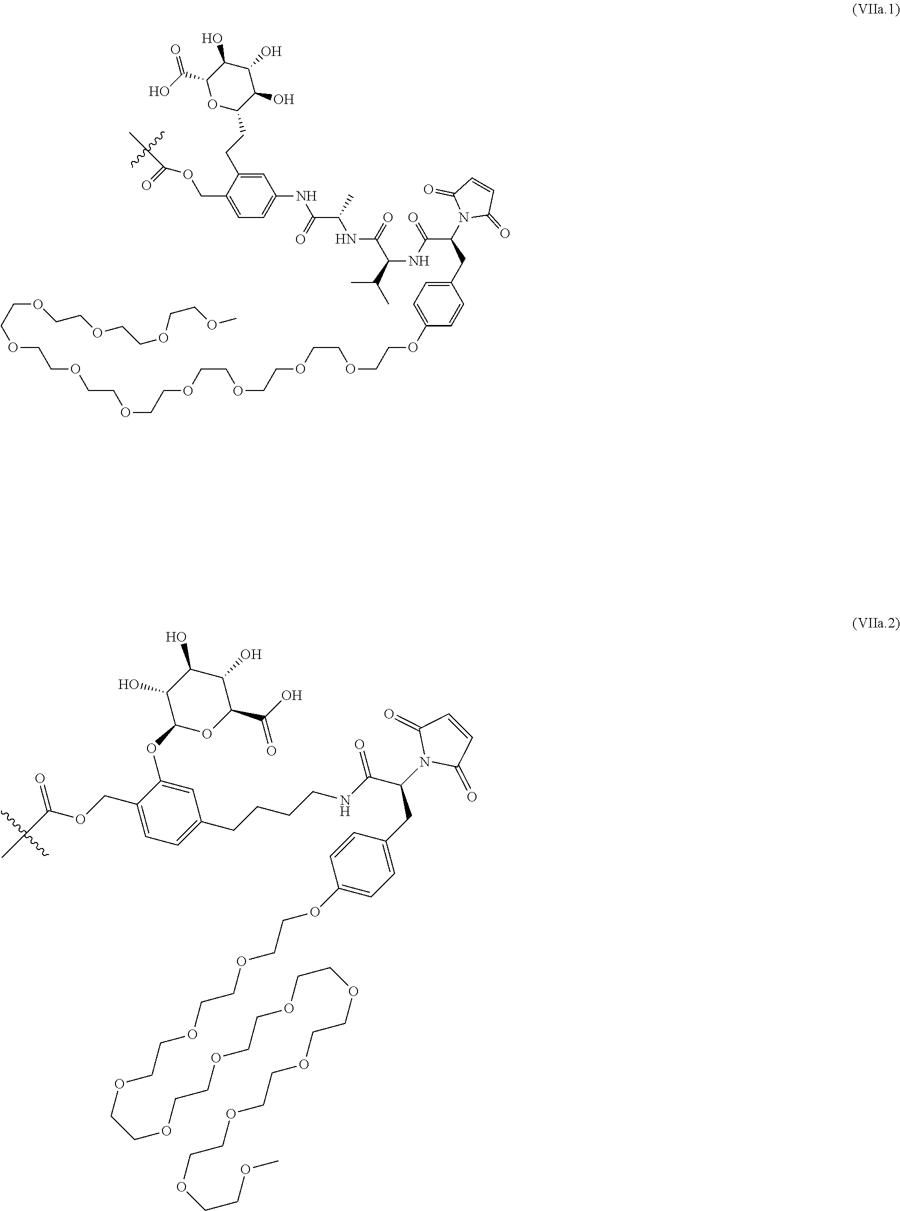 [SMILES: *C(=O)OCc1ccc(NC(=O)[C@H](C)NC(=O)[C@@H](NC(=O)[C@H](Cc2ccc(OCCOCCOCCOCCOCCOCCOCCOCCOCCOCCOCCOC)cc2)N2C(=O)C=CC2=O)C(C)C)cc1CC[C@@H]1O[C@H](C(=O)O)[C@@H](O)[C@H](O)[C@H]1O ; *C(=O)OCc1ccc(CCCCNC(=O)[C@H](Cc2ccc(OCCOCCOCCOCCOCCOCCOCCOCCOCCOCCOCCOC)cc2)N2C(=O)C=CC2=O)cc1O[C@@H]1O[C@H](C(=O)O)[C@@H](O)[C@H](O)[C@H]1O]

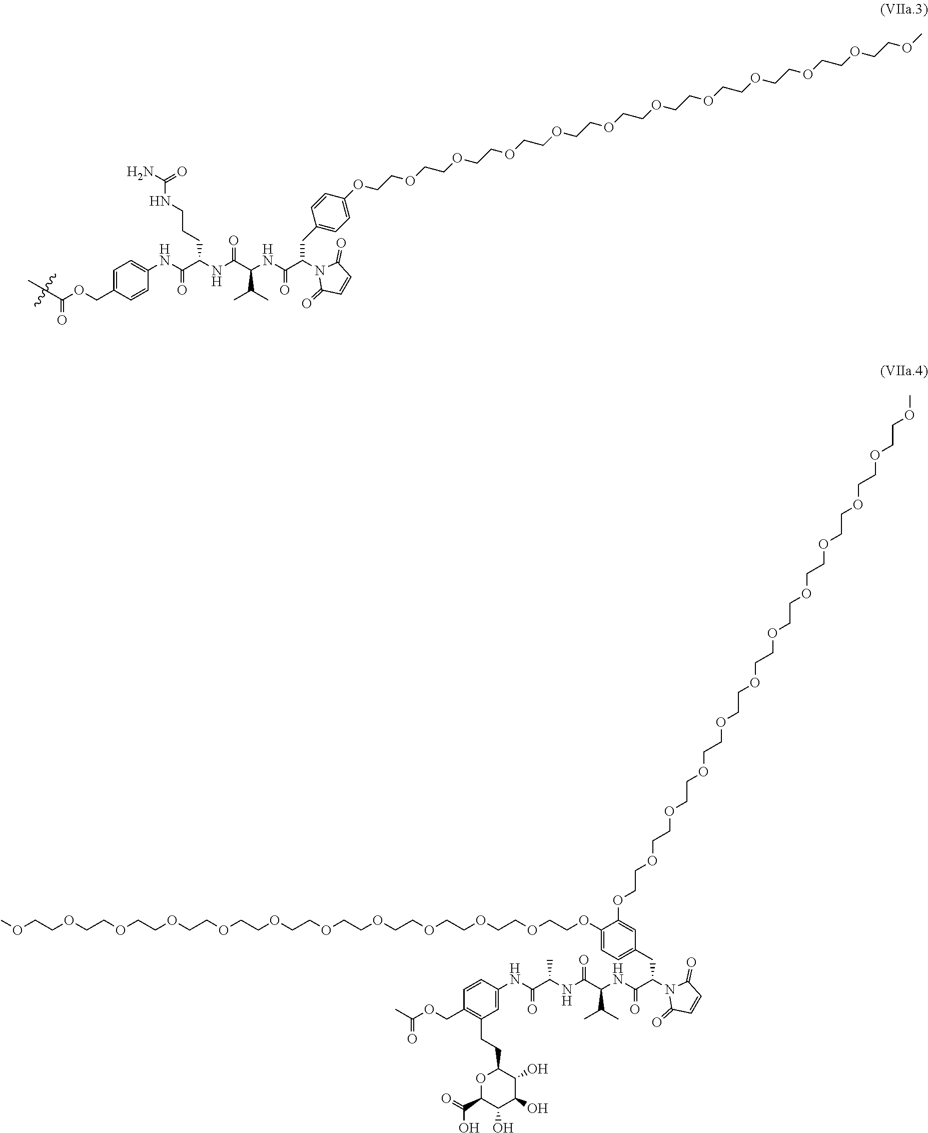

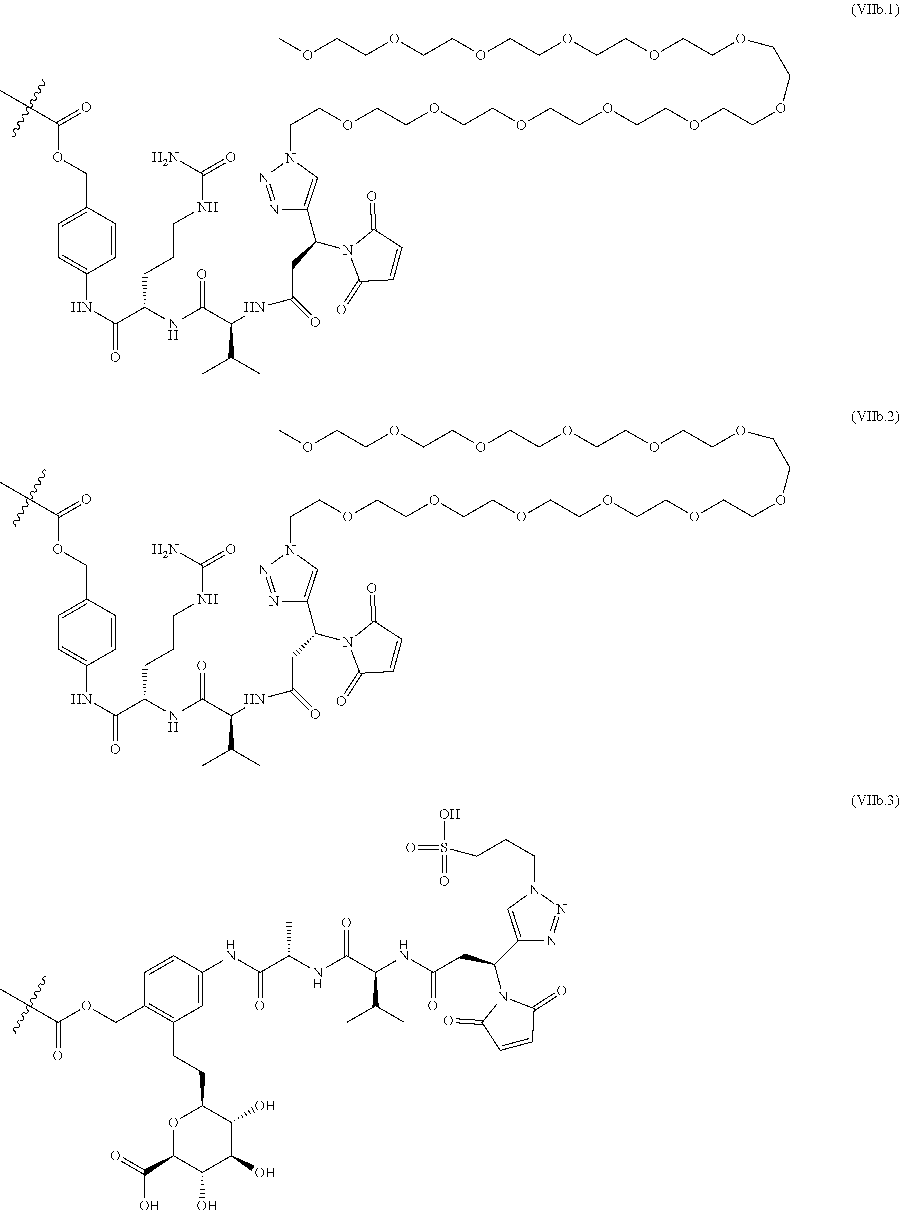 [SMILES: *C(=O)OCc1ccc(NC(=O)[C@H](CCCNC(N)=O)NC(=O)[C@@H](NC(=O)C[C@@H](c2cn(CCOCCOCCOCCOCCOCCOCCOCCOCCOCCOCCOCCOC)nn2)N2C(=O)C=CC2=O)C(C)C)cc1 ; *C(=O)OCc1ccc(NC(=O)[C@H](CCCNC(N)=O)NC(=O)[C@@H](NC(=O)C[C@H](c2cn(CCOCCOCCOCCOCCOCCOCCOCCOCCOCCOCCOCCOC)nn2)N2C(=O)C=CC2=O)C(C)C)cc1 ; *C(=O)OCc1ccc(NC(=O)[C@H](C)NC(=O)[C@@H](NC(=O)C[C@@H](c2cn(CCCS(=O)(=O)O)nn2)N2C(=O)C=CC2=O)C(C)C)cc1CC[C@@H]1O[C@H](C(=O)O)[C@@H](O)[C@H](O)[C@H]1O]

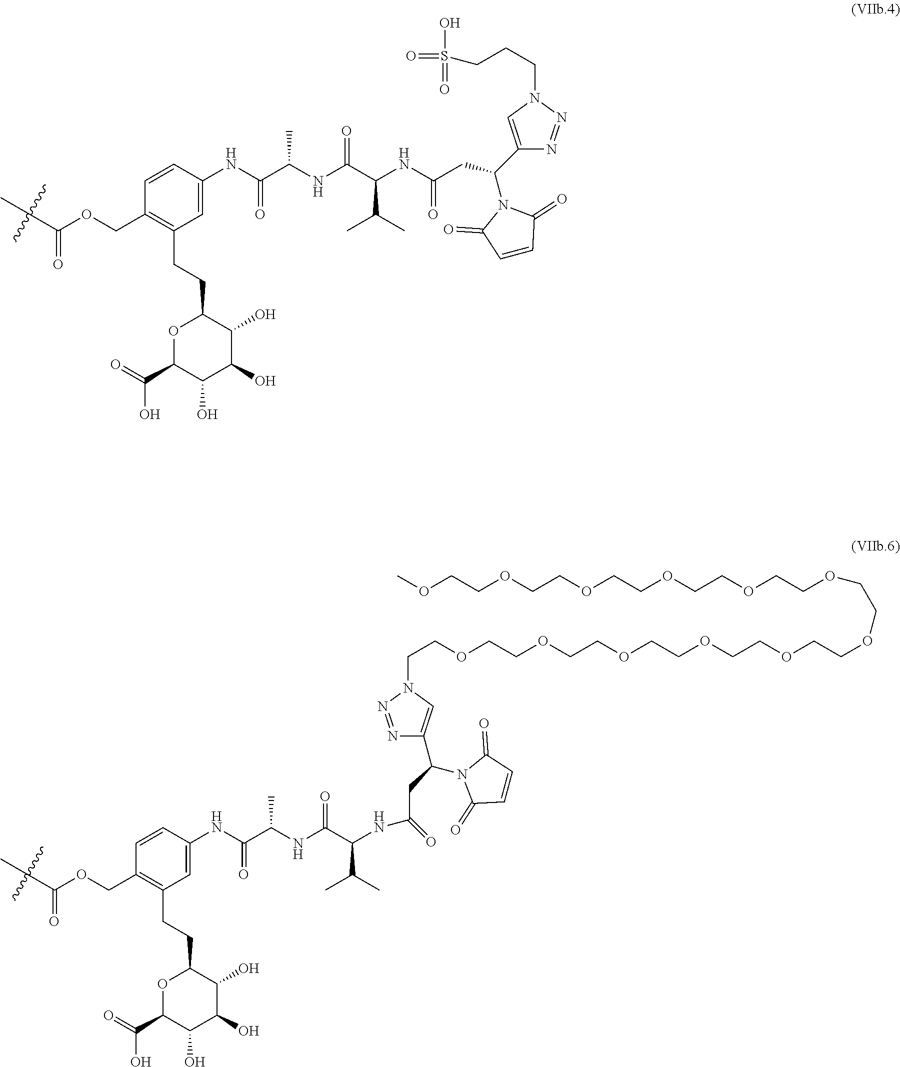 [SMILES: *C(=O)OCc1ccc(NC(=O)[C@H](C)NC(=O)[C@@H](NC(=O)C[C@H](c2cn(CCCS(=O)(=O)O)nn2)N2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)C[C@@H](c2cn(CCOCCOCCOCCOCCOCCOCCOCCOCCOCCOCCOCCOC)nn2)N2C(=O)C=CC2=O)C(C)C)cc1CC[C@@H]1O[C@H](C(=O)O)[C@@H](O)[C@H](O)[C@H]1O]

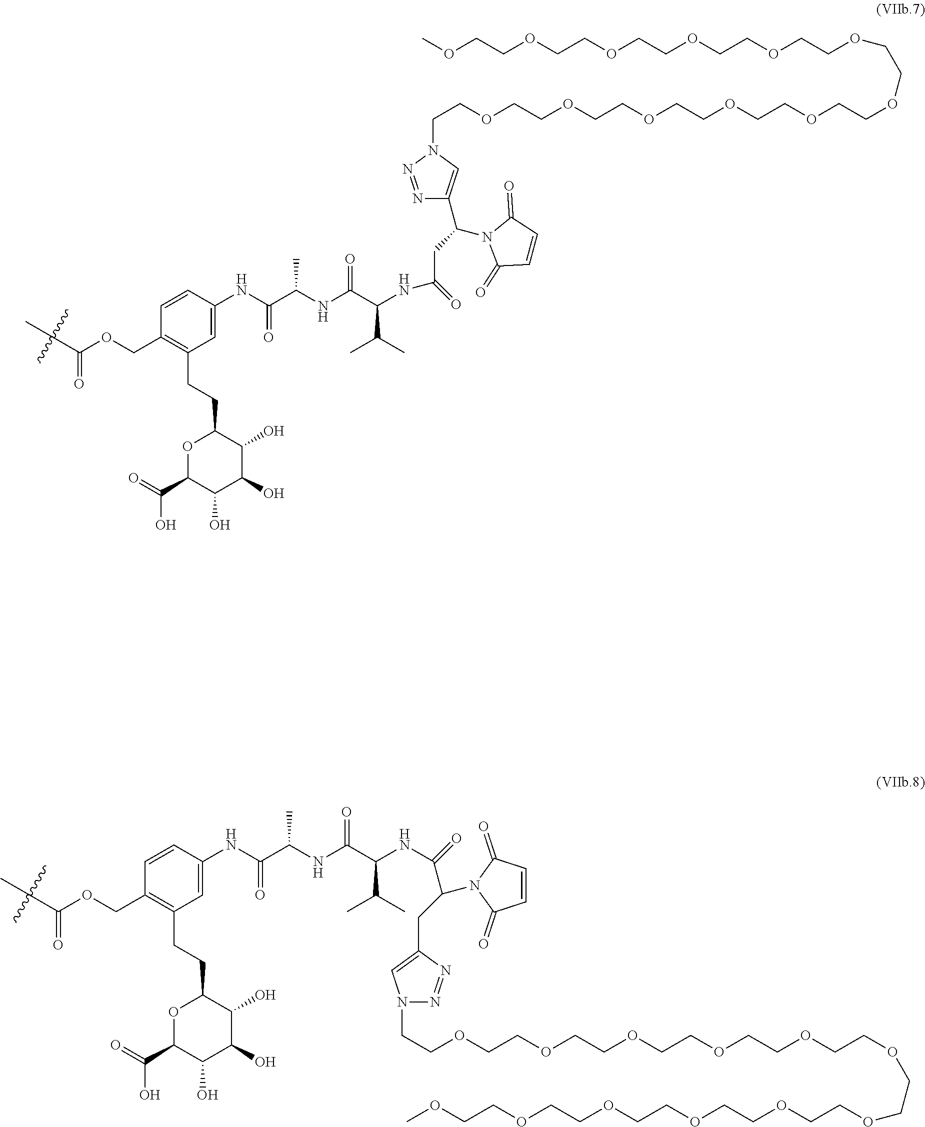 [SMILES: *C(=O)OCc1ccc(NC(=O)[C@H](C)NC(=O)[C@@H](NC(=O)C[C@H](c2cn(CCOCCOCCOCCOCCOCCOCCOCCOCCOCCOCCOCCOC)nn2)N2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)C(Cc2cn(CCOCCOCCOCCOCCOCCOCCOCCOCCOCCOCCOCCOC)nn2)N2C(=O)C=CC2=O)C(C)C)cc1CC[C@@H]1O[C@H](C(=O)O)[C@@H](O)[C@H](O)[C@H]1O]

Exemplary embodiments of linkers according to structural formula (VIIc) that can be included in the conjugates described herein can include the linkers illustrated below (as illustrated, the linkers can include a group suitable for covalently linking the linker in a conjugate):

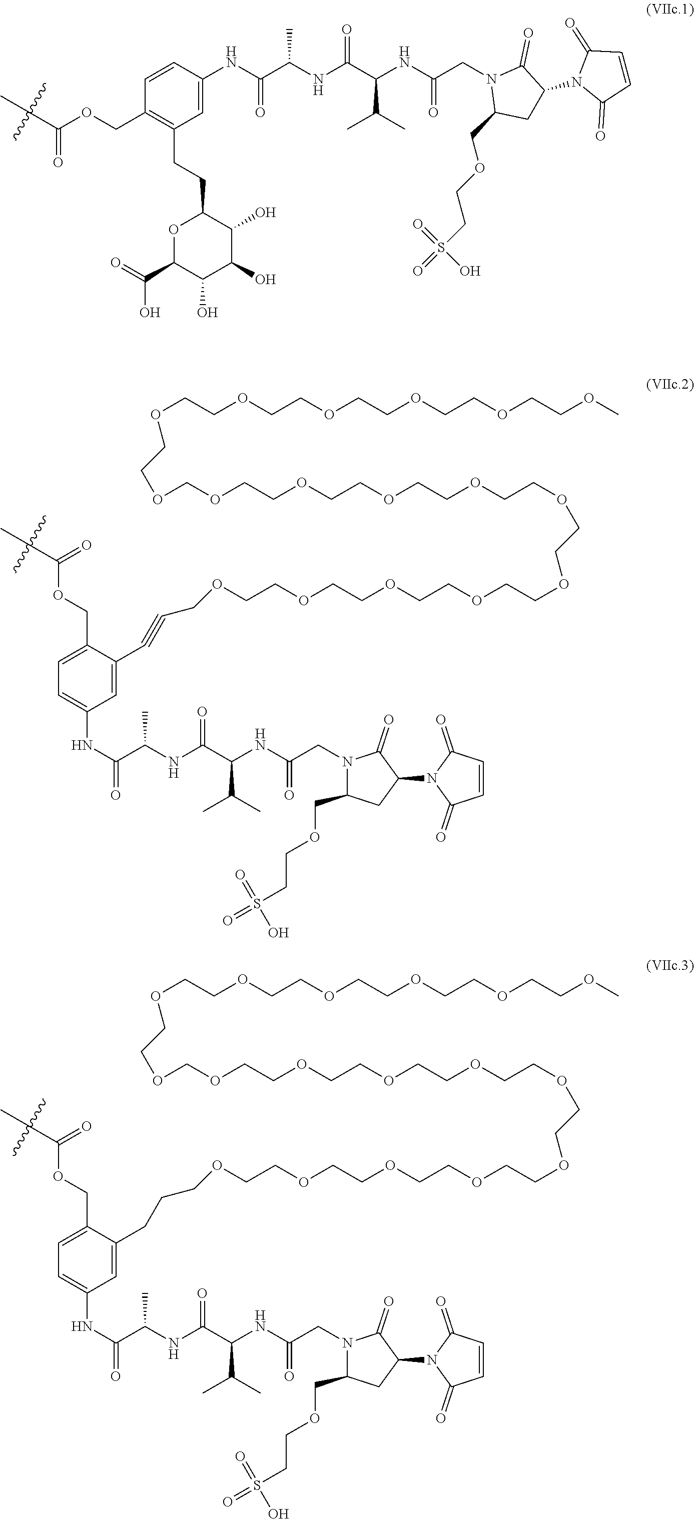 [SMILES: *C(=O)OCc1ccc(NC(=O)[C@H](C)NC(=O)[C@@H](NC(=O)CN2C(=O)[C@H](N3C(=O)C=CC3=O)C[C@H]2COCCS(=O)(=O)O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)CN2C(=O)[C@@H](N3C(=O)C=CC3=O)C[C@H]2COCCS(=O)(=O)O)C(C)C)cc1C#CCOCCOCCOCCOCCOCCOCCOCCOCCOCCOCOCCOCCOCCOCCOCCOCCOC ; *C(=O)OCc1ccc(NC(=O)[C@H](C)NC(=O)[C@@H](NC(=O)CN2C(=O)[C@@H](N3C(=O)C=CC3=O)C[C@H]2COCCS(=O)(=O)O)C(C)C)cc1CCCOCCOCCOCCOCCOCCOCCOCCOCCOCCOCOCCOCCOCCOCCOCCOCCOC]

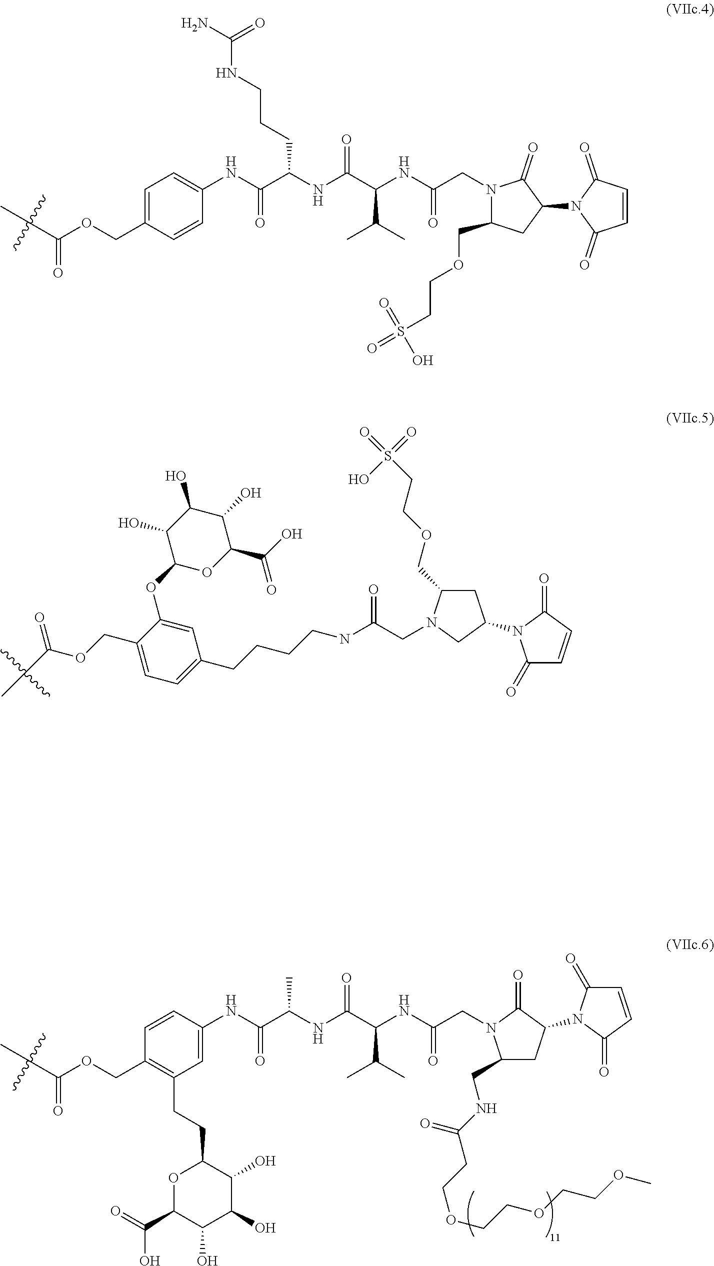

### Exemplary Syntheses of Myeloid Cell Agonist-Linkers

A myeloid cell agonist-linker compound can be synthesized by various methods before being attached to an antibody construct to form the conjugates as described herein. For example, a can be synthesized as shown in Scheme B1.

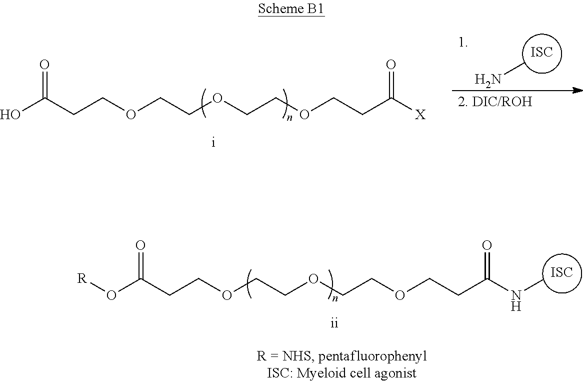

A PEGylated carboxylic acid (i) that has been activated for amide bond formation can be reacted with an appropriately substituted amine containing myeloid cell agonist to afford an intermediate amide. Formation of an activated ester (ii) can be achieved by reaction the intermediate amide-containing carboxylic using a reagent such as N-hydroxysuccinimide or pentafluorophenol in the presence of a coupling agent such as diisopropylcarbodiimide (DIC) to provide compounds (ii).

As another example, myeloid cell agonist-linkers can be synthesized as shown in Scheme B2.

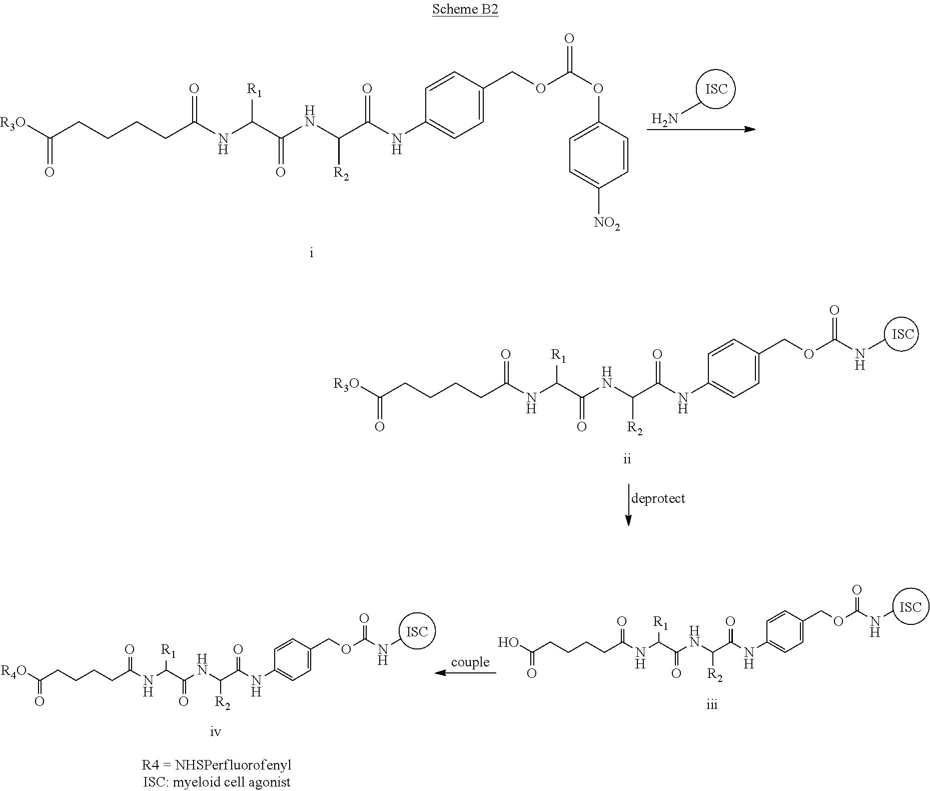

An activated carbonate such as (i) can be reacted with an appropriately substituted amine containing myeloid cell agonist to afford carbamates (ii) which can be deprotected using standard methods based on the nature of the R₃ ester group. The resulting carboxylic acid (iii) can then by coupled with an activating agent such as N-hydroxysuccinimide or pentafluorophenol to provide compounds (iv).

As an additional example, myeloid cell agonist-linker can be synthesized as shown in Scheme B3.

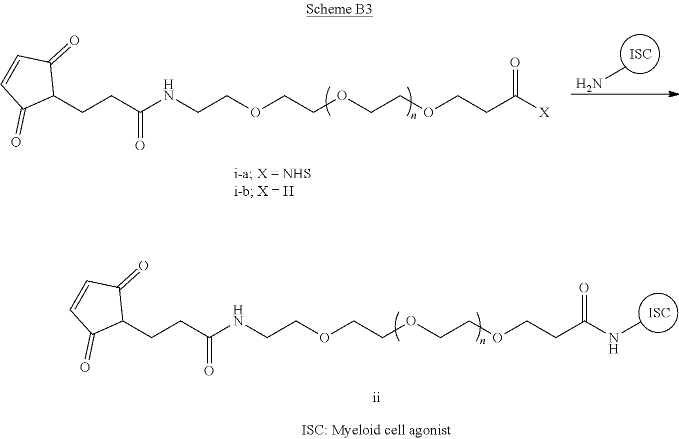

An activated carboxylic ester such as (i-a) can be reacted with an appropriately substituted amine containing myeloid cell agonist to afford amides (ii). Alternatively, carboxylic acids of type (i-b) can be coupled to an appropriately substituted amine containing myeloid cell agonist in the presence of an amide bond forming agent such as dicyclohexycarbodiimde (DCC) to provide the desired myeloid cell agonist-linker.

As an additional example, a myeloid cell agonist-linker can be synthesized as shown in Scheme B4.

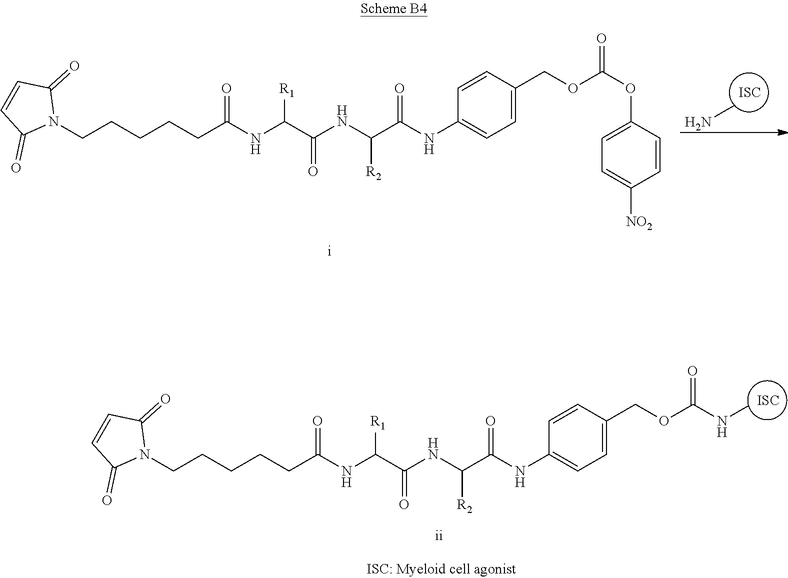

An activated carbonate such as (i) can be reacted with an appropriately substituted amine containing myeloid cell agonist to afford carbamates (ii) as the target myeloid cell agonist.

As an additional example, a myeloid cell agonist-linker can be synthesized as shown in Scheme B5.

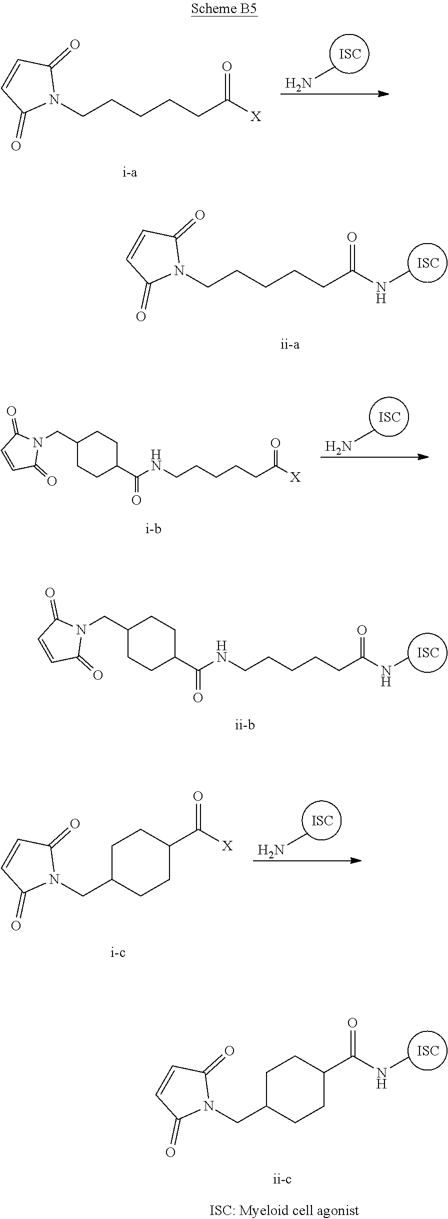

An activated carboxylic acid such as (i-a, i-b, i-c) can be reacted with an appropriately substituted amine containing myeloid cell agonist to afford amides (ii-a, ii-b, ii-c) as the target myeloid cell agonists.

These myeloid cell agonist-linkers can be made by various methods. It is understood that one skilled in the art may be able to make these compounds by similar methods or by combining other methods known to one skilled in the art. It is also understood that one skilled in the art would be able to make, in a similar manner as described herein by using the appropriate starting materials and modifying the synthetic route as needed. Starting materials and reagents can be obtained from commercial vendors or synthesized according to sources known to those skilled in the art or prepared as described herein.

### Conjugates

A conjugate as described herein comprise an antibody construct and at least one linker attached to at least one myeloid cell agonist. In some aspects, the present disclosure provides a conjugate represented by Formula I:

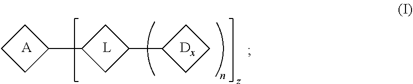

wherein:

A is the antibody construct,

L is the linker;

Dₓ is the myeloid cell agonist;

n is selected from 1 to 20; and

z is selected from 1 to 20.

In other aspects, the present disclosure provides a conjugate comprising at least one myeloid cell agonist (e.g., a compound or salt thereof), an antibody construct, and at least one linker, wherein each myeloid cell agonist is linked, i.e., covalently bound, to the antibody construct through a linker. The linker can be selected from a cleavable or non-cleavable linker. In some embodiments, the linker is cleavable. In alternative embodiments, the linker is non-cleavable. Linkers are further described in the present application in the preceding section, any one of which can be used to connect an antibody to a compound described herein.

In a conjugate, the drug loading is represented by the variable z. The variable z represents the number of myeloid cell agonist-linker molecules per antibody construct, or, when the variable n is equal to 1, the number of myeloid cell agonists per antibody construct. Depending on the context, z can represent the average number of myeloid cell agonist(-linker) molecules per antibody construct, also referred to the average drug loading. The variable z can range from 1 to 20, from 1-50 or from 1-100. In some conjugates, z is preferably from 1 to 8. In some preferred embodiments, when p represents the average drug loading, z ranges from about 2 to about 5. In some embodiments, z is about 2, about 3, about 4, or about 5. The average number of myeloid cell agonists per antibody construct in a preparation may be characterized by conventional means such as mass spectroscopy, HIC, ELISA assay, and HPLC.

A conjugate can comprise an antibody construct, a myeloid cell agonist, and a linker. A conjugate can comprise an antibody construct, a TLR7 agonist, and a linker. A conjugate can comprise an antibody construct, a TLR8 agonist, and a linker. A conjugate can comprise an antibody construct, a TLR7/8 agonist, and a linker. A conjugate can comprise an antibody construct, a benzazepine TLR7 and/or TLR8 agonist, and a linker. A conjugate can comprise an antibody construct, a ssRNA TLR7 and/or TLR8 agonist, and a linker. A conjugate can comprise an antibody construct, an imidazoquinolin TLR7 and/or TLR8 agonist, and a linker. A conjugate can comprise an antibody construct, a thiozoloquinolone TLR7 and/or TLR8 agonist, and a linker. In any of these examples, a reference to an agonist and a linker includes multiple agonists and/or linkers.

In some embodiments, the myeloid agonist is a TLR8 agonist selected from compounds 1.1-1.2, 1.4-1.20, 1.23-1.27, 1.29-1.46, 1.48, and 1.50-1.67 (Examples). In some embodiments, a myeloid agonist-linker compound (Linker-Payload) is selected from any of Linker-Payloads 2.1-2.17 or Linker-Payloads 2.20-2.39 (Examples). In some embodiments, the myeloid cell agonist is a TLR8 or TLR7 agonist selected from Category A Formulas (IA), (IB) (IIA),(IIB), (IIIA), and (IIIB), and Category B Formulas (IA), (IB) or (IC), respectively.

A conjugate can comprise an antibody construct with a wild-type Fc binding domain. A conjugate can comprise an antibody construct with a Fc binding domain variant. A conjugate can comprise an antibody construct with a Fc binding domain variant that increases the binding of the Fc binding domain to an Fc receptor. The Fc binding domain variant can comprise a substitution at more than one amino acid residue such as at 5 different amino acid residues including L235V/F243L/R292P/Y300L/P396L, as at 2 different amino acid residues including S239D/I332E, or as at 3 different amino acid residues including S298A/E333A/K334A as compared to a wild-type IgG1 Fc binding domain. The numbering of amino acids residues described herein is according to the EU index.

The linker can be one of the linkers described herein. A linker can be cleavable, non-cleavable, hydrophilic, or hydrophobic. A cleavable linker can be sensitive to enzymes. A cleavable linker can be cleaved by enzymes such as proteases. A cleavable linker can be a linker containing a valine-citrulline or a valine-alanine peptide. A valine-citrulline- or valine-alanine-containing linker can contain a pentafluorophenyl group. A valine-citrulline- or valine-alanine-containing linker can contain a succimide group or a maleimide group. A valine-citrulline- or valine-alanine-containing linker can contain a PABA group. A valine-citrulline- or valine-alanine-containing linker can contain a PABA group and a pentafluorophenyl group. A valine-citrulline-containing or valine-alanine-containing linker can contain a PABA group and a maleimide group. A valine-citrulline-containing or valine-alanine-containing linker can contain a PABA group and a succinimide group. A non-cleavable linker can be protease insensitive. A non-cleavable linker can contain a maleimide group. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group. A linker can be a combination of a maleimide group and one or more polyethylene glycol molecules. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonyl) linker. A linker can also comprise an alkylene, alkenylene, alkynylene, polyether, polyester, polyamide, polyamino acids, polypeptides, cleavable peptides, and/or aminobenzylcarbamate group. A linker can contain a maleimide at one end and an N-hydroxysuccinimidyl ester at the other end. A linker can contain a lysine with an N-terminal amine acetylated, and a valine-citrulline cleavage site.

A linker can have a linkage created by a microbial transglutaminase, wherein the link is created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be attached by a Sortase A enzyme fusing an LXPTG recognition motif (SEQ ID NO: 1) to an N-terminal GGG motif to regenerate a native amide bond. The linker created can therefore link a moiety attached to the LXPTG recognition motif (SEQ ID NO: 1) with a moiety attached to the N-terminal GGG motif. A linker can be a link created between an unnatural amino acid on one moiety reacting with oxime bond that was formed by modifying a ketone group with an alkoxyamine on another moiety. A moiety can be an antibody construct. A moiety can be a binding domain. A moiety can be an antibody. A moiety can be an myeloid cell agonist.

A conjugate can comprise an antibody construct comprising a target antigen binding domain and an Fc binding domain. The target antigen binding domain can specifically bind to a first antigen on a liver cell, wherein the antigen is a liver cell antigen. A first antigen can be expressed by a liver cell. For example, a first antigen can be a liver cell antigen. A liver cell antigen can be a molecular marker is preferentially expressed on a liver cell as compared to cells from other normal tissues. For example, a liver cell antigen can be expressed on a canalicular cell, Kupffer cell, hepatocyte, a stellate cell, or other liver cell type, or a combination thereof. The liver cell antigen can be a liver cell surface receptor. The liver cell antigen can be a hepatocyte antigen. The liver cell antigen can be expressed on a non-cancerous liver cell. The liver cell antigen can be expressed on a cell infected with a virus. The virus can be a liver virus. The virus can be a hepatitis virus. The virus can be HBV. The virus can be HCV. A liver cell antigen can include, but is not limited to, ASGR1, ASGR2, TRF2, UGT1A1, SLC22A7, SLC13A5, SLC22A1, and C9. In some embodiments, the liver cell antigen is selected from the group consisting of ASGR1, ASGR2, TRF2, UGT1A1, SLC22A7, SLC13A5, SLC22A1, and C9. In some embodiments, the liver cell antigen is selected from the group consisting of ASGR1, ASGR2, TRF2, UGT1A1, SLC22A7, SLC13A5, and SLC22A1. In some embodiments, the liver cell antigen is selected from the group consisting of ASGR1, ASGR2 and TRF2. In some embodiments, the target antigen binding domain can specifically bind to a liver cell antigen, wherein the liver cell antigen has an amino acid sequence that comprises at least 80% identity to an amino acid sequence of an antigen selected from the group consisting of ASGR1, ASGR2, TRF2, UGT1A1, SLC22A7, SLC13A5, SLC22A1, and C9. The target antigen binding domain can specifically bind to a liver cell antigen, wherein the liver cell antigen has an amino acid sequence that comprises at least 85%, 90%, 95%, 98%, or 100% identity to an amino acid sequence of an antigen selected from the group consisting of ASGR1, ASGR2, TRF2, UGT1A1, SLC22A7, SLC13A5, SLC22A1, and C9. In other embodiments, a first antigen is a viral antigen from a virus infecting a liver cell. A viral antigen can be a molecular marker of a virus, which is expressed on a liver cell when the liver cell is infected with the virus. For example, a viral antigen can be expressed on a canalicular cell, Kupffer cell, hepatocyte, a stellate cell, or other liver cell type, or a combination thereof. The virus can be a hepatitis virus, such as HBV or HCV. The virus can be HBV. The virus can be HCV. The viral antigen can be expressed on a non-cancerous liver cell infected with a virus. A viral antigen can include, but is not limited to, the components of HBV such as HBsAg, HBcAg, HBeAg, Hepatitis B virus DNA polymerase, or HBx, and components of HCV such as Core protein, E1 and E2, NS2, NS3, NS4A, NS4B, NS5A, or NS5B. In some embodiments, the viral antigen is selected from the group consisting of HBV components such as HBsAg, HBcAg, HBeAg, Hepatitis B virus DNA polymerase, or HBx, and components of HCV such as Core protein, E1 and E2, NS2, NS3, NS4A, NS4B, NS5A, or NS5B. In some embodiments, the viral antigen is HBsAg, HBcAg or HBeAg. In some embodiments, the the viral antigen is HBsAg. In some embodiments, the target antigen binding domain can specifically bind to a viral antigen from a virus infecting a liver cell, wherein the viral antigen has an amino acid sequence that comprises at least 80% identity to an amino acid sequence of an antigen selected from the group consisting of HBsAg, HBcAg, HBeAg, Hepatitis B virus DNA polymerase, HBx, Core protein, E1 and E2, NS2, NS3, NS4A, NS4B, NS5A, and NS5B. The target antigen binding domain can specifically bind to a viral antigen for a virus infecting a liver cell, wherein the viral antigen has an amino acid sequence that comprises at least 85%, 90%, 95%, 98%, or 100% identity to an amino acid sequence of an antigen selected from the group consisting of HBsAg, HBcAg, HBeAg, Hepatitis B virus DNA polymerase, HBx, Core protein, E1 and E2, NS2, NS3, NS4A, NS4B, NS5A, and NS5B.

The Kd for binding of a first antigen binding domain of a conjugate to a first antigen in the presence of a myeloid cell agonist can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the Kd for binding of the first antigen binding domain to the first antigen of a conjugate in the absence of the myeloid cell agonist. The Kd for binding of a first antigen binding domain of a conjugate to a first antigen in the presence of the myeloid cell agonist can be less than 10 nM. The Kd for binding of a first antigen binding domain of a conjugate to a first antigen in the presence of the myeloid cell agonist can be less than 100 nM, less than 50 nM, less than 20 nM, less than 5 nM, less than 1 nM, or less than 0.1 nM.

The conjugate can be capable of specifically binding to a single antigen. The conjugate can be capable of specifically binding to two or more antigens. The conjugate can comprise antibody construct comprising a second antigen binding domain. A second antigen binding domain can specifically bind to a second antigen. A second antigen can be expressed by a liver cell. For example, a second antigen can be a liver cell antigen. A liver cell antigen can be a molecular marker is preferentially expressed on a liver cell as compared to cells from other normal tissues. For example, a liver cell antigen can be expressed on a canalicular cell, Kupffer cell, hepatocyte, a stellate cell, or a combination thereof. The liver cell antigen can be a liver cell surface receptor. The liver cell antigen can be a hepatocyte antigen. The liver cell antigen can be expressed on a non-cancerous liver cell. The liver cell antigen can be expressed on a cell infected with a virus. The virus can be a hepatitis virus, such as HBV or HCV. The virus can be HBV. The virus can be HCV. A liver cell antigen can include, but is not limited to, ASGR1, ASGR2, TRF2, UGT1A1, SLC22A7, SLC13A5, SLC22A1, and C9. In some embodiments, the liver cell antigen is selected from the group consisting of ASGR1, ASGR2, TRF2, UGT1A1, SLC22A7, SLC13A5, SLC22A1, and C9. In some embodiments, the liver cell antigen is selected from the group consisting of ASGR1, ASGR2, TRF2, UGT1A1, SLC22A7, SLC13A5 and SLC22A1. In some embodiments, the liver cell antigen is selected from the group consisting of ASGR1, ASGR2 and TRF2. The second antigen binding domain can specifically bind to a liver cell antigen, wherein the liver cell antigen has an amino acid sequence that comprises at least 80% identity to an amino acid sequence of an antigen selected from the group consisting of ASGR1, ASGR2, TRF2, UGT1A1, SLC22A7, SLC13A5, SLC22A1, and C9. The second antigen binding domain can specifically bind to a liver cell antigen, wherein the liver cell antigen has an amino acid sequence that comprises at least 85%, 90%, 95%, 98%, or 100% identity to an amino acid sequence of an antigen selected from the group consisting of ASGR1, ASGR2, TRF2, UGT1A1, SLC22A7, SLC13A5, SLC22A1, and C9.

In other embodiments, a second antigen can be a viral antigen from a virus infecting a liver cell. A viral antigen can be a molecular marker of a virus, which is expressed on a liver cell when the liver cell is infected with the virus. For example, a viral antigen can be expressed on a canalicular cell, Kupffer cell, hepatocyte, a stellate cell, or a combination thereof, when infected by a virus. The virus can be a hepatitis virus, such as HBV or HCV. The virus can be HBV. The virus can be HCV. The viral antigen can be expressed on a non-cancerous liver cell infected with a virus. A viral antigen can include, but is not limited to, the components of HBV such as HBsAg, HBcAg, HBeAg, Hepatitis B virus DNA polymerase, or HBx, and components of HCV such as Core protein, E1 and E2, NS2, NS3, NS4A, NS4B, NS5A, or NS5B. In some embodiments, the viral antigen is selected from the group consisting of the components of HBV such as HBsAg, HBcAg, HBeAg, Hepatitis B virus DNA polymerase, or HBx, and the components of HCV such as Core protein, E1 and E2, NS2, NS3, NS4A, NS4B, NS5A, or NS5B. In some embodiments, the viral antigen is HBsAg, HBcAg or HBeAg. In some embodiments, the viral antigen is HBsAg. In some embodiments, the second antigen binding domain can specifically bind to a viral antigen from a virus infecting a liver cell, wherein the viral antigen has an amino acid sequence that comprises at least 80% identity to an amino acid sequence of an antigen selected from the group consisting of HBsAg, HBcAg, HBeAg, Hepatitis B virus DNA polymerase, HBx, Core protein, E1 and E2, NS2, NS3, NS4A, NS4B, NS5A, and NS5B. The second antigen binding domain can specifically bind to a viral antigen for a virus infecting a liver cell, wherein the viral antigen has an amino acid sequence that comprises at least 85%, 90%, 95%, 98%, or 100% identity to an amino acid sequence of an antigen selected from the group consisting of HBsAg, HBcAg, HBeAg, Hepatitis B virus DNA polymerase, HBx, Core protein, E1 and E2, NS2, NS3, NS4A, NS4B, NS5A, and NS5B.

The Kd for binding of a second antigen binding domain of a conjugate to the second antigen in the presence of a myeloid cell agonist can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the Kd for binding of the second antigen binding domain to the second antigen of a conjugate in the absence of the myeloid cell agonist. The Kd for binding of a second antigen binding domain of a conjugate to the second antigen in the presence of the myeloid cell agonist can be less than 10 nM. The Kd for binding of a second antigen binding domain of a conjugate to the second antigen in the presence of the myeloid cell agonist can be less than 100 nM, less than 50 nM, less than 20 nM, less than 5 nM, less than 1 nM, or less than 0.1 nM. In contrast, the Kd for binding of a second antigen binding domain of conjugate to a second antigen in the presence of the myeloid cell agonist when the first antigen binding domain is bound to the first antigen binding domain's antigen can be greater than 100 nM. The Kd for binding of a second antigen binding domain of a conjugate to a second antigen in the presence of the myeloid cell agonist when the first binding domain is bound to the first antigen binding domain's antigen can be greater than 100 nM, greater than 200 nM, greater than 300 nM, greater than 400 nM, greater than 500 nM, or greater than 1000 nM.

The conjugate can comprise an antibody construct comprising an Fc binding domain that can bind to an FcR when linked to a myeloid cell agonist. The conjugate can comprise an Fc binding domain that can bind to an FcR to initiate FcR-mediated signaling when linked to a myeloid cell agonist. The conjugate can bind to its antigen(s) when linked to a myeloid cell agonist. The conjugate can bind to its antigen(s) when linked to a myeloid cell agonist and the Fc binding domain of the conjugate can bind to an FcR when linked to a myeloid cell agonist. The conjugate can bind to its antigen when linked to a myeloid cell agonist and the Fc binding domain of the conjugate can bind to an FcR to initiate FcR-mediated signaling when linked to a myeloid cell agonist. The Fc binding domain linked to a myeloid cell agonist can be a Fc binding domain variant. The Fc binding domain variant can comprise a substitution at more than one amino acid residue, such as at 5 different amino acid residues including L235V/F243L/R292P/Y300L/P396L, as at 2 different amino acid residues including S239D/I332E, or as at 3 different amino acid residues including S298A/E333A/K334A. The Kd for binding of an Fc binding domain to a Fc receptor when the Fc binding domain is linked to a myeloid cell agonist can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the Kd for binding of the Fc binding domain to the Fc receptor in the absence of linking to the myeloid cell agonist. The Kd for binding of an Fc binding domain to an Fc receptor when linked to a myeloid cell agonist can be less than 10 nM. The Kd for binding of an Fc binding domain to an Fc receptor when linked to a myeloid cell agonist can be less than 100 nM, less than 50 nM, less than 20 nM, less than 5 nM, less than 1 nM, or less than 0.1 nM. In contrast, the Kd for binding of an Fc binding domain to an Fc receptor when linked to a myeloid cell agonist and when the first binding domain is bound to its antigen can be greater than 100 nM. The Kd for binding of an Fc binding domain to an Fc receptor when linked to a myeloid cell agonist and when the first binding domain is bound to its antigen can be greater than 100 nM, greater than 200 nM, greater than 300 nM, greater than 400 nM, greater than 500 nM, or greater than 1000 nM.

The myeloid cell agonist of the conjugate can be a TRL7 agonist and/or a TLR8 agonist. IN some embodiments, the myeloid cell agonist is a TLR7 agonist. In some embodiments, the myeloid cell agonist is a TLR8 agonist.

A linker can be connected to an antibody construct and to a myeloid cell agonist of a conjugate by a direct linkage between the antibody construct, the myeloid cell agonist and the linker. A direct linkage is a covalent bond.

A linker can be attached to an antibody construct at any suitable site, such as for example at a terminus of an amino acid sequence or at a side chain of a cysteine residue, an engineered cysteine residue, a lysine residue, a serine residue, a threonine residue, a tyrosine residue, an aspartic acid residue, a glutamic acid residue, a glutamine residue, an engineered glutamine residue, a selenocysteine residue, or a non-natural amino acid. Non-natural amino acids can include para-azidomethyl-1-phenylalanine (pAMF). An attachment site can also be at a residue containing an oxime bond that was formed by modifying a ketone group with an alkoxyamine on another moiety, and a reactive primary amine, such as a reactive primary amine at a C-terminal end of a protein or peptide, such as by using Sortase A linker, which can be created by a Sortase A enzyme fusing an LXPTG recognition motif (SEQ ID NO: 1) to an N-terminal GGG motif to regenerate a native amide bond. The linker created can therefore link a moiety attached to the LXPTG recognition motif (SEQ ID NO: 1) with a moiety attached to the N-terminal GGG motif

An attachment can be via any of a different types of bonds, for example but not limited to, an amide bond, an ester bond, an ether bond, a carbon-nitrogen bond, a carbon-carbon single, double or triple bond, a disulfide bond, or a thioether bond. A linker can have at least one functional group, which can be linked to the antibody construct (e.g., an antibody). Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups.

A linker can be connected to an antibody construct at a hinge cysteine of an antibody Fc region or domain. A linker can be connected to an antibody construct at a light chain constant domain lysine. A linker can be connected to an antibody construct at an engineered cysteine in the light chain. A linker can be connected to an antibody construct at an engineered light chain glutamine. A linker can be connected to an antibody construct at an unnatural amino acid engineered into the light chain. A linker can be connected to an antibody construct at a heavy chain constant domain lysine. A linker can be connected to an antibody construct at an engineered cysteine in the heavy chain. A linker can be connected to an antibody construct at an engineered heavy chain glutamine. A linker can be connected to an antibody construct an unnatural amino acid engineered into the heavy chain. Amino acids can be engineered into an amino acid sequence of an antibody construct as described herein, for example, and can be connected to a linker of a conjugate. Engineered amino acids can be added to a sequence of existing amino acids. Engineered amino acids can be substituted for one or more existing amino acids of a sequence of amino acids.

A linker can be conjugated to an antibody construct via a sulfhydryl group. A linker can be conjugated to an antibody construct via a primary amine. A linker can be a link created between an unnatural amino acid on an antibody construct reacting with oxime bond that was formed by modifying a ketone group with an alkoxyamine on a myeloid cell agonist. When a linker is connected to an antibody construct at the sites described herein, an Fc domain of the conjugate can bind to Fc receptors. When a linker is connected to an antibody construct at the sites described herein, the antigen binding domain of the conjugate can bind its antigen. When a linker is connected to an antibody construct at the sites described herein, a binding domain of the conjugate can bind its antigen.

An antibody with engineered reactive cysteine residues can be used to link a binding domain to the antibody. A linker can connect an antibody construct to a binding domain via Sortase A linker. A Sortase A linker can be created by a Sortase A enzyme fusing an LXPTG recognition motif (SEQ ID NO: 1) to an N-terminal GGG motif to regenerate a native amide bond. The linker created can therefore link an antibody construct attached to the LXPTG recognition motif (SEQ ID NO: 1) with a binding domain attached to the N-terminal GGG motif. A binding domain can be connected to a linker by a direct linkage. A direct linkage is a covalent bond. For example, a linker can be attached to a terminus of an amino acid sequence of a binding domain, or could be attached to a side chain modification to the binding domain, such as the side chain of a cysteine residue, an engineered cysteine residue, a lysine residue, a serine residue, a threonine residue, a tyrosine residue, an aspartic acid residue, a glutamic acid residue, a glutamine residue, an engineered glutamine residue, a selenocysteine residue, or a non-natural amino acid. Non-natural amino acids can include para-azidomethyl-1-phenylalanine (pAMF). An attachment can also be at a residue containing an oxime bond that was formed by modifying a ketone group with an alkoxyamine on another moiety, and a reactive primary amine, such as a reactive primary amine at a C-terminal end of a protein or peptide. An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the binding domain. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups. Amino acids can be engineered into an amino acid sequence of the binding domain. Engineered amino acids can be added to a sequence of existing amino acids. Engineered amino acids can be substituted for one or more existing amino acids of a sequence of amino acids. A linker can be conjugated to a binding domain via a sulfhydryl group. A linker can be conjugated to a binding domain via a primary amine. A binding domain can be conjugated to the C-terminal of an Fc domain of a conjugate.

An antibody or antibody construct with engineered reactive cysteine residues can be used to link a myeloid cell agonist to the antibody construct. A linker can connect an antibody construct to a myeloid cell agonist via linker. A linker can connect an antibody construct to a myeloid cell agonist via Sortase A linker. A Sortase A linker can be created by a Sortase A enzyme fusing an LXPTG recognition motif (SEQ ID NO: 1) to an N-terminal GGG motif to regenerate a native amide bond. The linker created can therefore link an antibody attached the LXPTG recognition motif (SEQ ID NO: 1) with a myeloid cell agonist attached to the N-terminal GGG motif. A linker can be a link created between an unnatural amino acid an antibody reacting with oxime bond that was formed by modifying a ketone group with an alkoxyamine on a myeloid cell agonist. The myeloid cell agonist can comprise one or more rings selected from carbocyclic and heterocyclic rings. The myeloid cell agonist can be covalently bound to a linker by a bond to an exocyclic carbon or nitrogen atom on the myeloid cell agonist. A linker can be conjugated to a myeloid cell agonist via an exocyclic nitrogen or carbon atom of a myeloid cell agonist.

A linker agonist complex can dissociate under physiological conditions to yield an active agonist.

A linker can be connected to a myeloid cell agonist by a direct linkage between the myeloid cell agonist and the linker. A linker can be attached to a TLR8 agonist by a direct linkage between the TLR8 agonist and the linker. A linker can be attached to a TLR7 agonist by a direct linkage between the TLR7 and the linker.

A direct linkage can be a covalent bond. For example, a linker can be attached to a terminus of an amino acid sequence of an antibody construct, or could be attached to a side chain modification to the antibody construct, such as example at a side chain of a cysteine residue, an engineered cysteine residue, a lysine residue, a serine residue, a threonine residue, a tyrosine residue, an aspartic acid residue a glutamic acid residue, a glutamine residue, an engineered glutamine residue, a selenocysteine residue, or a non-natural amino acid. Non-natural amino acids can include para-azidomethyl-1-phenylalanine (pAMF). An attachment can also be at a residue containing an oxime bond that was formed by modifying a ketone group with an alkoxyamine on another moiety, and a reactive primary amine, such as a reactive primary amine at a C-terminal end of a protein or peptide, such as by using Sortase A linker, which can be created by a Sortase A enzyme fusing an LXPTG recognition motif (SEQ ID NO: 1) to an N-terminal GGG motif to regenerate a native amide bond. The linker created can therefore link a moiety attached to the LXPTG recognition motif (SEQ ID NO: 1) with a moiety attached to the N-terminal GGG motif. An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the antibody construct. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups.

In some embodiments, a myeloid cell agonist-linker can be formed by conjugating a noncleavable maleimide-PEG4 linker containing a succinimide group with a myeloid cell agonist-linker.

An antibody construct of a conjugate can comprise an anti-ASGR1 antibody. An antibody construct of a conjugate can comprise an anti-ASGR2 antibody. An antibody construct of a conjugate can comprise an anti-TRF2 antibody. An antibody construct of a conjugate can comprise an anti-UGT1A1 antibody. An antibody construct of a conjugate can comprise an anti-SLC22A7 antibody. An antibody construct of a conjugate can comprise an anti-SLC13A5 antibody. An antibody construct of a conjugate can comprise an anti-SLC22A1 antibody. An antibody construct of a conjugate can comprise an anti-C9 antibody. An antibody construct of a conjugate can comprise an anti-HBV antigen antibody. An antibody construct of a conjugate can comprise an anti-HCV antigen antibody.

In a conjugate, an antibody construct can be linked to a myeloid cell agonist in such a way that the antibody construct can still bind to its antigen and the Fc binding domain of the antibody construct can still bind to an FcR, resulting in FcR-mediated signaling. In a conjugate, an antibody construct can be linked to a myeloid cell agonist in such a way that the linking does not interfere with ability of the antigen binding domain of the antibody construct to bind to its antigen, the ability of the Fc binding domain of the antibody construct to bind to an FcR, or with FcR-mediated signaling resulting from the Fc binding domain of the antibody construct binding to an FcR. In a conjugate, a myeloid cell agonist can be linked to an antibody construct in such a way the linking does not interfere with the ability of the myeloid cell agonist to bind to its receptor. A conjugate can produce stronger immune stimulation and a greater therapeutic window than components of the conjugate alone.

The specificity of the antigen binding domain to an antigen of a conjugate disclosed herein can be influenced by the presence of a myeloid cell agonist. The antigen binding domain of the conjugate can bind to its antigen with at least about 10%, about 20%, about 30%, about 40%, about 50%, about 60%, about 70%, about 80%, about 85%, about 90%, about 95%, or about 100% of a specificity of the antigen binding domain to the antigen in the absence of the myeloid cell agonist.

The specificity of the Fc binding domain to an Fc receptor of a conjugate disclosed herein can be influenced by the presence of a myeloid cell agonist. The Fc binding domain of the conjugate can bind to an Fc receptor with at least about 10%, about 20%, about 30%, about 40%, about 50%, about 60%, about 70%, about 80%, about 85%, about 90%, about 95%, or about 100% of a specificity of the Fc binding domain to the Fc receptor in the absence of the myeloid cell agonist.

The affinity of the antigen binding domain to an antigen of a conjugate disclosed herein can be influenced by the presence of a myeloid cell agonist. The antigen binding domain of the conjugate can bind to an antigen with at least about 1%, about 5%, about 10%, about 20%, about 30%, about 40%, about 50%, about 60%, about 70%, about 80%, about 85%, about 90%, about 95%, or about 100% of an affinity of the antigen binding domain to the antigen in the absence of the myeloid cell agonist.

The affinity of the Fc binding domain to an Fc receptor of a conjugate disclosed herein can be influenced by the presence of a myeloid cell agonist. The Fc binding domain of the conjugate can bind to an Fc receptor with at least about 1%, about 5%, about 10%, about 20%, about 30%, about 40%, about 50%, about 60%, about 70%, about 80%, about 85%, about 90%, about 95%, or about 100% of an affinity of the Fc binding domain to the Fc receptor in the absence of the myeloid cell agonist.

The K_{d} for binding of an antigen binding domain to its antigen in the presence of a myeloid cell agonist can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the antigen binding domain to the antigen in the absence of the myeloid cell agonist.

The K_{d} for binding of an Fc binding domain to a Fc receptor in the presence of a myeloid cell agonist can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the Fc binding domain to the Fc receptor in the absence of the myeloid cell agonist.

Affinity can be the strength of the sum total of noncovalent interactions between a single binding site of a molecule, for example, an antibody, and the binding partner of the molecule, for example, an antigen. The affinity can also measure the strength of an interaction between an Fc binding domain of an antibody or antibody construct and the Fc receptor. Unless indicated otherwise, as used herein, “binding affinity” can refer to intrinsic binding affinity which reflects a 1:1 interaction between members of a binding pair (e.g., antibody and antigen or Fc binding domain and Fc receptor). The affinity of a molecule X for its partner Y can generally be represented by the dissociation constant (K_{d}). Affinity can be measured by common methods known in the art, including those described herein. Specific illustrative and exemplary embodiments for measuring binding affinity are described in the following.

In some embodiments, an antibody or antibody construct provided herein can have a dissociation constant (K_{d}) of about 1 μM, about 100 nM, about 10 nM, about 5 nM, about 2 nM, about 1 nM, about 0.5 nM, about 0.1 nM, about 0.05 nM, about 0.01 nM, or about 0.001 nM or less (e.g., 10⁻⁸M or less, e.g., from 10⁻⁸M to 10¹³ M, e.g., from 10⁻⁹M to 10⁻¹³M). An affinity matured antibody can be an antibody with one or more alterations in one or more complementarity determining regions (CDRs), compared to a parent antibody, which may not possess such alterations, such alterations resulting in an improvement in the affinity of the antibody for antigen. These antibodies can bind to their antigen with a K_{d} of about 5×10⁻⁹M, about 2×10⁻⁹M, about 1×10⁻⁹M, about 5×10⁻¹M, about 2×10⁻⁹ M, about 1×10⁻¹⁰M, about 5×10⁻¹¹M, about 1×10⁻¹¹ M, about 5×10⁻¹² M, about 1×10⁻¹²M, or less. In some embodiments, the conjugate can have an increased affinity of at least 1.5-fold, 2-fold, 2.5-fold, 3-fold, 4-fold, 5-fold, 10-fold, 20-fold, or greater as compared to a conjugate without alterations in one or more complementarity determining regions.

K_{d} can be measured by any suitable assay. For example, K_{d} can be measured by a radiolabeled antigen binding assay (RIA). For example, K_{d} can be measured using surface plasmon resonance assays (e.g., using a BIACORE®-2000 or a BIACORE®-3000).

The molar ratio of a conjugate refers to the average number of myeloid cell agonists conjugated to the antibody construct in a preparation of a conjugate. The molar ratio can be determined, for example, by Liquid Chromatography/Mass Spectrometry (LC/MS), in which the number of myeloid cell agonists conjugated to the antibody construct can be directly determined. Additionally, as non-limiting examples, the molar ratio can be determined based on hydrophobic interaction chromatography (HIC) peak area, by liquid chromatography coupled to electrospray ionization mass spectrometry (LC-ESI-MS), by UV/Vis spectroscopy, by reversed-phase-HPLC (RP-HPLC), or by matrix-assisted laser desorption/ionization time of flight mass spectrometry (MALDI-TOF-MS).

In some embodiments, the molar ratio of myeloid cell agonists to antibody construct can be less than 8. In other embodiments, the molar ratio of myeloid cell agonists to antibody construct can be 8, 7, 6, 5, 4, 3, 2, or 1.

These conjugates can be made by various methods. It is understood that one skilled in the art may be able to make these compounds by similar methods or by combining other methods known to one skilled in the art. It is also understood that one skilled in the art would be able to make, in a similar manner as described herein by using the appropriate starting materials and modifying the synthetic route as needed. Starting materials and reagents can be obtained from commercial vendors or synthesized according to sources known to those skilled in the art or prepared as described herein.

### Pharmaceutical Formulations

The conjugates described herein are useful as pharmaceutical compositions for administration to a subject in need thereof. Pharmaceutical compositions can comprise the conjugates described herein and one or more pharmaceutically acceptable carriers, diluents, excipients, stabilizers, dispersing agents, suspending agents, and/or thickening agents. A pharmaceutical composition can comprise any conjugate described herein. A pharmaceutical composition can further comprise buffers, antibiotics, steroids, carbohydrates, drugs (e.g., chemotherapy drugs), radiation, polypeptides, chelators, adjuvants and/or preservatives.

In a pharmaceutical composition, the conjugates can have an average drug loading. The drug loading, p, is the average number of myeloid cell agonist-linker molecules per antibody construct, or the number of myeloid cell agonists per antibody construct. The variable z can range ranges from 1 to 20, or 1-100. In some conjugates, z is preferably from 1 to 8. The average number of myeloid cell agonists per antibody construct in a preparation may be characterized by conventional means such as mass spectroscopy, HIC, ELISA assay, and HPLC.

Pharmaceutical compositions can be formulated using one or more physiologically-acceptable carriers comprising excipients and auxiliaries. Formulation can be modified depending upon the route of administration chosen. Pharmaceutical compositions comprising a conjugate as described herein can be manufactured, for example, by lyophilizing the conjugate, mixing, dissolving, emulsifying, encapsulating or entrapping the conjugate. The pharmaceutical compositions can also include the conjugates described herein in a free-base form or pharmaceutically-acceptable salt form.

Methods for formulation of the pharmaceutical compositions can include formulating any of the conjugates described herein with one or more inert, pharmaceutically-acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compositions described herein can be lyophilized or in powder form for re-constitution with a suitable vehicle, e.g., sterile pyrogen-free water, before use

Pharmaceutical compositions of the conjugates described herein can comprise at least a conjugate as an active ingredient, respectively. The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as described herein often further can comprise more than one active compound as necessary for the particular indication being treated. The active compounds can have complementary activities that do not adversely affect each other. For example, the pharmaceutical composition can also comprise a cytotoxic agent, cytokine, growth-inhibitory agent, anti-hormonal agent, anti-angiogenic agent, and/or cardioprotectant. Such molecules can be present in combination in amounts that are effective for the purpose intended.

The pharmaceutical compositions and formulations can be sterilized. Sterilization can be accomplished by filtration through sterile filtration.

The pharmaceutical compositions described herein can be formulated for administration as an injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the pharmaceutical compositions described herein can be lyophilized or in powder form for reconstitution with a suitable vehicle, e.g., sterile pyrogen-free water, before use.

For parenteral administration, the conjugates can be formulated in a unit dosage injectable form (e.g., use letter solution, suspension, emulsion) in association with a pharmaceutically acceptable parenteral vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can also be prepared. Examples of sustained-release preparations can include semipermeable matrices of solid hydrophobic polymers that can contain the antibody, and these matrices can be in the form of shaped articles (e.g., films or microcapsules). Examples of sustained-release matrices can include polyesters, hydrogels (e.g., poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations of the compositions described herein can be prepared for storage by mixing a conjugate with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

### Therapeutic Applications

The conjugates, pharmaceutical compositions, and methods of the present disclosure can be useful for treating a plurality of different subjects including, but are not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof.

The conjugates, pharmaceutical compositions, and methods described herein can be useful as a therapeutic, for example a treatment that can be administered to a subject in need thereof. A therapeutic effect can be obtained in a subject by reduction, suppression, remission, or eradication of a disease state, including, but not limited to, a symptom thereof. A therapeutic effect in a subject having a disease or condition, or pre-disposed to have or is beginning to have the disease or condition, can be obtained by a reduction, a suppression, a prevention, a remission, or an eradication of the condition or disease, or pre-condition or pre-disease state.

In practicing the methods described herein, therapeutically-effective amounts of the conjugates, or pharmaceutical compositions described herein can be administered to a subject in need thereof, often for treating and/or preventing a condition or progression thereof. A pharmaceutical composition can affect the physiology of the subject, such as the immune system, inflammatory response, or other physiologic affect. A therapeutically-effective amount can vary widely depending on the severity of the disease, the age and relative health of the subject, the potency of the compounds used, and other factors.

Treat and/or treating can refer to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treat can be used herein to refer to a method that results in some level of treatment or amelioration of the disease or condition, and can contemplate a range of results directed to that end, including but not restricted to prevention of the condition entirely.

Prevent, preventing and the like can refer to the prevention of the disease or condition, e.g., viral infection, in the patient. For example, if an individual at risk of contracting a viral infection is treated with the methods of the present disclosure and does not later become infected with the virus, then the disease has been prevented, at least over a period of time, in that individual.

A therapeutically effective amount can be the amount of conjugates or pharmaceutical compositions or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment, and can be ascertainable by one skilled in the art using known techniques.

The conjugates or pharmaceutical compositions described herein that can be used in therapy can be formulated and dosages established in a fashion consistent with good medical practice taking into account the disorder to be treated, the condition of the individual patient, the site of delivery of the conjugate, or pharmaceutical composition, the method of administration and other factors known to practitioners. The conjugates or pharmaceutical compositions can be prepared according to the description of preparation described herein.

One of ordinary skill in the art would understand that the amount, duration and frequency of administration of a pharmaceutical composition or conjugate described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

The methods, conjugates and pharmaceutical compositions described herein can be for administration to a subject in need thereof. Often, administration of the conjugates, or pharmaceutical compositions can include routes of administration, non-limiting examples of administration routes include intravenous, intraarterial, subcutaneous, subdural, intramuscular, intracranial, intrasternal, intratumoral, or intraperitoneally. Additionally, a pharmaceutical composition, or conjugate can be administered to a subject by additional routes of administration, for example, by inhalation, oral, dermal, intranasal, or intrathecal administration.

Pharmaceutical compositions or conjugates of the present disclosure can be administered to a subject in need thereof in a first administration, and in one or more additional administrations. The one or more additional administrations can be administered to the subject in need thereof minutes, hours, days, weeks or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week or more than once per month. The conjugates or pharmaceutical compositions can be administered to the subject in need thereof in cycles of 21 days, 14 days, 10 days, 7 days, 4 days or daily over a period of one to seven days.

### Increased Dosages and Reduced Side-Effects

In certain embodiments, using a conjugate of this disclosure can allow administration of the conjugate at greater levels of myeloid cell agonist in the form of the conjugate than the level of myeloid cell agonist alone. For example, the conjugate can be administered at a level higher than the maximum tolerated dose for that myeloid cell agonist administered in the absence of the being conjugated to the antibody construct in the conjugate. In certain embodiments, administration of the conjugate can be associated with fewer side effects than when administered as the myeloid cell agonist alone.

### Diseases, Conditions and the Like

The conjugates, pharmaceutical compositions, and methods provided herein can be useful for the treatment of a plurality of diseases, conditions, preventing a disease or a condition in a subject or other therapeutic applications for subjects in need thereof. Often the conjugates, antibody constructs, pharmaceutical compositions, and methods provided herein can be useful for treatment of liver viral diseases, such as Hepatitis B and Hepatitis C.

The invention further provides any conjugates disclosed herein for use in a method of treatment of the human or animal body by therapy. Therapy may be by any mechanism disclosed herein, such as by modulation (e.g., stimulation) of the immune system. The invention provides any conjugate disclosed herein for use in stimulation of the immune system or immunotherapy, including for example enhancing an immune response. The invention further provides any conjugate disclosed herein for prevention or treatment of any condition disclosed herein, for example viral infection. The invention also provides any conjugate disclosed herein for obtaining any clinical outcome disclosed herein for any condition disclosed herein, such as reducing Hepatitus B or Hepatitus C infection in vivo. The invention also provides use of any conjugate disclosed herein in the manufacture of a medicament for preventing or treating any condition disclosed herein.

### General Synthetic Schemes and Examples

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

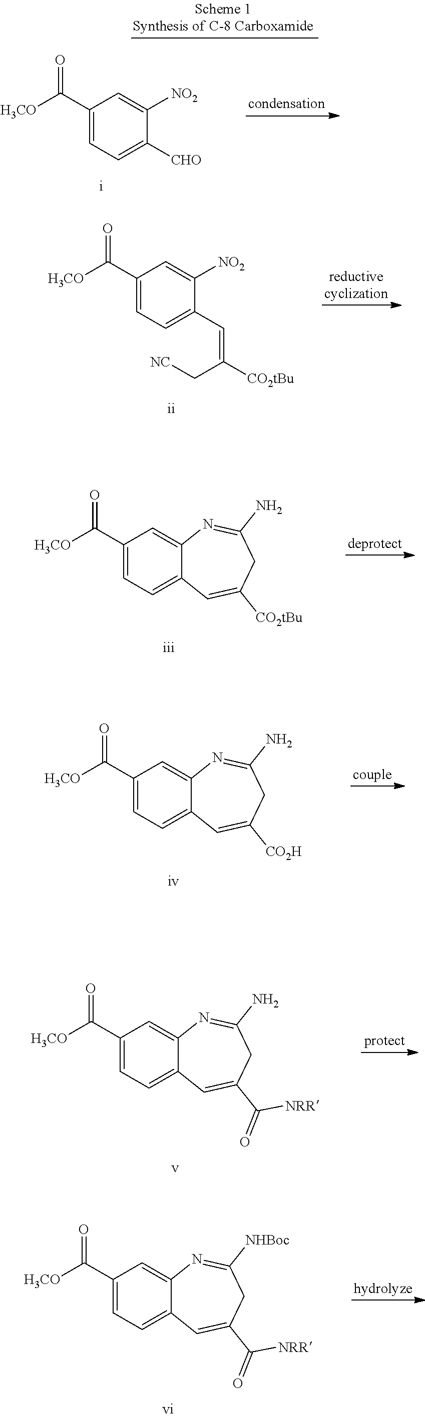

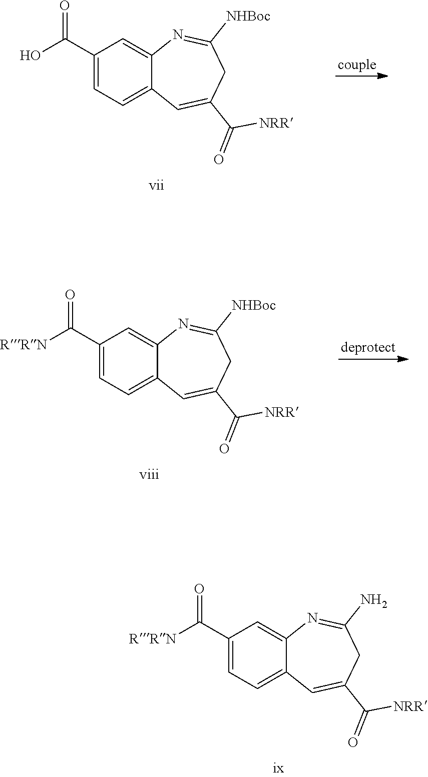

React an aldehyde (i) with an appropriately Wittig reagent, such as tert-butyl 3-cyano-2-(triphenylphosphorylidene)propanoate, at elevated temperatures to afford an olefin (ii), which undergoes reductive cyclization by treating the olefin (ii) with a reducing agent, such as iron powder in hot acetic acid, to afford azepines (iii). Deprotect the C-4 ester group by using a strong acid such as HCl to give compounds (iv), which is in turn coupled with a substituted amine using a coupling agent, such as BOP reagent. Protect the 2-amino substituent of compounds (v) with a tert-butoxycarbonyl group. Hydrolyze the resulting compounds (vi) with reagents such as LiOH in a mixture of THF and methanol to afford compounds (vii). Convert the C-8 carboxylic acid of (vii) to the amide group using known reagents such as HBTU and a tertiary amine base. Acid-mediated deprotection of compounds (viii) using a reagent such as TFA in dichloromethane provides the target compounds (ix).

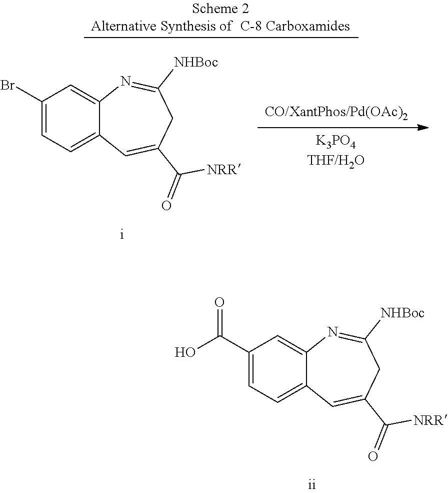

React (i) under standard conditions used for the carbonylation of aryl halides such as carbon monoxide, a palladium catalyst such as Pd(OAc)₂ and a ligand such as 4,5-bis(diphenylphosphino)-9,9-dimethylxanthene (XantPhos) and a base such as potassium phosphate in a mixture of THF and water to provide carboxylic acids (ii). Conversion to final products can then be carried out in a manner similar to that described in Scheme 1 (vii→ix).

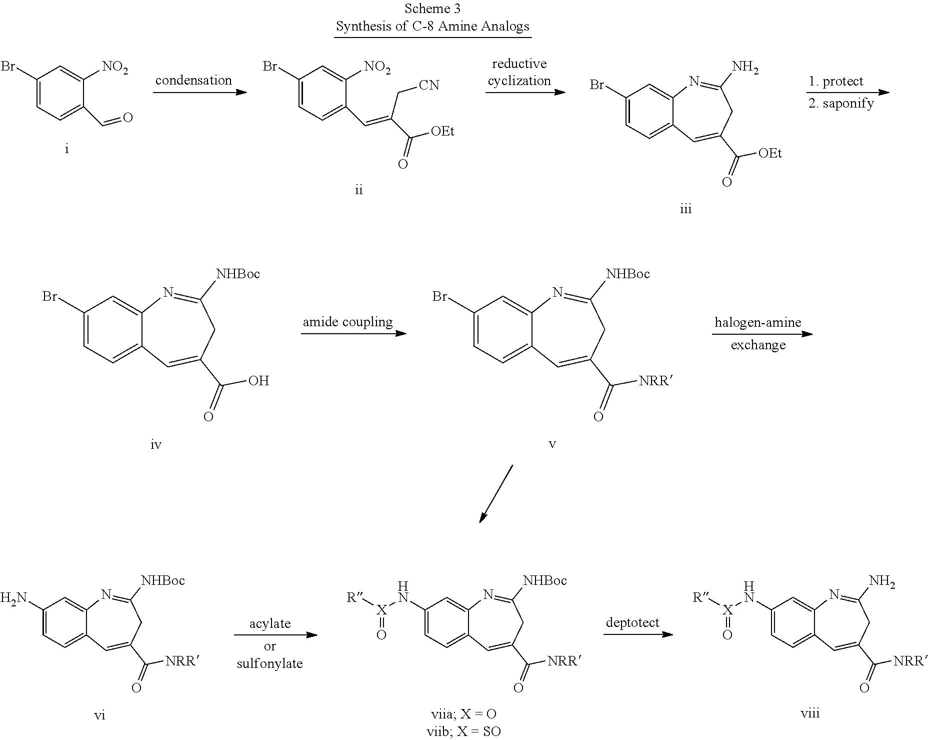

React an aldehyde (i) with an appropriately Wittig reagent, such as ethyl 3-cyano-2-(triphenylphosphorylidene)propanoate, at ambient temperature to afford an olefin (ii), which undergoes reductive cyclization by treating the olefin (ii) with a reducing agent, such as iron powder in hot acetic acid, to afford azepines (iii). Protect the C-2 amine group by using Boc anhydride to give compounds (iii), which is in turn saponified with an alkaline metal hydroxide such as LiOH to afford the carboxylic acid which is coupled with a substituted amine using a coupling agent, such as BOP reagent to provide compounds (iv). Convert the C-8 carboxylic acid of (v) to the amide group using known reagents such as EDCI/HOBT and a tertiary amine base. Halogen-amine exchange can be effected using standard methodology such as copper-mediated or palladium-catalyzed couplings (benzophenone imine/Pd(II)) to provide C-8 anilines (vi). Functionalization of amines (vi) by acylation or sulfonylation provides anilides (X═C) or sulfonamides (X═SO) compounds (vii). Alternatively, compounds (vii) can be prepared directly through a palladium-mediated coupling of bromide (v) and an appropriately substituted amide or sulfonamide. Acid-mediated deprotection of compounds (vii) using a reagent such as TFA in dichloromethane provides the target compounds (viii).

### Scheme 4

### Synthesis of Linker-Payloads

A linker-payload (LP) can be synthesized by various methods. For example, LP compounds can be synthesized as shown in Scheme 4-1.

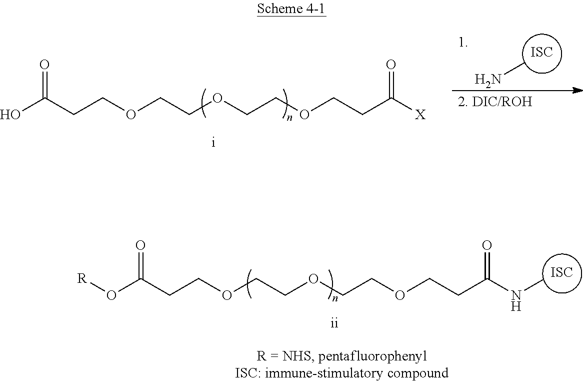

A PEGylated carboxylic acid (i) that has been activated for amide bond formation can be reacted with an appropriately substituted amine containing immune-stimulatory compound to afford an intermediate amide. Formation of an activated ester (ii) can be achieved by reaction the intermediate amide-containing carboxylic using a reagent such as N-hydroxysuccinimide or pentafluorophenol in the presence of a coupling agent such as diisopropylcarbodiimide (DIC) to provide compounds (ii).

An LP can be synthesized as shown in Scheme 4-2.

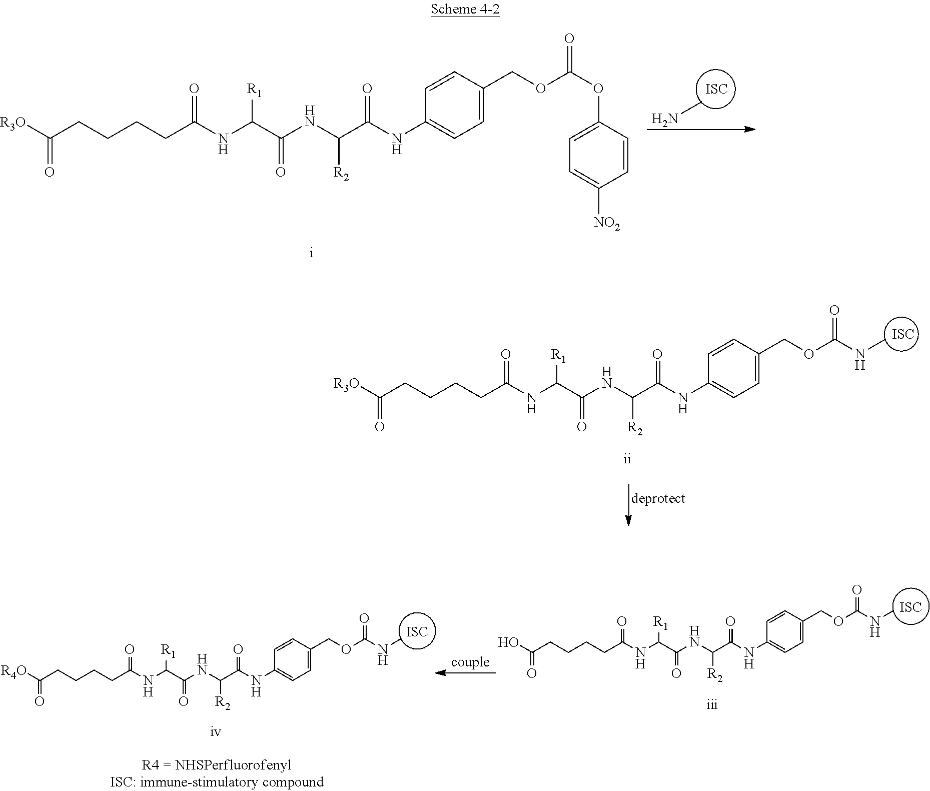

An activated carbonate such as (i) can be reacted with an appropriately substituted amine containing immune-stimulatory compound to afford carbamates (ii) which can be deprotected using standard methods based on the nature of the R₃ ester group. The resulting carboxylic acid (iii) can then by coupled with an activating agent such as N-hydroxysuccinimide or pentafluorophenol to provide compounds (iv).

An LP compound can be synthesized as shown in Scheme 4-3.

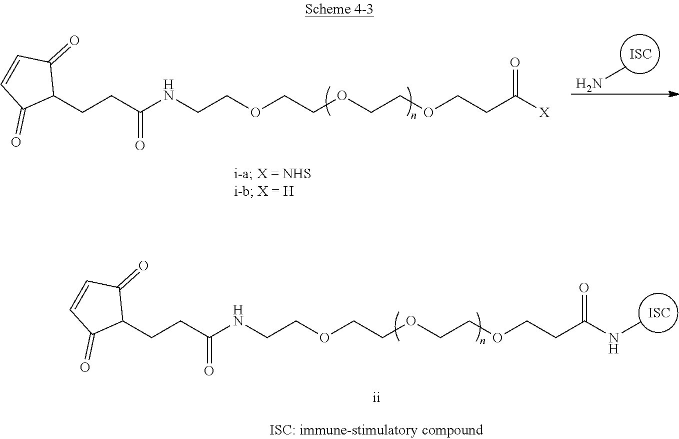

An activated carboxylic ester such as (i-a) can be reacted with an appropriately substituted amine containing immune-modstimulatory compound to afford amides (ii). Alternatively, carboxylic acids of type (i-b) can be coupled to an appropriately substituted amine containing immune-stimulatory compound in the presence of an amide bond forming agent such as dicyclohexycarbodiimde (DCC) to provide the desired LP.

An LP compound can be synthesized by various methods such as that shown in Scheme 4-4.

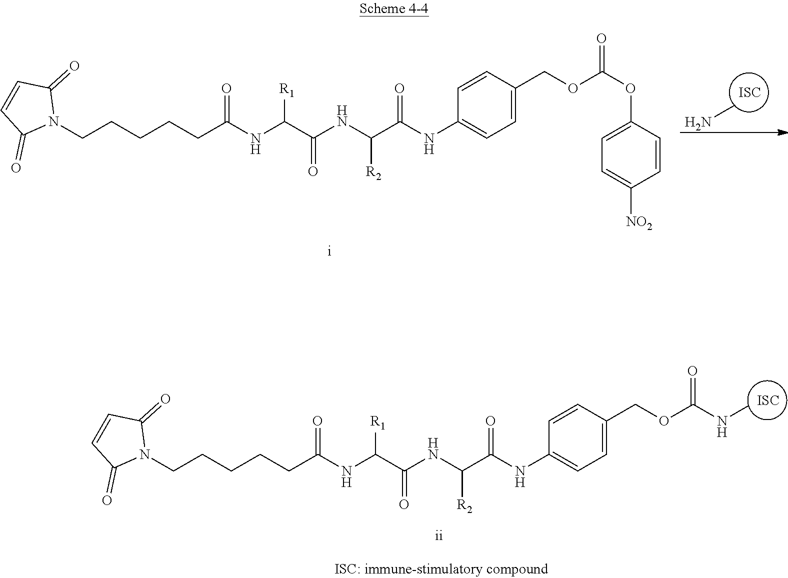

An activated carbonate such as (i) can be reacted with an appropriately substituted amine containing immune-modstimulatory compound to afford carbamates (ii) as the target ISC.

An LP compound can also be synthesized as shown in Scheme 4-5.

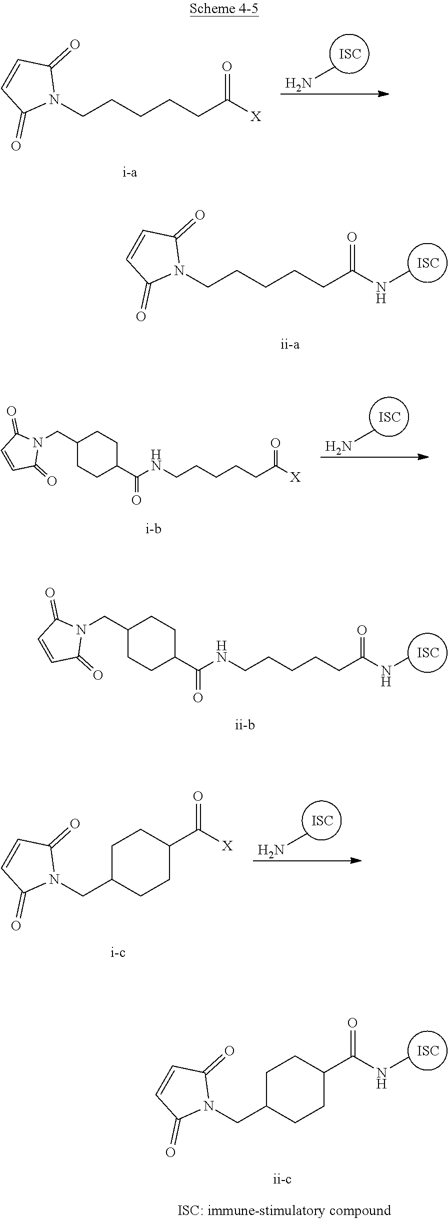

An activated carboxylic acid such as (i-a, i-b, i-c) can be reacted with an appropriately substituted amine containing immune-stimulatory compound to afford amides (ii-a, ii-b, ii-c) as the target linkered payloads (LPs).

### EXAMPLES

The following examples are included to further describe some embodiments of the present disclosure, and should not be used to limit the scope of the disclosure

### Example 1

### General Process for Identifying Liver-Specific Antigens

A compendium of normal tissue expression data, for example an RNA-Seq transcriptomic database such as GTEx, is used to create segregated sets of samples by tissue type. This database is then partitioned into test (tissues with desired expression) and control (tissues with undesired expression) subsets, in this case liver versus all other tissues, and an analysis of variance test such as the Kruskal-Wallis one-way analysis of variance is performed to identify genes that are significantly differentially expressed between the two sample sets. Because the resulting list of genes may include examples which while over-expressed in the test group when compared to the normal group as a whole may still have higher expression in individual tissue types than desired, a second filtering step is applied wherein those genes whose average tissue-specific expression for any specific tissue in the control tissue set is above a desired cutoff are removed from further analysis. If desired, a further filtering step can be applied to sort and restrict genes returned based on absolute minimum average expression in the test set, absolute maximum average expression in the control set and/or the ratio of the test-to-control expression. From this resulting list, a final filtering step is performed as desired to include or exclude genes based on the cellular localization of the protein product. For example, it may be desired to include only proteins expressed on the cell surface, or of those single-pass surface proteins. When selecting the final list, additional consideration is also given to expression patterns of the antigen in one or more specific diseases, or in liver cell subsets. For example, in the case of liver cell subsets the expression of the antigen in question would be compared in available liver cell subsets (such as hepatocytes, Kupffer cells, or hepatic stellate cells) and antigens removed, retained, or prioritized in consideration depending on these expression patterns. Where possible, protein-level confirmation of expression of candidate genes is performed by examining available immunohistochemistry data such as that found in the Human Protein Atlas.

### Example 2

### Synthesis of 2-amino-/N⁴,N⁴-dipropyl-N⁸-(1,2,3,4-tetrahydroquinolin-7-yl)-3H-benzo[b]azepine-4,8-dicarboxamide TFA Salt (Compound 1.1)

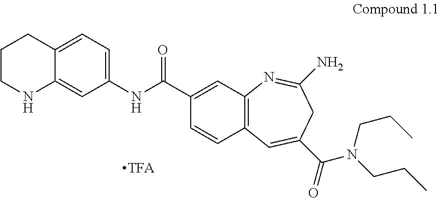 [SMILES: CCCN(CCC)C(=O)C1=Cc2ccc(C(=O)Nc3ccc4c(c3)NCCC4)cc2N=C(N)C1]

Step A: Preparation of Int 1.1a

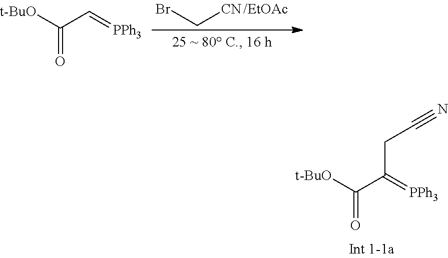

Bromoacetonitrile (8.60 g, 71.7 mmol, 4.78 mL) was added to a solution of tert-butyl (triphenylphosphorylidine)acetate (45.0 g, 119 mmol, 1.00 eq) in EtOAc (260 mL) at 25° C. The reaction was heated at 80° C. for 16 h after which time TLC (DCM:MeOH=10:1; R_{f}=0.4) and LCMS showed the reaction was complete. The mixture was cooled, filtered and washed with EtOAc (200 mL) and concentrated to afford crude Int 1.1a as a red solid which was used directly without purification.

Step B: Preparation of Int 1.1b

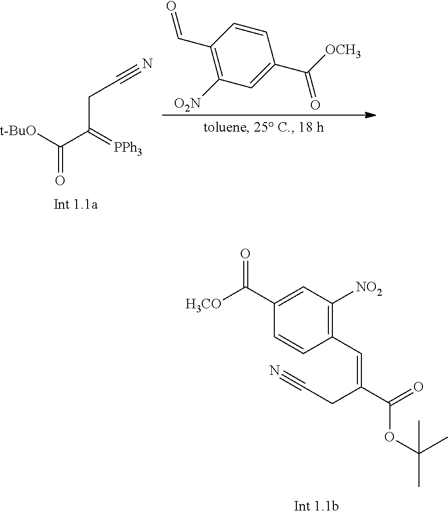

A solution of Int 1.1a (11.4 g, 54.4 mmol, 1.00 eq) and methyl 4-formyl-3-nitrobenzoate (24.8 g, 59.8 mmol, 1.10 eq) in toluene (200 mL) was stirred at 25° C. for 18 h. TLC (petroleum ether:EtOAc=1:2) showed the reaction was completed and the mixture was concentrated to afford crude product which was purified by silica gel chromatography (petroleum ether: EtOAc=10:1 to 8:1 to 4:1) to give Int 1.1b (11.3 g) as yellow solid. ¹H NMR (CDCl₃) δ 8.86 (d, J=1.3 Hz, 1H), 8.40 (dd, J=7.9, 1.3 Hz, 1H), 8.11 (s, 1H), 7.54 (d, J=7.9 Hz, 1H), 3.97-4.05 (m, 3H), 3.27 (s, 2H), 1.60 ppm (s, 9H).

Step C: Preparation of Int 1.1c

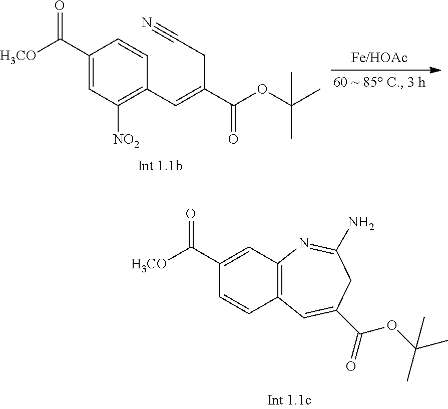 [SMILES: COC(=O)c1ccc(/C=C(\CC#N)C(=O)OC(C)(C)C)c([N+](=O)[O-])c1 ; COC(=O)c1ccc2c(c1)N=C(N)CC(C(=O)OC(C)(C)C)=C2]

Iron powder (6.79 g, 122 mmol) was added to a solution of Int 1.1b (23.4 g, 20.3 mmol, 1.00 eq) in glacial acetic acid (230 mL) at 60° C. The mixture was stirred at 85° C. for 3 h. TLC (petroleum ether: EtOAc=1:2; R_{f}=0.43) showed the reaction was completed and the mixture was cooled, filtered, washed with acetic acid (100 mL×2) and concentrated. The crude residue was diluted with EtOAc (100 mL) and washed with aq. NaHCO₃ (50 mL×3) and dried over Na₂SO₄, filtered and concentrated. The residue was purified by silica gel chromatography to afford 15.9 g of the Int 1.1c as yellow solid. ¹H NMR (CDCl₃) δ 7.95 (s, 1H), 7.76 (dd, J=8.2, 1.5 Hz, 1H), 7.70 (s, 1H), 7.46 (d, J=8.2 Hz, 1H), 3.93 (s, 3H), 2.99 (s, 2H), 1.56 (s, 9H).

Step D: Preparation of Int 1.1d

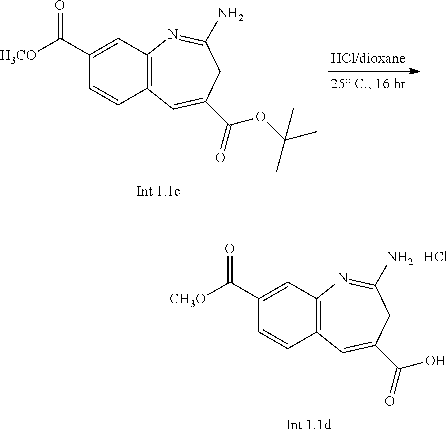

A solution of Int 1.1c (8.00 g, 25.3 mmol) in HCl/dioxane (160 mL) was stirred at 25° C. for 16 h after which time LCMS showed the reaction to be complete. The mixture was concentrated to afford 12.5 g of Int 1.1d as light yellow solid which was used directly without purification. ¹H NMR (DMSO-d₆) δ 13.43 (br s, 1H), 13.00 (br s, 1H), 10.20 (s, 1H), 9.22 (s, 1H), 7.96 (s, 1H), 7.85-7.92 (m, 2H), 7.78-7.83 (m, 1H), 3.90 (s, 3H), 3.52 (s, 2H).

Step E: Preparation of Int 1.1e

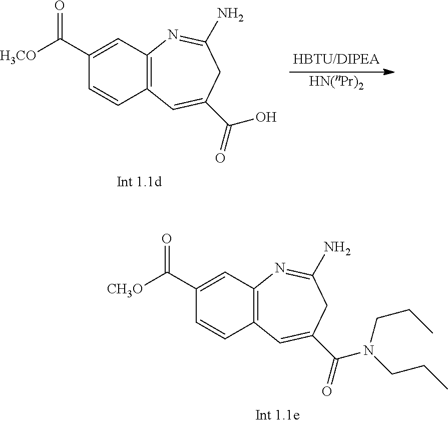

5.0 g (13.3 mmol) of HBTU and 7.7 mL (44.4 mmol) of DIPEA were added to a solution containing 3.3 g (11.1 mmol) of Int 1.1d in 60 mL of DMF at 0° C. After 5 minutes, 2.2 g (21.7 mmol) of di-n-propylamine was added and the reaction was stirred to room temperature overnight. The reaction was quenched with 20 mL of saturated NH₄Cl and then 20 mL of water. The mixture was extracted with EtOAc (3 ×30 mL) and the combined organic extracts were washed with brine (2×) then dried over Na₂SO₄. After removal of the drying agent and concentration of the EtOAc solution, the residue was purified on silica gel (80 g column; 0% to 20% methanol/DCM) to afford 3.0 g of Int 1.1e. ¹H NMR (CDCl₃) δ 7.92 (d, J=1.5 Hz, 1H), 7.86 (dd, J=8.2, 1.5 Hz, 1H), 7.38 (d, J=8.2 Hz, 1H), 6.89 (s, 1H), 3.92 (s, 3H), 3.39 (t, J=7.5 Hz, 4H), 3.22 (s, 2H), 1.68 (m, 4H), 0.91 (bs, 6H). ESI, m/z 343 [M+H].

Step F: Preparation of Int 1.1f

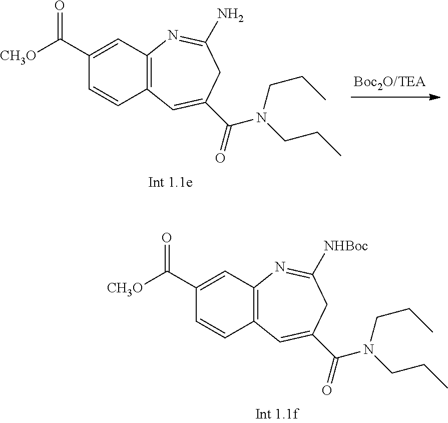

A solution containing 1.8 g (5.3 mmol) of Int 1.1e in 30 mL of dichloromethane was cooled to 0° C. and treated with 2.2 mL (7.9 mmol) of TEA and then 1.7 g (7.9 mmol) of Boc₂O. The reaction mixture was stirred to room temperature overnight and then quenched with 10 mL of water. The layers were separated and the aqueous was back extracted with dichloromethane (3×30 mL). The combined organic extracts were washed with brine and dried over Na₂SO₄. The solvent was removed and the residue was purified by silica gel chromatography (80 g column; 0% to 75% EtOAc/Hexanes) to afford the desired Int 1.1f as a white solid.

Step G: Preparation of Int 1.1g

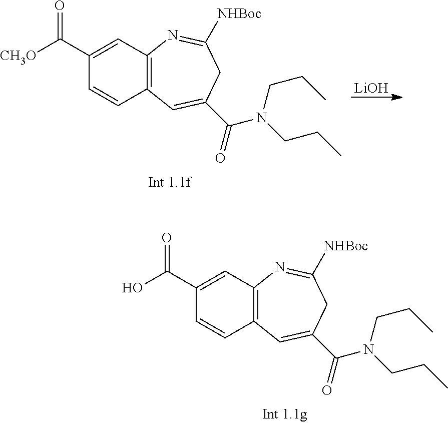

A solution containing 500 mg (1.13 mmol) of Int 1.1f in 10 mL of a 1:1 mixture of THF and water was cooled to 0° C. and treated with 1.7 mL (1.7 mmol) of 1N LiOH. After stirring for 16 h, ice chips were added, followed by enough 5% citric acid solution to effect a precipitate (pH˜5.5). The resulting mixture was washed three times with EtOAc and the combined organic extracts were washed with brine and dried over Na₂SO₄. The solution was evaporated to afford 419 mg of Int 1.1g as a pale yellow solid, which was used without purification.

Step H: Preparation of Compound 1.1

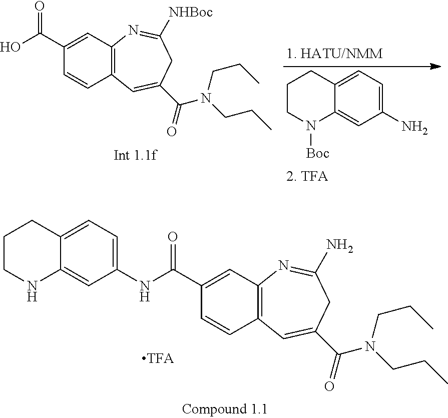

46 mg (0.12 mmol) of HATU was added to a solution containing 43 mg (0.10 mmol) of Int 1.1f in 1.0 mL of DMF. The reaction mixture was stirred for 5 minutes and then treated with 30 mg (0.12 mmol) of 7-N-Boc-amino-1,2,3,4-tetrahydroquinoline and 0.022 mL (0.20 mmol) of NMM. The reaction mixture was stirred for 16 h then treated with 5 mL of saturated NH₄Cl solution and 5 mL of water. The resulting mixture was extracted three times with EtOAc and the combined organics were washed with brine then dried over Na₂SO₄. After evaporation of the solvent, the crude oil was dissolved in 3 mL of DCM and then cooled to 0° C. Then, 0.6 mL of TFA was added to the mixture. The mixture was stirred for 4 h, evaporated and the resulting residue was purified by reverse phase chromatography to afford the TFA salt of Compound 1.1 as a white solid. ¹H NMR (CD₃OD) δ 7.96 (s, 1H), 7.95 (s, 1H), 7.85 (d, J=2.4 Hz, 1H), 7.79 (d, J=8.8 Hz, 1H), 7.38 (d, J=7.5 Hz, 1H), 7.25 (d, J=7.5 Hz, 1H), 7.10 (s, 1H), 3.55 (t, J=7.5 Hz, 6H), 3.33 (m, 2H), 2.90 (t, J=6.6 Hz, 2H), 2.10 (m, 1H), 1.69 (m, 4H), 0.77 (bs, 6H). LCMS [M+H]=460.25.

### Example 3

### Myeloid Agonist Benazepine Compounds

Table 1 shows benzazepine compounds that are myeloid agonists. Compounds 1.2-1.67 can be prepared in manner similar to that used for the synthesis of Compound 1.1 (Example 2) by using Intermediate 1.1f and an appropriately substituted amine or other methods known to the skilled artisan.

TABLE 1
Compounds 1.1-1.67

| Cmpd | Structure and IUPAC | ¹H NMR | M + 1 |
|---|---|---|---|
| 1.1 | 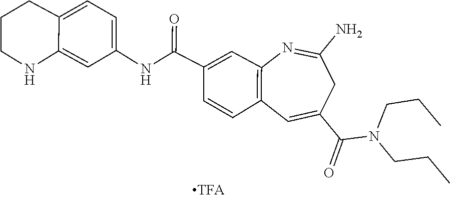 [SMILES: CCCN(CCC)C(=O)C1=Cc2ccc(C(=O)Nc3ccc4c(c3)NCCC4)cc2N=C(N)C1] | (CD₃OD) δ 7.96 (s, 1H), 7.95 (s, 1H), 7.85 (d, J = 2.4 Hz, 1H), 7.79 (d, J = 8.8 Hz, 1H), 7.38 (d, J = 7.5 Hz, 1H), 7.25 (d, J = 7.5 Hz, 1H), 7.10 (s, 1H), 3.55 (t, J = 7.5 Hz, 6H), 3.33 (m, 2 H), 2.90 (t, J = 6.6 Hz, 2H), 2.10 (m, 1H), | 460.3 |
| | | 1.69 (m, 4H), 0.77 | |
| | 2-amino-N4,N4-dipropyl-N8-(1,2,3,4- | (bs, 6H). | |
| | tetrahydroquinolin-7-yl)-3H-benzo[b]azepine-4,8- | | |
| | dicarboxamide TFA salt | | |
| 1.2 | 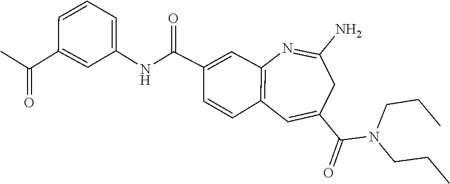 [SMILES: CCCN(CCC)C(=O)C1=Cc2ccc(C(=O)Nc3cccc(C(C)=O)c3)cc2N=C(N)C1] | (CD₃OD) δ 8.35 (s, 1H), 7.97 (dd, J = 1.5, 8.0 Hz, 1H), 7.79 (dd, J = 1.5, 8.8 Hz), 1H), 7.75 (d, J = 1.5 Hz, 1H), 7.63 (dd, J = 1.5, 7.5 Hz, 1H), 7.51 (m, 2H), 6.92 (s, 1H), 3.43 (t, J = 7.5 Hz, 4H), 2.63 (s, 3H), 1.70 (m, 4H), 0.96 | 446.9 |
| | | (bs, 3H), 0.87 (bs, | |
| | N⁸-(3-acetylphenyl)-2-amino-N⁴,N⁴-dipropyl-3H- | 3H). | |
| | benzo[b]azepine-4,8-dicarboxamide | | |
| 1.3 | 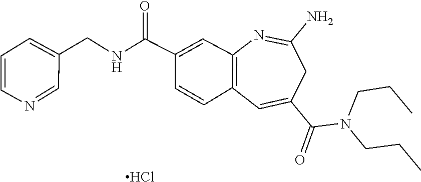 [SMILES: CCCN(CCC)C(=O)C1=Cc2ccc(C(=O)NCc3cccnc3)cc2N=C(N)C1 ; Cl] | (CD₃OD) δ 8.93 (s, 1H), 8.80 (d, J = 5.5 Hz, 1H), 8.68 (d, J = 8.5 Hz, 1H), 8.11 (m, 1H), 7.97 (d, J = 1.5 Hz, 1H), 7.89 (dd, J = 1.5, 7.5 Hz, 1H), 7.67 (d, J = 7.5 Hz, 1H), 7.08 (s, 1H), 4.84 (s, 2H), 3.44 (bs, 4H), 3.25 | 419.9 |
| | | (s, 2H), 1.69 (q, | |
| | 2-amino-N⁴,N⁴-dipropyl-N⁸-(pyridin-3-ylmethyl)-3H- | J = 7.5 Hz, 4H), 0.92 | |
| | benzo[b]azepine-4,8-dicarboxamide HCl salt | (bs, 3H), 0.90 (bs, | |
| | | 3H). | |
| 1.4 | 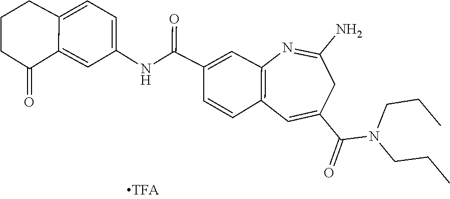 [SMILES: CCCN(CCC)C(=O)C1=Cc2ccc(C(=O)Nc3ccc4c(c3)C(=O)CCC4)cc2N=C(N)C1] | (CD₃OD) δ 8.27 (s, 1H), 7.97 (m, 3H), 7.71 (d, J = 7.5 Hz, 1H), 7.37 (d, J = 7.5 Hz, 1H), 7.11 (s, 1H), 3.55 (m, 4H), 3.28 (s, 2H), 3.00 (t, J = 7.5 Hz, 2H), 2.69 (t, J = 7.5 Hz, 2H), 2.15 (m, 2H), 1.70 (q, J = 7.5 Hz, 4H), 0.98 | 473.2 |
| | | (bs, 6H). | |
| | 2-amino-N⁸-(8-oxo-5,6,7,8-tetrahydronaphthalen-2- | | |
| | yl)-N⁴,N⁴-dipropyl-3H-benzo[b]azepine-4,8- | | |
| | dicarboxamide trifluoroacetate salt | | |
| 1.5 | 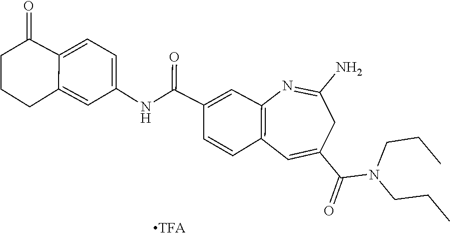 [SMILES: CCCN(CCC)C(=O)C1=Cc2ccc(C(=O)Nc3ccc4c(c3)CCCC4=O)cc2N=C(N)C1] | (CD₃OD) δ 7.99 (m, 3H), 7.85 (s, 1H), 7.71 (m, 2H), 7.15 (s, 1H), 3.50 (m, 4H), 3.30 (s, 2H), 3.03 (t, J = 7.5 Hz, 2H), 2.65 (t, J = 7.5 Hz, 2H), 2.15 (m, 2H), 1.73 (q, J = 7.5 Hz, 4H), 0.97 (bs, 6H). | 473.1 |
| | 2-amino-N⁸-(5-oxo-5,6,7,8-tetrahydronaphthalen-2- | | |
| | yl)-N⁴,N⁴-dipropyl-3H-benzo[b]azepine-4,8- | | |
| | dicarboxamide trifluoroacetate salt | | |
| 1.6 | 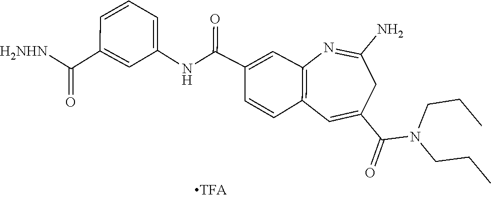 | (CD₃OD) δ 8.41 (s, 1H), 7.99 (m, 2H), 7.67 (m, 2H), 7.57 t, J = 8.0 Hz, 1H), 7.12 (s, 1H), 3.65 (m, 5H), 1.66 (m, 4H), 0.96 (bs, 6H). | |
| | 2-amino-N⁸-(3-(hydrazinecarbonyl)phenyl)-N⁴,N⁴- | | |
| | dipropyl-3H-benzo[b]azepine-4,8-dicarboxamide | | |
| | trifluoroacetate salt | | |
| 1.7 | 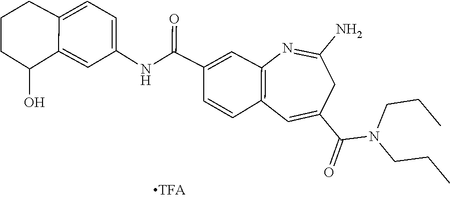 [SMILES: CCCN(CCC)C(=O)C1=Cc2ccc(C(=O)Nc3ccc4c(c3)C(O)CCC4)cc2N=C(N)C1] | (DMSO-d₆) δ 10.1 (s, 1H), 7.86 (s, 1H), 7.72 (s, 1H), 7.56 (d, J = 7.7 Hz, H), 7.49 (d, J = 7.5 Hz, 1H, 7.38 (d, J = 7.5 Hz, 1H), 7.00 (d, J = 8.1 Hz, 1H), 6.87 (s, 1H), 6.77 (s, 1H), 5.10 (bs, 1H), 4.54 (s, 1H), 3.28 (m, 4H), 3.28 (s, 2H), | 475.2 |
| | | 2.66 (m, 4H), 1.88 | |
| | 2-amino-N⁸-(8-hydroxy-5,6,7,8- | (m, 2H), 1.66-1.32 | |
| | tetrahydronaphthalen-2-yl)-N4,N4-dipropyl-3H- | (m, 6H), 0.87 (bs, | |
| | benzo[b]azepine-4,8-dicarboxamide trifluoroacetate | 6H). | |
| | salt | | |
| 1.8 | 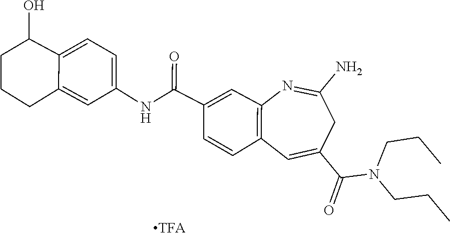 [SMILES: CCCN(CCC)C(=O)C1=Cc2ccc(C(=O)Nc3ccc4c(c3)CCCC4O)cc2N=C(N)C1] | (CD₃CN) δ 14.0 (bs, 1H), 11.0 (bs, 1H), 8.86 (s, 1H), 7.87 (s, 1H), 7.85 (d, J = 7.7 Hz, 1H), 7.62 (m, 2H), 7.42 (d, J = 7.5 Hz, 1H), 6.98 (s, 1H), 6.77 (s, 1H), 4.68 (s, 1H), 3.28 (m, 4H), 3.15 (m, 4H), 2.76 (m, 2H), 1.88 (m, 3H), 1.61 (m, 4H), 0.92 (bs, 6H). | 475.2 |
| | 2-amino-N⁸-(5-hydroxy-5,6,7,8- | | |
| | tetrahydronaphthalen-2-yl)-N⁴,N⁴-dipropyl-3H- | | |
| | benzo[b]azepine-4,8-dicarboxamide trifluoroacetate | | |
| | salt | | |
| 1.9 | 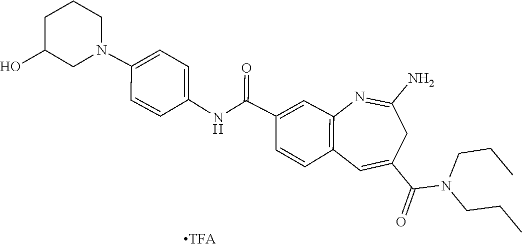 [SMILES: CCCN(CCC)C(=O)C1=Cc2ccc(C(=O)Nc3ccc(N4CCCC(O)C4)cc3)cc2N=C(N)C1] | (CD₃OD) δ 9.98 (s, 1H), 7.63 (m, 2H), 7.44 (d, J = 8.4 Hz, 1H), 7.39 (d, J = 8.4 Hz, 1H), 6.90 (m, 3H), 6.75 (s, 1H), 4.77 (d, J = 8.4 Hz, 1H), 3.56 (m, 2H), 3.44 (m, 1H), 2.70-2.50 (m, 3H), 1.88 (m, 1H), 1.70 (m, 1H), 1.60 (m, 4H), 1.22 (m, 2H), 0.85 (bs, 6H). | |
| | 2-amino-N⁸-(4-(3-hydroxypiperidin-1-yl)phenyl)- | | |
| | N⁴,N⁴-dipropyl-3H-benzo[b]azepine-4,8- | | |
| | dicarboxamide trifluoroacetate salt | | |
| 1.10 | 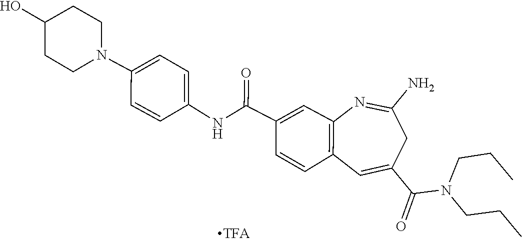 [SMILES: CCCN(CCC)C(=O)C1=Cc2ccc(C(=O)Nc3ccc(N4CCC(O)CC4)cc3)cc2N=C(N)C1] | (CD₃OD) δ 7.95 (m, 4H), 7.71 (d, J = 8.0 Hz, 1H), 7.55 (d, J = 8.8 Hz, 2H), 7.10 (s, 1H), 4.05 (m, 1H), 3.80 (m, 2H), 3.50 (m, 4H), 3.33 (s, 2H), 2.25 (m, 2H), 1.97 (m, 2H), 1.73 (q, J = 7.5 Hz, 4H), 0.97 (bs, 6H). | |
| | 2-amino-N⁸-(4-(4-hydroxypiperidin-1-yl)phenyl)- | | |
| | N⁴,N⁴-dipropyl-3H-benzo[b]azepine-4,8- | | |
| | dicarboxamide trifluoroacetate salt | | |
| 1.11 | 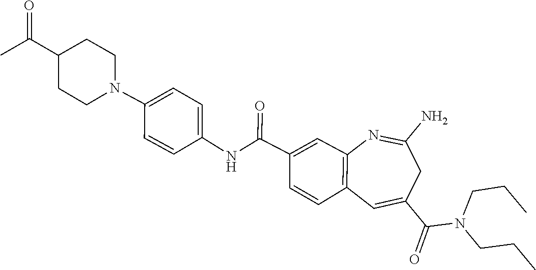 [SMILES: CCCN(CCC)C(=O)C1=Cc2ccc(C(=O)Nc3ccc(N4CCC(C(C)=O)CC4)cc3)cc2N=C(N)C1] | (CD₃OD) δ 7.95 (m, 4H), 7.71 (d, J = 8.0 Hz, 1H), 7.55 (d, J = 8.8 Hz, 2H), 7.10 (s, 1H), 4.05 (m, 1H), 3.80 (m, 2H), 3.50 (m, 4H), 3.33 (s, 2H), 2.25 (m, 2H), 2.15 (s, 3H), 1.97 (m, 2H), 1.73 (q, J = 7.5 Hz, 4H), 0.97 (bs, 6H). | |
| | N⁸-(4-(4-acetylpiperidin-1-yl)phenyl)-2-amino- | | |
| | N⁴,N⁴-dipropyl-3H-benzo[b]azepine-4,8- | | |
| | dicarboxamide | | |
| 1.12 | 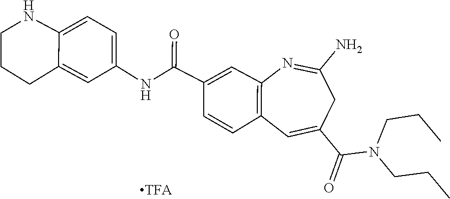 [SMILES: CCCN(CCC)C(=O)C1=Cc2ccc(C(=O)Nc3ccc4c(c3)CCCN4)cc2N=C(N)C1] | (CD₃OD) δ 7.95 (m, 2H), 7.71 (m, 3H), 7.18 (m, 1H), 7.11 (s, 1H), 3.43 (m, 4H), 3.50 (m, 2H), 3.28 (s, 2H), 2.96 (t, J = 7.5 Hz, 2H), 2.15 (m, 2H), 1.73 (q, J = 7.5 Hz, 4H), 0.97 (bs, 6H). | 460.2 |
| | 2-amino-N⁴,N⁴-dipropyl-N⁸-(1,2,3,4- | | |
| | tetrahydroquinolin-6-yl)-3H-benzo[b]azepine-4,8- | | |
| | dicarboxamide trifluoroacetate salt | | |
| 1.13 | 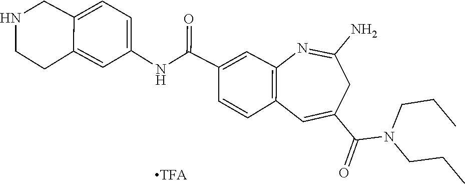 [SMILES: CCCN(CCC)C(=O)C1=Cc2ccc(C(=O)Nc3ccc4c(c3)CCNC4)cc2N=C(N)C1] | (CD₃OD) δ 7.95 (m, 2H), 7.71-7.61 (m, 3H), 7.27 (d, J = 8.4 Hz, 1H), 7.13 (s, 1H), 4.38 (s, 2H), 3.58-3.45 (m, 6H), 3.40 (s, 2H), 3.15 (t, J = 6.6 Hz, 2H), 1.71 (q, J = 7.5 Hz, 4H), 0.96 (bs, 6H). | 460.2 |
| | 2-amino-N⁴,N⁴-dipropyl-N⁸-(1,2,3,4- | | |
| | tetrahydroisoquinolin-6-yl)-3H-benzo[b]azepine-4,8- | | |
| | dicarboxamide trifluoroacetate salt | | |
| 1.14 | 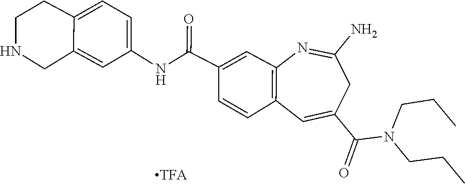 [SMILES: CCCN(CCC)C(=O)C1=Cc2ccc(C(=O)Nc3ccc4c(c3)CNCC4)cc2N=C(N)C1] | (CD₃OD) δ 7.95 (m, 2H), 7.71-7.61 (m, 3H), 7.27 (d, J = 8.4 Hz, 1H), 7.13 (s, 1H), 4.39 (s, 2H), 3.58-3.45 (m, 6H), 3.40 (s, 2H), 3.14 (t, J = 6.6 Hz, 2H), 1.74 (q, J = 7.5 Hz, 4H), 0.95 (bs, 6H). | 460.2 |
| | 2-amino-N⁴,N⁴-dipropyl-N⁸-(1,2,3,4-tetrahydro- | | |
| | isoquinolin-7-yl)-3H-benzo[b]azepine-4,8- | | |
| | dicarboxamide trifluoroacetate salt | | |
| 1.15 | 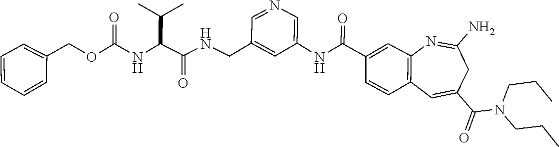 [SMILES: CCCN(CCC)C(=O)C1=Cc2ccc(C(=O)Nc3cncc(CNC(=O)[C@@H](NC(=O)OCc4ccccc4)C(C)C)c3)cc2N=C(N)C1] | (CD₃OD) δ 8.80 (d, J = 2.1 Hz, 1H), 8.29 (s, 1H), 8.21 (s, 1H), 7.72 (s, 1H), 7.58 (dd, J = 1.5, 8.2 Hz, 1H), 7.33-7.23 (m, 5H), 6.90 (s, 1H), 5.11 (d, J = 6.8 Hz, 2H), 4.44 (s, 2H), | 668.3 |
| | | 3.98 (d, J = 7.0 Hz, | |
| | benzyl (S)-(1-(((5-(2-amino-4-(dipropyl-carbamoyl)- | 1H), 3.43 (m, 4H), | |
| | 3H-benzo[b]azepine-8-carboxamido)pyridin-3- | 2.11 (m, 1H), 1.66 | |
| | yl)methyl)amino)-3-methyl-1-oxobutan-2- | (m, 4H), 1.0-0.95 (m, | |
| | yl)carbamate | 12H). | |
| 1.16 | 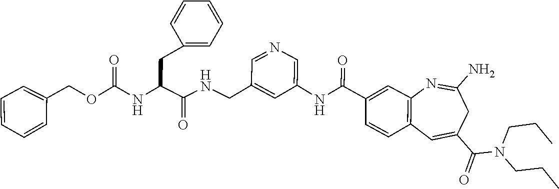 [SMILES: CCCN(CCC)C(=O)C1=Cc2ccc(C(=O)Nc3cncc(CNC(=O)[C@H](Cc4ccccc4)NC(=O)OCc4ccccc4)c3)cc2N=C(N)C1] | (CD₃OD) δ 8.80 (d, J = 2.1 Hz, 1H), 8.21 (s, 1H), 8.11 (s, 1H), 7.72 (s, 1H), 7.61 (dd, J = 1.5, 8.2 Hz, 1H), 7.45 (d, = 8.2 Hz, 1H), 7.33-7.11 (m, 10H), 6.90 (s, 1H), 5.00 (q, J = 12.6 Hz, 2H), 4.35 (m, 3H), 3.43 (m, 4H), 3.12 (m, 1H), 2.89 (m, | 716.3 |
| | | 2H), 1.66 (m, 4H), | |
| | benzyl (S)-(1-(((5-(2-amino-4-(dipropyl-carbamoyl)- | 1.0-0.85 (m, 6H). | |
| | 3H-benzo[b]azepine-8-carboxamido)pyridin-3- | | |
| | yl)methyl)amino)-1-oxo-3-phenylpropan-2- | | |
| | yl)carbamate | | |
| 1.17 | 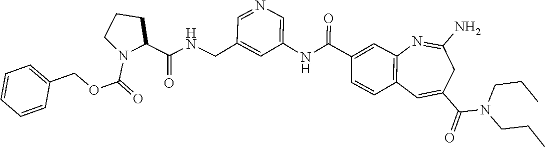 [SMILES: CCCN(CCC)C(=O)C1=Cc2ccc(C(=O)Nc3cncc(CNC(=O)[C@@H]4CCCN4C(=O)OCc4ccccc4)c3)cc2N=C(N)C1] | (CD₃OD) δ 8.82 (d, J = 2.1 Hz, 1H), 8.69 (s, 1H), 8.33-8.21 (m, 2H), 7.70 (d, J = 17 Hz, 1H), 7.57 (dd, J = 1.5, 8.2 Hz, 1H), 7.45 (d, J = 8.1 Hz, 1H), 7.40- 7.21 (m, 5H), 6.90 (s, | 666.5 |
| | | 1H), 5.00 (q, | |
| | benzyl (S)-2-(((5-(2-amino-4-(dipropyl-carbamoyl)- | J = 12.6 Hz, 2H), 4.49 | |
| | 3H-benzo[b]azepine-8-carboxamido)pyridin-3- | (s, 1H), 4.35 (m, | |
| | yl)methyl)carbamoyl)pyrrolidine-1-carboxylate | 2H), 3.63-3.53 (m, | |
| | | 2H), 3.45 (m, 4H), | |
| | | 2.85 (m, 1H), 2.31 | |
| | | (m, 1H), 2.10-1.86 | |
| | | (m, 3H), 1.65 (m, | |
| | | 4H), 1.0-0.85 (m, | |
| | | 6H). | |
| 1.18 | 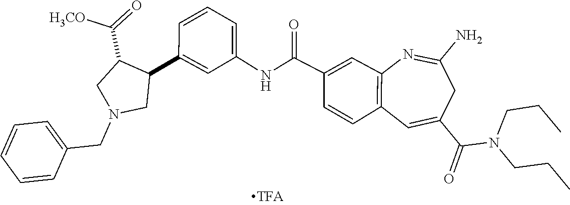 | (CD₃OD) δ 7.96 (m, 2H), 7.89 (bs, 1H), 7.70 (d, 8.2 Hz, 2H), 7.55 (m, 6H), 7.42 (t, J = 7.5 Hz, 1H), 7.22 (d, J = 7.0 Hz, 1H), 7.11 (s, 1H), 4.53 (s, 2H), 3.90 (m, 3H), 3.70 (s, 3H), 3.51 (m, 4H), 3.37 (s, 2H), 1.70 (q, J = 7.5 Hz, 4H), 1.0-0.85 (m, 6H). | 622.2 |
| | methyl (3R,4S)-4-(3-(2-amino-4- | | |
| | (dipropylcarbamoyl)-3H-benzo[b]azepine-8- | | |
| | carboxamido)phenyl)-1-benzylpyrrolidine-3- | | |
| | carboxylate trifluoroacetate salt | | |
| 1.19 | 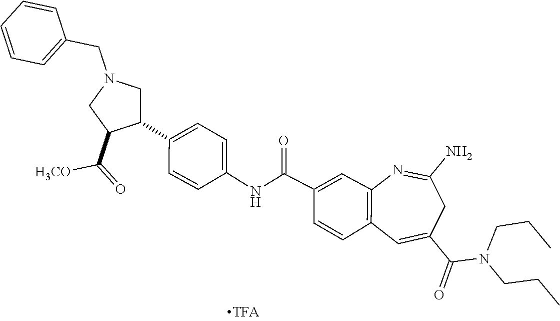 | (CD₃OD) δ 7.95 (m, 2H), 7.75 (d, 8.2 Hz, 2H), 7.69 (d, J = 8.5 Hz, 1H), 7.75 (m, 1H), 7.51 (m, 5H), 7.40 (d, J = 7.5 Hz, 1H), 7.10 (s, 1H), 4.51 (s, 2H), 3.90 (m, 3H), 3.68 (s, 3H), 3.51 (m, 4H), 3.37 (s, 2H), 1.70 (q, J = 7.5 Hz, 4H), 0.99- 0.92 (m, 6H). | 622.2 |
| | methyl (3R,4S)-4-(4-(2-amino-4-(dipropyl- | | |
| | carbamoyl)-3H-benzo[b]azepine-8- | | |
| | carboxamido)phenyl)-1-benzylpyrrolidine-3- | | |
| | carboxylate trifluoroacetate salt | | |
| 1.20 | 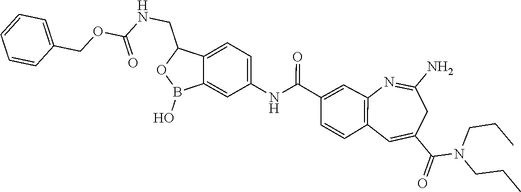 [SMILES: CCCN(CCC)C(=O)C1=Cc2ccc(C(=O)Nc3ccc4c(c3)B(O)OC4CNC(=O)OCc3ccccc3)cc2N=C(N)C1] | | 624.3 |
| | benzyl ((6-(2-amino-4-(dipropylcarbamoyl)-3H- | | |
| | benzo[b]azepine-8-carboxamido)-1-hydroxy-1,3- | | |
| | dihydrobenzo[c][1,2]oxaborol-3-yl)methyl)carbamate | | |
| 1.21 | 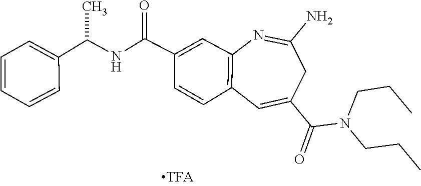 [SMILES: CCCN(CCC)C(=O)C1=Cc2ccc(C(=O)N[C@@H](C)c3ccccc3)cc2N=C(N)C1] | (CD₃OD) δ 7.82 (d, 8.1 Hz, 1H), 7.81 (s, 1H), 7.45 (d, J = 8.1 Hz, 2H), 7.34 (t, J = 7.5 Hz, 2H), 7.27 (d, J = 7.5 Hz, 1H), 7.07 (s, 1H), 5.25 (q, J = 7.0 Hz, 1H), 3.45 (m, 4H), 3.33 (s, 2H), 1.74 (q, J = 7.5 Hz, 4H), 1.52 (d, J = 7.1 Hz, 3H), | 433.2 |
| | (S)-2-amino-N⁸-(1-phenylethyl)-N⁴,N⁴-dipropyl-3H- | 0.94 (bs, 3H), 0.91 | |
| | benzo[b]azepine-4,8-dicarboxamide trifluoroacetate | (bs, 3H). | |
| | salt | | |
| 1.22 | 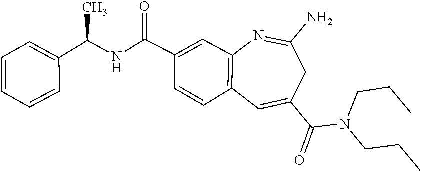 [SMILES: CCCN(CCC)C(=O)C1=Cc2ccc(C(=O)N[C@H](C)c3ccccc3)cc2N=C(N)C1] | (CD₃OD) δ 7.82 (d, 8.1 Hz, 1H), 7.81 (s, 1H), 7.45 (d, J = 8.1 Hz, 2H), 7.34 (t, J = 7.5 Hz, 2H), 7.27 (d, J = 7.5 Hz, 1H), 7.07 (s, 1H), 5.25 (q, J = 7.0 Hz, 1H), 3.45 (m, 4H), 3.33 (s, 2H), 1.74 (q, J = 7.5 Hz, 4H), 1.52 | 433.2 |
| | (R)-2-amino-N⁸-(1-phenylethyl)-N⁴,N⁴-dipropyl-3H- | (d, J = 7.1 Hz, 3H), | |
| | benzo[b]azepine-4,8-dicarboxamide trifluoroacetate | 0.94 (bs, 3H), 0.91 | |
| | salt | (bs, 3H). | |
| 1.23 | 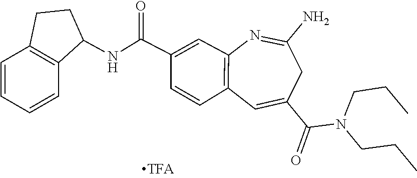 [SMILES: CCCN(CCC)C(=O)C1=Cc2ccc(C(=O)NC3CCc4ccccc43)cc2N=C(N)C1] | (CD₃OD) δ 7.89 (s, 1H), 7.81 (dd, J = 1.8, 8.2 Hz, 1H), 7.61 (d, J = 8.2 Hz, 1H), 7.34 (t, J = 7.5 Hz, 2H), 7.21 (m, 2H), 7.07 (s, 1H), 5.65 (q, J = 7.8 Hz, 1H), 3.48 (m, 4H), 3.28 (s, 2H), 3.01 (m, 1H), 2.95 (m, 1H), 2.62 | 445.1 |
| | | (m, 1H), 2.02 (m, | |
| | 2-amino-N⁸-(2,3-dihydro-1H-inden-1-yl)-N⁴,N⁴- | 1H), 1.68 (q, | |
| | dipropyl-3H-benzo[b]azepine-4,8-dicarboxamide | J = 7.4 Hz, 4H), 0.94 | |
| | trifluoroacetate salt | (bs, 3H), 0.91 (bs, | |
| | | 3H). | |
| 1.24 | 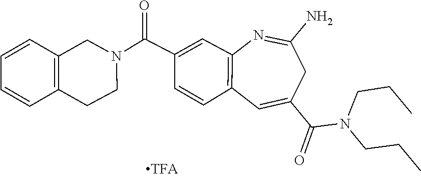 [SMILES: CCCN(CCC)C(=O)C1=Cc2ccc(C(=O)N3CCc4ccccc4C3)cc2N=C(N)C1] | (CD₃OD) δ 7.64 (d, J = 8.4 Hz, 1H), 7.44 (m, 2H), 7.23 (m, 4H), 7.06 (s, 1H), 4.64, (m, 1H), 4.00 (m, 1H), 3.71 (m, 1H), 3.48 (m, 4H), 3.28 (s, 2H), 3.01 (m, 1H), 2.95 (m, 1H), 2.62 (m, 1H), 1.98 (s, 1H), 1.71 (q, | 445.1 |
| | | J = 7.4 Hz, 4H), 1.01 | |
| | 2-amino-N,N-dipropyl-8-(1,2,3,4- | (bs, 3H), 0.95 (bs, | |
| | tetrahydroisoquinoline-2-carbonyl)-3H- | 3H). | |
| | benzo[b]azepine-4-carboxamide trifluoroacetate salt | | |
| 1.25 | 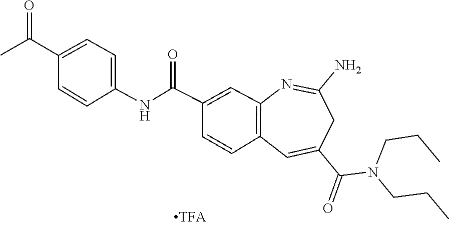 [SMILES: CCCN(CCC)C(=O)C1=Cc2ccc(C(=O)Nc3ccc(C(C)=O)cc3)cc2N=C(N)C1] | (CD₃OD) δ 8.04 (d, J = 8.4 Hz, 1H), 7.94 (d, J = 8.0 Hz, 2H), 7.90 (d, J = 8.8 Hz, 2H), 7.69 (d, J = 8.0 Hz, 1H), 7.10 (s, 1H), 3.43 (m, 4H), 3.28 (s, 2H), 2.60 (s, 3H), 1.71 (q, J = 7.5 Hz, 4H), 0.96 (bs, 3H), 0.92 (bs, 3H). | 447.2 |
| | N⁸-(4-acetylphenyl)-2-amino-N⁴,N⁴-dipropyl-3H- | | |
| | benzo[b]azepine-4,8-dicarboxamide trifluoroacetate | | |
| | salt | | |
| 1.26 | 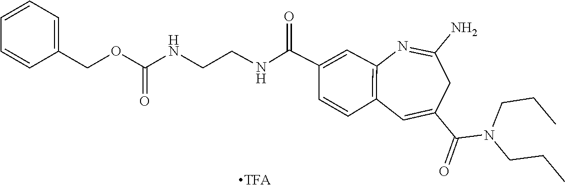 [SMILES: CCCN(CCC)C(=O)C1=Cc2ccc(C(=O)NCCNC(=O)OCc3ccccc3)cc2N=C(N)C1] | (CD₃OD) δ 7.80 (d, J = 1.5 Hz, 1H), 7.74 (dd, J = 1.5, 8.0 Hz, 2H), 7.60 (d, J = 8.0 Hz, 1H), 7.31- 7.23 (m, 5H), 7.07 (s, 1H), 5.06 (s, 2H), 3.53-3.38 (m, 8H), 3.28 (s, 2H), 1.69 (q, J = 7.5 Hz, 4H), 0.95 (bs, 3H), 0.91 (bs, 3H). | 505.8 |
| | benzyl (2-(2-amino-4-(dipropylcarbamoyl)-3H- | | |
| | benzo[b]azepine-8-carboxamido)ethyl)carbamate | | |
| | trifluoroacetate salt | | |
| 1.27 | 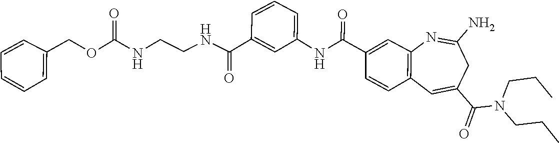 [SMILES: CCCN(CCC)C(=O)C1=Cc2ccc(C(=O)Nc3cccc(C(=O)NCCNC(=O)OCc4ccccc4)c3)cc2N=C(N)C1] | (CD₃OD) δ 7.82- 7.75 (m, 4H), 7.74 (d, J = 8.0 Hz, 2H), 7.60 (d, J = 8.0 Hz, 1H), 7.45 (m, 1H), 7.31-7.23 (m, 5H), 7.07 (s, 1H), 5.06 (s, 2H), 3.53-3.38 (m, 8H), 3.28 (s, 2H), | 625.4 |
| | | 1.69 (q, J = 7.5 Hz, | |
| | benzyl (2-(3-(2-amino-4-(dipropylcarbamoyl)-3H- | 4H), 0.95 (bs, 3H), | |
| | benzo[b]azepine-8- | 0.91 (bs, 3H). | |
| | carboxamido)benzamido)ethyl)carbamate | | |
| 1.28 | 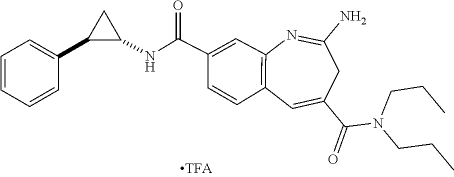 | (CD₃OD) δ 7.83 (s, 1H), 7.79 (d, 8.1 Hz, 1H), 7.61 (d, J = 8.1 Hz, 2H), 7.27 (t, J = 7.5 Hz, 2H), 7.19 (d, J = 7.5 Hz, 1H), 7.06 (s, 1H), 3.45 (m, 4H), 3.28 (s, 2H), 3.00 (m, 1H), 2.21 (m, 1H), 1.68 (q, J = 7.5 Hz, 4H), | 444.8 |
| | | 1.35 (m, 2H), 0.96 | |
| | 2-amino-N⁸-((1S,2R)-2-phenylcyclopropyl)-N⁴,N⁴- | (bs, 3H), 0.91 (bs, | |
| | dipropyl-3H-benzo[b]azepine-4,8-dicarboxamide | 3H). | |
| | trifluoroacetate salt | | |
| 1.29 | 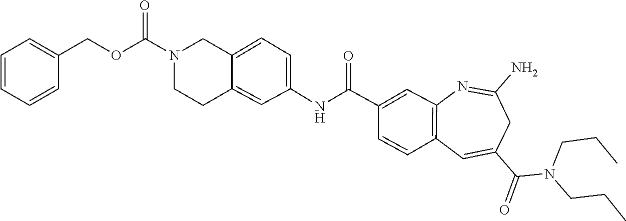 [SMILES: CCCN(CCC)C(=O)C1=Cc2ccc(C(=O)Nc3ccc4c(c3)CCN(C(=O)OCc3ccccc3)C4)cc2N=C(N)C1] | (CD₃OD) δ 7.95 (m, 2H), 7.71-7.61 (m, 3H), 7.27 (d, J = 8.4 Hz, 1H), 7.23 (m, 5H), 7.13 (s, 1H), 5.06 (s, 2H), 4.38 (s, 2H), 4.11 (s, 2H), 3.58 (t, J = 7.5 Hz, 2H), 3.28 (s, 2H), 3.15 (t, J = 6.6 Hz, 2H), 1.71 (q, J = 7.5 Hz, 4H), 0.96 (bs, 6H). | 594.4 |
| | benzyl 6-(2-amino-4-(dipropylcarbamoyl)-3H- | | |
| | benzo[b]azepine-8-carboxamido)-3,4- | | |
| | dihydroisoquinoline-2(1H)-carboxylate | | |
| 1.30 | 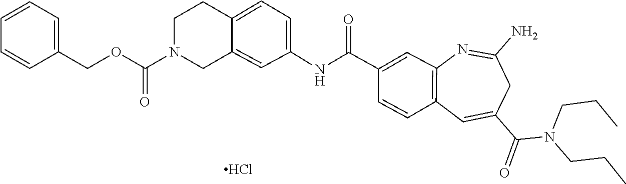 [SMILES: CCCN(CCC)C(=O)C1=Cc2ccc(C(=O)Nc3ccc4c(c3)CN(C(=O)OCc3ccccc3)CC4)cc2N=C(N)C1 ; Cl] | (CD₃OD) δ 7.95 (m, 2H), 7.71-7.61 (m, 3H), 7.27 (d, J = 8.4 Hz, 1H), 7.23 (m, 5H), 7.13 (s, 1H), 5.06 (s, 2H), 4.38 (s, 2H), 4.11 (s, 2H), 3.58 (t, J = 7.5 Hz, 2H), 3.28 (s, 2H), 3.15 (t, J = 6.6 Hz, 2H), 1.71 | 594.4 |
| | | (q, J = 7.5 Hz, 4H), | |
| | benzyl 7-(2-amino-4-(dipropylcarbamoyl)-3H- | 0.96 (bs, 6H). | |
| | benzo[b]azepine-8-carboxamido)-3,4- | | |
| | dihydroisoquinoline-2(1H)-carboxylate HCl salt | | |
| 1.31 | 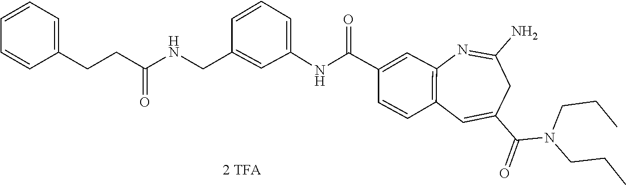 [SMILES: CCCN(CCC)C(=O)C1=Cc2ccc(C(=O)Nc3cccc(CNC(=O)CCc4ccccc4)c3)cc2N=C(N)C1 ; "2 TFA"] | (CD₃OD) δ 9.15 (s, 1H), 8.63 (bs, 1H), 8.42 (s, 1H), 8.29 (s, 1H), 8.02-7.99 (m, 2H), 7.71 (d, J = 8.5 Hz, 1H), 7.23- 7.10 (m, 6H), 4.45 (s, 2H), 3.44 (m, 4H), 3.37 (s, 2H), 2.94 (t, J = 7.5 Hz, 2H), 2.57 (t, J = 7.5 Hz, 2H), | 566.3 |
| | | 1.61 (q, J = 7.5 Hz, | |
| | 2-amino-N⁸-(3-((3- | 4H), 0.96 (bs, 3H), | |
| | phenylpropanamido)methyl)phenyl)-N⁴,N⁴-dipropyl- | 0.91 (bs, 3H). | |
| | 3H-benzo[b]azepine-4,8-dicarboxamide bis TFA salt | | |
| 1.32 | 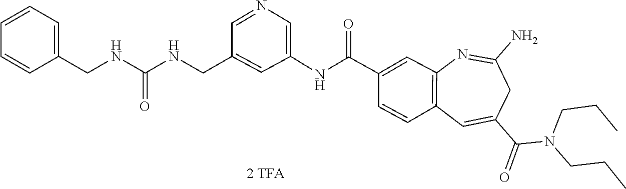 [SMILES: CCCN(CCC)C(=O)C1=Cc2ccc(C(=O)Nc3cncc(CNC(=O)NCc4ccccc4)c3)cc2N=C(N)C1 ; "2 TFA"] | (CD₃OD) δ 9.15 (s, 1H), 8.47 (s, 1H), 8.42 (s, 1H), 8.29 (s, 1H), 8.02-7.99 (m, 2H), 7.72 (d, J = 8.0 Hz, 1H), 7.33 (m, 4H), 7.22 (m, 1H), 7.12 (s, 1H), 4.45 (s, 2H), 4.33 (s, 2H), 3.54 (m, 4H), 3.37 (s, 2H), 1.71 (q, J = 7.5 Hz, 4H), 0.97 | 568.3 |
| | | (bs, 3H), 0.92 (bs, | |
| | 2-amino-N⁸-(5-((3-benzylureido)methyl)pyridin-3- | 3H). | |
| | yl)-N⁴,N⁴-dipropyl-3H-benzo[b]azepine-4,8- | | |
| | dicarboxamide bis TFA salt | | |
| 1.33 | 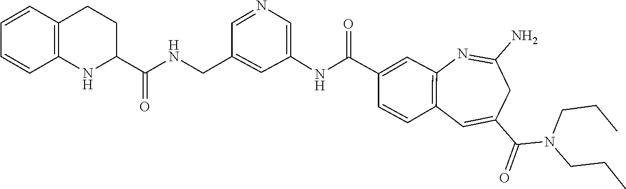 [SMILES: CCCN(CCC)C(=O)C1=Cc2ccc(C(=O)Nc3cncc(CNC(=O)C4CCc5ccccc5N4)c3)cc2N=C(N)C1] | (CD₃OD) δ 8.77 (s, 1H), 8.42 (s, 1H), 8.22 (s, 1H), 8.19 (m, 1H), 7.71 (s, 1H), 7.59 (dd, J = 8.1, 1.8 Hz, 1H), 7.44 (d, J = 8.1 Hz, 1H), 6.99- 6.84 (m, 3H), 6.66 (d, J = 8.0 Hz, 1H), 6.55 (t, J = 7.3 Hz, 1H), 4.51 (s, 2H), 4.00 (t, J = 5.2 Hz, | 593.3 |
| | | 1H), 3.44 (m, 4H), | |
| | 2-amino-N⁴,N⁴-dipropyl-N⁸-(5-((1,2,3,4- | 2.85 (s, 2H), 2.74 (m, | |
| | tetrahydroquinoline-2-carboxamido)methyl)pyridin- | 1H), 2.51 (m, 1H), | |
| | 3-yl)-3H-benzo[b]azepine-4,8-dicarboxamide | 2.25 (m, 1H), 1.91 | |
| | | (m, 1H), 1.67 (m, | |
| | | 4H), 0.96 (bs, 3H), | |
| | | 0.91 (bs, 3H). | |
| 1.34 | 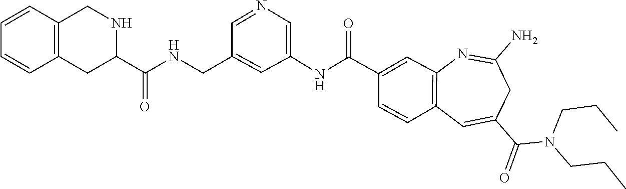 [SMILES: CCCN(CCC)C(=O)C1=Cc2ccc(C(=O)Nc3cncc(CNC(=O)C4Cc5ccccc5CN4)c3)cc2N=C(N)C1] | (CD₃OD) δ 8.80 (d, J = 2.4 Hz, 1H), 8.28 (d, J = 2.1 Hz, 1H), 8.25 (t, J = 2.1 Hz, 1H), 7.72 (d, J = 1.9 Hz, 1H), 7.61 (dd, J = 1.9, 8.1 Hz, 1H), 7.47 (d, J = 8.2 Hz, 1H), 7.13 (m, 3H), 7.05 (m, 1H), 6.90 (s, 1H), 4.50 (s, 2H), 4.05 (q, | 594.4 |
| | | J = 6.1 Hz, 2H), 3.63 | |
| | 2-amino-N⁴,N⁴-dipropyl-N⁸-(5-((1,2,3,4- | (dd, J = 4.7, 10.5 Hz, | |
| | tetrahydroisoquinoline-3-carboxamido)- | 1H), 3.43 (m, 4H), | |
| | methyl)pyridin-3-yl)-3H-benzo[b]azepine-4,8- | 3.05 (dd, J = 4.7, | |
| | dicarboxamide | 16.0 Hz, 1H), 3.02 | |
| | | (m, 1H), 2.83 (d, | |
| | | J = 16.6 Hz, 1H), 1.71 | |
| | | (m, 4H), 1.0-0.85 (m, | |
| | | 6H). | |
| 1.35 | 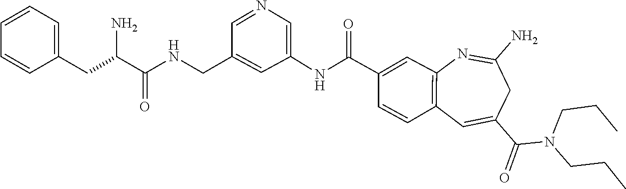 [SMILES: CCCN(CCC)C(=O)C1=Cc2ccc(C(=O)Nc3cncc(CNC(=O)[C@@H](N)Cc4ccccc4)c3)cc2N=C(N)C1] | (CD₃OD) δ 8.78 (d, J = 2.3 Hz, 1H), 8.15 (s, 1H), 8.11 (s, 1H, 7.72 (s, 1H), 7.61 (dd, J = 1.5, 8.2 Hz, 1H), 7.45 (d, = 8.2 Hz, 1H), 7.23-7.15 (m, 5H), 6.90 (s, 1H), 4.44 (q, J = 12.6 Hz, 2H), 3.63 (t, J = 7.5 Hz, 1H), 3.43 (m, 4H), 2.99 (m, | 582.2 |
| | | 1H), 2.89 (m, 2H), | |
| | (S)-2-amino-N⁸-(5-((2-amino-3- | 1.66 (m, 4H), 1.0- | |
| | phenylpropanamido)methyl)pyridin-3-yl)-N⁴,N⁴- | 0.85 (m, 6H). | |
| | dipropyl-3H-benzo[b]azepine-4,8-dicarboxamide | | |
| 1.36 | 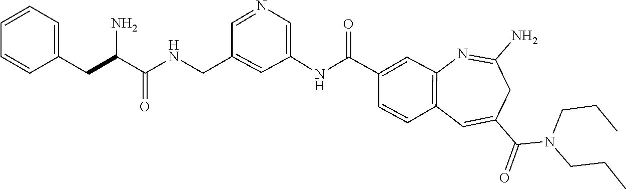 [SMILES: CCCN(CCC)C(=O)C1=Cc2ccc(C(=O)Nc3cncc(CNC(=O)[C@H](N)Cc4ccccc4)c3)cc2N=C(N)C1] | (CD₃OD) δ 8.78 (d, J = 2.3 Hz, 1H), 8.15 (s, 1H), 8.11 (s, 1H), 7.72 (s, 1H), 7.61 (dd, J = 1.5, 8.2 Hz, 1H), 7.45 (d, = 8.2 Hz, 1H), 7.23-7.15 (m, 5H), 6.90 (s, 1H), 4.41 (d, J = 15.0 Hz, 1H), 4.34 (d, J = 15.0 Hz, 1H), 3.63 (t, J = 7.5 Hz, 1H), | 582.2 |
| | | 3.43 (m, 4H), 2.99 | |
| | (R)-2-amino-N⁸-(5-((2-amino-3-phenyl- | (m, 1H), 2.89 (m, | |
| | propanamido)-methyl)pyridine-3-yl)-N⁴,N⁴⁻dipropyl- | 2H), 1.66 (m, 4H), | |
| | 3H-benzo[b]azepine-4,8-dicarboxamide | 1.0-0.85 (m, 6H). | |
| 1.37 | 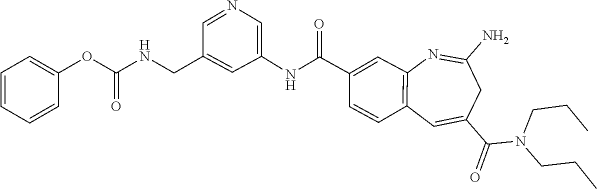 [SMILES: CCCN(CCC)C(=O)C1=Cc2ccc(C(=O)Nc3cncc(CNC(=O)Oc4ccccc4)c3)cc2N=C(N)C1] | (CD₃OD) δ 8.85 (d, J = 2.3 Hz, 1H), 8.35 (s, 1H), 8.31 (s, 1H), 7.72 (s, 1H), 7.95 (m, 2H), 7.72 (d, = 8.5 Hz, 1H), 7.41 (m, 2H), 7.21 (t, J = 7.0 Hz, 1H), 7.15 (d, J = 7.5 Hz, 1H), 7.09 (s, 1H), 4.49 (s, 2H), 3.49 (m, 4H), 1.70 (m, 4H), 1.0-0.85 (m, | 555.2 |
| | | 6H). | |
| | Phenyl ((5-(2-amino-4-(dipropylcarbamoyl)-3H- | | |
| | benzo[b]azepine-8-carboxamido)pyridin-3- | | |
| | yl)methyl)carbamate | | |
| 1.38 | 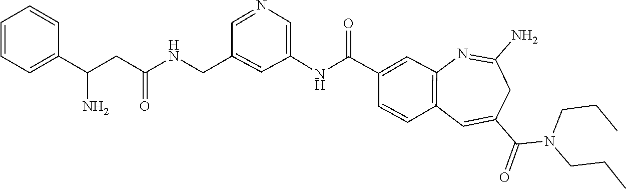 [SMILES: CCCN(CCC)C(=O)C1=Cc2ccc(C(=O)Nc3cncc(CNC(=O)CC(N)c4ccccc4)c3)cc2N=C(N)C1] | (CD₃OD) δ 8.75 (d, J = 2.1 Hz, 1H), 8.15 (s, 1H), 8.11 (s, 1H), 7.72 (d, J = 2.0 Hz, 1H), 7.61 (dd, J = 1.5, 8.2 Hz, 1H), 7.47 (d, = 8.2 Hz, 1H), 7.33- 7.15 (m, 5H), 6.90 (s, 1H), 4.41 (m, 3H), 3.43 (m, 4H), 2.89 (m, 2H), 2.67 (m, 2H), 1.66 (m, 4H), | 582.2 |
| | | 1.0-0.85 (m, 6H). | |
| | 2-amino-N⁸-(5-((3-amino-3-phenyl- | | |
| | propanamido)methyl)-pyridin-3-yl)-N⁴,N⁴⁻dipropyl- | | |
| | 3H-benzo[b]azepine-4,8-dicarboxamide | | |
| 1.39 | 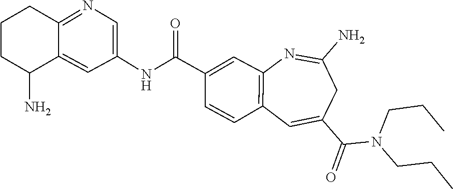 [SMILES: CCCN(CCC)C(=O)C1=Cc2ccc(C(=O)Nc3cnc4c(c3)C(N)CCC4)cc2N=C(N)C1] | (DMSO) δ 10.3 (s, 1H), 8.68 (s, 1H), 8.26 (s, 1H), 7.68 (s, 1H), 7.50 (d, J = 8.4 Hz, 1H), 7.41 (d, J = 8.4 Hz, 1H), 6.89 (bs, 2H), 6.78 (s, 1H), 3.81 (m, 1H), 3.43 (m, 4H), 2.75 (m, 4H), 1.99 (m, 2H), 1.75 (m, 1H), 1.61 (m, 5H). | 475.3 |
| | | 0.88 (bs, 6H). | |
| | 2-amino-N⁸-(5-amino-5,6,7,8-tetrahydro-quinolin-3- | | |
| | yl)-N4,N4-dipropyl-3H-benzo-[b]azepine-4,8- | | |
| | dicarbox-amide | | |
| 1.40 | 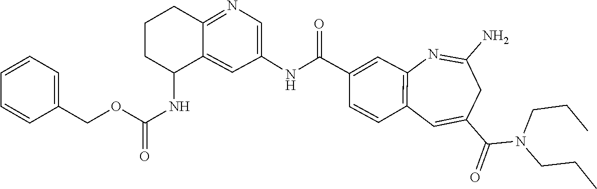 [SMILES: CCCN(CCC)C(=O)C1=Cc2ccc(C(=O)Nc3cnc4c(c3)C(NC(=O)OCc3ccccc3)CCC4)cc2N=C(N)C1] | (CD₃OD) δ 9.25 (d, J = 2.1 Hz, 1H), 8.55 (s, 1H), 8.11 (s, 1H), 8.00 (d, J = 2.0 Hz, 1H), 7.61 (dd, J = 1.5, 8.2 Hz, 1H), 7.33- 7.15 (m, 5H), 7.10 (s, 1H), 5.25 (m, 4H), 5.05 (m, 1H), 3.63- 3.55 (m, 4H), 3.12 (m, 2H), 2.22 (m, 2H), 1.98 (m, 2H), | 609.3 |
| | | 1.66 (m, 4H), 1.0- | |
| | Benzyl (3-(2-amino-4-(dipropylcarbamoyl)-3H- | 0.85 (m, 6H). | |
| | benzo[b]azepine-8-carboxamido)-5,6,7,8- | | |
| | tetrahydroquinolin-5-yl)carbamate | | |
| 1.41 | 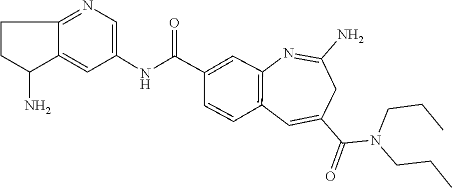 [SMILES: CCCN(CCC)C(=O)C1=Cc2ccc(C(=O)Nc3cnc4c(c3)C(N)CC4)cc2N=C(N)C1] | (CD₃OD) δ 8.75 (d, J = 2.1 Hz, 1H), 8.55 (s, 1H), 7.70 (s, 1H), 7.61 (d, J = 2.0 Hz, 1H), 7.50 (dd, J = 1.5, 8.2 Hz, 1H), 6.90 (s, 1H), 4.70 (m, 1H), 3.63-3.55 (m, 4H), 3.20-2.95 (m, 2H), 2.75 (m, 1H), 2.02 (m, 1H), 1.66 (m, 4H), 1.0-0.85 (m, | 461.4 |
| | | 6H). | |
| | 2-amino-N⁸-(5-amino-6,7-dihydro-5H- | | |
| | cyclopenta[b]pyridin-3-yl)-N⁴,N⁴-dipropyl-3H- | | |
| | benzo[b]azepine-4,8-dicarboxamide | | |
| 1.42 | 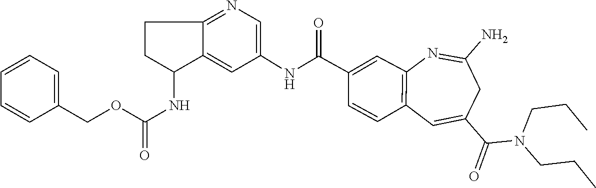 [SMILES: CCCN(CCC)C(=O)C1=Cc2ccc(C(=O)Nc3cnc4c(c3)C(NC(=O)OCc3ccccc3)CC4)cc2N=C(N)C1] | (CD₃OD) δ 8.72 (d, J = 2.1 Hz, 1H), 8.11 (s, 1H), 8.05 (s, 1H), 8.00 (d, J = 2.0 Hz, 1H), 7.61 (dd, J = 1.5, 8.2 Hz, 1H), 7.33- 7.15 (m, 5H), 7.66 (d, 1H), 7.44-7.20 (m, 5H), 7.10 (s, 1H), 5.25 (m, 1H), 5.15 (s, 1H), 3.63- 3.55 (m, 4H), 3.10- | 595.4 |
| | | 2.95 (m, 2H), 2.00 | |
| | Benzyl (3-(2-amino-4-(dipropylcarbamoyl)-3H- | 1.66 (m, 4H), 1.0- | |
| | benzo[b]azepine-8-carboxamido)-6,7-dihydro-5H- | 0.85 (m, 6H). | |
| | cyclopenta[b]pyridin-5-yl)carbamate | | |
| 1.43 | 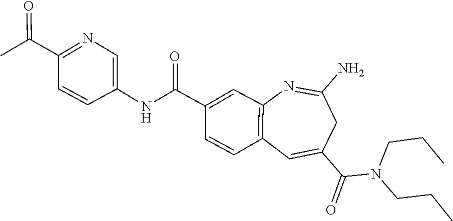 [SMILES: CCCN(CCC)C(=O)C1=Cc2ccc(C(=O)Nc3ccc(C(C)=O)nc3)cc2N=C(N)C1] | (DMSO) δ 12.3 (s, 1H), 10.9 (s, 1H), 9.89 (s, 1H), 9.17 9s, 1H), 9.06 (s, 1H), 8.45 (d, J = 8.8 Hz, 1H), 8.05-7.95 (m, 3H), 7.77 (d, J = 8.0 Hz, 1H), 7.05 (s, 1H), 3.44 (m, 6H), 2.60 (s, 3H), 1.65 (m, 4H), 0.90 (m, 6H). | 448.2 |
| | N⁸-(6-acetylpyridin-3-yl)-2-amino-N⁴,N⁴-dipropyl- | | |
| | 3H-benzo[b]azepine-4,8-dicarboxamide | | |
| 1.44 | 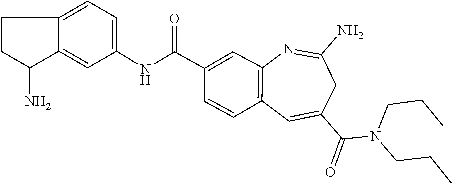 [SMILES: CCCN(CCC)C(=O)C1=Cc2ccc(C(=O)Nc3ccc4c(c3)C(N)CC4)cc2N=C(N)C1] | (DMSO) δ 10.1 (s, 1H), 7.90 (s, 1H), 7.75 (s, 1H), 7.50- 7.40 (m, 3H), 7.17 (d, J = 8.4 Hz, 1H), 6.90 (bs, 1H), 6.68 (s, 1H), 4.25 (m, 1H), 3.50-3.30 (m, 6H), 2.85-2.65 (m, 4H), 2.40 (m, 1H), 1.65-1.55 (m, 5H), | 460.3 |
| | | 0.85 (bs, 6H). | |
| | 2-amino-N⁸-(3-amino-2,3-dihydro-1H-inden-5-yl)- | | |
| | N⁴,N⁴-dipropyl-3H-benzo[b]azepine-4,8- | | |
| | dicarboxamide | | |
| 1.45 | 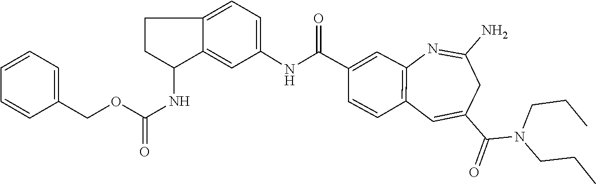 [SMILES: CCCN(CCC)C(=O)C1=Cc2ccc(C(=O)Nc3ccc4c(c3)C(NC(=O)OCc3ccccc3)CC4)cc2N=C(N)C1] | (DMSO) δ 10.1 (s, 1H), 7.72-7.55 (m, 3H), 7.50-7.40 (m, 5H), 7.17 (d, J = 8.4 Hz, 1H), 6.90 (bs, 1H), 6.88 (s, 1H), 5.15 (m, 3H), 3.40 (m, 4H), 2.85- 2.65 (m, 4H), 2.40 (m, 1H), 1.80 (m, 1H), 1.65-1.55 (m, | 594.3 |
| | | 4H), 0.85 (bs, 6H). | |
| | Benzyl (6-(2-amino-4-(dipropylcarbamoyl)-3H- | | |
| | benzo[b]azepine-8-carboxamido)-2,3-dihydro-1H- | | |
| | inden-1-yl)carbamate | | |
| 1.46 | 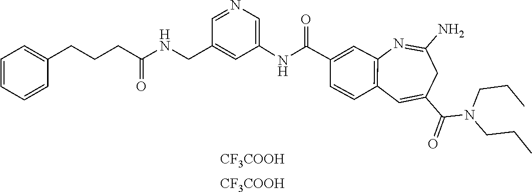 [SMILES: CCCN(CCC)C(=O)C1=Cc2ccc(C(=O)Nc3cncc(CNC(=O)CCCc4ccccc4)c3)cc2N=C(N)C1 ; "CF3COOH" ; "CF3COOH"] | (CD₃OD) δ 9.15 (d, J = 2.1 Hz, 1H), 8.51 (s, 1H), 8.43 (s, 1H), 8.00 (s, 1H), 7.96 (dd, J = 8.4, 2.1 Hz, 1H), 7.71 (d, J = 8.5 Hz, 1H), 7.25- 7.11 (m, 6H), 4.50 (s, 2H), 3.46 (m, 4H), 3.37 (s, 2H), 2.64 (t, J = 7.5 Hz, 2H), 2.31 (t, J = 7.5 Hz, 2H), | 581.2 |
| | | 1.95 (m, 2H), 1.69 | |
| | 2-amino-N⁸-(5-((4- | (m, 4H), 0.96 (bs, | |
| | phenylbutanamido)methyl)pyridin-3-yl)-N⁴,N⁴- | 3H), 0.92 (bs, 3H). | |
| | dipropyl-3H-benzo[b]azepine-4,8-dicarboxamide bis | | |
| | TFA salt | | |
| 1.47 | 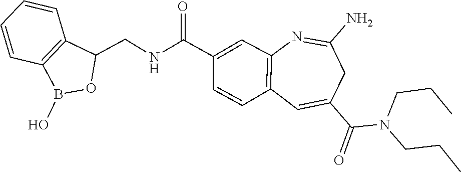 [SMILES: CCCN(CCC)C(=O)C1=Cc2ccc(C(=O)NCC3OB(O)c4ccccc43)cc2N=C(N)C1] | (CD₃OD) δ 7.78 (d, J = 1.5 Hz, 1H), 7.73 (dd, J = 1.5, 8.5 Hz, 1H), 7.66 (d, J = 7.0 Hz, 1H), 7.62 (d, J = 8.5 Hz 1H), 7.47 (m, 2H), 7.37 (m, 1H), 7.07 (s, 1H), 5.44 (dd, J = 3.5, 8.5 Hz, 1H), 4.00 (dd, J = 3.5, 14.0 Hz, 1H), | 473.2 |
| | | 3.6-3.4 (m, 7H), 1.69 | |
| | 2-amino-N⁸-((1-hydroxy-1,3-dihydro- | (m, 4H), 0.95 (bs, | |
| | benzo[c][1,2]oxaborol-3-yl)methyl)-N4,N4-dipropyl- | 3H), 0.91 (bs, 3H). | |
| | 3H-benzo[b]azepine-4,8-dicarboxamide | | |
| 1.48 | 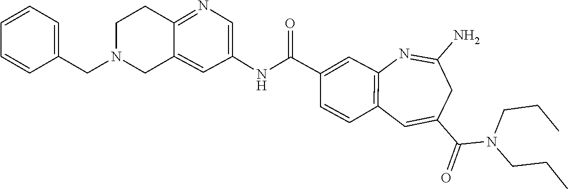 [SMILES: CCCN(CCC)C(=O)C1=Cc2ccc(C(=O)Nc3cnc4c(c3)CN(Cc3ccccc3)CC4)cc2N=C(N)C1] | (CD₃OD) δ 9.33 (s, 1H), 8.89 (s, 1H), 8.09 (s, 1H), 8.05 (d, J = 8.4 Hz, 1H), 7.74 (d, J = 8.4 Hz, 1H), 7.31-7.22 (m, 5H), 7.12 (s, 1H), 4.40 (s, 2H), 4.37 (s, 2H), 3.48 (m, 4H), 3.38- 3.28 (m, 8H), 1.71 q, J = 7.5 Hz, 4H), 0.97-0.92 (bs, 6H). | 551.3 |
| | 2-amino-N⁸-(6-benzyl-5,6,7,8-tetrahydro-1,6- | | |
| | naphthyridin-3-yl)-N⁴,N⁴-dipropyl-3H- | | |
| | benzo[b]azepine-4,8-dicarboxamide | | |
| 1.49 | 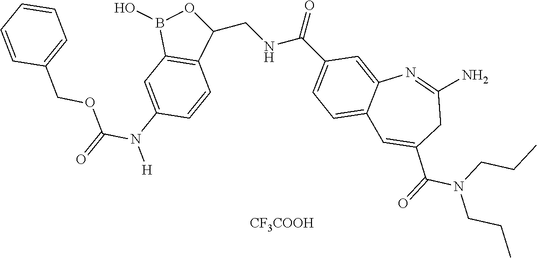 [SMILES: CCCN(CCC)C(=O)C1=Cc2ccc(C(=O)NCC3OB(O)c4cc(NC(=O)OCc5ccccc5)ccc43)cc2N=C(N)C1 ; "CF3COOH"] | (DMSO) δ 12.0 (s, 1H), 9.82 (s, 1H), 9.29 (s, 1H), 8.98 (s, 1H), 8.92 (m, 1H), 7.88-7.83 (m, 3H), 7.65 (d, J = 8.5 Hz, 1H), 7.50 (dd, J = 8.0, 0 Hz, 1H), 7.45-7.33 (m, 6H), 7.01 (s, 1H), 5.30 (m, 1H), 5.15 (s, 2H), 3.70 (m, 1H), 4.40- 3.30 (m, 5H), 1.58 (m, 4H), 0.89 (bs, 3H), 0.80 (bs, 3H). | 624 |
| | benzyl (3-((2-amino-4-(dipropylcarbamoyl)-3H- | | |
| | benzo[b]azepine-8-carboxamido)methyl)-1-hydroxy- | | |
| | 1,3-dihydrobenzo[c][1,2]oxaborol-6-yl)carbamate | | |
| | TFA salt | | |
| 1.50 | 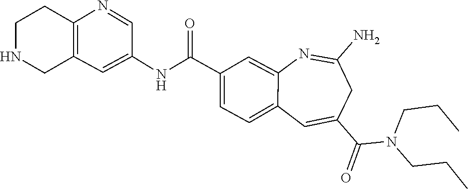 [SMILES: CCCN(CCC)C(=O)C1=Cc2ccc(C(=O)Nc3cnc4c(c3)CNCC4)cc2N=C(N)C1] | (CD₃OD) δ 8.68 (s, 1H), 8.00 (s, 1H), 7.70 (s, 1H), 7.58 (dd, J = 8.4, 2.1 Hz, 1H), 7.47 (d, J = 8.4 Hz, 1H), 6.91 (s, 1H), 4.08 (s, 2H), 3.38-3.28 (m, 6H), 2.95 (t, J = 3.0 Hz, 2H), 1.71 (q, J = 7.5 Hz, 4H), 0.97- 0.92 (bs, 6H). | 461 |
| | 2-amino-N⁴,N⁴-dipropyl-N⁸-(5,6,7,8-tetrahydro-1,6- | | |
| | naphthyridin-3-yl)-3H-benzo[b]azepine-4,8- | | |
| | dicarboxamide | | |
| 1.51 | 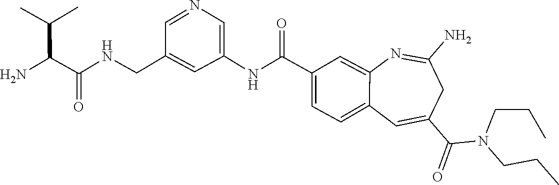 [SMILES: CCCN(CCC)C(=O)C1=Cc2ccc(C(=O)Nc3cncc(CNC(=O)[C@@H](N)C(C)C)c3)cc2N=C(N)C1] | (CD₃OD) δ 8.78 (d, J = 2.3 Hz, 1H), 8.33 (s, 1H), 8.31 (s, 1H), 7.72 (s, 1H), 7.57 (dd, J = 1.5, 8.2 Hz, 1H), 7.45 (d, = 8.4 Hz, 1H), 6.90 (s, 1H), 4.48 (s, 2H), 3.43 (m, 4H), 2.00 (m, 1H), 1.66 (m, 4H), 1.0- 0.85 (m, 12H). | 534 |
| | (S)-2-amino-N⁸-(5-((2-amino-3- | | |
| | methylbutanamido)methyl)pyridin-3-yl)-N⁴,N⁴- | | |
| | dipropyl-3H-benzo[b]azepine-4,8-dicarboxamide | | |
| 1.52 | 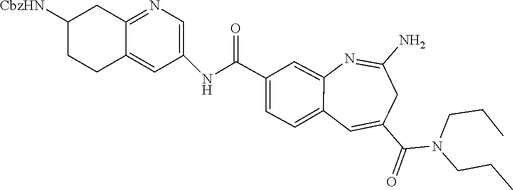 [SMILES: CCCN(CCC)C(=O)C1=Cc2ccc(C(=O)Nc3cnc4c(c3)CCC(NC(=O)OCc3ccccc3)C4)cc2N=C(N)C1] | (DMSO) δ 10.3 (s, 1H), 8.70 (s, 1H), 7.98 (s, 1H), 7.66 (s, 1H), 7.52-7.35 (m, 6H), 6.90 (bs, 1H), 6.88 (s, 1H), 5.15 (s, 2H), 3.80 (m, 1H), 3.43 (m, 4H), 3.00- 2.65 (m, 6H), 2.02 (m, 1H), 1.65-1.55 (m, 4H), 0.85 (bs, 6H). | 609.3 |
| | benzyl (3-(2-amino-4-(dipropylcarbamoyl)-3H- | | |
| | benzo[b]azepine-8-carboxamido)-5,6,7,8- | | |
| | tetrahydroquinolin-7-yl)carbamate | | |
| 1.53 | 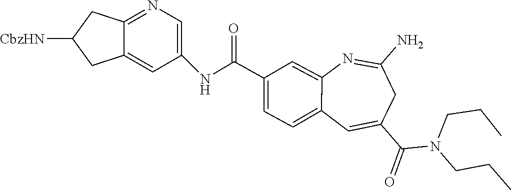 [SMILES: CCCN(CCC)C(=O)C1=Cc2ccc(C(=O)Nc3cnc4c(c3)CC(NC(=O)OCc3ccccc3)C4)cc2N=C(N)C1] | (CD₃OD) δ 9.05 (m, 1H), 8.45 (m, 1H), 7.98 (m, 1H), 7.66 (m, 1H), 7.22-7.35 (m, 5H), 7.10 (s, 1H), 5.09 (s, 2H), 4.73 (m, 1H), 3.43 (m, 4H), 3.00-2.65 (m, 2H), 1.72-1.62 (m, 4H), 0.85 (bs, 6H). | 595.3 |
| | benzyl (3-(2-amino-4-(dipropylcarbamoyl)-3H- | | |
| | benzo[b]azepine-8-carboxamido)-6,7-dihydro-5H- | | |
| | cyclopenta[b]pyridin-6-yl)carbamate | | |
| 1.54 | 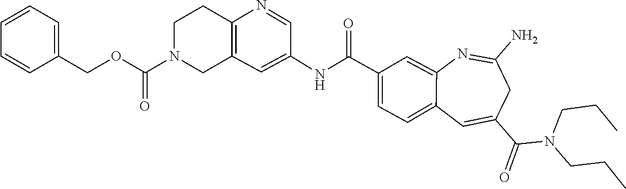 [SMILES: CCCN(CCC)C(=O)C1=Cc2ccc(C(=O)Nc3cnc4c(c3)CN(C(=O)OCc3ccccc3)CC4)cc2N=C(N)C1] | (CD₃OD) δ 8.70 (s, 1H), 8.05 (s, 1H), 7.93 (m, 2H), 7.66 (d, J = 7.8 Hz, 1H), 7.42-7.31 (m, 5H), 7.08 (s, 1H), 5.19 (s, 2H), 4.73 (m, 2H), 3.85 (bs, 2H), 3.43 (m, 4H), 3.00-2.95 (m, 2H), 1.72-1.62 (m, 4H), 0.85 (bs, 6H). | 595 |
| | benzyl 3-(2-amino-4-(dipropylcarbamoyl)-3H- | | |
| | benzo[b]azepine-8-carboxamido)-7,8-dihydro-1,6- | | |
| | naphthyridine-6(5H)-carboxylate | | |
| 1.55 | 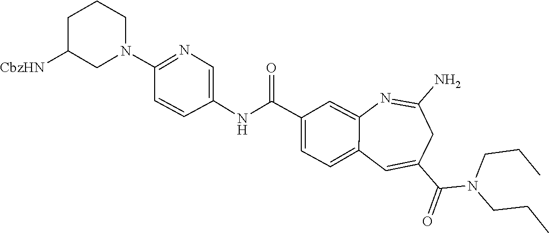 [SMILES: CCCN(CCC)C(=O)C1=Cc2ccc(C(=O)Nc3ccc(N4CCCC(NC(=O)OCc5ccccc5)C4)nc3)cc2N=C(N)C1] | (DMSO-d⁶) δ 12.2 (bs, 1H), 10.2 (s, 1H), 8.50 (s, 1H), 8.00-7.75 (m, 3H), 7.65 (d, J = 7.8 Hz, 1H), 7.43-7.25 (m, 5H), 7.01 (s, 1H), 6.82 (d, J = 8.8 Hz, 1H), 5.04 (s, 2H), 4.21 (d, J = 12 Hz, 1H), 4.04 (d, J = 12 Hz, 1H), 3.55- 3.00 (m, 7H), 2.80- 2.70 (m, 2H), 2.00- | 638.3 |
| | | 1.40 (m, 8H), 0.85 | |
| | benzyl (1-(5-(2-amino-4-(dipropylcarbamoyl)-3H- | (bs, 6H). | |
| | benzo[b]azepine-8-carboxamido)pyridin-2- | | |
| | yl)piperidin-3-yl)carbamate | | |
| 1.56 | 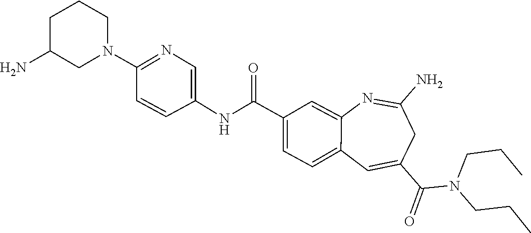 [SMILES: CCCN(CCC)C(=O)C1=Cc2ccc(C(=O)Nc3ccc(N4CCCC(N)C4)nc3)cc2N=C(N)C1] | (DMSO-d⁶) δ 10.2 (s, 1H), 8.48 (s, 1H), 7.98 (d, J = 7.2 Hz, 1H), 7.65 (s, 1H), 7.45 (m, 2H), 6.82 (d, J = 8.2 Hz, 1H), 4.21 (d, J = 12 Hz, 1H), 3.94 (d, J = 12 Hz, 1H), 2.80- 2.70 (m, 4H), 2.00- 1.40 (m, 10H), 0.85 (bs, 6H). | 504.2 |
| | 2-amino-N⁸-(6-(3-aminopiperidin-1-yl)pyridin-3-yl)- | | |
| | N⁴,N⁴-dipropyl-3H-benzo[b]azepine-4,8- | | |
| | dicarboxamide | | |
| 1.57 | 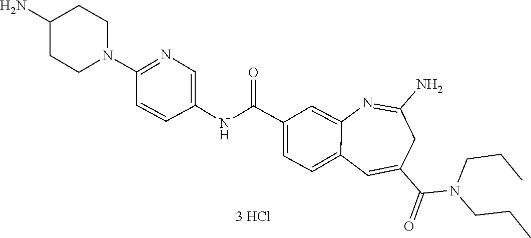 [SMILES: CCCN(CCC)C(=O)C1=Cc2ccc(C(=O)Nc3ccc(N4CCC(N)CC4)nc3)cc2N=C(N)C1] | (CD₃OD) δ 8.65 (s, 1H), 8.16 (m, 1H), 8.00-7.96 (m, 2H), 7.70 (d, J = 8.0 Hz, 1H), 7.32 (m, 1H), 7.11 (s, 1H), 4.33 (d, J = 13.5 Hz, 2H), 3.47- 3.40 (m, 5H), 2.17 (m 2H), 1.72 (m, 6H), 0.94 (m, 6H). | 504.6 |
| | 2-amino-N⁸-(6-(4-aminopiperidin-1-yl)pyridin-3-yl)- | | |
| | N⁴,N⁴-dipropyl-3H-benzo[b]azepine-4,8- | | |
| | dicarboxamide tris HCl salt | | |
| 1.58 | 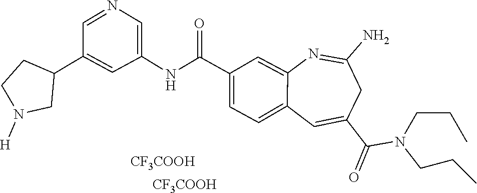 [SMILES: CCCN(CCC)C(=O)C1=Cc2ccc(C(=O)Nc3cncc(C4CCNC4)c3)cc2N=C(N)C1 ; "CF3COOH" ; "CF3COOH"] | (CD₃OD) δ 8.84 (J = 1.5 Hz, 1H), 8.42 (d, J = 1.5 Hz, 1H), 8.37 (d, J = 1.5 Hz, 1H), 8.01-7.98 (m, 2H), 7.71 (d, J = 8.0 Hz, 1H), 7.11 (s, 1H), 4.80 (m, 1H), 3.83 (m, 1H), 7.73-3.60 (m, 2H), 3.52-3.44 (m, 2H), 2.57 (m, 1H), 2.18 | 475 |
| | | (m, 1H), 1.71 (q, | |
| | 2-amino-N⁴,N⁴-dipropyl-N⁸-(5-(pyrrolidin-3- | J = 7.5 Hz, 4H), 0.97 | |
| | yl)pyridin-3-yl)-3H-benzo[b]azepine-4,8- | (bs, 3H), 0.92 (bs, | |
| | dicarboxamide | 3H). | |
| 1.59 | 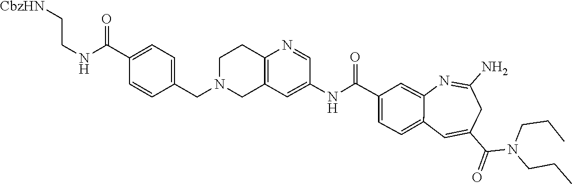 [SMILES: CCCN(CCC)C(=O)C1=Cc2ccc(C(=O)Nc3cnc4c(c3)CN(Cc3ccc(C(=O)NCCNC(=O)OCc5ccccc5)cc3)CC4)cc2N=C(N)C1] | (CD₃OD) δ 8.71 (J = 1.5 Hz, 1H), 8.05 (bs, 1H), 7.95 (m, 2H), 7.87 (m, 2H), 7.70 (d, J = 9.0 Hz, 1H), 7.57 (d, J = 7.5 Hz, 1H), 7.32- 7.25 (m, 5H), 7.11 (s, 1H), 5.06 (s, 2H), 3.51-3.46 (m, 6H), 3.37 (m, 4H), 1.69 | 771 |
| | | (q, J = 7.5 Hz, 4H), | |
| | benzyl (2-(4-((3-(2-amino-4-(dipropylcarbamoyl)- | 0.96 (bs, 3H), 0.92 | |
| | 3H-benzo[b]azepine-8-carboxamido)-7,8-dihydro- | (bs, 3H). | |
| | 1,6-naphthyridin-6(5H)- | | |
| | yl)methyl)benzamido)ethyl)carbamate | | |
| 1.60 | 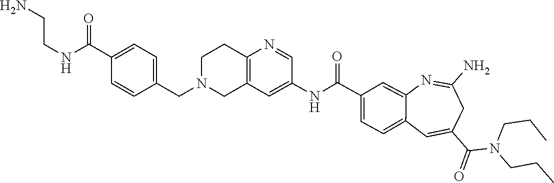 [SMILES: CCCN(CCC)C(=O)C1=Cc2ccc(C(=O)Nc3cnc4c(c3)CN(Cc3ccc(C(=O)NCCN)cc3)CC4)cc2N=C(N)C1] | (CD₃OD) δ 8.65 (J = 2.5 Hz, 1H), 7.95 (J = 2.5 Hz, 1H), 7.85 (d, J = 8.5 Hz, 2H), 7.68 (d, J = 2.0 Hz, 1H), 7.58-7.53 (m, 3H), 7.44 (d, J = 8.5 Hz, 1H), 6.89 (s, 1H), 3.82 (s, 2H), 3.70 (s, 2H), 3.53 (t, J = 6.0 Hz, 2H), 3.42 | 637.6 |
| | | (m, 4H), 3.00-2.89 | |
| | 2-amino-N⁸-(6-(4-((2-aminoethyl)carbamoyl)benzyl)- | (m, 6H), 1.67 (m, | |
| | 5,6,7,8-tetrahydro-1,6-naphthyridin-3-yl)-N⁴,N⁴- | 4H), 0.95-0.87 (m, | |
| | dipropyl-3H-benzo[b]azepine-4,8-dicarboxamide | 6H). | |
| 1.61 | 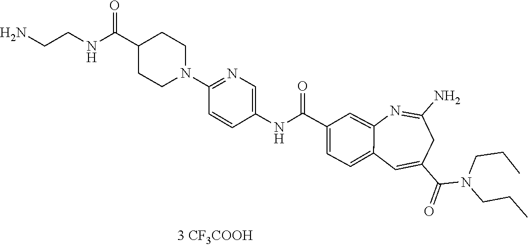 [SMILES: CCCN(CCC)C(=O)C1=Cc2ccc(C(=O)Nc3ccc(N4CCC(C(=O)NCCN)CC4)nc3)cc2N=C(N)C1] | (CD₃OD) δ 8.57 (d, J = 2.5 Hz, 1H), 8.06 (dd, J = 8.0, 2.5 Hz, 1H), 7.96 (s, 1H), 7.94 (d, J = 8.0 Hz, 1H), 7.70 (d, J = 8.0 Hz, 1H), 7.21 (d, J = 8.0 Hz, 1H), 7.11 (s, 1H), 4.25 (d, J = 13.5 Hz, 2H), 3.48- 3.44 (m, 6H), 3.17 (m, 2H), 3.06 (t, J = 6.0 Hz, 2H), 2.57 (m, 1H), 1.99-1.95 (m, 2H), 1.82-1.79 (m, 2H), 1.73-1.66 | 575.6 |
| | | (m, 4H), 0.97 (bs, | |
| | 2-amino-N⁸-(6-(4-((2- | 3H), 0.91 (bs, 3H). | |
| | aminoethyl)carbamoyl)piperidin-1-yl)pyridin-3-yl)- | | |
| | N⁴,N⁴-dipropyl-3H-benzo[b]azepine-4,8- | | |
| | dicarboxamide tris TFA salt | | |
| 1.62 | 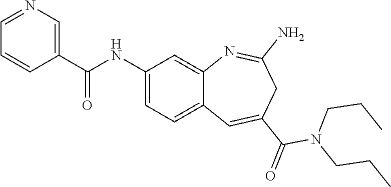 [SMILES: CCCN(CCC)C(=O)C1=Cc2ccc(NC(=O)c3cccnc3)cc2N=C(N)C1] | | |
| | 2-amino-8-(nicotinamido)-N,N-dipropyl-3H- | | |
| | benzo[d]azepine-4-carboxamide | | |
| 1.63 | 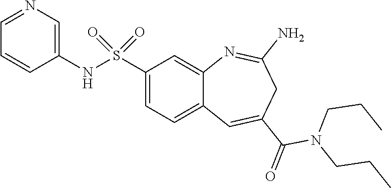 [SMILES: CCCN(CCC)C(=O)C1=Cc2ccc(S(=O)(=O)Nc3cccnc3)cc2N=C(N)C1] | | |
| | 2-amino-N,N-dipropyl-8-(N-(pyridin-3- | | |
| | yl)sulfamoyl)-3H-benzo[b]azepine-4-carboxamide | | |
| 1.64 | 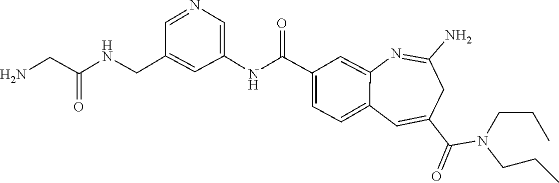 [SMILES: CCCN(CCC)C(=O)C1=Cc2ccc(C(=O)Nc3cncc(CNC(=O)CN)c3)cc2N=C(N)C1] | ¹H NMR (DMSO-d⁶) δ 10.4 (s, 1H), 8.85 (d, J = 2.4 Hz, 1H), 8.44 (t, J = 6.0 Hz, 1H), 8.23 (d, 2.0 Hz, 1H), 8.13 (d, t, J = 2.0 Hz, 1H), 7.68 (d, J = 2.0 Hz, 1H), 7.50 (dd, J = 2.0, 8.0 Hz, 1H), 7.41 (d, J = 8.0 Hz, 1H), 6.91 | 492.3 |
| | | (bs, 2H), 6.79 (s, | |
| | 2-amino-N8-(5-((2-aminoacetamido)methyl)pyridin- | 1H), 4.33 (d, | |
| | 3-yl)-N4,N4-dipropyl-3H-benzo[b]azepine-4,8- | J = 5.6 Hz, 1H), 3.33 | |
| | dicarboxamide | (m, 2H), 3.15 (s, | |
| | | 1H), 2.73 (s, 1H), | |
| | | 1.78 (bs, 1H), 1.56 | |
| | | (m, 4H), 0.84 (bs, | |
| | | 6H). | |
| 1.65 | 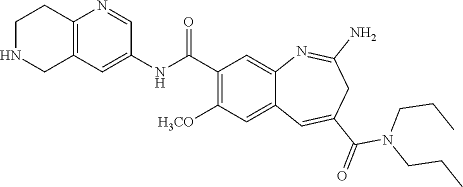 [SMILES: CCCN(CCC)C(=O)C1=Cc2cc(OC)c(C(=O)Nc3cnc4c(c3)CNCC4)cc2N=C(N)C1] | | |
| | 2-amino-7-methoxy-N4,N4-dipropyl-N8-(5,6,7,8- | | |
| | tetrahydro-1,6-naphthyridin-3-yl)-3H- | | |
| | benzo[b]azepine-4,8-dicarboxamide | | |
| 1.66 | 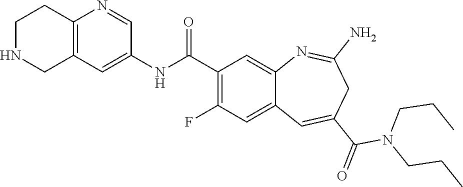 [SMILES: CCCN(CCC)C(=O)C1=Cc2cc(F)c(C(=O)Nc3cnc4c(c3)CNCC4)cc2N=C(N)C1] | | |
| | 2-amino-7-fluoro-N4,N4-dipropyl-N8-(5,6,7,8- | | |
| | tetrahydro-1,6-naphthyridin-3-yl)-3H- | | |
| | benzo[d]azepine-4,8-dicarboxamide | | |
| 1.67 | 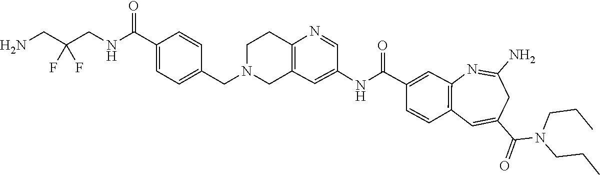 [SMILES: CCCN(CCC)C(=O)C1=Cc2ccc(C(=O)Nc3cnc4c(c3)CN(Cc3ccc(C(=O)NCC(F)(F)CN)cc3)CC4)cc2N=C(N)C1] | | |
| | 2-amino-N8-(6-(4-((3-amino-2,2- | | |
| | difluoropropyl)carbamoyl)benzyl)-5,6,7,8-tetrahydro- | | |
| | 1,6-naphthyridin-3-yl)-N4,N4-dipropyl-3H- | | |
| | benzo[d]azepine-4,8-dicarboxamide | | |

### Example 4

### Synthesis of 8-Substituted Anilides

### Preparation of 2-amino-8-(nicotinamido)-N,N-dipropyl-3H-benzo[b]azepine-4-carboxamide (Compound 1.62)

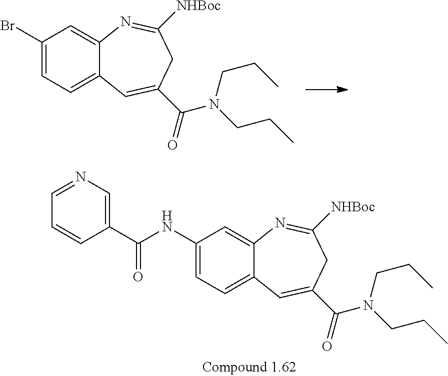 [SMILES: CCCN(CCC)C(=O)C1=Cc2ccc(Br)cc2N=C(NC(=O)OC(C)(C)C)C1 ; CCCN(CCC)C(=O)C1=Cc2ccc(NC(=O)c3cccnc3)cc2N=C(NC(=O)OC(C)(C)C)C1]

Step A: Preparation of Compound 1.62

To a solution containing 46 mg (0.10 mmol) of tert-butyl (8-bromo-4-(dipropylcarbamoyl)-3H-benzo[b]azepin-2-yl)carbamate in 5 mL of DMF was added 65 mg (0.20 mmol) of Cs₂CO₃ and 15 mg (0.12 mmol) of nicotinamide. The solution was degassed then treated with 18 mg (0.2 equiv.) of BrettPhos Pd G3 and 11 mg (0.2 equiv.) of BrettPhos and heated at 90° C. for 12 h. The reaction mixture was cooled and chromatographed by preparative HPLC to afford 6 mg of the desired coupled and deprotected compound as an off-white solid. ¹H NMR (DMSO-d⁶) δ 10.4 (s, 1H), 9.10 (d, J=1.6 Hz, 1H), 8.76 (d, J=8.0 Hz, 1H), 8.28 (d, J=8.0 Hz, 1H), 7.55 (m, 1H), 7.52 (s, 1H), 7.36 (d, J=8.2 Hz, 1H), 7.27 (d, J=8.0 Hz, 1H), 6.80 (bs, 1H), 6.68 (s, 1H), 3.44 (m, 4H), 2.69 (m, 1H), 1.54 (m, 4H), 0.89 (bs, 6H). LCMS (M+H)=406.2.

### Example 5

### Synthesis of 8-Substituted Sulfonamides

### Preparation of 2-amino-N,N-dipropyl-8-(N-(pyridin-3-yl)sulfamoyl)-3H-benzo[b]azepine-4-carboxamide (Compound 1.63)

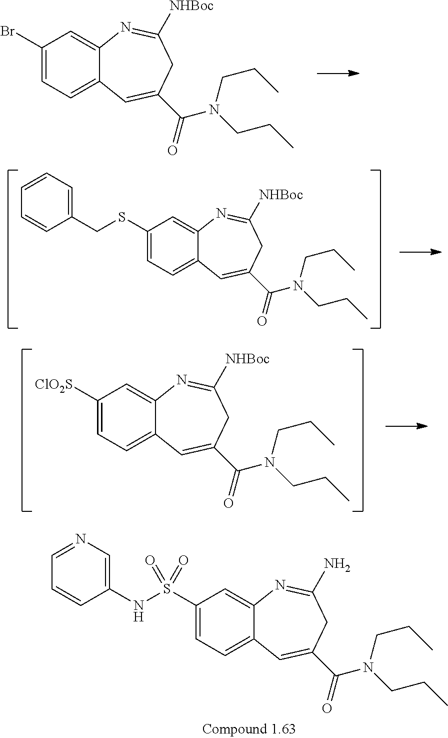

Step A: Preparation of Compound 1.63

To a solution containing 460 mg (1.0 mmol) of tert-butyl (8-bromo-4-(dipropylcarbamoyl)-3H-benzo[b]azepin-2-yl)carbamate in 50 mL of dioxane was added 210 mg (2.0 mmol) of N,N-diisopropylethylamine and 140 mg (1.2 mmol) of benzylthiol. The solution was degassed then treated with 180 mg (0.20 mmol) of Pd₂(dba)₃ and 116 mg (0.20 mmol) of XantPhos and heated at 90° C. for 6 h. The reaction mixture was cooled and filtered through Celite then chromatographed by reverse phase chromatography to afford 250 mg of the desired thiol ether which was immediately dissolved in DCM (20 ml) and acetic acid (0.5 ml). The resulting solution was cooled in an ice water bath and 1,3-dichloro-5,5-dimethy 2-imidazolidinedione (197 mg, 1.0 mmol) was added. After 2 h the mixture was extracted with DCM and brine and the organics were dried and evaporated. The residue was dissolved in MeCN and treated with 1-methyl-1H-imidazole and 3-aminopyridine at 0° C. and stirred to room temperature over 2 h. The solution was extracted with brine and dried over Na₂SO₄. The residue was then dissolved in 4 mL of DCM and treated with 1 mL of TFA and stirred for 2 h. Evaporation of the solvent and purification by reverse phase HPLC afforded 30 mg of the desired compound 1.63. ¹H NMR (DMSO-d⁶) δ 10.5 (bs, 1H), 8.32 (s, 1H), 8.25 (d, J=2.0 Hz, 1H), 7.54 (d, 8.0 Hz, 1H), 7.52 (d, J=8.0 Hz, 1H), 7.45 (s, 1H), 7.22 (dd, J=8.0, 2.0 Hz, 1H), 7.07 (m, 2H), 6.73 (s, 1H), 3.30 (m, 4H), 2.95 (s, 2H), 2.11 (s, 1H), 1.54 (m, 4H), 0.85 (bs, 6H). LCMS (M+H)=442.1.

### Example 6

### Synthesis of Linker-Modified Payloads (LP)

### Preparation of 4-((S)-2-((S)-2-(6-(2,5-dioxo-2,5-dihydro-1H-pyrrol-1-yl)hexanamido)-3-methylbutanamido)-5-ureidopentanamido)benzyl ((5-(2-amino-4-(dipropyl-carbamoyl)-3H-benzo[b]azepine-8-carboxamido)pyridin-3-yl)methyl)carbamate (Compound-Linker 2.1)

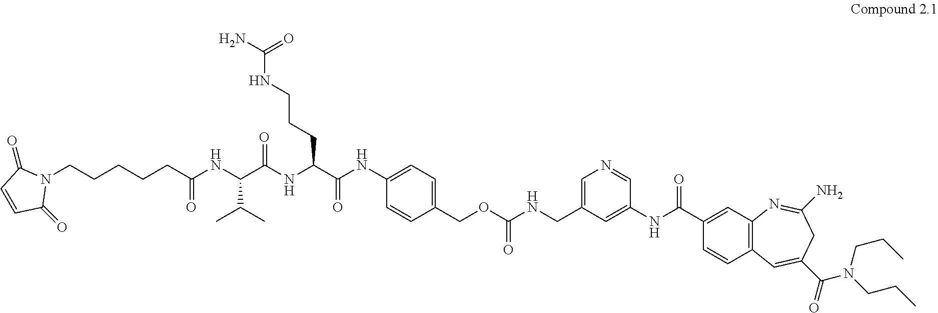 [SMILES: CCCN(CCC)C(=O)C1=Cc2ccc(C(=O)Nc3cncc(CNC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1]

Step A: Preparation of Compound 2.1

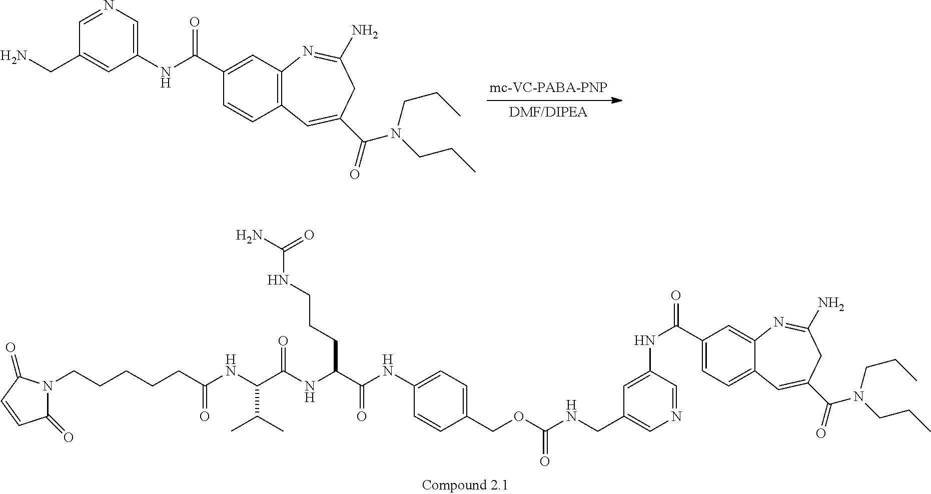 [SMILES: CCCN(CCC)C(=O)C1=Cc2ccc(C(=O)Nc3cncc(CN)c3)cc2N=C(N)C1 ; CCCN(CCC)C(=O)C1=Cc2ccc(C(=O)Nc3cncc(CNC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1]

54 mg (0.07 mmol) of MC-Val-Cit-PAB-PNP (CAS No. 159857-81-5) was added to a solution containing 40 mg (0.07 mmol) of 2-amino-N⁸-(5-(aminomethyl)pyridin-3-yl)-N⁴,N⁴-dipropyl-3H-benzo[b]azepine-4,8-dicarboxamide in 1.0 mL of DMF and 32 μL (0.18 mmol) of DIPEA. The reaction mixture was stirred for 16 h then purified directly by reverse phase chromatography (no TFA). The clean fractions were lyophilized to afford 60 mg (71%) of the desired product which was dissolved in 5 mL of DCM and treated with 1 mL of TFA at room temperature. The mixture was stirred for 45 minutes and then evaporated. The resulting residue was purified by reverse phase chromatography (no TFA) to afford 34 mg (62%) of Compound-Linker 2.1 as a white solid. ¹H NMR (CD₃OD) δ 8.81 (s, 1H), 8.25 (s, 1H), 8.21 (s, 1H), 7.72 (s, 1H), 7.58 (m, 2H), 7.45 (d, J=8.2 Hz, 2H), 7.33 (d, J=8.4 Hz, 2H), 6.91 (s, 1H), 6.75 (s, 2H), 5.08 (s, 2H), 4.49 (m, 1H), 4.39 (m, 2H), 4.14 (d, J=6.5 Hz, 1H), 3.47 (t, J=7.1 Hz, 2H), 3.42 (m, 4H), 3.15 (m, 1H), 3.10 (m, 1H), 2.27 (t, J=7.4 Hz, 2H), 2.05 (m, 1H), 1.88 (m, 1H), 1.75-1.52 (m, 13H), 1.31 (m, 2H), 0.97 (t, J=6.5 Hz, 6H). LCMS [M+H]=1033.

### Example 7

### Synthesis of Linker-Modified Payloads (LP) With Myeloid Agonists

### Preparation of 2-amino-N⁸-(5-((6-(4-((2,5-dioxo-2,5-dihydro-1H-pyrrol-1-yl)methyl)cyclohexane-1-carboxamido)hexanamido)methyl)pyridin-3-yl)-N⁴,N⁴-dipropyl-3H-benzo[b]azepine-4,8-dicarboxamide (Compound-Linker 2.2)

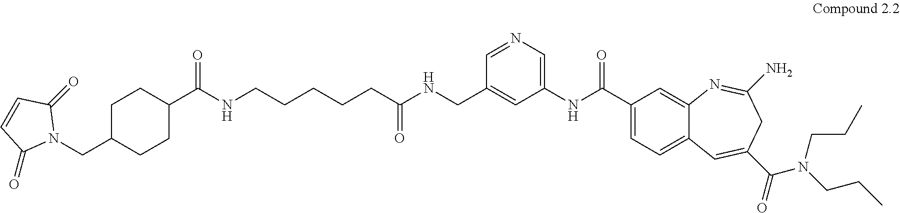 [SMILES: CCCN(CCC)C(=O)C1=Cc2ccc(C(=O)Nc3cncc(CNC(=O)CCCCCNC(=O)C4CCC(CN5C(=O)C=CC5=O)CC4)c3)cc2N=C(N)C1]

Step A: Preparation of Compound 2.2

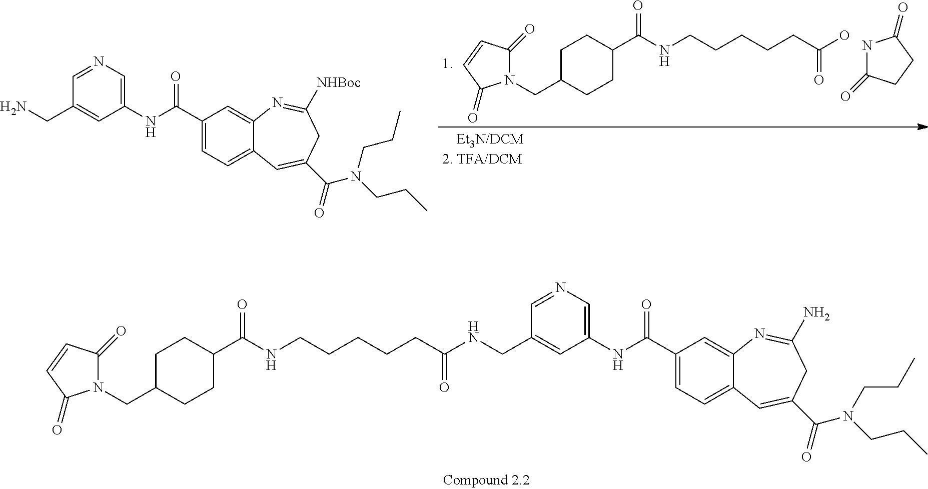

50 mg (0.11 mmol) of N-succinimidyl 6-[[4-(maleimidomethyl)cyclohexyl]carboxamido] caproate (CAS No. 125559-00-4) was added to a solution containing 60 mg (0.11 mmol) of 2-amino-N⁸-(5-(aminomethyl)pyridin-3-yl)-N⁴,N⁴-dipropyl-3H-benzo[b]azepine-4,8-dicarboxamide in 2.0 mL of DCM and 15 μL (0.11 mmol) of triethylamine. The reaction mixture was stirred for 16 h and then purified directly by reverse phase chromatography (no TFA). The clean fractions were lyophilized to afford the desired product which was dissolved in 5 mL of DCM and treated with 1 mL of TFA at room temperature. The mixture was stirred for 2 h and then evaporated. The resulting residue was purified by reverse phase chromatography (no TFA) to afford 49 mg of Compound-Linker 2.2 as a white solid. ¹H NMR (CD₃OD) δ 8.78 (s, 1H), 8.25 (s, 2H), 7.70 (d, J=1.8 Hz, 1H), 7.58 (dd, J=1.8, 8.1 Hz, 1H), 7.46 (d, J=8.3 Hz, 1H), 6.91 (s, 1H), 6.77 (s, 2H), 4.42 (s, 2H), 3.43 (m, 4H), 3.13 (t, J=6.9 Hz, 2H), 2.85 (d, J=16.6 Hz, 1H), 2.29 (t, J=7.3 Hz, 2H), 2.05 (m, 1H), 1.8-1.6 (m, 12H), 1.51 (m, 1H), 1.37 (m, 4H), 1.11-0.84 (m, 9H). LCMS (M+H)=767.

### Example 8

### Synthesis of Linker-Modified Payloads (LP)

### Example 8A

### Preparation of 2-amino-N⁸-(5-((6-(4-((2,5-dioxo-2,5-dihydro-1H-pyrrol-1-yl)methyl)cyclohexane-1-carboxamido)hexanamido)methyl)pyridin-3-yl)-N⁴,N⁴-dipropyl-3H-benzo[b]azepine-4,8-dicarboxamide (Compound-Linker 2.3)

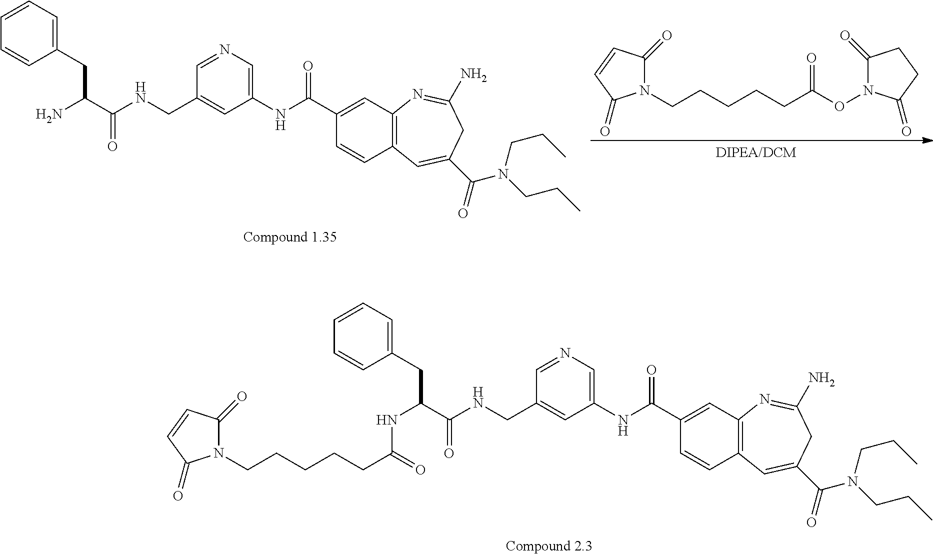

A solution containing 58 mg (0.10 mmol) of Compound 1.35 and 30 mg (0.1 mmol) of 2,5-dioxopyrrolidin-1-yl 6-(2,5-dioxo-2,5-dihydro-1H-pyrrol-1-yl)hexanoate in 2 mL of DCM was treated with 0.07 mL (0.4 mmol) of DIPEA and the reaction was stirred for 4 h at room temperature. The reaction mixture was purified without work-up by reverse phase chromatography to provide 28 mg of Compound-Linker 2.3 as a white solid. ¹H NMR (CD₃OD) δ 8.81 (d, J=2.3 Hz, 1H), 8.19 (d, J=1.9 Hz, 1H), 8.08 (t, J=2.1 Hz, 1H), 7.90 (m, 2H), 7.64 (dd, J=1.9, 8.1 Hz, 1H), 7.25-7.15 (m, 5H), 7.06 (s, 1H), 6.77 (s, 2H), 4.62-4.57 (m, 3H), 4.39 (s, 2H), 3.45-3.40 (m, 4H), 3.39 (t, J=7.5 Hz, 2H), 3.10 (m, 1H), 2.90 (m, 1H), 2.16 (t, J=7.5 Hz, 2H), 1.70 (m, 4H), 1.50 (m, 4H), 1.10 (m, 4H), 0.95 (m, 6H). LCMS (M+H)=775.8. The following compound-linkers 2.4 to 2.7 could be prepared in a manner similar to that described for Compound-Linker 2.3 above by reacting Compound 1.35 with an appropriately substituted linker group.

Compound-Linker 2.4

### (S)-2-amino-N⁸-(5-((2-(6-((4-(2,5-dioxo-2,5-dihydro-1H-pyrrol-1-yl)methyl)cyclohexane-1-carboxamido)hexanamido)-3-phenylpropanamido)methyl)pyridin-3-yl)-N⁴,N⁴-dipropyl-3H-benzo[b]azepine-4,8-dicarboxamide

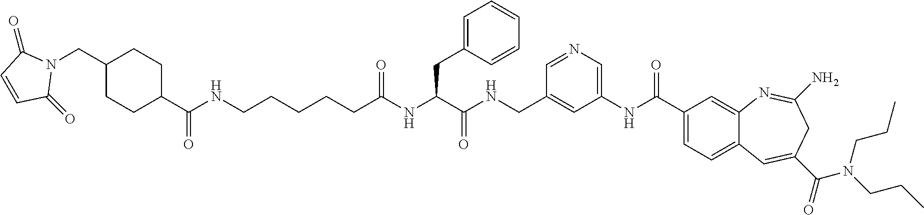 [SMILES: CCCN(CCC)C(=O)C1=Cc2ccc(C(=O)Nc3cncc(CNC(=O)[C@H](Cc4ccccc4)NC(=O)CCCCCNC(=O)C4CCC(CN5C(=O)C=CC5=O)CC4)c3)cc2N=C(N)C1]

From LC-smcc to afford a white solid. ¹H NMR (CD₃OD) δ 8.79 (d, J=2.0 Hz, 1H), 8.17 (d, J=2.0 Hz, 1H), 8.09 (t, J=2.0 Hz, 1H), 7.78 (s, 1H), 7.69 (m, 1H), 7.55 (m, 1H), 7.25-7.15 (m, 5H), 6.96 (s, 1H), 6.79 (s, 2H), 4.62-4.57 (m, 1H), 4.38 (s, 2H), 3.45-3.40 (m, 6H), 3.14 (m, 1H), 3.05 (t, J=7.5 Hz, 2H), 2.90 (m, 1H), 2.18 (t, J=7.5 Hz, 2H), 2.10 (m, 1H), 1.80-1.60 (m, 10H), 1.50-1.30 (m, 6H), 1.20-1.10 (m, 3H), 0.95 (m, 6H). LCMS (M+H)=914.9.

Compound-Linker 2.5

### (S)-2-amino-N⁸-(5-(4-benzyl-24-(2,5-dioxo-2,5-dihydro-1H-pyrrol-1-yl)-3,6,22-trioxo-9,12,15,18-tetraoxa-2,5,21-triazatetracosyl)pyridin-3-yl)-N⁴,N⁴-dipropyl-3H-benzo[b]azepine-4,8-dicarboxamide

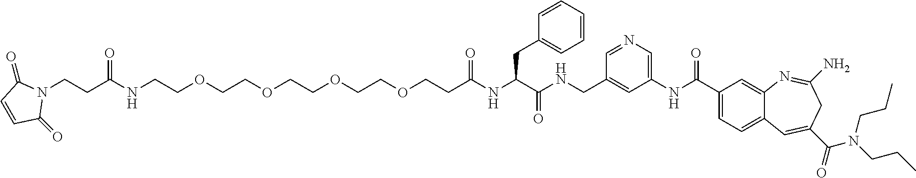 [SMILES: CCCN(CCC)C(=O)C1=Cc2ccc(C(=O)Nc3cncc(CNC(=O)[C@H](Cc4ccccc4)NC(=O)CCOCCOCCOCCOCCNC(=O)CCN4C(=O)C=CC4=O)c3)cc2N=C(N)C1]

From mal-PEG4-NHS to afford a white solid. ¹H NMR (CD₃OD) δ 8.91 (d, J=2.0 Hz, 1H), 8.24 (d, J=2.0 Hz, 1H), 8.15 (t, J=2.0 Hz, 1H), 8.01-7.98 (m, 2H), 7.72 (d, 8.0 Hz, 1H), 7.25-7.15 (m, 5H), 7.12 (s, 1H), 6.78 (s, 2H), 4.60 (m, 1H), 4.43 (s, 2H), 3.73 (t, J=7.5 Hz, 2H), 3.70-3.40 (m, 20H), 3.39 (s, 2H), 3.15 (m, 1H), 2.95 (m, 1H), 2.45 (t, J=7.5 Hz, 2H), 1.70 (q, J=7.5 Hz, 4H), 0.97-0.91 (m, 6H). LCMS (M+H)=980.9.

Compound-Linker 2.6

### (S)-2-amino-N⁸-(5-((2-(4-(4-(2,5-dioxo-2,5-dihydro-1H-pyrrol-1-yl)phenyl)butanamido)-3-phenylpropanamido)methyl)pyridin-3-yl)-N⁴,N⁴-dipropyl-3H-benzo[b]azepine-4,8-dicarboxamide, trifluoroacetate salt

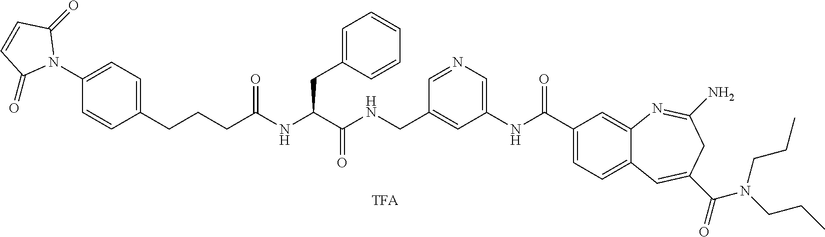 [SMILES: CCCN(CCC)C(=O)C1=Cc2ccc(C(=O)Nc3cncc(CNC(=O)[C@H](Cc4ccccc4)NC(=O)CCCc4ccc(N5C(=O)C=CC5=O)cc4)c3)cc2N=C(N)C1]

From SMPB NHS ester to afford a white solid. ¹H NMR (CD₃OD) δ 8.95 (d, J=2.0 Hz, 1H), 8.63 (d, J=2.0 Hz, 1H), 8.28 (s, 1H), 8.24 (m, 2H), 7.98 (m, 2H), 7.70 (d, J=9.0 Hz, 1H), 7.25-7.15 (m, 9H), 7.16 (s, 1H), 6.94 (s, 2H), 4.60 (m, 1H), 4.51-4.37 (m, 2H), 3.15 (m, 1H), 2.91 (m, 1H), 2.51 (t, J=7.5 Hz, 2H), 2.22 (m, 2H), 1.81 (t, J=7.5 Hz, 2H), 1.70 (q, J=7.5 Hz, 4H), 0.95 (m, 6H). LCMS (M+H)=823.8.

Compound-Linker 2.7

### 4-((S)-2-((S)-2-(6-(2,5-dioxo-2,5-dihydro-1H-pyrrol-1-yl)hexanamido)-3-methylbutanamido)-5-ureidopentanamido)benzyl ((S)-1-(((5-(2-amino-4-(dipropylcarbamoyl)-3H-benzo[b]azepine-8-carboxamido)pyridin-3-yl)methyl)amino)-1-oxo-3-phenylpropan-2-yl)carbamate

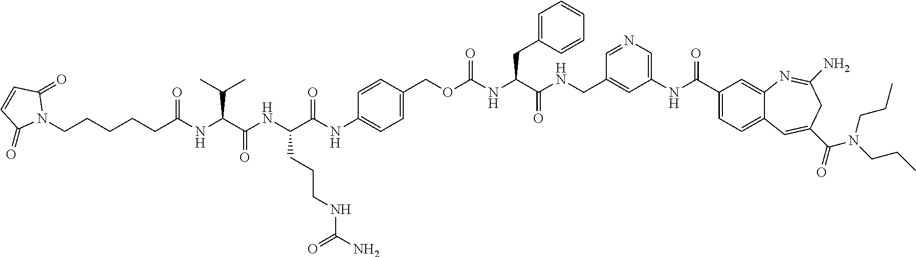 [SMILES: CCCN(CCC)C(=O)C1=Cc2ccc(C(=O)Nc3cncc(CNC(=O)[C@H](Cc4ccccc4)NC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1]

From mc-VC-PABA-PNP to afford a white solid. ¹H NMR (CD₃OD) δ 8.78 (s, 1H), 8.21 (s, 1H), 8.11 (s, 1H), 7.89 (m, 2H), 7.64 (dd, J=1.9, 8.1 Hz, 1H), 7.49 (d, J=8.0 Hz, 2H), 7.25-7.15 (m, 7H), 7.06 (s, 1H), 6.77 (s, 2H), 4.96 (s, 2H), 4.48 (m, 1H), 4.49-4.34 (m, 3H), 4.14 (d, J=7.5 Hz, 1H), 3.46-3.44 (m, 6H), 3.22 (m, 1H), 3.11 (m, 1H), 2.90 (m, 1H), 2.33-2.25 (m, 2H), 2.08 (m, 1H), 1.91 (m, 1H), 1.75-1.50 (m, 13H), 1.30 (m, 2H), 1.00-0.85 (m, 12H). LCMS (M+H)=1181.4.

Compound-Linker 2.8

### 4-((R)-2-((R)-2-(5-(2,5-dioxo-2,5-dihydro-1H-pyrrol-1-yl)pentanamido)-3-methylbutanamido)-5-ureidopentanamido)benzyl (2-(1-(5-(2-amino-4-(dipropylcarbamoyl)-3H-benzo[b]azepine-8-carboxamido)pyridin-2-yl)piperidine-4-carboxamido)ethyl)carbamate

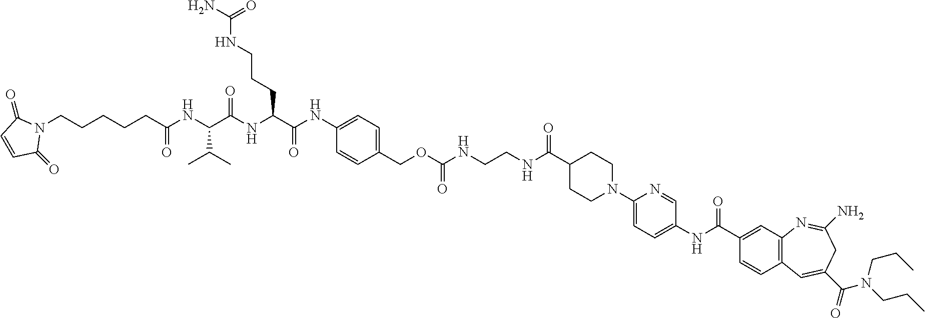 [SMILES: CCCN(CCC)C(=O)C1=Cc2ccc(C(=O)Nc3ccc(N4CCC(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)CC4)nc3)cc2N=C(N)C1]

From Compound 1.61 and mc-VC-PABA-PNP to afford a white solid. ¹H NMR (CD₃OD) δ 10.1 (s, 1H), 9.49 (s, 1H), 9.33 (bs, 2H), 7.88 (d, J=8.0 Hz, 1H), 7.80 (s, 1H), 7.64 (s, 1H), 7.61 (s, 1H), 7.45 (d, J=8.0 Hz, 1H), 7.35 (d, J=8.0 Hz, 1H), 7.02 (s, 1H), 6.85-6.80 (m, 2H), 6.75 (s, 1H), 4.25 m, 2H), 3.54-3.34 (m, 10H), 3.05 (s, 4H), 2.85-2.75 (m, 4H), 2.44 (m, 1H), 1.99 (m, 1H), 1.70-1.60 (m, 12H), 0.95 (bs, 6H).

Compound-Linker 2.9

### 4-((R)-2-((R)-2-(5-(2,5-dioxo-2,5-dihydro-1H-pyrrol-1-yl)pentanamido)-3-methylbutanamido)-5-ureidopentanamido)benzyl (1-(5-(2-amino-4-(dipropylcarbamoyl)-3H-benzo[b]azepine-8-carboxamido)pyridin-2-yl)piperidin-4-yl)carbamate

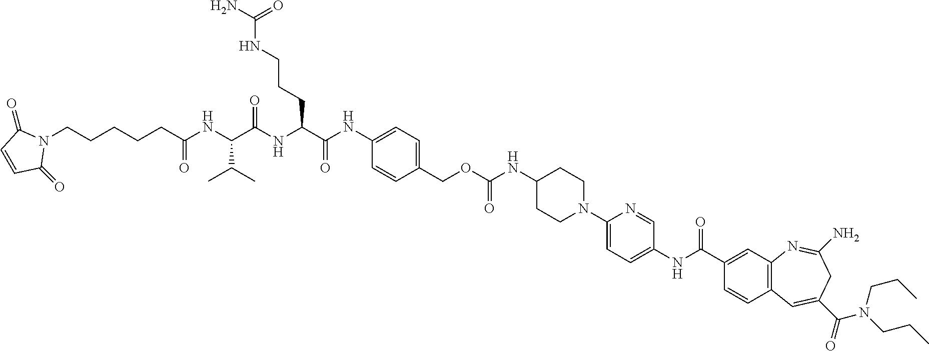 [SMILES: CCCN(CCC)C(=O)C1=Cc2ccc(C(=O)Nc3ccc(N4CCC(NC(=O)OCc5ccc(NC(=O)[C@H](CCCNC(N)=O)NC(=O)[C@@H](NC(=O)CCCCCN6C(=O)C=CC6=O)C(C)C)cc5)CC4)nc3)cc2N=C(N)C1]

From Compound 1.57 and mc-VC-PABA-PNP to afford a white solid. ¹H NMR (CD₃OD) δ 8.37 (d, J=2.5 Hz, 1H), 7.88 (dd, J=8.0, 2.5 Hz, 1H), 7.57-7.54 (m, 3H), 7.43 (d, J=8.0 Hz, 1H), 7.31 (d, J=8.0 Hz, 2H), 6.89 (s, 1H), 6.85-6.80 (m, 1H), 6.78 (s, 2H), 5.03 (s, 2H), 4.45 (m, 2H), 4.12 (m, 3H), 3.65 (m, 1H), 3.54 (t, J=7.5 Hz, 2H), 3.44 (m, 4H), 3.20-2.96 (m, 4H), 2.26 (t, J=7.5 Hz, 2H), 2.05 (m, 1H), 1.99-1.50 (m, 18H), 1.30 (m, 2H), 0.97 (t, J=7.5 Hz, 6H), 0.89 (bs, 6H).

Compound-Linker 2.20

### 4-((S)-2-((S)-2-(6-(2,5-dioxo-2,5-dihydro-1H-pyrrol-1-yl)hexanamido)-3-methylbutanamido)-5-ureidopentanamido)benzyl (2-(((5-(2-amino-4-(dipropylcarbamoyl)-3H-benzo[b]azepine-8-carboxamido)pyridin-3-yl)methyl)amino)-2-oxoethyl)carbamate

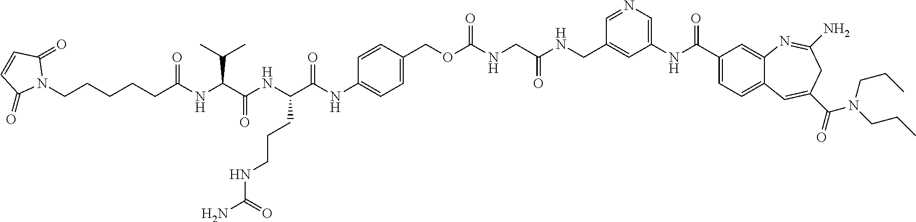 [SMILES: CCCN(CCC)C(=O)C1=Cc2ccc(C(=O)Nc3cncc(CNC(=O)CNC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1]

From Compound 1.64 and mc-VC-PABA-PNP to afford a white solid. ¹H NMR (CD₃OD) • 8.81 (s, 1H), 8.25 (s, 1H), 8.21 (s, 1H), 7.72 (s, 1H), 7.58 (m, 2H), 7.45 (d, J=8.2 Hz, 2H), 7.33 (d, J=8.4 Hz, 2H), 6.91 (s, 1H), 6.75 (s, 2H), 4.96 (s, 2H), 4.48 (m, 1H), 4.49-4.34 (m, 3H), 4.14 (d, J=7.5 Hz, 1H), 3.46-3.44 (m, 6H), 3.22 (m, 1H), 3.11 (m, 1H), 2.90 (m, 1H), 2.33-2.25 (m, 2H), 2.08 (m, 1H), 1.91 (m, 1H), 1.75-1.50 (m, 13H), 1.30 (m, 2H), 1.00-0.85 (m, 12H). LCMS (M+H)=1090.2.

Compound-Linker 2.21

### 2-amino-N8-(6-(4-((2-(4-((2,5-dioxo-2,5-dihydro-1H-pyrrol-1-yl)methyl)cyclohexane-1-carboxamido)ethyl)carbamoyl)piperidin-1-yl)pyridin-3-yl)-N4,N4-dipropyl-3H-benzo[b]azepine-4,8-dicarboxamide

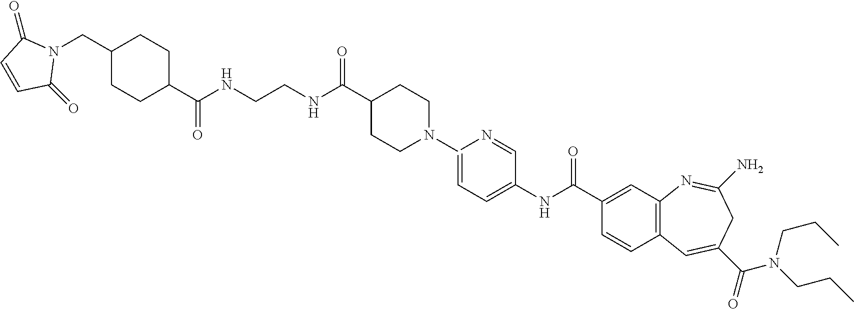 [SMILES: CCCN(CCC)C(=O)C1=Cc2ccc(C(=O)Nc3ccc(N4CCC(C(=O)NCCNC(=O)C5CCC(CN6C(=O)C=CC6=O)CC5)CC4)nc3)cc2N=C(N)C1]

From Compound 1.61 and succinimidyl 4-(N-maleimidomethyl)cyclohexane-1-carboxylate to provide a white solid. ¹H NMR (DMSO-d6) δ 10.1 (s, 1H), 8.46 (s, 1H), 8.61 (bs, 2H), 7.92 (dd, J=8.0, 2.5 Hz, 1H), 7.81 (m, 1H), 7.72 (m, 1H), 7.61 (s, 1H), 7.53 (d, J=8.0 Hz, 1H), 7.41 (d, J=8.0 Hz, 2H), 7.03 (s, 2H), 6.85-6.80 (m, 2H), 6.78 (s, 1H), 4.25 (m, 2H), 3.65 (m, 1H), 3.54 (t, J=7.5 Hz, 2H), 3.44 (m, 4H), 3.20-2.96 (m, 4H), 2.26 (t, J=7.5 Hz, 2H), 2.05 (m, 1H), 1.99-1.50 (m, 18H), 1.30 (m, 2H), 0.97 (t, J=7.5 Hz, 6H), 0.89 (bs, 6H). LCMS (M+H)=794.5.

### Example 8B

### Synthesis of 4-((S)-2-((S)-2-(6-(2,5-dioxo-2,5-dihydro-1H-pyrrol-1-yl)hexanamido)-3-methylbutanamido)-5-ureidopentanamido)benzyl (2-(4-((3-(2-amino-4-(dipropylcarbamoyl)-3H-benzo[b]azepine-8-carboxamido)-7,8-dihydro-1,6-naphthyridin-6(5H)-yl)methyl)benzamido)ethyl)carbamate (Compound-Linker 2.10)

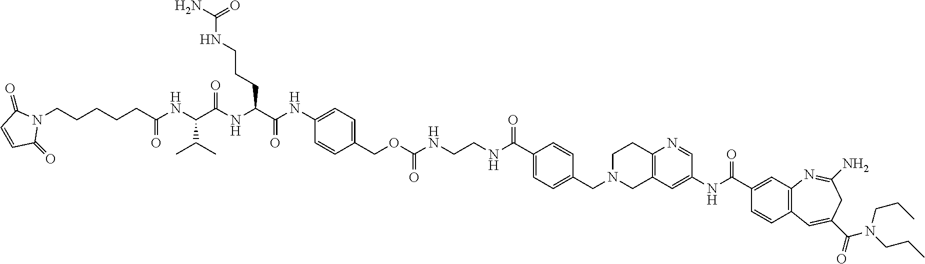 [SMILES: CCCN(CCC)C(=O)C1=Cc2ccc(C(=O)Nc3cnc4c(c3)CN(Cc3ccc(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)cc3)CC4)cc2N=C(N)C1]

Step A: Preparation of Int 7B-1

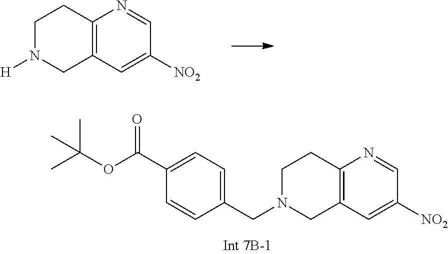 [SMILES: O=[N+]([O-])c1cnc2c(c1)CNCC2 ; CC(C)(C)OC(=O)c1ccc(CN2CCc3ncc([N+](=O)[O-])cc3C2)cc1]

To a stirred solution of 3-nitro-5,6,7,8-tetrahydro-1,6-naphthyridine dihydrochloride (1.0 g, 3.97 mmol) and tert-butyl 4-(bromomethyl)benzoate (1.18 g, 4.36 mmol) in DMF (40 mL) cooled in an ice-water bath was added dropwise TEA (2.76 mL, 19.8 mmol). The resulting clear solution was stirred overnight while cooling bath expired. LC-MS showed mostly desired product with small amount of SM remaining. The reaction mixture was concentrated in vacuo and the residue was diluted with water (45 mL) and saturated NaHCO₃ solution (5 mL) then extracted with EtOAc (3×). The combined extracts were dried (Na₂SO₄), filtered, and concentrated. The residue was absorbed on silica gel and purified by flash column chromatography (ISCO Gold 40 g; dry load, 0-20% CH₂Cl₂/MeOH) to afford 1.32 g of tert-butyl 4-((3-nitro-7,8-dihydro-1,6-naphthyridin-6(5H)-yl)methyl)benzoate as an orange colored syrup. ¹H NMR (DMSO-d⁶) δ 9.15 (d, J=2.5 Hz, 1H), 8.36 (d, J=2.5 Hz, 1H), 7.88 (d, J=8.0 Hz, 2H), 7.49 (d, J=8.0 Hz, 2H), 4.00 (s, 3H), 3.79 (s, 2H), 3.71 (s, 2H), 3.04 (m, 2H), 2.85 (m, 2H), 1.55 (s, 9H).

Step B: Preparation of Int 7B-2

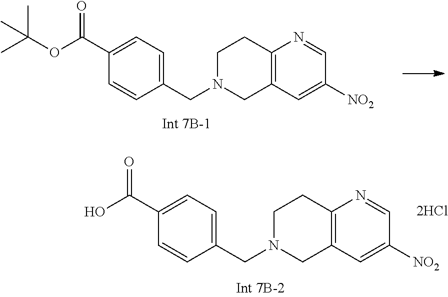 [SMILES: CC(C)(C)OC(=O)c1ccc(CN2CCc3ncc([N+](=O)[O-])cc3C2)cc1 ; O=C(O)c1ccc(CN2CCc3ncc([N+](=O)[O-])cc3C2)cc1]

To a stirred solution of tert-butyl 4-((3-nitro-7,8-dihydro-1,6-naphthyridin-6(5H)-yl)methyl)benzoate (1.32 g, 3.57 mmol) in 27 mL of DCM was added 4M HCl (9 mL, 36.0 mmol) in dioxane at room temperature. The reaction mixture was stirred for 3 h then concentrated under reduced pressure. The residue dried in vacuo to afford a light yellow solid which was used directly without further purification. ¹H NMR (CD₃OD) δ 9.33 (d, J=2.5 Hz, 1H), 8.53 (d, J=2.5 Hz, 1H), 8.19 (d, J=8.0 Hz, 2H), 7.72 (d, J=8.0 Hz, 2H), 4.82 (m, 2H), 4.66 (m, 2H), 4.61 (s, 2H), 3.44 (m, 2H).

Step C: Preparation of Int 7B-3

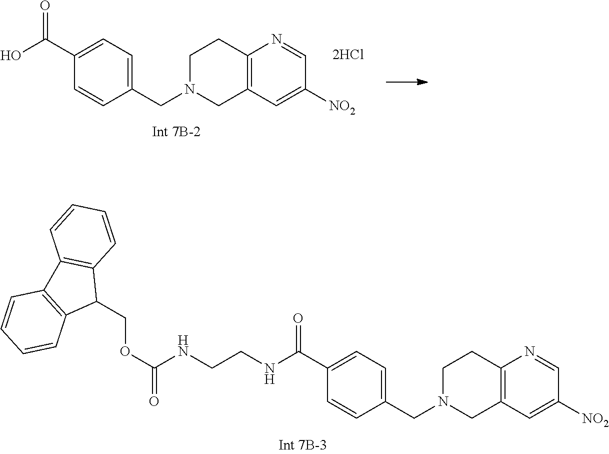 [SMILES: O=C(O)c1ccc(CN2CCc3ncc([N+](=O)[O-])cc3C2)cc1 ; O=C(NCCNC(=O)c1ccc(CN2CCc3ncc([N+](=O)[O-])cc3C2)cc1)OCC1c2ccccc2-c2ccccc21]

To a stirred solution of 4-((3-nitro-7,8-dihydro-1,6-naphthyridin-6(5H)-yl)methyl)benzoic acid dihydrochloride (1.28 g, 3.32 mmol), (9H-fluoren-9-yl)methyl (2-aminoethyl)carbamate hydrochloride (1.060 g, 3.32 mmol), and diisopropylethylamine (4.65 ml, 26.6 mmol) in 30 mL of DCM cooled in an ice-water bath was added dropwise 2,4,6-tripropyl-1,3,5,2,4,6-trioxatriphosphinane 2,4,6-trioxide (T3P®; 3.0 ml, 5.0 mmol). The mixture was stirred overnight while the cooling bath expired. The reaction mixture was partitioned between saturated NaHCO₃ and EtOAc. The aqueous layer was extracted with EtOAc (2×) and the combined organic extracts were washed with brine, dried over Na₂SO₄, filtered and concentrated to give 2.2 g of the desired product as an orange-red solid.

Step D: Preparation of Int 7B-4

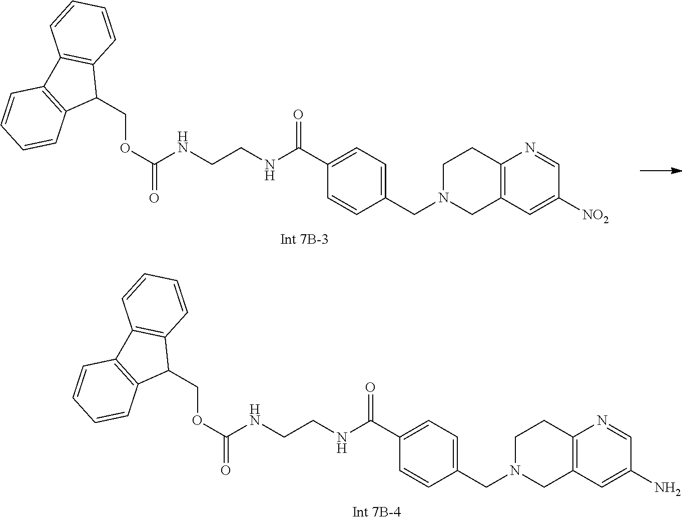 [SMILES: O=C(NCCNC(=O)c1ccc(CN2CCc3ncc([N+](=O)[O-])cc3C2)cc1)OCC1c2ccccc2-c2ccccc21 ; Nc1cnc2c(c1)CN(Cc1ccc(C(=O)NCCNC(=O)OCC3c4ccccc4-c4ccccc43)cc1)CC2]

A mixture of (9H-fluoren-9-yl)methyl (2-(4-((3-nitro-7,8-dihydro-1,6-naphthyridin-6(5H)-yl)methyl)benzamido)ethyl)carbamate (2.0 g, 3.5 mmol) and iron (1.930 g, 34.6 mmol) in acetic acid (30 mL)/water (3 mL) was stirred at 50° C. for 45 min. The reaction mixture was cooled to room temperature, filtered and concentrated. The residue was diluted with saturated NaHCO₃ (90 mL) and EtOAc (90 mL). The precipitate was collected, washed with water and EtOAc, and dried in vacuo to afford 1.9 g of a yellow-brown solid which was suspended in 1:1 CH₂Cl₂/MeOH and absorbed on silica gel. Purification by flash column chromatography (ISCO Gold 80 g; dry load, 0-50% B in CH₂Cl₂ gradient, B: 80:18:2 CH₂Cl₂/MeOH/conc. NH₄OH) gave 1.12 g of the desired product as an off-white solid.

Step E: Preparation of Int 7B-5

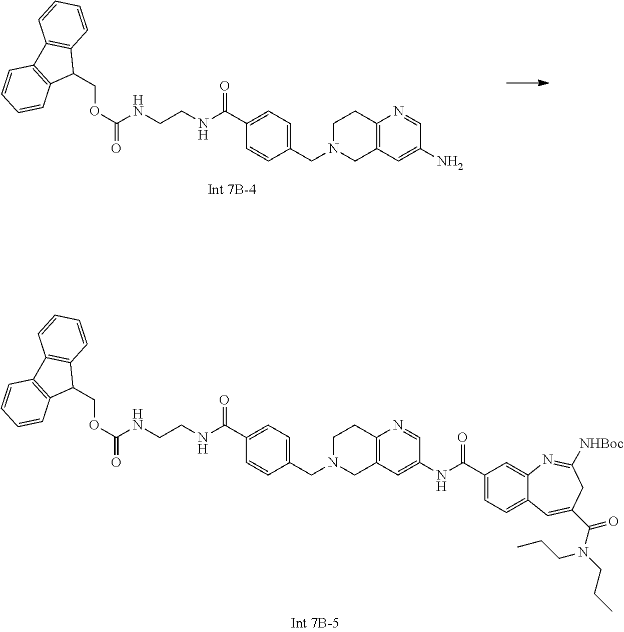 [SMILES: Nc1cnc2c(c1)CN(Cc1ccc(C(=O)NCCNC(=O)OCC3c4ccccc4-c4ccccc43)cc1)CC2 ; CCCN(CCC)C(=O)C1=Cc2ccc(C(=O)Nc3cnc4c(c3)CN(Cc3ccc(C(=O)NCCNC(=O)OCC5c6ccccc6-c6ccccc65)cc3)CC4)cc2N=C(NC(=O)OC(C)(C)C)C1]

To a stirred solution of 2-((tert-butoxycarbonyl)amino)-4-(dipropylcarbamoyl)-3H-benzo[b]azepine-8-carboxylic acid (350 mg, 0.815 mmol) in DMF (5 mL) at rt was added HATU (341 mg, 0.896 mmol). The reaction was stirred for 15 min before the addition of 669 mg (1.22 mmol) of (9H-fluoren-9-yl)methyl (2-(4-((3-amino-7,8-dihydro-1,6-naphthyridin-6(5H)-yl)methyl)benzamido)ethyl)carbamate in DMF (11 mL) was added. The reaction was stirred for 35 min before the addition of 0.427 mL (2.44 mmol) of Hunig's base. The resulting yellow solution was stirred for 18 h then concentrated in vacuo. The residue was purified by flash column chromatography (ISCO Gold 40 g; dry load, 0-50% B in CH₂Cl₂ gradient, B: 80:18:2 CH₂Cl₂/MeOH/conc. NH₄OH) to afford 435 mg of the desired product as a light yellow solid.

Step F: Preparation of Int 7B-6

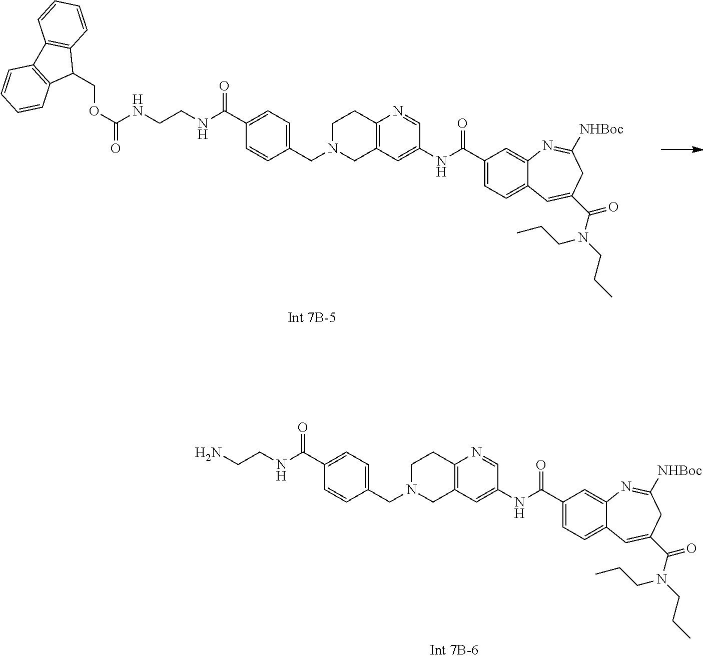 [SMILES: CCCN(CCC)C(=O)C1=Cc2ccc(C(=O)Nc3cnc4c(c3)CN(Cc3ccc(C(=O)NCCNC(=O)OCC5c6ccccc6-c6ccccc65)cc3)CC4)cc2N=C(NC(=O)OC(C)(C)C)C1 ; CCCN(CCC)C(=O)C1=Cc2ccc(C(=O)Nc3cnc4c(c3)CN(Cc3ccc(C(=O)NCCN)cc3)CC4)cc2N=C(NC(=O)OC(C)(C)C)C1]

To a stirred solution of tert-butyl (8-((6-(4-((2-((((9H-fluoren-9-yl)methoxy)carbonyl)amino)ethyl)carbamoyl)benzyl)-5,6,7,8-tetrahydro-1,6-naphthyridin-3-yl)carbamoyl)-4-(dipropylcarbamoyl)-3H-benzo[b]azepin-2-yl)carbamate (435 mg, 0.454 mmol) in 3.6 mL of DMF was added 0.90 mL (9.1 mmol) of piperidine at room temperature. The reaction was stirred for 1 h then concentrated. The residue was purified by flash column chromatography (ISCO Gold 24 g, 0-50% B in CH₂Cl₂ gradient, B: 80:18:2 CH₂Cl₂/MeOH/conc. NH₄OH) to afford 241 mg of the desired product as a light yellow solid.

Step G: Preparation of Compound-Linker 2.10

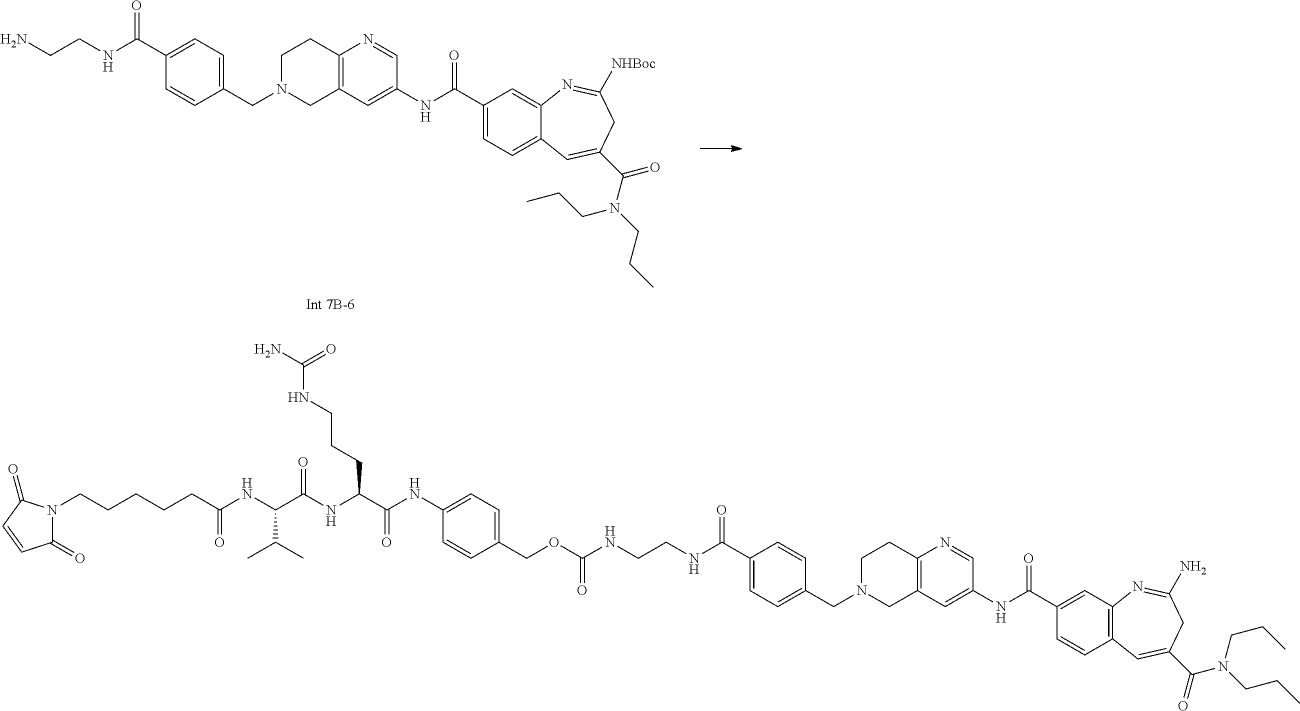 [SMILES: CCCN(CCC)C(=O)C1=Cc2ccc(C(=O)Nc3cnc4c(c3)CN(Cc3ccc(C(=O)NCCN)cc3)CC4)cc2N=C(NC(=O)OC(C)(C)C)C1 ; CCCN(CCC)C(=O)C1=Cc2ccc(C(=O)Nc3cnc4c(c3)CN(Cc3ccc(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)cc3)CC4)cc2N=C(N)C1]

To a stirred solution of tert-butyl (8-((6-(4-((2-aminoethyl)carbamoyl)benzyl)-5,6,7,8-tetrahydro-1,6-naphthyridin-3-yl)carbamoyl)-4-(dipropylcarbamoyl)-3H-benzo[b]azepin-2-yl)carbamate (80 mg, 0.109 mmol) and Hunig's base (0.057 mL, 0.326 mmol) in DMF (3.4 mL) under nitrogen cooled in an ice-water bath was added dropwise a solution of 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (80 mg, 0.109 mmol) in DMF (2 mL). The reaction was stirred overnight while cooling bath expired. The reaction mixture was then concentrated and the residue neutralized with saturated NaHCO₃ and purified by reverse phase column (Gold C18 30 g; 5-60% CH₃CN in water, no TFA). Fractions pooled, concentrated to afford 100 mg of an off-yellow solid which was directly dissolved in 50 mL of DCM and treated with 10 mL of TFA. The resulting solution was stirred for 1 h then concentrated under reduced pressure. The residue was dried in vacuo, neutralized with saturated NaHCO₃, and purified by reverse phase column chromatography (ISCO Gold C18 30 g; 5-70% MeCN in water gradient, no TFA). Major fractions were combined and lyophilized to provide 22 mg of an off-white solid. ¹H NMR (CD₃OD) δ 8.67 (d, J=2.5 Hz, 1H), 7.91 (d, J=2.5 Hz, 1H), 7.80 (d, J=8.0 Hz, 1H), 7.69 (d, J=2.5 Hz, 1H), 7.58-7.50 (m, 5H), 7.45 (d, J=8.0 Hz, 1H), 7.26 (d, J=8.5 Hz, 2H), 6.89 (s, 1H), 6.77 (s, 2H), 5.04 (s, 2H), 4.90 (m, 1H), 4.14 (d, J=7.5 Hz, 1H), 3.81 (s, 2H), 3.69 (s, 2H), 3.51-3.40 (m, 8H), 3.34 (m, 2H), 3.22 (m, 1H), 3.11 (m, 2H), 2.97 (m, 2H), 2.90 (m, 3H), 2.25 (t, J=7.5 Hz, 2H), 2.06 (m, 1H), 1.88 (m, 1H), 1.75-1.52 (m, 12H), 1.28 (m, 2H), 0.95 (t, J=7.5 Hz, 6H), 0.89 (bs, 6H). LCMS (M+H)=1235.9.

### Example 8C

### Synthesis of 4-((S)-2-((S)-2-(6-(2,5-dioxo-2,5-dihydro-1H-pyrrol-1-yl)hexanamido)-3-methylbutanamido)-5-ureidopentanamido)benzyl (2-(4-((3-(2-amino-4-(dipropylcarbamoyl)-3H-benzo[b]azepine-8-carboxamido)-7,8-dihydro-1,6-naphthyridin-6(5H)-yl)methyl)benzamido)ethyl)carbamate (Compound-Linker 2.11)

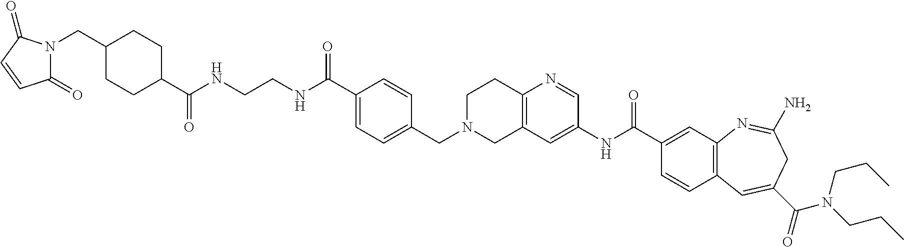 [SMILES: CCCN(CCC)C(=O)C1=Cc2ccc(C(=O)Nc3cnc4c(c3)CN(Cc3ccc(C(=O)NCCNC(=O)C5CCC(CN6C(=O)C=CC6=O)CC5)cc3)CC4)cc2N=C(N)C1]

Step A: Preparation of Compound-Linker 2.11

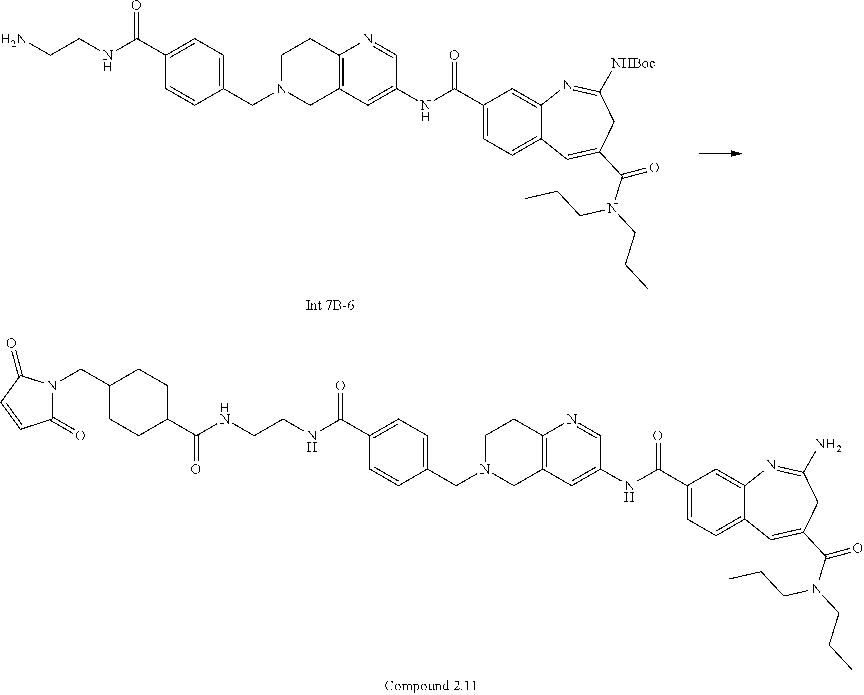 [SMILES: CCCN(CCC)C(=O)C1=Cc2ccc(C(=O)Nc3cnc4c(c3)CN(Cc3ccc(C(=O)NCCN)cc3)CC4)cc2N=C(NC(=O)OC(C)(C)C)C1 ; CCCN(CCC)C(=O)C1=Cc2ccc(C(=O)Nc3cnc4c(c3)CN(Cc3ccc(C(=O)NCCNC(=O)C5CCC(CN6C(=O)C=CC6=O)CC5)cc3)CC4)cc2N=C(N)C1]

A solution of 84.5 mg (0.115 mmol) of tert-butyl (8-((6-(4-((2-aminoethyl)carbamoyl)benzyl)-5,6,7,8-tetrahydro-1,6-naphthyridin-3-yl)carbamoyl)-4-(dipropylcarbamoyl)-3H-benzo[b]azepin-2-yl)carbamate from step F above, 2,5-dioxopyrrolidin-1-yl 4-((2,5-dioxo-2,5-dihydro-1H-pyrrol-1-yl)methyl)cyclohexane-1-carboxylate (38.3 mg, 0.115 mmol), and Hunig's base (0.040 mL, 0.229 mmol) in DCM (2.5 mL) was stirred at rt for 16 h. The reaction mixture was concentrated to dryness and the residue was purified by reverse phase column chromatography (ISCO Gold C18 100 g, 5-70% MeCN in water gradient, no TFA). The desired fractions were pooled and concentrated to provide 79 mg of the desired product as a yellow solid which was subsequently dissolved in 2.5 mL of DCM at rt then treated with TFA (500 μL, 6.49 mmol). After 1 h, the reaction mixture was concentrated, the residue dried in vacuo, neutralized with saturated NaHCO₃, and purified by reverse phase column chromatography (ISCO Gold C18 100 g; 5-60% MeCN in water gradient, no TFA). The main fractions were pooled and concentrated. The residue was lyophilized from MeCN/water to afford 25 mg of the desired product as an off-white solid. ¹H NMR (CD₃OD) δ 8.67 (d, J=2.5 Hz, 1H), 7.91 (d, J=2.5 Hz, 1H), 7.80 (d, J=8.0 Hz, 1H), 7.68 (d, J=2.5 Hz, 1H), 7.55 (dd, J=2.0, 8.0 Hz, 1H), 7.53 (d, J=8.0 Hz, 2H), 7.45 (d, J=8.0 Hz, 1H), 6.89 (s, 1H), 6.77 (s, 2H), 4.57 (s, 1H), 3.81 (s, 2H), 3.49-3.38 (m, 8H), 3.00 (m, 2H), 2.90 (m, 2H), 2.84 (m, 1H), 2.11 (m, 1H), 1.88 (m, 1H), 1.70-1.58 (m, 8H), 1.39 (m, 2H), 1.0-0.89 (m, 10H). LCMS (M+H)=856.8.

### Example 8D

### Synthesis of Perfluorophenyl 4-((3-((2-(4-((3-(2-amino-4-(dipropylcarbamoyl)-3H-benzo[b]azepine-8-carboxamido)-7,8-dihydro-1,6-naphthyridin-6(5H)-yl)methyl)benzamido)ethyl)thio)-2,5-dioxopyrrolidin-1-yl)methyl)cyclohexane-1-carboxylate tris TFA salt (Compound-Linker 2.12)

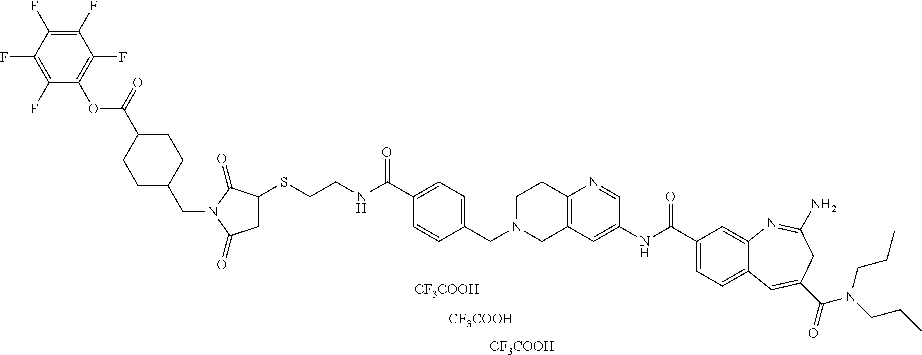 [SMILES: CCCN(CCC)C(=O)C1=Cc2ccc(C(=O)Nc3cnc4c(c3)CN(Cc3ccc(C(=O)NCCSC5CC(=O)N(CC6CCC(C(=O)Oc7c(F)c(F)c(F)c(F)c7F)CC6)C5=O)cc3)CC4)cc2N=C(N)C1 ; "CF3COOH" ; "CF3COOH" ; "CF3COOH"]

Preparation of Compound-Linker 2.12

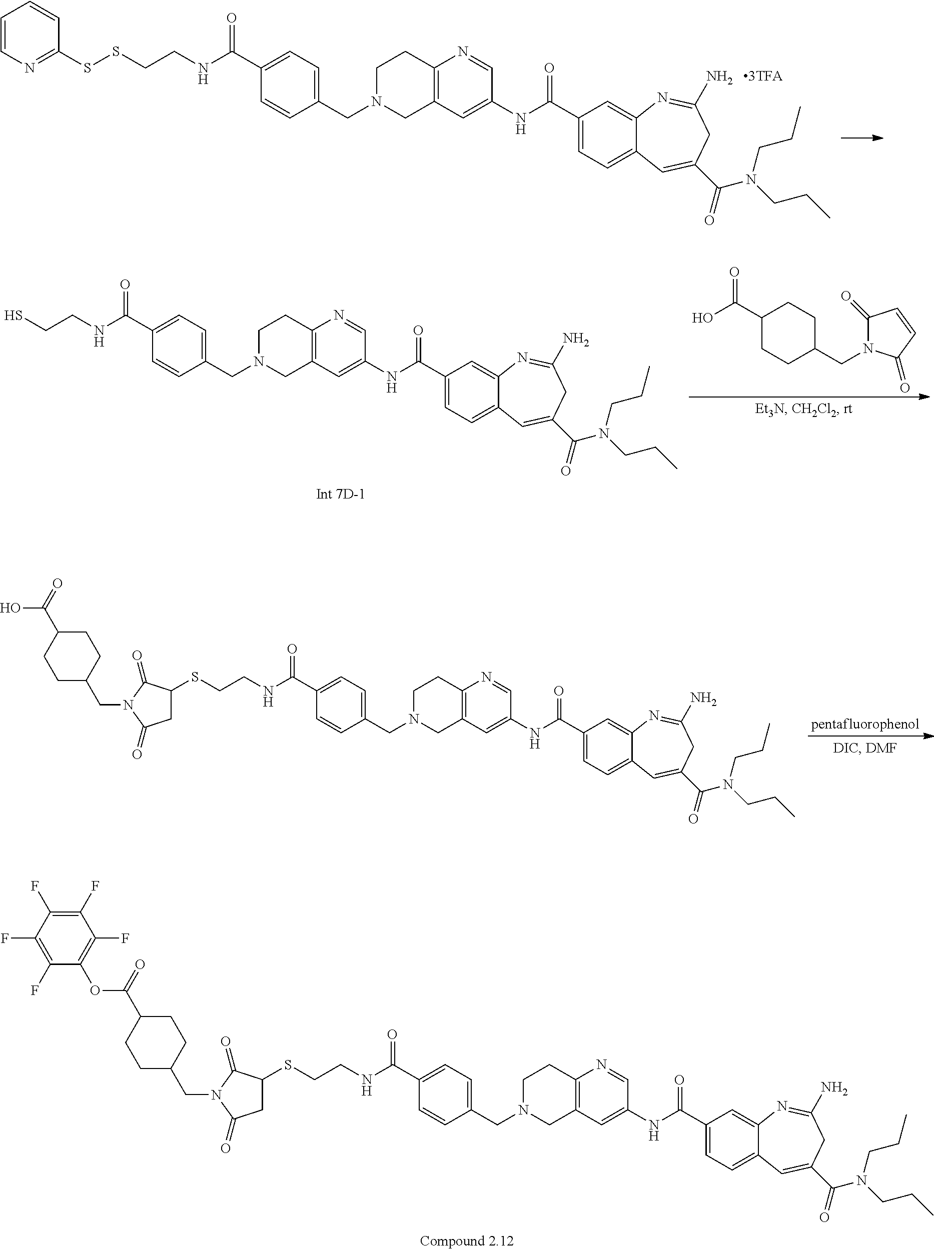 [SMILES: CCCN(CCC)C(=O)C1=Cc2ccc(C(=O)Nc3cnc4c(c3)CN(Cc3ccc(C(=O)NCCSSc5ccccn5)cc3)CC4)cc2N=C(N)C1 ; CCCN(CCC)C(=O)C1=Cc2ccc(C(=O)Nc3cnc4c(c3)CN(Cc3ccc(C(=O)NCCS)cc3)CC4)cc2N=C(N)C1 ; O=C(O)C1CCC(CN2C(=O)C=CC2=O)CC1 ; CCCN(CCC)C(=O)C1=Cc2ccc(C(=O)Nc3cnc4c(c3)CN(Cc3ccc(C(=O)NCCSC5CC(=O)N(CC6CCC(C(=O)O)CC6)C5=O)cc3)CC4)cc2N=C(N)C1 ; CCCN(CCC)C(=O)C1=Cc2ccc(C(=O)Nc3cnc4c(c3)CN(Cc3ccc(C(=O)NCCSC5CC(=O)N(CC6CCC(C(=O)Oc7c(F)c(F)c(F)c(F)c7F)CC6)C5=O)cc3)CC4)cc2N=C(N)C1]

A solution of 2-amino-N⁴,N⁴-dipropyl-N⁸-(6-(4-((2-(pyridin-2-yldisulfanyl)ethyl)carbamoyl)benzyl)-5,6,7,8-tetrahydro-1,6-naphthyridin-3-yl)-3H-benzo[b]azepine-4,8-dicarboxamide (100 mg, 0.090 mmol) (tri-TFA salt) and 3,3′,3″-phosphanetriyltripropionic acid hydrochloride (38.9 mg, 0.136 mmol) in 3 mL of 1:1 acetonitrile/water was stirred at room temperature for 0.5 h. The reaction mixture was concentrated in vacuo to dryness to provide Int 7D-1 a yellow foamy solid which was used directly without further any purification. This intermediate was converted to the final Compound-Linker 2.12 according to the scheme above. ¹H NMR (CD₃OD) δ 8.77 (d, J=2.0 Hz, 1H), 8.22 (d, J=2.5 Hz, 1H), 8.00-7.95 (m, 3H), 7.10 (s, 1H), 4.57 (bs, 2H), 4.47 (bs, 2H), 4.11 (dd, J=9.0, 3.5 Hz, 1H), 3.76-3.62 (m, 3H), 3.45-3.35 (m, 4H), 3.40-3.35 (m, 4H), 3.24-3.18 (m, 4H), 2.98 (m, 1H), 2.71 (m, 1H), 2.54 (d, J=3.5 Hz, 0.5H), 2.50 (d, J=3.5 Hz, 0.5H), 2.15 (m, 2H), 1.83-1.79 (m, 2H), 1.74-1.64 (m, 6H), 1.55-1.45 (m, 2H), 1.17-1.10 (m, 2H), 0.96 (bs, 3H), 0.91 (bs, 3H). LCMS (M+H)=1057.7.

### Example 8E

### Preparation of 4-((S)-2-((S)-2-(6-(2,5-dioxo-2,5-dihydro-1H-pyrrol-1-yl)hexanamido)-3-methylbutanamido)-5-ureidopentanamido)benzyl 3-(2-amino-4-(dipropylcarbamoyl)-7-methoxy-3H-benzo[b]azepine-8-carboxamido)-7,8-dihydro-1,6-naphthyridine-6(5H)-carboxylate (Compound-Linker 2.14)

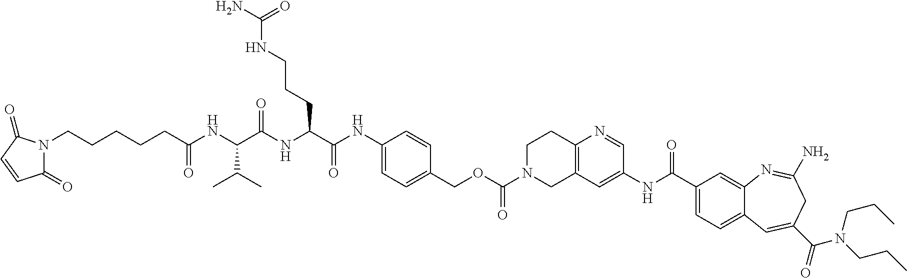 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1]

Prepared in a manner similar to Compound 1.1 using 2-amino-4-(dipropylcarbamoyl)-3H-benzo[b]azepine-8-carboxylic acid and commercially available tert-Butyl 3-amino-7,8-dihydro-1,6-naphthyridine-6(5H)-carboxylate (CAS No. 355819-02-2).

¹H NMR (DMSO-d⁶) δ 12.1 (s, 1H), 10.4 (s, 1H), 10.0 (s, 1H), 9.14 (s, 1H), 8.73 (d, J=2.4 Hz, 1H), 8.08 (d, J=7.6 Hz, 2H), 7.80 (d, J=8.8 Hz, 1H), 7.70 (s, 1H), 7.60 (d, J=8.4 Hz, 2H), 7.41 (s, 1H), 7.34 (d, J=8.8 Hz, 2H), 7.03 (s, 1H), 7.00 (s, 1H), 5.99 (bs, 1H), 5.07 (s, 2H), 4.65 (m, 4H), 4.40 (m, 2H), 4.21 (m, 2H), 3.97 (s, 3H), 3.74 (bt, 2H), 3.37 (t, J=6.8 Hz, 5H), 3.29 (s, 2H), 3.11-2.95 (m, 4H), 2.22-1.95 (m, 4H), 1.60-1.15 (m, 12H), 0.88 (d, J=7.0 Hz, 6H), 0.82 (d, J=7.0 Hz, 6H). LCMS [M+H]=1090.5.

### Example 8F

### Preparation of 4-((S)-2-((S)-2-(6-(2,5-dioxo-2,5-dihydro-1H-pyrrol-1-yl)hexanamido)-3-methylbutanamido)-5-ureidopentanamido)benzyl 3-(2-amino-4-(dipropylcarbamoyl)-7-methoxy-3H-benzo[b]azepine-8-carboxamido)-7,8-dihydro-1,6-naphthyridine-6(5H)-carboxylate (Compound-Linker 2.15)

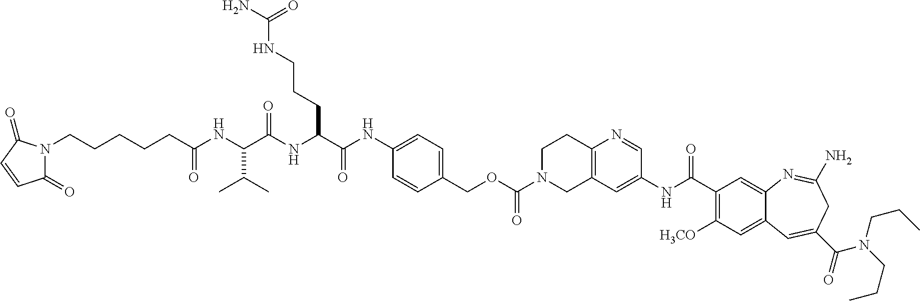 [SMILES: CCCN(CCC)C(=O)C1=Cc2cc(OC)c(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1]

Prepared in a manner similar to Compound 1.1 starting from 2-amino-4-(dipropylcarbamoyl)-7-methoxy-3H-benzo[b]azepine-8-carboxylic acid.

### Example 8G

### Preparation of 4-((S)-2-((S)-2-(6-(2,5-dioxo-2,5-dihydro-1H-pyrrol-1-yl)hexanamido)-3-methylbutanamido)-5-ureidopentanamido)benzyl 3-(2-amino-4-(dipropylcarbamoyl)-7-fluoro-3H-benzo[b]azepine-8-carboxamido)-7,8-dihydro-1,6-naphthyridine-6(5H)-carboxylate (Compound-Linker 2.16)

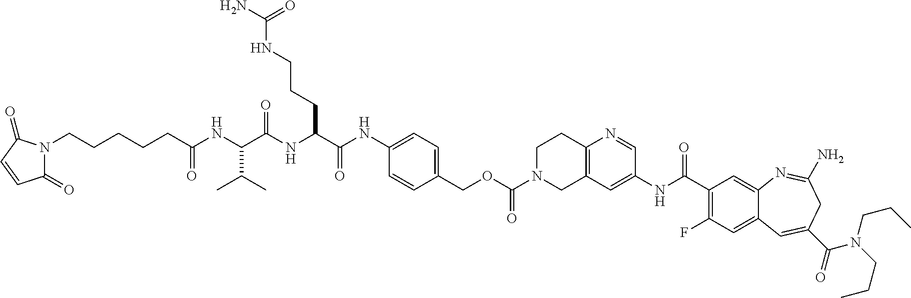 [SMILES: CCCN(CCC)C(=O)C1=Cc2cc(F)c(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1]

Prepared in a manner similar to Compound 1.1 starting from 2-amino-4-(dipropylcarbamoyl)-7-fluoro-3H-benzo[b]azepine-8-carboxylic acid.

¹H NMR (DMSO-d⁶) δ 12.2 (s, 1H), 10.8 (s, 1H), 10.0 (s, 1H), 9.89 (s, 1H), 9.27 (s, 1H), 8.66 (s, 1H), 8.08 (d, J=2.4 Hz, 1H), 8.03 (s, 1H), 7.80 (d, J=8.8 Hz, 2H), 7.70-7.64 (m, 2H), 7.60 (d, J=8.4 Hz, 1H), 7.34 (d, J=8.8 Hz, 2H), 7.02 (s, 1H), 7.00 (s, 2H), 5.99 (bs, 1H), 5.07 (s, 2H), 4.65 (m, 4H), 4.40 (m, 2H), 4.21 (m, 2H), 3.73 (bt, 2H), 3.36 (m, 5H), 3.29 (s, 2H), 3.11-2.95 (m, 4H), 2.22-1.95 (m, 4H), 1.60-1.15 (m, 12H), 0.88 (d, J=7.0 Hz, 6H), 0.82 (d, J=7.0 Hz, 6H). LCMS [M+H]=1079.5.

### Example 8H

### Preparation of 4-((S)-2-((S)-2-(6-(2,5-dioxo-2,5-dihydro-1H-pyrrol-1-yl)hexanamido)-3-methylbutanamido)-5-ureidopentanamido)benzyl (3-(4-((3-(2-amino-4-(dipropylcarbamoyl)-3H-benzo[b]azepine-8-carboxamido)-7,8-dihydro-1,6-naphthyridin-6(5H)-yl)methyl)benzamido)-2,2-difluoropropyl)carbamate (Compound-Linker 2.17)

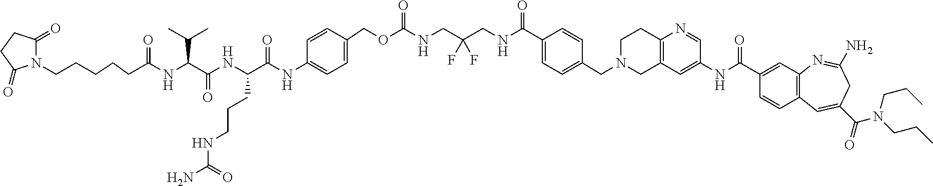 [SMILES: CCCN(CCC)C(=O)C1=Cc2ccc(C(=O)Nc3cnc4c(c3)CN(Cc3ccc(C(=O)NCC(F)(F)CNC(=O)OCc5ccc(NC(=O)[C@H](CCCNC(N)=O)NC(=O)[C@@H](NC(=O)CCCCCN6C(=O)CCC6=O)C(C)C)cc5)cc3)CC4)cc2N=C(N)C1]

Prepared in a manner similar to Compound 1.1.

Table 2 shows Compound-Linkers 2.1-2.21.

TABLE 2
Compound-Linkers 2.1-2.21

| Compound- | |
|---|---|
| Linkers | Structure |
| 2.1 | 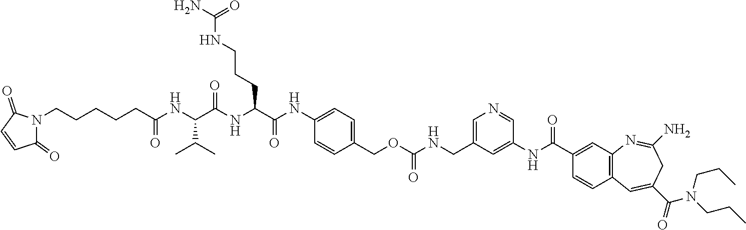 [SMILES: CCCN(CCC)C(=O)C1=Cc2ccc(C(=O)Nc3cncc(CNC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1] |
| 2.2 | 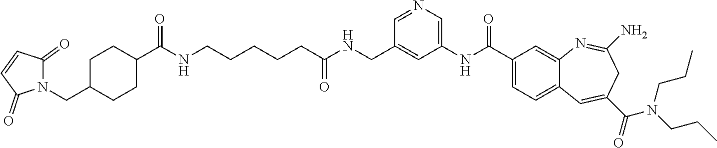 [SMILES: CCCN(CCC)C(=O)C1=Cc2ccc(C(=O)Nc3cncc(CNC(=O)CCCCCNC(=O)C4CCC(CN5C(=O)C=CC5=O)CC4)c3)cc2N=C(N)C1] |
| 2.3 | 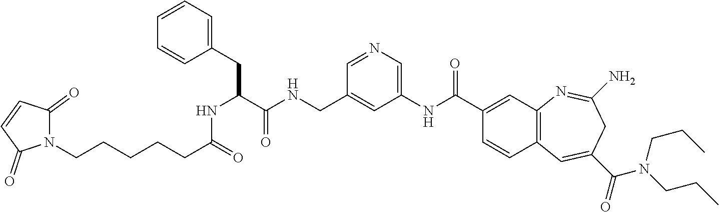 [SMILES: CCCN(CCC)C(=O)C1=Cc2ccc(C(=O)Nc3cncc(CNC(=O)[C@H](Cc4ccccc4)NC(=O)CCCCCN4C(=O)C=CC4=O)c3)cc2N=C(N)C1] |
| 2.4 | 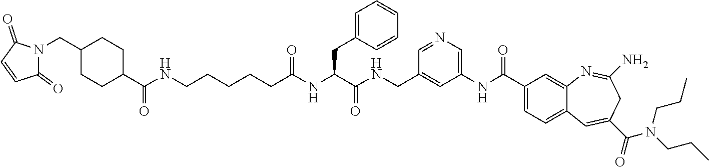 [SMILES: CCCN(CCC)C(=O)C1=Cc2ccc(C(=O)Nc3cncc(CNC(=O)[C@H](Cc4ccccc4)NC(=O)CCCCCNC(=O)C4CCC(CN5C(=O)C=CC5=O)CC4)c3)cc2N=C(N)C1] |
| 2.5 | 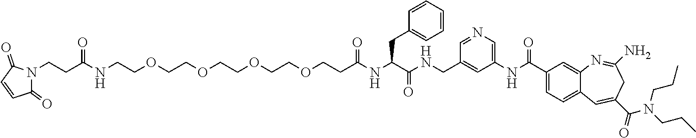 [SMILES: CCCN(CCC)C(=O)C1=Cc2ccc(C(=O)Nc3cncc(CNC(=O)[C@H](Cc4ccccc4)NC(=O)CCOCCOCCOCCOCCNC(=O)CCN4C(=O)C=CC4=O)c3)cc2N=C(N)C1] |
| 2.6 | 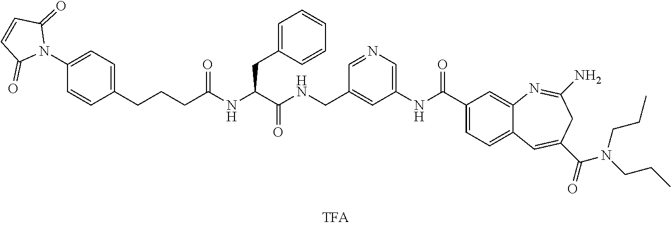 [SMILES: CCCN(CCC)C(=O)C1=Cc2ccc(C(=O)Nc3cncc(CNC(=O)[C@H](Cc4ccccc4)NC(=O)CCCc4ccc(N5C(=O)C=CC5=O)cc4)c3)cc2N=C(N)C1] |
| 2.7 | 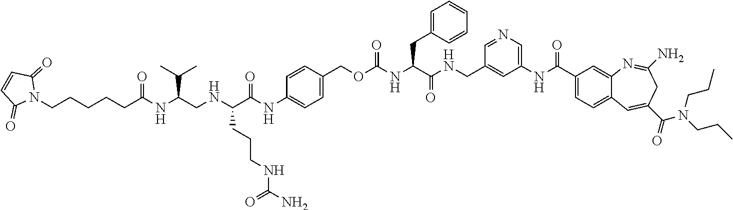 [SMILES: CCCN(CCC)C(=O)C1=Cc2ccc(C(=O)Nc3cncc(CNC(=O)[C@H](Cc4ccccc4)NC(=O)OCc4ccc(NC(=O)[C@H](CCCNC(N)=O)NC[C@@H](NC(=O)CCCCCN5C(=O)C=CC5=O)C(C)C)cc4)c3)cc2N=C(N)C1] |
| 2.8 | 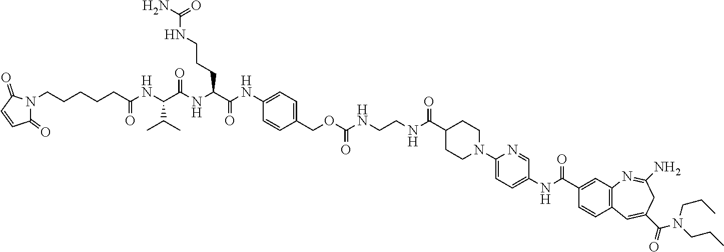 [SMILES: CCCN(CCC)C(=O)C1=Cc2ccc(C(=O)Nc3ccc(N4CCC(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)CC4)nc3)cc2N=C(N)C1] |
| 2.9 | 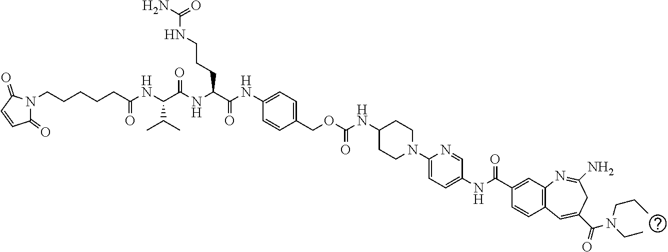 [SMILES: CCCN(CC)C(=O)C1=Cc2ccc(C(=O)Nc3ccc(N4CCC(NC(=O)OCc5ccc(NC(=O)[C@H](CCCNC(N)=O)NC(=O)[C@@H](NC(=O)CCCCCN6C(=O)C=CC6=O)C(C)C)cc5)CC4)nc3)cc2N=C(N)C1] |
| 2.10 | 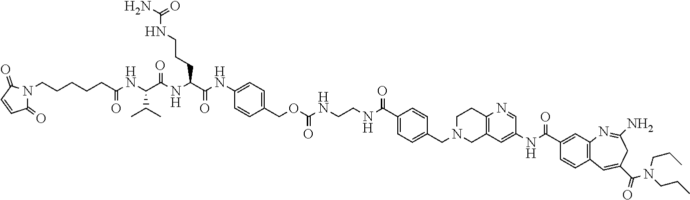 [SMILES: CCCN(CCC)C(=O)C1=Cc2ccc(C(=O)Nc3cnc4c(c3)CN(Cc3ccc(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)cc3)CC4)cc2N=C(N)C1] |
| 2.11 | 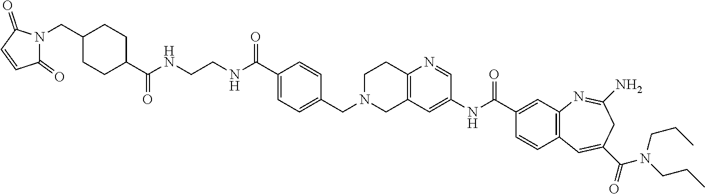 [SMILES: CCCN(CCC)C(=O)C1=Cc2ccc(C(=O)Nc3cnc4c(c3)CN(Cc3ccc(C(=O)NCCNC(=O)C5CCC(CN6C(=O)C=CC6=O)CC5)cc3)CC4)cc2N=C(N)C1] |
| 2.12 | 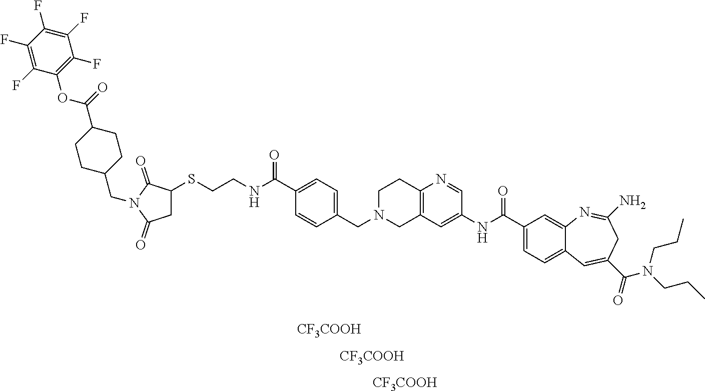 [SMILES: CCCN(CCC)C(=O)C1=Cc2ccc(C(=O)Nc3cnc4c(c3)CN(Cc3ccc(C(=O)NCCSC5CC(=O)N(CC6CCC(C(=O)Oc7c(F)c(F)c(F)c(F)c7F)CC6)C5=O)cc3)CC4)cc2N=C(N)C1 ; "CF3COOH" ; "CF3COOH" ; "CF3COOH"] |
| 2.14 | 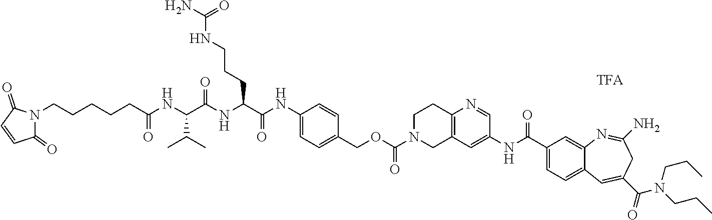 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1] |
| 2.15 | 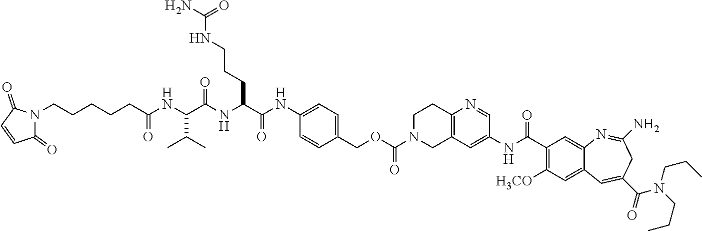 [SMILES: CCCN(CCC)C(=O)C1=Cc2cc(OC)c(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1] |
| 2.16 | 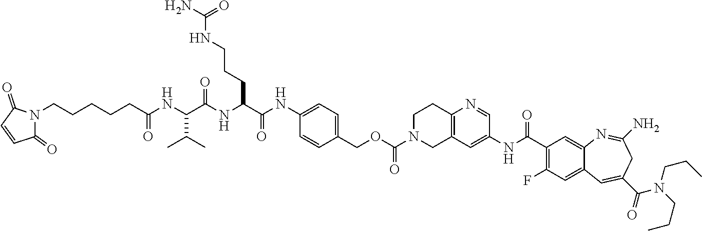 [SMILES: CCCN(CCC)C(=O)C1=Cc2cc(F)c(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1] |
| 2.17 | 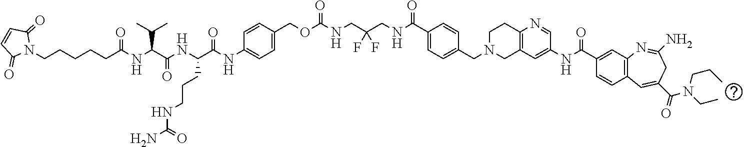 [SMILES: CCCN(CC)C(=O)C1=Cc2ccc(C(=O)Nc3cnc4c(c3)CN(Cc3ccc(C(=O)NCC(F)(F)CNC(=O)OCc5ccc(NC(=O)[C@H](CCCNC(N)=O)NC(=O)[C@@H](NC(=O)CCCCCN6C(=O)C=CC6=O)C(C)C)cc5)cc3)CC4)cc2N=C(N)C1] |
| 2.20 | 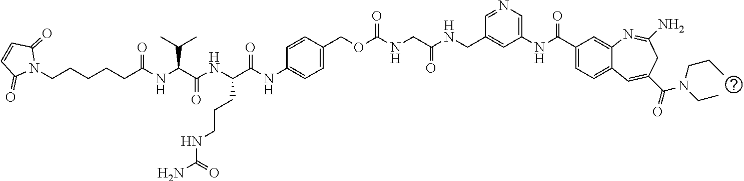 [SMILES: CCCN(CC)C(=O)C1=Cc2ccc(C(=O)Nc3cncc(CNC(=O)CNC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1] |
| 2.21 | 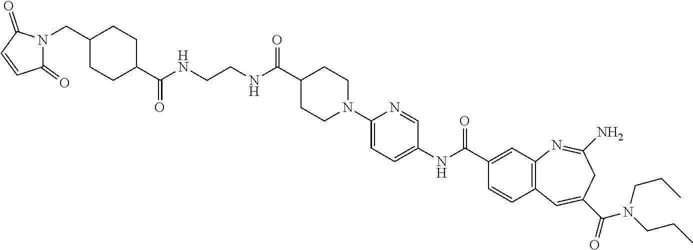 [SMILES: CCCN(CCC)C(=O)C1=Cc2ccc(C(=O)Nc3ccc(N4CCC(C(=O)NCCNC(=O)C5CCC(CN6C(=O)C=CC6=O)CC5)CC4)nc3)cc2N=C(N)C1] |
| 2.22 | 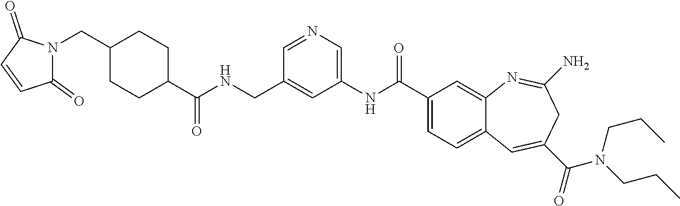 [SMILES: CCCN(CCC)C(=O)C1=Cc2ccc(C(=O)Nc3cncc(CNC(=O)C4CCC(CN5C(=O)C=CC5=O)CC4)c3)cc2N=C(N)C1] |

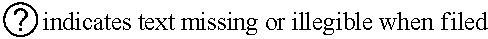

Table 3 shows Compound-Linkers 2.23-2.38 these compound linkers can be made using the methods described herein in combination with knowledge in the art.

TABLE 3
Compound-Linkers 2.23-2.38

| Compound- | |
|---|---|
| Linkers | Structure |
| 2.23 | 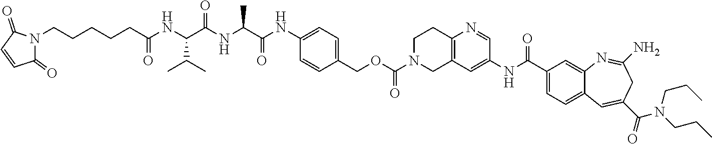 [SMILES: CCCN(CCC)C(=O)C1=Cc2ccc(C(=O)Nc3cnc4c(c3)CN(C(=O)OCc3ccc(NC(=O)[C@H](C)NC(=O)[C@@H](NC(=O)CCCCCN5C(=O)C=CC5=O)C(C)C)cc3)CC4)cc2N=C(N)C1] |
| 2.24 | 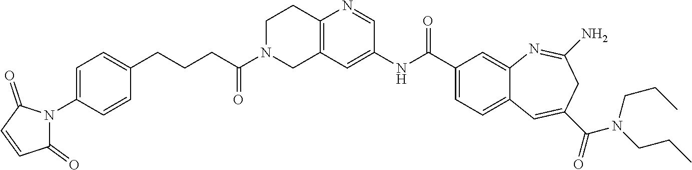 [SMILES: CCCN(CCC)C(=O)C1=Cc2ccc(C(=O)Nc3cnc4c(c3)CN(C(=O)CCCc3ccc(N5C(=O)C=CC5=O)cc3)CC4)cc2N=C(N)C1] |
| 2.25 | 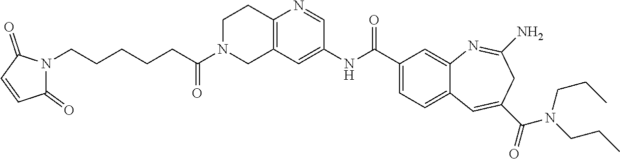 [SMILES: CCCN(CCC)C(=O)C1=Cc2ccc(C(=O)Nc3cnc4c(c3)CN(C(=O)CCCCCN3C(=O)C=CC3=O)CC4)cc2N=C(N)C1] |
| 2.26 | 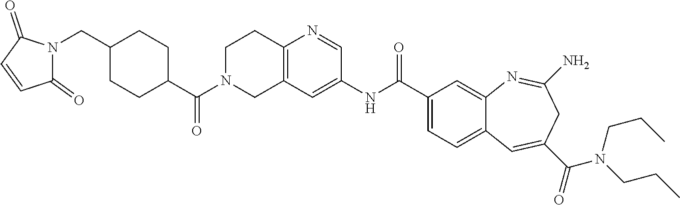 [SMILES: CCCN(CCC)C(=O)C1=Cc2ccc(C(=O)Nc3cnc4c(c3)CN(C(=O)C3CCC(CN5C(=O)C=CC5=O)CC3)CC4)cc2N=C(N)C1] |
| 2.27 | 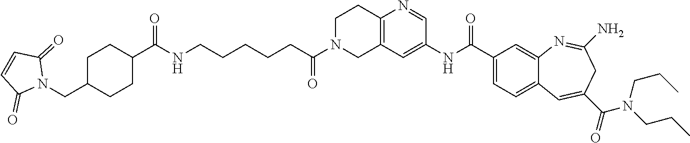 [SMILES: CCCN(CCC)C(=O)C1=Cc2ccc(C(=O)Nc3cnc4c(c3)CN(C(=O)CCCCCNC(=O)C3CCC(CN5C(=O)C=CC5=O)CC3)CC4)cc2N=C(N)C1] |
| 2.28 | 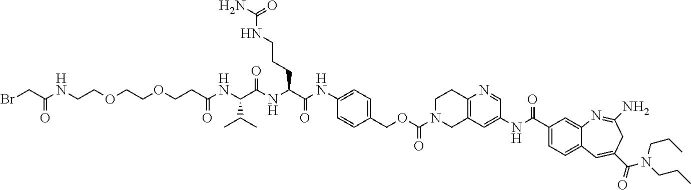 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCOCCOCCNC(=O)CBr)C(C)C)cc3)CC4)cc2N=C(N)C1] |
| 2.29 | 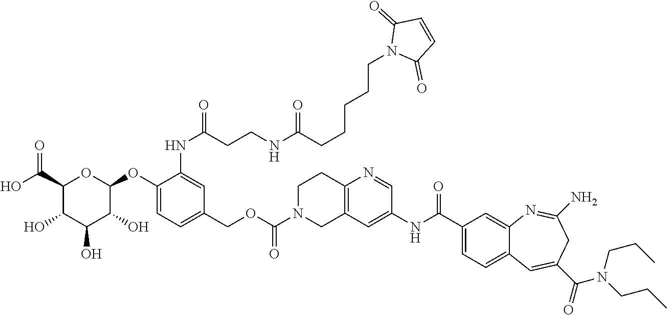 [SMILES: CCCN(CCC)C(=O)C1=Cc2ccc(C(=O)Nc3cnc4c(c3)CN(C(=O)OCc3ccc(O[C@@H]5O[C@H](C(=O)O)[C@@H](O)[C@H](O)[C@H]5O)c(NC(=O)CCNC(=O)CCCCCN5C(=O)C=CC5=O)c3)CC4)cc2N=C(N)C1] |
| 2.30 | 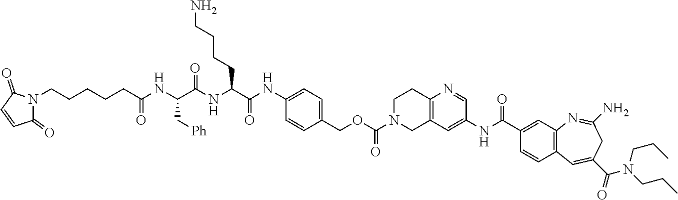 [SMILES: CCCN(CCC)C(=O)C1=Cc2ccc(C(=O)Nc3cnc4c(c3)CN(C(=O)OCc3ccc(NC(=O)[C@H](CCCCN)NC(=O)[C@H](Cc5ccccc5)NC(=O)CCCCCN5C(=O)C=CC5=O)cc3)CC4)cc2N=C(N)C1] |
| 2.31 | 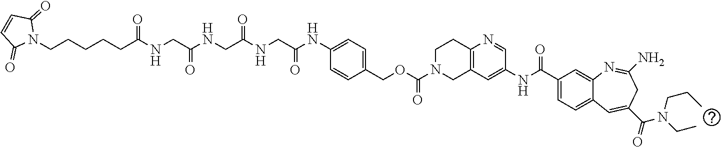 [SMILES: CCCN(CC)C(=O)C1=Cc2ccc(C(=O)Nc3cnc4c(c3)CN(C(=O)OCc3ccc(NC(=O)CNC(=O)CNC(=O)CNC(=O)CCCCCN5C(=O)C=CC5=O)cc3)CC4)cc2N=C(N)C1] |
| 2.32 | 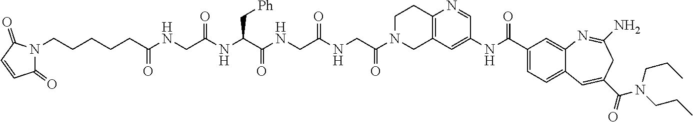 [SMILES: CCCN(CCC)C(=O)C1=Cc2ccc(C(=O)Nc3cnc4c(c3)CN(C(=O)CNC(=O)CNC(=O)[C@H](Cc3ccccc3)NC(=O)CNC(=O)CCCCCN3C(=O)C=CC3=O)CC4)cc2N=C(N)C1] |
| 2.33 | 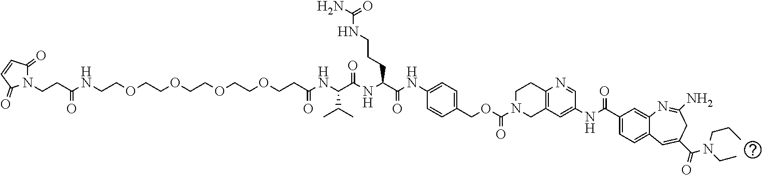 [SMILES: CCCN(CC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCOCCOCCOCCOCCNC(=O)CCN5C(=O)C=CC5=O)C(C)C)cc3)CC4)cc2N=C(N)C1] |
| 2.34 | 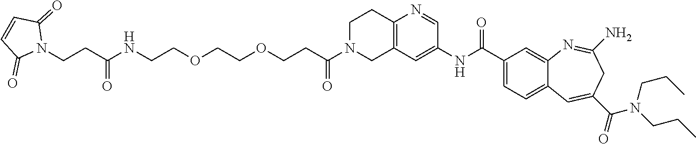 [SMILES: CCCN(CCC)C(=O)C1=Cc2ccc(C(=O)Nc3cnc4c(c3)CN(C(=O)CCOCCOCCNC(=O)CCN3C(=O)C=CC3=O)CC4)cc2N=C(N)C1] |
| 2.35 | 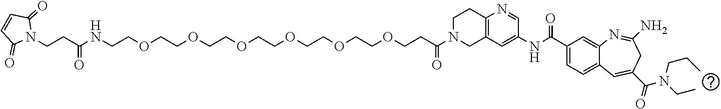 [SMILES: CCCN(CC)C(=O)C1=Cc2ccc(C(=O)Nc3cnc4c(c3)CN(C(=O)CCOCCOCCOCCOCCOCCOCCNC(=O)CCN3C(=O)C=CC3=O)CC4)cc2N=C(N)C1] |
| 2.36 | 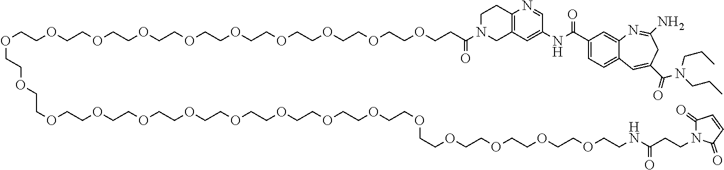 [SMILES: CCCN(CCC)C(=O)C1=Cc2ccc(C(=O)Nc3cnc4c(c3)CN(C(=O)CCOCCOCCOCCOCCOCCOCCOCCOCCOCCOCCOCCOCCOCCOCCOCCOCCOCCOCCOCCOCCOCCOCCOCCOCCNC(=O)CCN3C(=O)C=CC3=O)CC4)cc2N=C(N)C1] |
| 2.37 | 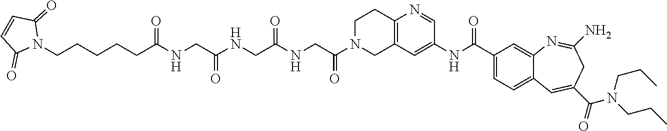 [SMILES: CCCN(CCC)C(=O)C1=Cc2ccc(C(=O)Nc3cnc4c(c3)CN(C(=O)CNC(=O)CNC(=O)CNC(=O)CCCCCN3C(=O)C=CC3=O)CC4)cc2N=C(N)C1] |
| 2.38 | 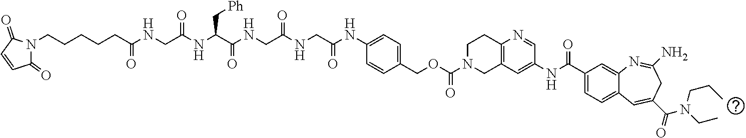 [SMILES: CCCN(CC)C(=O)C1=Cc2ccc(C(=O)Nc3cnc4c(c3)CN(C(=O)OCc3ccc(NC(=O)CNC(=O)CNC(=O)[C@H](Cc5ccccc5)NC(=O)CNC(=O)CCCCCN5C(=O)C=CC5=O)cc3)CC4)cc2N=C(N)C1] |

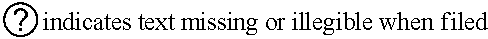

### Example 9

### Linking Antibody Constructs to Myeloid Cell Agonists Via a Linker

This example shows different methods of linking an antibody construct to a myeloid cell agonist via a linker to form a conjugate.

A linker, such as a maleimidocaproyl)-(valine-citrulline)-(para-aminobenzyloxycarbonyl) linker or disulfide linker (e.g., as disclosed in formulas Ig to Il) can be first attached to a myeloid cell agonist to form a myeloid cell agonist-linker compound. Subsequently, a myeloid cell agonist-linker is conjugated to an antibody construct.

A linker is attached to an antibody construct, in which the linker is a disulfide linker (e.g., in formula Ig-Il) or a hydrazone linker to form a linker-antibody construct. Subsequently, a myeloid cell agonist is conjugated to the linker linked with the antibody construct.

### Example 10

### Lysine-Based Bioconjugation

The antibody construct is exchanged into an appropriate buffer, for example, phosphate, borate, PBS, or Tris-Acetate, at a concentration of about 2 mg/mL to about 10 mg/mL. An appropriate number of equivalents of the myeloid cell agonist-linker are added as a solution with stirring. Dependent on the physical properties of the myeloid cell agonist-linker construct, a co-solvent can be introduced prior to the addition of the myeloid cell agonist-linker construct to facilitate solubility. The reaction is stirred at room temperature for 2 hours to about 12 hours depending on the observed reactivity. The progression of the reaction is monitored by LC-MS. Once the reaction is deemed complete, the remaining myeloid cell agonist-linker constructs are removed by applicable methods and the lysine-linked myeloid cell agonist conjugate is exchanged into the desired formulation buffer.

Lysine-linked conjugates are synthesized starting with 10 mg of antibody construct (mAb) and 10 equivalents of myeloid cell agonist-linker using the conditions described in Scheme 34 below (ADC=conjugate; ATAC=myeloid cell agonist-linker). Monomer content and drug-antibody ratios can be determined by methods described herein.

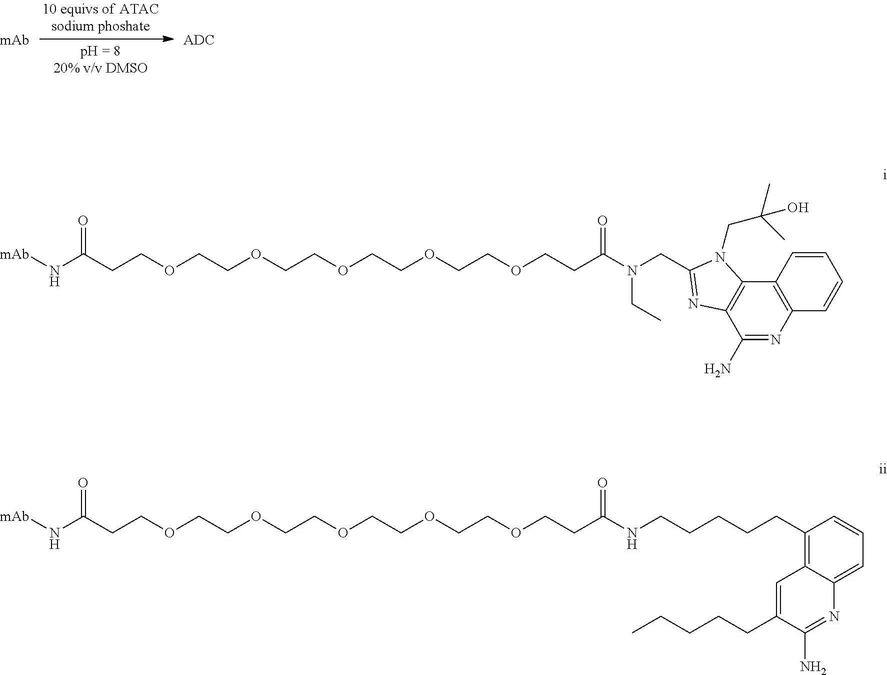

### Example 11

### Cysteine-Based Bioconjugation to Interchain Disulfides

The antibody is exchanged into an appropriate buffer, for example, phosphate, borate, PBS, or Tris-Acetate, at a concentration of about 2 mg/mL to about 10 mg/mL with an appropriate number of equivalents of a reducing agent, for example, dithiothreitol or tris(2-carboxyethyl)phosphine. The resultant solution is stirred for an appropriate amount of time and temperature to effect the desired reduction. The myeloid cell agonist-linker construct is added as a solution with stirring. Dependent on the physical properties of the myeloid cell agonist-linker construct, a co-solvent is introduced prior to the addition of the myeloid cell agonist-linker construct to facilitate solubility. The reaction is stirred at room temperature for about 1 hour to about 12 hours depending on the observed reactivity. The progression of the reaction is monitored by liquid chromatography-mass spectrometry (LC-MS). Once the reaction is deemed complete, the remaining free immune stimulatory compound-linker construct is removed by applicable methods and the conjugate is exchanged into the desired formulation buffer. Such cysteine-based conjugates are synthesized starting with 10 mg of antibody construct (mAb) and 7 equivalents of compound-linker using the conditions described in Scheme 35 below. Monomer content and drug-antibody ratios can be determined by methods described herein.

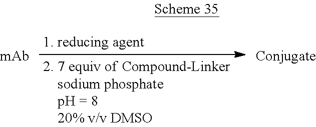

### Example 12

### Conjugation to Engineered/Site-Specific Cysteines

The antibody construct is exchanged into an appropriate buffer, for example, phosphate, HEPES, borate, PBS, or Tris-Acetate, at a concentration of about 2 mg/mL to about 10 mg/mL. To the solution is added an appropriate number of equivalents (10 to about 40) of an appropriate reducing agent, for example, dithiothreitol or tris(2-carboxyethyl)phosphine. The reaction is then incubated at room temperature for about 1 hour to about 12 hours depending on the observed reactivity. The reduced antibody construct is then exchanged into an appropriate buffer, for example, phosphate, HEPES, borate, PBS, or Tris-Acetate, at a concentration of about 2 mg/mL to about 10 mg/mL (for the purposes of removing the previously described reducing agent). To the solution is added an appropriate number of equivalents (10 to about 40) of an appropriate oxidizing agent, for example, dehydroascorbic acid. The reaction is then incubated at room temperature for about 1 hour to about 5 hours depending on the observed reactivity. The myeloid cell agonist-linker construct is added as a solution with stirring. Dependent on the physical properties of the compound-linker construct, a co-solvent can be introduced prior to the addition of the compound-linker construct to facilitate solubility. The reaction is stirred at room temperature for 2 hours to about 12 hours depending on the observed reactivity. The progression of the reaction is monitored by LC-MS, hydrophobic interaction chromatography, or other appropriate means. Once the reaction is deemed complete, the remaining myeloid cell agonist-linker constructs are removed by applicable methods and the site-specific cysteine-linked conjugate is exchanged into the desired formulation buffer.

Site-specific cysteine-linked conjugates are synthesized starting with 10 mg of antibody construct (mAb) and 7 equivalents of compound-linker using the conditions described in the Scheme below. Monomer content and drug-antibody ratios can be determined by methods described herein and known in the art.

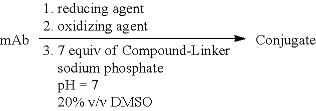

### Example 13

### Determination of K_{d} Values

K_{d} is measured using surface plasmon resonance assays using a BIACORE®-2000 or a BIACORE®-3000 (BIAcore, Inc., Piscataway, N.J.) at 25° C. with immobilized antigen CM5 chips at ^{˜}10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/mL (^{˜}0.2 μM) before injection at a flow rate of 5 μL/minute to achieve approximately 10 response units (RU) of coupled protein. Following the injection of antigen, 1 M ethanolamine is injected to block unreacted groups. For kinetics measurements, two-fold serial dilutions of Fab or conjugate (0.78 nM to 500 nM) are injected in PBS with 0.05% polysorbate 20 (TWEEN-20™) surfactant (PBST) at 25° C. at a flow rate of approximately 25 μL/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, e.g., Chen et al., J. Mol. Biol. 293:865-881 (1999). If the on-rate exceeds 10⁶ M−1 s−1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form or conjugate form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### Example 14

### Determination of Molar Ratio

This example illustrates one method by which the molar ratio is determined. One microgram of conjugate is injected into an LC/MS such as an Agilent 6550 iFunnel Q-TOF equipped with an Agilent Dual Jet Stream ESI source coupled with Agilent 1290 Infinity UHPLC system. Raw data is obtained and is deconvoluted with software such as Agilent MassHunter Qualitative Analysis Software with BioConfirm using the Maximum Entropy deconvolution algorithm. The average mass of intact conjugates is calculated by the software, which can use top peak height at 25% for the calculation. This data is then imported into another program to calculate the molar ratio of the myeloid cell agonist:conjugate, such as Agilent molar ratio calculator.

### Example 15

### Additional Method for Determination of Molar Ratio

Another method for determination of molar ratio is as follows. First, 10 μL of a 5 mg/mL solution of a conjugate is injected into an HPLC system set-up with a TOSOH TSKgel Butyl-NPR™ hydrophobic interaction chromatography (HIC) column (2.5 μM particle size, 4.6 mm×35 mm) attached. Then, over the course of 18 minutes, a method is run in which the mobile phase gradient is run from 100% mobile phase A to 100% mobile phase B over the course of 12 minutes, followed by a six minute re-equilibration at 100% mobile phase A. The flow rate is 0.8 mL/min and the detector is set at 280 nM. Mobile phase A is 1.5 M ammonium sulfate, 25 mM sodium phosphate (pH 7). Mobile phase B is 25% isopropanol in 25 mM sodium phosphate (pH 7). Post-run, the chromatogram is integrated and the molar ratio is determined by summing the weighted peak area.

### Example 16

### Production of Exemplary ASGR1 Antibodies

ASGR1 4A2 Heavy and Light chain DNA was cloned into separate expression vectors for human IgG1 antibody production. The heavy chain vector construct and the light chain vector construct were transiently co-expressed in the ExpiCHO™ system to generate 4A2 IgG1 antibody. The protein from the harvested ExpiCHO™ supernatant was purified to homogeneity using protein A column on GE AKTA Pure™ system and confirmed for purity using analytical HPLC-SEC. If needed, additional purification was performed on a preparative SEC column to remove aggregates. ASGR1 72G9 and 176H4 IgG1 antibodies were created in the same manner. The sequences for the heavy chain and lights chains for the 4A2, 72G9 and 176H4 antibodies are set forth in WO2017/058944, incorporated herein by reference in its entirety and for all purposes.

### Example 17

### Production of Exemplary ASGR1 Antibodies With Fc Domain Mutations

4A2 Fc variants were generated in similar manner as described in Example 16. The human IgG1 null Fc domain comprised of the following mutations: L234A, L235A, G237A, and K322A based on EU numbering. The IgG1 SELF domain comprised of the following mutations: S267E and L328F based on EU numbering. The LF variant contained the L328F mutation. Both the IgG1 SELF and LF constructs also contained the K322A mutation to disable CDC function without affecting FcgR binding. The following constructs were generated:

a. 4A2 IgG1 SELF K322A

b. 4A2 IgG1 LF K322A

c. 4A2 IgG1 null

d. 4A2 IgG1 null SELF

e. 4A2 IgG1 null L328F

### Example 18

### Production of Exemplary Cysteine Engineered ASGR1 Antibodies

Two cysteine engineered variants (4A2 IgG1 A114C-HC and 4A2 IgG1 V205C-LC) were also generated and the protein produced in a similar manner as described above in Example 15. 4A2 IgG1 A114C-HC contained the Alanine to Cysteine mutation on the heavy chain at position 114 based on EU numbering. 4A2 V205C-LC contained the Valine to Cysteine mutation on the light chain at position 205. Binding data confirmed that the mutation on the Fc variants and the cysteine engineered variants did not impact binding to ASGR1

### Example 19

### Characterization of Cysteine Engineered ASGR1 4A2 Exemplary Antibodies Using ELISA FcgR Binding Assay

ELISA plates were coated with 50 ul of various recombinant Fcg receptors (human and Marmota) at 0.1 ug/ml diluted in 1×PBS. Following overnight incubation, the plates were washed with the wash buffer (1×PBS+0.05% Tween 20), blocked with the blocking buffer (1% nonfat milk in PBS) and incubated for 1 hour at room temperature. The ELISA plates were then washed with the wash buffer and ASGR1 test samples (naked mAb or cysteine engineered Ab) were added from a concentration of 100 ug/ml diluted 3 fold. The plates were then incubated for 1 hour at room temperature and washed again 3× with wash buffer. 100 ul of the HRP goat anti-human Fc antibody diluted 1:1000 fold was next added into each well and incubated for another hour. The plates were washed 4× with the wash buffer. 50 ul of the TMB ELISA substrate was next added for color development and the reaction stop by adding 50 ul of 2M sulfuric acid. The plates were then read on a plate reader at 450 nm wavelength.

When 4A2 IgG1 and the cysteine engineered variants (see Example 18) were compared across different Human and Marmota Fcg receptors (Human FcgR2a, Human FcgR2b, Marmota FcgR1, Marmota FcgR2b, and Marmota FcgR3a), the binding affinity were all similar indicating that the cysteine mutation introduced for the cysteine engineered variants did not affect FcgR binding (data not shown).

### Example 20

### Characterization of ASGR1 4A2 Conjugates Binding to Human FcgR1 Receptors

4A2 IgG1 and conjugates were analyzed for human FcγR1 interaction analysis using an Octet Red 96™ instrument. The conjugates were 2.14 conjugates (2.14 conjugates refer to the 2.14 compound linker conjugated to antibody via the interchain disulfide bonds with an average DAR between 3 and 5. If A114C or V205C is noted, the 2.14 conjugate is a 2.14 compound linker conjugated to antibody via the introduced cysteine with an average DAR of about 2. Unless otherwise noted, the conjugated antibody is the 4A2 antibody.) The antibody or conjugates were immobilized on anti-human Fc biosensors and incubated with varying concentration of monomeric FcγR1 ranging from 1.2 nM to 1 μM in PBS. The experiments were performed using five steps: (1) baseline acquisition (60 s); (2) antibody or conjugate loading onto anti-human Fc biosensor (120 s); (3) second baseline acquisition (60 s); (4) association of interacting protein for kₒₙ measurement (120 s); and (5) dissociation of interacting FcγR1 for k_{off} measurement (300 s). The interacting monomeric FcγR1 was used at 5-6 concentrations of a 3-fold concentration series. The data were analyzed using Octet Data Analysis Software 9.0 (ForteBio)™ and fit to the 1:1 binding model. Equilibrium dissociation constants (K_{D}) were calculated by the ratio of kₒₙ to k_{off}. As can be seen from the table below, the conjugates containing SELF, LF mutations had similar KD as the conjugates with wild type Fc and the naked antibody. Only conjugates containing the null Fc mutations had very weak binding to human FcγR1. Data is shown in Table 4 below:

TABLE 4

| Antibody/Conjugates | KD (in nM) |
|---|---|
| 4A2IgG1 | 0.28 |
| 4A2 IgG1 2.14 Conjugate | 0.29 |
| 4A2 IgG1 SELF- 2.14 Conjugate | 0.19 |
| 4A2 IgG1 LF - 2.14 Conjugate | 0.34 |
| 4A2 IgG1null SELF- 2.14 Conjugate | Very weak binding |
| 4A2 IgG1null LF - 2.14 Conjugate | Very weak binding |

### Example 21

### Characterization of ASGR1 4A2 Conjugates Binding to FcgRs Before and After Conjugation

ELISA plates were coated with 50 ul of various recombinant Fcg receptors (human and Marmota) at 0.1 ug/ml diluted in 1×PBS. Following overnight incubation, the plates were washed with the wash buffer (1×PBS+0.05% Tween 20), blocked with the blocking buffer (1% nonfat milk in PBS) and incubated for 1 hour at room temperature. The ELISA plates were then washed with the wash buffer and ASGR1 test samples (naked mAb or conjugate) were added from a concentration of 100 ug/ml diluted 3-fold. The plates then incubated for 1 hour at room temperature and washed again 3× with wash buffer. 100 ul of the HRP goat anti-human Fc antibody diluted 1:1000 fold was next added into each well and incubated for another hour. The plates were washed 4× with the wash buffer. 50 ul of the TMB ELISA substrate was next added for color development and the reaction stop by adding 50 ul of 2M sulfuric acid. The plates were then read on a plate reader at 450 nm wavelength. Binding curves for each protein was compared before and after conjugation. Table 5 compared the binding of naked 4A2 Fc variants and 2.14 conjugates of these Fc variants to the 3 Marmota FcgRs (R1, R2b and R3a). The data showed that the bindings were similar implying that the conjugation of TLR8 small molecule agonist to the antibody did not modify the binding of 4A2 IgG1 or its Fc variants to Marmota Fcgamma receptors. Similar observation was also made with the binding to the different human FcgRs (R1, R21, R2b and R3) as shown in Table 6. Conjugating the TLR8 agonist (compound 1.50) to 4A2 or its Fc variants did not significantly impact binding to the human FcgRs. In general, the conjugate Fc variants showed similar binding to human and marmota FcgRI and FcgR3 based on the ELISA assay. However, the SELF and LF mutation in the Fc null background only restored binding to human FcgR2b but not Marmota FcgR2b.

TABLE 5

| | marmota | | marmota | | marmota | |
|---|---|---|---|---|---|---|
| | FcgR1 | | FcgR2b | | FcgR3a | |
| | | After | | After | | After |
| 4A2 Fc | Before | (con- | Before | (con- | Before | (con- |
| Variants | (mAbs) | jugate) | (mAbs) | jugate) | (mAbs) | jugate) |
| ASGR1-4A2 | + | + | + | + | + | + |
| IgG1 | | | | | | |
| ASGR1 4A2 | + | + | ++ | ++ | − | − |
| IgG1 SELF | | | | | | |
| K322A | | | | | | |
| ASGR1 4A2 | − | − | − | − | − | − |
| IgG1 null SELF | | | | | | |
| ASGR1 4A2 | + | + | ++ | ++ | − | − |
| IgG1 LF | | | | | | |
| K322A | | | | | | |
| ASGR1 4A2 | − | − | − | − | − | − |
| IgG1 null LF | | | | | | |

TABLE 6

| | Human | | Human | | Human | | Human | |
|---|---|---|---|---|---|---|---|---|
| | FcgR1 | | FcgR2a | | FcgR2b | | FcgR3 | |
| | | After | | After | | After | | After |
| 4A2 Fc | Before | (con- | Before | (con- | Before | (con- | Before | (con- |
| Variants | (mAbs) | jugate) | (mAbs) | jugate) | (mAbs) | jugate) | (mAbs) | jugate) |
| ASGR1-4A2 IgG1 | + | + | + | + | + | + | + | + |
| ASGR1 4A2 | + | + | ++ | ++ | ++ | ++ | − | − |
| IgG1 SELF | | | | | | | | |
| K322A | | | | | | | | |
| ASGR1 4A2 | residual | residual | ++ | ++ | ++ | ++ | − | − |
| IgG1 null SELF | | | | | | | | |
| ASGR1 4A2 | + | + | ++ | ++ | ++ | ++ | residual | − |
| IgG1 LF K322A | | | | | | | | |
| ASGR1 4A2 | − | − | ++ | ++ | + | + | − | − |
| IgG1 null LF | | | | | | | | |

### Example 22

### Conjugate Activity Assay in PBMC-Hepatocyte Co-Culture Assay

The PBMC-hepatocyte co-culture assay was performed as follows. PBMCs were isolated from human blood by density gradient centrifugation, resuspended in complete RPMI, and plated in 96-well flat bottom microtiter plates (125,000/well). ASGR1 positive HepG2 cells or ASGR1 negative SKBR3 cells were then added (25,000/well) along with titrating concentrations of ASGR1-TLR8 agonist conjugate. The ASGR1-TLR8 agonist conjugate was a 2.14 Conjugate. After overnight culture, supernatants were harvested, and TNFα levels were determined by AlphaLISA.

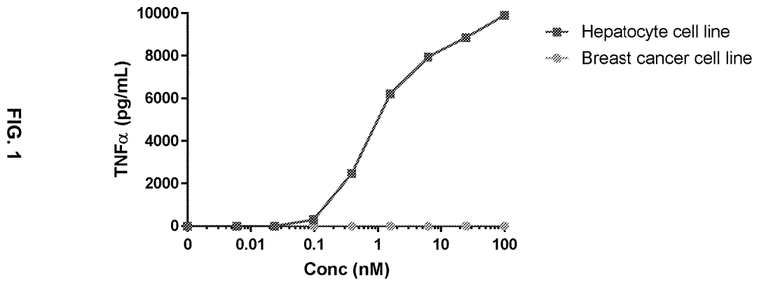

Referring to FIG. 1, the ASGR1-TLR8 agonist conjugate was active on the ASGR1 positive hepatocyte cell line, but not on the ASGR1 negative breast cancer cell line, as measured by TNFα production.

### Example 23

### TNFα Production by PBMCs was Induced by Immune Stimulatory Conjugates

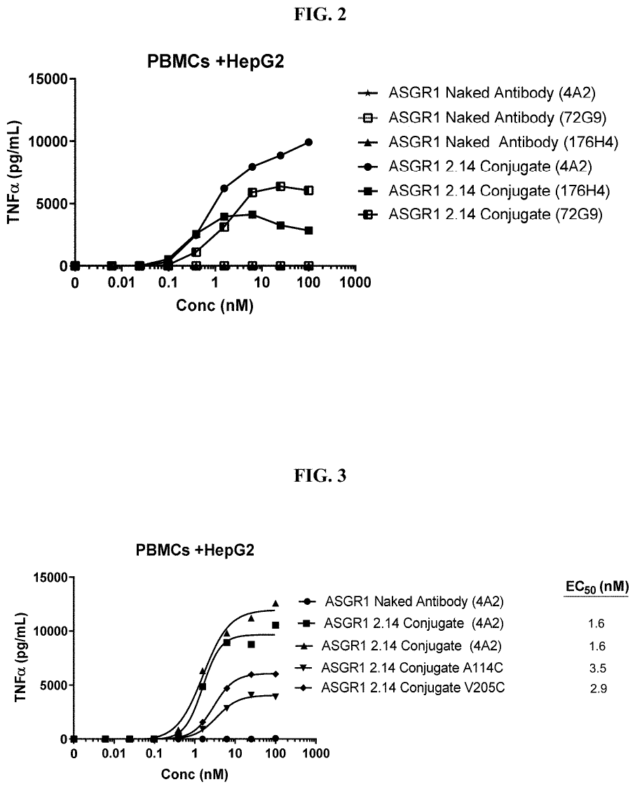

PBMCs were isolated from human blood by Ficoll gradient centrifugation, resuspended in cRPMI, and plated in 96-well flat bottom microtiter plates (125,000/well). HepG2 (ASGR1+) cells were then added (25,000/well) along with titrating concentrations of ASGR1-TLR8 conjugates or unconjugated parental antibodies as controls. After overnight culture, supernatants were harvested, and TNFα levels were determined by AlphaLISA. FIG. 2 illustrates that the ASGR1-TLR8 2.14 conjugate activates human PBMC in the presence of an ASGR1-expressing hepatocyte cell line.

### Example 24

### TNFα Production by PBMCs was Induced by Cysteine Engineered Conjugates

PBMCs were isolated from human blood by Ficoll gradient centrifugation, resuspended in cRPMI, and plated in 96-well flat bottom microtiter plates (125,000/well). HepG2 (ASGR1+) cells were then added (25,000/well) along with titrating concentrations of 2.14 conjugates, 2.14 cysteine engineered conjugates (2.14 cysteine engineered conjugates have an average DAR of about 2 and are conjugated via the introduced cysteine residue) or unconjugated parental antibodies as controls. After overnight culture, supernatants were harvested, and TNFα levels were determined by AlphaLISA. As shown by FIG. 3, ASGR1 cysteine engineered conjugates activate human PBMC in the presence of an ASGR1-expressing hepatocyte cell line.

### Example 25

### TNFα Production by PBMCs was Induced by TLR8 Conjugates Having Varied Linkers

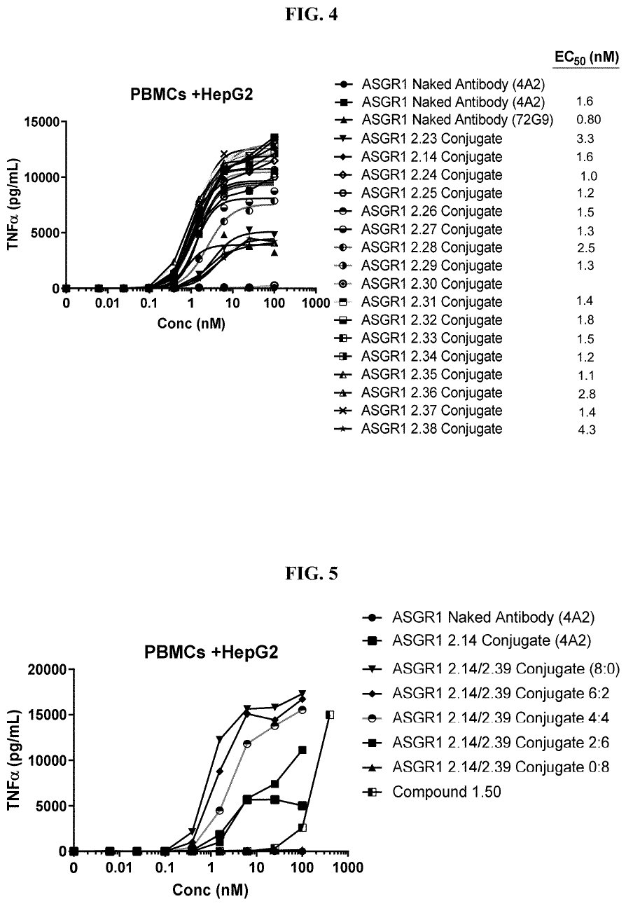

PBMCs were isolated from human blood by Ficoll gradient centrifugation, resuspended in cRPMI, and plated in 96-well flat bottom microtiter plates (125,000/well). HepG2 (ASGR1+) cells were then added (25,000/well) along with titrating concentrations of ASGR1-TLR8 conjugates with differing linkers or unconjugated parental antibodies as controls. After overnight culture, supernatants were harvested, and TNFα levels were determined by AlphaLISA. The conjugate numbers in the legend refer to the compound-linkers in Table 3. The results demonstrate that ASGR1-TLR8 conjugates display potent activity with a variety of linker variants including cleavable and non-cleavable linkers. Both the magnitude and potency as measured by EC50 can be altered by linker choice. See FIG. 4.

### Example 26

### TNFα Production by PBMCs was Induced by Mixed TLR8-TLR7 Conjugates

PBMCs were isolated from human blood by Ficoll gradient centrifugation, resuspended in cRPMI, and plated in 96-well flat bottom microtiter plates (125,000/well). HepG2 (ASGR1+) cells were then added (25,000/well) along with titrating concentrations of ASGR1 conjugates with various ratios TLR8:TLR7 agonist payload. After overnight culture, supernatants were harvested, and TNFα levels were determined by AlphaLISA. The conjugates were 2.14 TLR8 conjugates and TLR7 2.39 conjugates (i.e., the 2.39 compound linker conjugated to the ASGR1 4A2 antibody via the interchain disulfides having an average DAR of 4). FIG. 5 shows that mixed TLR8-TLR7 ASGR1 conjugates induce TNF-a production from human PBMC in hepatocyte co-culture.

The TLR7 compound-linker used in this example and example 27 is shown below:

| Compound | |
|---|---|
| Linkers | Structure |
| 2.39 | 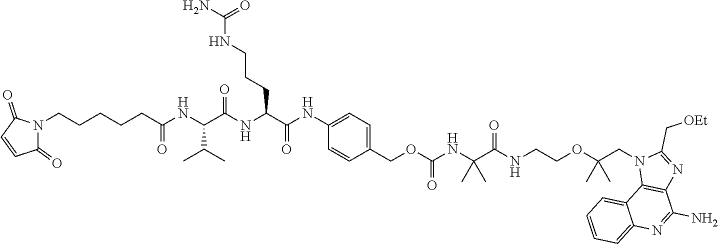 [SMILES: CCOCc1nc2c(N)nc3ccccc3c2n1CC(C)(C)OCCNC(=O)C(C)(C)NC(=O)OCc1ccc(NC(=O)[C@H](CCCNC(N)=O)NC(=O)[C@@H](NC(=O)CCCCCN2C(=O)C=CC2=O)C(C)C)cc1] |
| | 4-((S)-2-((S)-2-(6-(2,5-dioxo-2,5-dihydro-1H-pyrrol-1-yl)hexanamido)-3-methylbutanamido)-5- |
| | ureidopentanamido)benzyl(1-((2-((1-(4-amino-2-(ethoxymethyl)-1H-imidazo[4,5-c]quinolin-1-yl)-2- |
| | methylpropan-2-yl)oxy)ethyl)amino)-2-methyl-1-oxopropan-2yl)carbamate |

### Example 27

### Cytokine Production by PBMCs was Induced by Mixed TLR8-TLR7 Conjugates

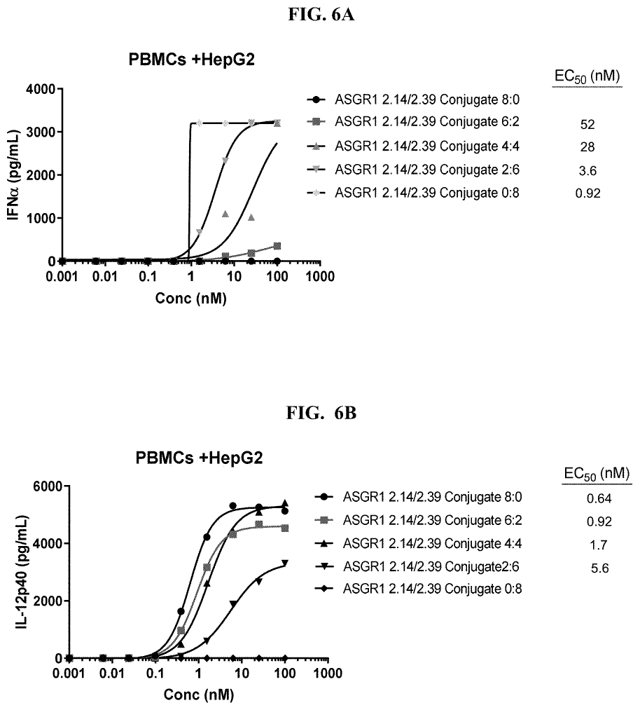

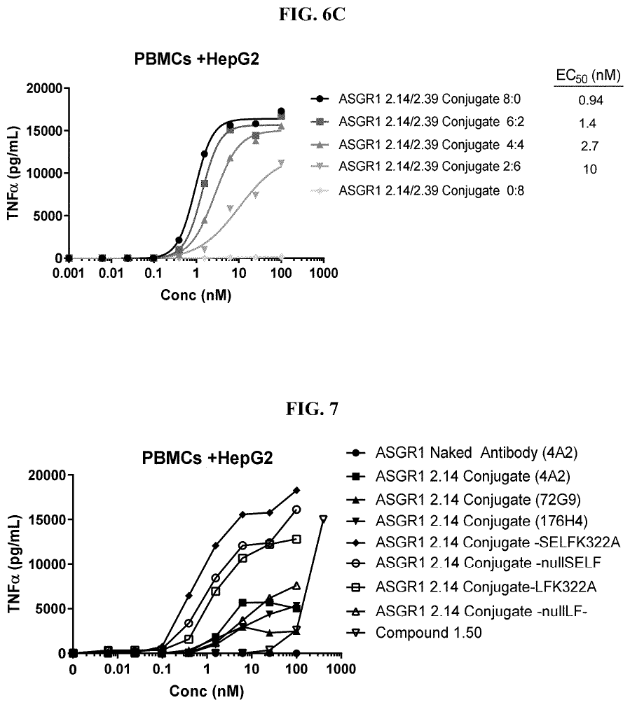

Dual payload TLR8-TLR7 ASGR1 conjugates induce cytokine production from human PBMC in hepatocyte co-culture. PBMCs were isolated from human blood by Ficoll gradient centrifugation, resuspended in cRPMI, and plated in 96-well flat bottom microtiter plates (125,000/well). HepG2 (ASGR1+) cells were then added (25,000/well) along with titrating concentrations of ASGR1 conjugates with various ratios TLR8:TLR7 agonist payload. After overnight culture, supernatants were harvested, and TNFα, IL012p40, and IFNα levels were determined by AlphaLISA. The results demonstrate that each TLR ligand was active in the mixed conjugates with IFNa production by TLR7 activation and TNFa and IL12 p40 production by TLR8 activation. See FIGS. 6A-C.

### Example 28

### TNFα Production by PBMCs Induced by ASGR1 Conjugates is Fc-Dependent

ASGR1 conjugate activation of human PBMC is Fc-dependent. PBMCs were isolated from human blood by Ficoll gradient centrifugation, resuspended in cRPMI, and plated in 96-well flat bottom microtiter plates (125,000/well). HepG2 (ASGR1+) cells were then added (25,000/well) along with titrating concentrations of 2.14 conjugates with differing Fc regions or with unconjugated parental antibodies as controls. After overnight culture, supernatants were harvested, and TNFα levels were determined by AlphaLISA. The results demonstrate that for ASGR1-TLR8 conjugates, increased avidity for FcgR2 can further increase activity and increased FcgR2 avidity can support activation in the absence of FcgR1 and FcgR3 binding. See FIG. 7.

### Example 29

### TLR7 and TLR8 Small Molecules Induce Marmota TNFα Expression

Total RNA was extracted from stimulated monax PBMC using the RNeasy®Mini kit (Qiagen, Maryland, MD) according to the manufacturer's protocol. Briefly, cells were pelleted and resuspended in 600 ul RLT buffer. The sample was vortexed and then run through a QIAshredder column. RNA was extracted from the supernatant and eluted with a 30 uL volume. RNA purity was assessed (A260/A280) using a Nanodrop one spectrophotometer (Thermofisher Scientific, Waltham, Mass., USA). For removal of genomic DNA, 800 ng RNA was incubated with 2 units DNase I (RNase-free) (New England Biolabs, Ipswich, Mass., USA) in DNase reaction buffer at 37° C. for 10 min. The reaction was terminated incubation at 75° C. for 10 min. cDNA was generated using the Superscript III First Strand synthesis System for RT-PCR (Invitrogen, Carlsbad, Calif., USA), according to the manufacturer's protocol.

The generation of oligonucleotide dimers for each TaqMan primer pair was assessed using Power SYBR®Green PCR Master-Mix (Foster City, Calif., Applied Biosystems) with melting curve analysis, according to the manufacturer's instructions. Primers which resulted in oligonucleotide dimer generation were designed and tested for monax TNFa. The forward and reverse primers used were: 5′CCTGCAAACGGGCTATACCTT3′ (SEQ ID NO: 7) and 5′GTGTGGGTGAGGAGCACGTA3′ (SEQ ID NO: 8) respectively. The probe used was 5′6FAM-CAGCCTTGGCCCTTGAAGAGGACCT-TAM3′ (SEQ ID NO: 9). All real time PCR assays were performed using the TaqMan®Fast Universal PCR MasterMix (2×), No AmpErase UNG (Applied Biosystems), according to the manufacturer's protocol. One uL cDNA sample was assayed per reaction. Each reaction consisted of 1 cycle of 95° C. for 20 s, followed by 50 cycles of 95° C. for 3 s and 60° C. for 30 s. Realtime PCR runs for monax TNFα gene included TNFα cDNA standards, no template control and test samples. All real time PCR reactions were run on an Step One Plus Fast Real-Time PCR system using the Step One Software, Version 2.3 (Applied Biosystems). Data were analyzed using the Step One Software to generate Ct value for each sample.

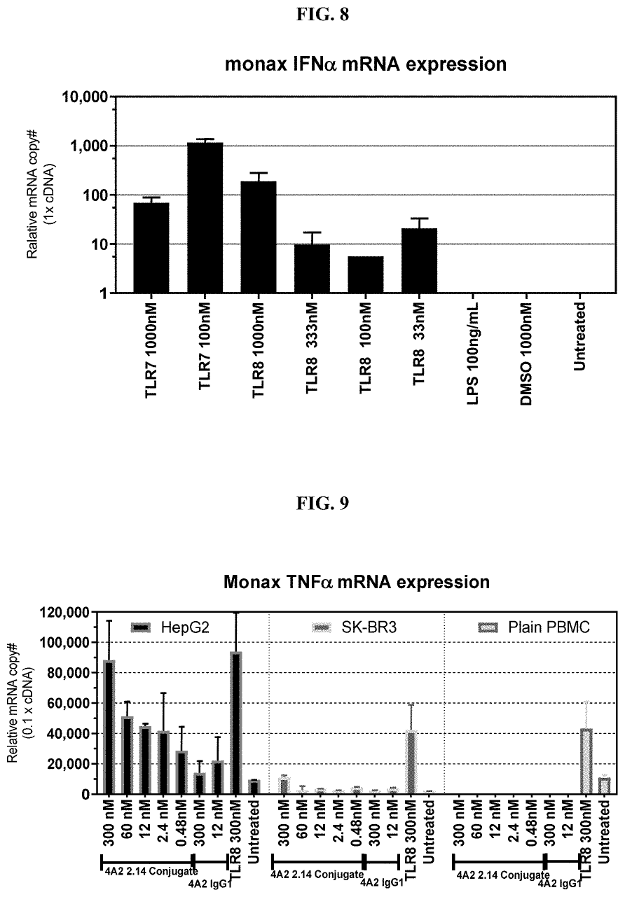

A plot of Ct versus log copy number standard curve was generated using synthesized monax TNFα cDNA standard. The Ct value for each data point was then determined and normalized to the standard curve to obtain the copy number. A plot of copy number per ng of mRNA for different TLR7 and 8 compounds were generated (see FIG. 8). This figure shows potent activation of monax TNFα gene by TLR7 and TLR8. The TLR8 small molecule is compound 1.50 and the TLR7 small molecule is

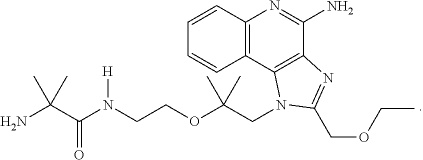 [SMILES: CCOCc1nc2c(N)nc3ccccc3c2n1CC(C)(C)OCCNC(=O)C(C)(C)N]

### Example 30

### ASGR1 TLR8 Conjugates Conditionally Activate Marmota PBMCs in the Presence of Target Cells

Woodchuck Peripheral Blood Mononuclear cells (wPBMCs) purchased from iQ Biosciences (cat #IQB-WPB102) were thawed and plated out at 5:1 ratio with HepG2 cells, SK-BR-3 cells, or by themselves in a 24-well plate and combined with serial dilutions of a 2.14 conjugate, naked antibody, or compound 1.50 at 300 nM in 10% FBS RPMI containing media and incubated together for 24 hours. After incubation the wPBMCs were lysed and total RNA purified using Qiagen RNeasy kit.

Total RNA was extracted from stimulated monax PBMC using the RNeasy®Mini kit (Qiagen, Maryland, MD) according to the manufacturer's protocol. Briefly, cells were pelleted and resuspended in 600 ul RLT buffer. The sample was vortexed and then run through a QIAshredder column. RNA was extracted from the supernatant and eluted with a 30 uL volume. RNA purity was assessed (A260/A280) using a Nanodrop one spectrophotometer (Thermofisher Scientific, Waltham, Mass., USA). For removal of genomic DNA, 800 ng RNA was incubated with 2 units DNase I (RNase-free) (New England Biolabs, Ipswich, Mass., USA) in DNase reaction buffer at 37° C. for 10 min. The reaction was terminated incubation at 75° C. for 10 min. cDNA was generated using the Superscript III First Strand synthesis System for RT-PCR (Invitrogen, Carlsbad, Calif., USA), according to the manufacturer's protocol.

The generation of oligonucleotide dimers for each TaqMan primer pair was assessed using Power SYBR®Green PCR Master-Mix (Foster City, Calif., Applied Biosystems) with melting curve analysis, according to the manufacturer's instructions. Primers which resulted in oligonucleotide dimer generation were designed and tested for monax TNFa. The forward and reverse primers used were: 5′CCTGCAAACGGGCTATACCTT3′ (SEQ ID NO: 7) and 5′GTGTGGGTGAGGAGCACGTA3′ (SEQ ID NO: 8) respectively. The probe used was 5′6FAM-CAGCCTTGGCCCTTGAAGAGGACCT-TAM3′ (SEQ ID NO: 9). All real time PCR assays were performed using the TaqMan®Fast Universal PCR MasterMix (2×), No AmpErase UNG (Applied Biosystems), according to the manufacturer's protocol. One uL cDNA sample was assayed per reaction. Each reaction consisted of 1 cycle of 95° C. for 20 s, followed by 50 cycles of 95° C. for 3 s and 60° C. for 30 s. Realtime PCR runs for monax TNFα gene included TNFα cDNA standards, no template control and test samples. All real time PCR reactions were run on an Step One Plus Fast Real-Time PCR system using the Step One Software, Version 2.3 (Applied Biosystems). Data were analyzed using the Step One Software to generate Ct value for each sample.

The Ct values for each of the conjugate treated samples were generated and the copy number determined from the standard curve. The copy number relative to the control sample was plotted against the different cell treatment. Monax TNFα gene activation is only observed when ASRG1+ target cell (HepG2) and PBMCs are present. SKBR3 (ASGR1 negative cell line) plus Monax PBMC or Monax PBMC alone did not activate TNFα gene. See FIG. 9.

### Example 31

### ASGR1 Conjugate

This example describes use of an ASGR1 conjugate to treat Hepatitis B. A conjugate comprising an antibody construct comprising a targeting binding domain that specifically binds to ASGR1 and an Fc binding domain covalently attached to the targeting binding domain, a TLR7 agonist, and a linker covalently attaching the TLR7 agonist to the antibody construct is produced.

The ASGR1 conjugates are administered to a subject having a Hepatitis B viral infection. The subject can be a human or non-human animal. The ASGR1 conjugates boost the immune response against the Hepatitis B virus.

### Example 32

### ASGR1 Conjugate

This example describes use of dual payload TLR8-TLR7 ASGR1 conjugate to treat Hepatitis B. A conjugate comprising an antibody construct comprising a targeting binding domain that specifically binds to ASGR1 and an Fc binding domain covalently attached to the targeting binding domain, a TLR7 agonist, and a linker covalently attaching the TLR7 agonist to the antibody construct and a TLR8 agonist, and a linker covalently attaching the TLR8 agonist to the the antibody construct is produced.

The ASGR1 conjugates are administered to a subject having a Hepatitis B viral infection. The subject can be a human or non-human animal. The ASGR1 conjugates boost the immune response against the Hepatitis B virus.

### Example 33

### ASGR2 Conjugate

This example describes use of an ASGR2 conjugate to treat Hepatitis B in a subject. A conjugate comprising an antibody construct comprising a targeting binding domain that specifically binds to ASGR2 and an Fc binding domain covalently attached to the targeting binding domain, a TLR7 agonist, and a linker covalently attaching the TLR7 agonist to the antibody construct is produced.

The ASGR2 conjugates are administered to a subject having a Hepatitis B viral infection. The subject can be a human or non-human animal. The ASGR2 conjugates boost the immune response against the Hepatitis B virus.

### Example 34

### TRF2 Conjugate

This example describes use of a TRF2 conjugate to treat Hepatitis B in a subject. A conjugate comprising an antibody construct comprising a targeting binding domain that specifically binds to TRF2 and an Fc binding domain covalently attached to the targeting binding domain, a TLR7 agonist, and a linker covalently attaching the TLR7 agonist to the antibody construct is produced.

The TRF2 conjugates are administered to a subject having a Hepatitis B viral infection. The subject can be a human or non-human animal. The TRF2 conjugates boost the immune response against the Hepatitis B virus.

### Example 35

### ASGR1 Conjugate

This example describes use of an ASGR1 conjugate to treat Hepatitis B in a subject. A conjugate comprising an antibody construct comprising a targeting binding domain that specifically binds to ASGR1 and an Fc binding domain covalently attached to the targeting binding domain, a TLR8 agonist, and a linker covalently attaching the TLR8 agonist to the antibody construct is produced.

The ASGR1 conjugates are administered to a subject having a Hepatitis B viral infection. The subject can be a human or non-human animal. The ASGR1 conjugates boost the immune response against the Hepatitis B virus.

### Example 36

### ASGR2 Conjugate

This example describes use of an ASGR2 conjugate to treat Hepatitis B in a subject. A conjugate comprising an antibody construct comprising a targeting binding domain that specifically binds to ASGR2 and an Fc binding domain covalently attached to the targeting binding domain, a TLR8 agonist, and a linker covalently attaching the TLR8 agonist to the antibody construct is produced.

The ASGR2 conjugates are administered to a subject having a Hepatitis B viral infection. The subject can be a human or non-human animal. The ASGR2 conjugates boost the immune response against the Hepatitis B virus.

### Example 37

### TRF2 Conjugate

This example describes use of a TRF2 conjugate to treat Hepatitis B in a subject. A conjugate comprising an antibody construct comprising a targeting binding domain that specifically binds to TRF2 and an Fc binding domain covalently attached to the targeting binding domain, a TLR8 agonist, and a linker covalently attaching the TLR8 agonist to the antibody construct is produced.

The TRF2 conjugates are administered to a subject having a Hepatitis B viral infection. The subject can be a human or non-human animal. The TRF2 conjugates boost the immune response against the Hepatitis B virus.

### Example 38

### ASGR1 Conjugate

This example describes use of an ASGR1 conjugate to treat Hepatitis C in a subject. A conjugate comprising an antibody construct comprising a targeting binding domain that specifically binds to ASGR1 and an Fc binding domain covalently attached to the targeting binding domain, a TLR7 agonist, and a linker covalently attaching the TLR7 agonist to the antibody construct is produced.

The ASGR1 conjugates are administered to a subject having a Hepatitis C viral infection. The subject can be a human or non-human animal. The ASGR1 conjugates boost the immune response against the Hepatitis C virus.

### Example 39

### ASGR2 Conjugate

This example describes use of an ASGR2 conjugate to treat Hepatitis C in a subject. A conjugate comprising an antibody construct comprising a targeting binding domain that specifically binds to ASGR2 and an Fc binding domain covalently attached to the targeting binding domain, a TLR7 agonist, and a linker covalently attaching the TLR7 agonist to the antibody construct is produced.

The ASGR2 conjugates are administered to a subject having a Hepatitis C viral infection. The subject can be a human or non-human animal. The ASGR2 conjugates boost the immune response against the Hepatitis C virus.

### Example 40

### TRF2 Conjugate

This example describes use of a TRF2 conjugate to treat Hepatitis C in a subject. A conjugate comprising an antibody construct comprising a targeting binding domain that specifically binds to TRF2 and an Fc binding domain covalently attached to the targeting binding domain, a TLR7 agonist, and a linker covalently attaching the TLR7 agonist to the antibody construct is produced.

The TRF2 conjugates are administered to a subject having a Hepatitis C viral infection. The subject can be a human or non-human animal. The TRF2 conjugates boost the immune response against the Hepatitis C virus.

### Example 41

### ASGR1 Conjugate

This example describes use of an ASGR1 conjugate to treat Hepatitis C in a subject. A conjugate comprising an antibody construct comprising a targeting binding domain that specifically binds to ASGR1 and an Fc binding domain covalently attached to the targeting binding domain, a TLR8 agonist, and a linker covalently attaching the TLR8 agonist to the antibody construct is produced.

The ASGR1 conjugates are administered to a subject having a Hepatitis C viral infection. The subject can be a human or non-human animal. The ASGR1 conjugates boost the immune response against the Hepatitis C virus.

### Example 42

### ASGR2 Conjugate

This example describes use of an ASGR2 conjugate to treat Hepatitis C in a subject. A conjugate comprising an antibody construct comprising a targeting binding domain that specifically binds to ASGR2 and an Fc binding domain covalently attached to the targeting binding domain, a TLR8 agonist, and a linker covalently attaching the TLR8 agonist to the antibody construct is produced.

The ASGR2 conjugates are administered to a subject having a Hepatitis C viral infection. The subject can be a human or non-human animal. The ASGR2 conjugates boost the immune response against the Hepatitis C virus.

### Example 43

### TRF2 Conjugate

This example describes use of a TRF2 conjugate to treat Hepatitis C in a subject. A conjugate comprising an antibody construct comprising a targeting binding domain that specifically binds to TRF2 and an Fc binding domain covalently attached to the targeting binding domain, a TLR8 agonist, and a linker covalently attaching the TLR8 agonist to the antibody construct is produced.

The TRF2 conjugates are administered to a subject having a Hepatitis C viral infection. The subject can be a human or non-human animal. The TRF2 conjugates boost the immune response against the Hepatitis C virus.

### Example 44

### Hepatitis B Viral Antigen Conjugate

This example describes use of a Hepatitis B viral antigen conjugate to treat Hepatitis B in a subject. A conjugate comprising an antibody construct comprising a targeting binding domain that specifically binds to a Hepatitis B surface viral antigen and an Fc binding domain covalently attached to the targeting binding domain, a TLR7 agonist, and a linker covalently attaching the TLR7 agonist to the antibody construct is produced.

The Hepatitis B viral antigen conjugates are administered to a subject having a Hepatitis B viral infection. The subject can be a human or non-human animal. The Hepatitis B viral antigen conjugates boost the immune response against the Hepatitis B virus.

### Example 45

### Hepatitis B Viral Antigen Conjugate

This example describes use of a Hepatitis B viral antigen conjugate to treat Hepatitis B in a subject. A conjugate comprising an antibody construct comprising a targeting binding domain that specifically binds to a Hepatitis B surface viral antigen and an Fc binding domain covalently attached to the targeting binding domain, a TLR8 agonist, and a linker covalently attaching the TLR8 agonist to the antibody construct is produced.

The Hepatitis B viral antigen conjugates are administered to a subject having a Hepatitis B viral infection. The subject can be a human or non-human animal. The Hepatitis B viral antigen conjugates boost the immune response against the Hepatitis B virus.

### Example 46

### Hepatitis C Viral Antigen Conjugate

This example describes use of a Hepatitis C viral antigen conjugate to treat Hepatitis C in a subject. A conjugate comprising an antibody construct comprising a targeting binding domain that specifically binds to a Hepatitis C viral antigen and an Fc binding domain covalently attached to the targeting binding domain, a TLR7 agonist, and a linker covalently attaching the TLR7 agonist to the antibody construct is produced.

The Hepatitis C viral antigen conjugates are administered to a subject having a Hepatitis C viral infection. The subject can be a human or non-human animal. The Hepatitis C viral antigen conjugates boost the immune response against the Hepatitis C virus.

### Example 47

### Hepatitis C Viral Antigen Conjugate

This example describes use of a Hepatitis C viral antigen conjugate to treat Hepatitis C in a subject. A conjugate comprising an antibody construct comprising a targeting binding domain that specifically binds to a Hepatitis C viral antigen and an Fc binding domain covalently attached to the targeting binding domain, a TLR8 agonist, and a linker covalently attaching the TLR8 agonist to the antibody construct is produced.

The Hepatitis C viral antigen conjugates are administered to a subject having a Hepatitis C viral infection. The subject can be a human or non-human animal. The Hepatitis C viral antigen conjugates boost the immune response against the Hepatitis C virus.

### Example 48

### TNFα Expression by PBMCs is Induced by Myeloid Cell Agonist Conjugates

This example shows that myeloid cell agonist conjugates can increase production of a pro-inflammatory cytokine, TNFα, by PBMCs in the presence of cells expressing a liver cell antigen.

PBMCs are isolated from woodchuck blood by standard methods. Briefly, PBMCs are isolated by Ficoll gradient centrifugation, resuspended in RPMI, and plated in 96-well flat bottom microtiter plates (˜125,000/well). Recombinant cells (e.g., HEK) expressing a human liver antigen (e.g., ASGR2) are then added (˜25,000/well) along with titrating concentrations of conjugates or unconjugated parental antibodies as controls. The conjugates contain an antibody against the liver cell antigen conjugated to a TLR8 benzazepine agonist. After overnight culture, supernatants are harvested, and TNFα levels are determined by AlphaLISA. Expression of TNFα is increased in the presence of the conjugates.

### Example 49

### Chronic Woodchuck Hepatitis Virus Infection Model

This example describes use of an ASGR1 or ASGR2 conjugate to treat chronic Woodchuck Hepatitis virus (WHV) infections in woodchucks. Woodchucks with chronic WHV infections are treated intravenously with an anti-ASGR1 or anti-ASGR2 conjugate including an anti-ASGR1 or ASGR2 antibody conjugated TLR8 benzazepine agonists, a control conjugate, or the TLR8 benzazepine agonist alone. Each group of woodchucks has 3-5 animals. The anti-ASGR1 or anti-ASGR2 antibodies bind to the corresponding woodchuck protein. Escalating doses of the anti-ASGR1 or anti-ASGR2 conjugates, control conjugates, or TLR8 benzazepine agonists are administered and the level of WHV DNA or WHV surface antigen is monitored. The anti-ASGR1 or ASGR2 conjugates boost the immune response against WHV.

### Example 50

### Chronic Woodchuck Hepatitis Virus Infection Model

This example describes use of an anti-woodchuck hepatitis conjugate to treat chronic Woodchuck Hepatitis virus (WHV) infections in woodchucks. Woodchucks with chronic WHV infections are treated intravenously with an anti-WHV conjugate including an anti-WHV surface antigen antibody conjugated TLR8 benzazepine agonists, a control conjugate, or the TLR8 benzazepine agonist alone. Each group of woodchucks has 3-5 animals. Escalating doses of the anti-WHV conjugates, control conjugate, or TLR8 benzazepine agonists are administered and the level of WHV DNA or WHV surface antigen is monitored. The anti-WHV conjugates boost the immune response against WHV.

While aspects of the present disclosure have been shown and described herein, it will be apparent to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

1. A conjugate, comprising:
a) an antibody construct comprising
i) a target antigen binding domain that specifically binds to a liver cell antigen, wherein the liver cell antigen is selected from the group consisting of ASGR1, ASGR2, TRF2, UGT1A1, SLC22A7, SLC13A5, SLC22A1, and C9; and
ii) an Fc binding domain covalently attached to the target antigen binding domain;
b) a myeloid cell agonist selected from a TLR7 agonist or a TLR8 agonist, wherein the TLR7 agonist comprises a compound of Category B Formulas (IA), (IB), and (IC) and wherein the TLR8 agonist comprises a compound of Category A Formulas (IA), (IB), (IIA), (IIB), (IIIA), and (IIIB); and
c) a linker covalently attached to the myeloid cell agonist and to the antibody construct.

2. The conjugate of claim 1, wherein the conjugate is represented by Formula (I): 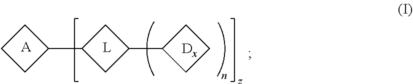
wherein:
A is the antibody construct,
L is the linker;
Dₓ is the myeloid cell agonist,
n is selected from 1 to 20; and
z is selected from 1 to 20.

3. (canceled)

4. The conjugate of claim 1, wherein the liver cell antigen is expressed on a canalicular cell, Kupffer cell, hepatocyte, or any combination thereof.

5. The conjugate of claim 1, wherein the liver cell antigen is a hepatocyte antigen.

6. (canceled)

7. The conjugate of claim 1, wherein the liver cell antigen is selected from the group consisting of ASGR1, ASGR2, and TRF2.

8. The conjugate of claim 1, wherein the liver cell antigen is ASGR1.

9.-37. (canceled)

38. The conjugate of claim 1, wherein the TLR8 agonist comprises any one of compounds 1.1-1.67 or a salt thereof.

39.-40. (canceled)

41. The conjugate of claim 1, wherein the conjugate comprises a compound-linker and wherein the compound-linker comprises any one of compound-linkers 2.1-2.39 or a salt thereof.

42. The conjugate of claim 1, wherein the Fc binding domain is an IgG Fc region.

43. The conjugate of claim 1, wherein the Fc binding domain is an IgG1 Fc region.

44. The conjugate of claim 1, wherein the Fc binding domain is an Fc binding domain variant comprising one or more amino acid substitutions in an IgG Fc region as compared to an amino acid sequence of a wild-type IgG Fc region.

45. The conjugate of claim 44, wherein the Fc binding domain variant has increased affinity to one or more Fcγ receptors as compared to the wild-type IgG Fc region.

46. The conjugate of claim 1, wherein the Fc binding domain is a non-antibody scaffold.

47. The conjugate of claim 1, wherein the target antigen binding domain comprises an immunoglobulin heavy chain variable region or an antigen binding fragment thereof and an immunoglobulin light chain variable region or an antigen binding fragment thereof.

48. The conjugate of claim 1, wherein the target antigen binding domain comprises a single chain variable region fragment (scFv).

49.-50. (canceled)

51. The conjugate of claim 1, wherein the Fc binding domain is covalently attached to the target antigen binding domain:
a) as an Fc binding domain target antigen binding domain fusion protein; or
b) by conjugation via a second linker.

52. The conjugate of claim 1, wherein the antibody construct has a Kd for binding of the Fc binding domain to an Fc receptor in the presence of the myeloid cell agonist and wherein the K_{d} for binding of the Fc binding domain to the Fc receptor in the presence of the myeloid cell agonist is no greater than about 100 times a K_{d} for binding of the Fc binding domain to the Fc receptor in the absence of the myeloid cell agonist.

53. The conjugate of claim 1, wherein n is 1 and z is from 1 to 8.

54. A pharmaceutical composition comprising the conjugate of claim 1 and a pharmaceutically acceptable carrier.

55. A method of treating a subject having a liver viral infection, comprising administering to the subject an effective dose of the conjugate of claim 1 or the pharmaceutical composition of claim 54.

56. The method of claim 55, wherein the subject has a Hepatitis B infection.

57. The method of claim 55, wherein the subject does not have cancer.

58. The method of claim 55, wherein the conjugate is administered systemically.

59. The method of claim 55, wherein the conjugate is administered intravenously, cutaneously, subcutaneously, or injected at a site of the viral infection.

60.-62. (canceled)

63. The conjugate of claim 1, wherein the TLR8 agonist comprises any one of compounds 1.1, 1.50, 1.62, 1.63 or a salt thereof.

64. The conjugate of claim 1, wherein the conjugate comprises a compound-linker and wherein the compound-linker comprises any one of compound-linkers 2.14, 2.15, 2.16, 2.17, 2.20 or a salt thereof.

65. The conjugate of claim 1, wherein the linker is represented by formula: 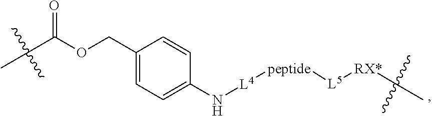
wherein L⁴ represents the C-terminal of the peptide and L⁵ is selected from a bond, alkylene and heteroalkylene,
wherein L⁵ is optionally substituted with one or more groups independently selected from R³²; RX* comprises a bond, a succinimide moiety, or a hydrolyzed succinimide moiety bound to a residue of the antibody construct,
wherein 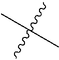
on RX* represents the point of attachment to the residue of the antibody construct; and, R³² is independently selected at each occurrence from halogen, —OH, —CN, —O-alkyl, —SH, ═O, ═S, —NH₂, —NO₂; and C₁₋₁₀ alkyl, C₂₋₁₀ alkenyl, and C₂₋₁₀ alkynyl, each of C₁₋₁₀alkyl, C₂₋₁₀ alkenyl, and C₂₋₁₀ alkynyl is optionally substituted with one or more substituents independently selected from halogen, —OH, —CN, —O-alkyl, —SH, ═O, ═S, —NH₂, —NO₂.

66. The conjugate of claim 65, wherein RX* comprises a succinamide moiety or a hydrolyzed succinimide moiety and is bound to a cysteine residue of the antibody construct.


SEQUENCE LISTING (9 sequences)

SEQ ID NO: 1 - protein, 5 aa, Unknown
  source: Recognition motif sequence
  MOD_RES (3)..(3): Any amino acid
LPXTG

SEQ ID NO: 2 - DNA, 10 bp, Artificial Sequence
  source: Synthetic oligonucleotide
gggggggggg

SEQ ID NO: 3 - protein, 25 aa, Artificial Sequence
  source: Synthetic peptide
  SITE (1)..(25): This sequence may encompass 2-5 ′Gly Gly Gly Gly Ser′ repeating units
GGGGSGGGGSGGGGSGGGGSGGGGS

SEQ ID NO: 4 - protein, 25 aa, Artificial Sequence
  source: Synthetic peptide
  SITE (1)..(25): This sequence may encompass 3-5 ′Gly Gly Gly Gly Ser′ repeating units“
GGGGSGGGGSGGGGSGGGGSGGGGS

SEQ ID NO: 5 - protein, 4 aa, Artificial Sequence
  source: Synthetic peptide
GFLG

SEQ ID NO: 6 - protein, 4 aa, Artificial Sequence
  source: Synthetic peptide
ALAL

SEQ ID NO: 7 - DNA, 21 bp, Artificial Sequence
  source: Synthetic primer
cctgcaaacgggctatacctt

SEQ ID NO: 8 - DNA, 20 bp, Artificial Sequence
  source: Synthetic primer
gtgtgggtgaggagcacgta

SEQ ID NO: 9 - DNA, 25 bp, Artificial Sequence
  source: Synthetic probe
cagccttggcccttgaagaggacct